(19) United States
(12) Patent Application Publication
(10) Pub. No.: US 2025/0152734 A1
(43) Pub. Date: May 15, 2025

(54) USES AND FORMULATIONS OF CAMPTOTHECIN (CPT)-CONTAINING NANOPARTICLES

(71) Applicant: DANTARI, INC., Thousand Oaks, CA (US)

(72) Inventors: Andrew T. Marth, Thousand Oaks, CA (US); Robert J. Lamm, Thousand Oaks, CA (US); Emily A. Wyent, Sherman Oaks, CA (US); Timothy Hagerty, Westake Village, CA (US); Richard Markus, Newbury Park, CA (US)

(21) Appl. No.: 18/838,199

(22) PCT Filed: Feb. 13, 2023
(86) PCT No.: PCT/US2023/062457

§ 371 Date: Aug. 13, 2024

Related U.S. Application Data
(60) Provisional application No. 63/309,810, filed on Feb. 14, 2022.

(51) Int. Cl. A61K 47/69; A61K 9/08; A61K 31/454; A61K 31/502; A61K 31/5025; A61K 31/55; A61K 47/54; A61P 35/00

(52) U.S. Cl. CPC A61K 47/6935; A61K 9/08; A61K 31/454; A61K 31/502; A61K 31/5025; A61K 31/55; A61K 47/542; A61P 35/00

(57) ABSTRACT

Disclosed herein are compositions, formulations, and methods that include nanoparticles comprising camptothecin-conjugated mucic acid polymers.

### CROSS REFERENCE TO RELATED APPLICATIONS

This application claims the benefit, under 35 U.S.C. § 119 (e) (1), of U.S. provisional patent application No. 63/309,810 filed on Feb. 14, 2022; the disclosure of which, including all drawings and text, is incorporated by reference in its entirety herein.

### FIELD OF THE INVENTION

The invention relates generally to formulations and uses of nanoparticles comprising camptothecin conjugated to a mucic acid polymer.

### BACKGROUND

Breast cancer is the second most common cancer among women in the United States. Despite advances in screening and treatment, breast cancer remains the second leading cause of cancer death among women. Moreover, the efficacy of various treatments diverges amongst breast cancer subtypes at various stages of progression. This creates a complex picture for pathologists and oncologists in diagnosing, treating, and predicting recurrence in breast cancer patients.

Genomic DNA undergoes frequent challenges by both endogenous and exogenous DNA-damaging agents which can result in different types of DNA lesions. These lesions can block DNA replication and transcription and, if not repaired or if repaired incorrectly, may produce mutations that threaten the survival of individual cells or even the whole organism. The inability to accurately repair complex DNA damage and resolve DNA replication stress leads to genomic instability and contributes to cancer etiology, but also make cancer cells more vulnerable to DNA-damaging therapeutic agents.

One of several DNA repair pathways, homologous recombination (HR) repair, involves a series of interrelated pathways that function to repair DNA double-stranded breaks and interstrand crosslinks. Defects in HR repair are observed in various cancers. Breast cancer gene type 1 (BRCA1) and BRCA type 2 (BRCA2) have long been known to encode proteins that play a key role in HR repair and mutations in one or both of these genes place patients at greater risk for development of breast, ovarian, prostate, melanoma, and pancreatic cancers. Tumor cells with defective HR repair show increased sensitivity to chemotherapeutic agents, which suggests that HR proficient tumor cells might be sensitized to chemotherapeutics if HR repair could be therapeutically inactivated.

Topoisomerase inhibitors damage DNA by binding to topoisomerase I which prevents the unwinding of DNA required for replication. This results in a stalled replication fork that can be repaired by poly (ADP-ribose) polymerase (PARP). PARP inhibitors block PARP enzyme DNA repair activity and have been shown to induce apoptosis in tumor cells. In addition to direct tumor killing, inhibition of PARP also increases the cytotoxicity of chemotherapeutic drugs, and increases the cytotoxicity of DNA-damaging agents such as topoisomerase inhibitors, camptothecin (CPT) and its derivatives.

Unfortunately, while CPT is known for its anti-tumor activity, including over a wide spectrum of human cancers, it has poor water solubility, low plasma stability, and dose-limiting toxicity.

### SUMMARY

The present invention provides methods, compositions, and uses of camptothecin (CPT) conjugated nanoparticles. In particular, the present invention is directed to compositions, formulations, and methods of use that include formulations of nanoparticles comprising 20 (S)-camptothecin covalently attached to a mucic acid-based polymer carrier via a glycyl linkage (MAP-Gly-CPT) comprising Formula (I):

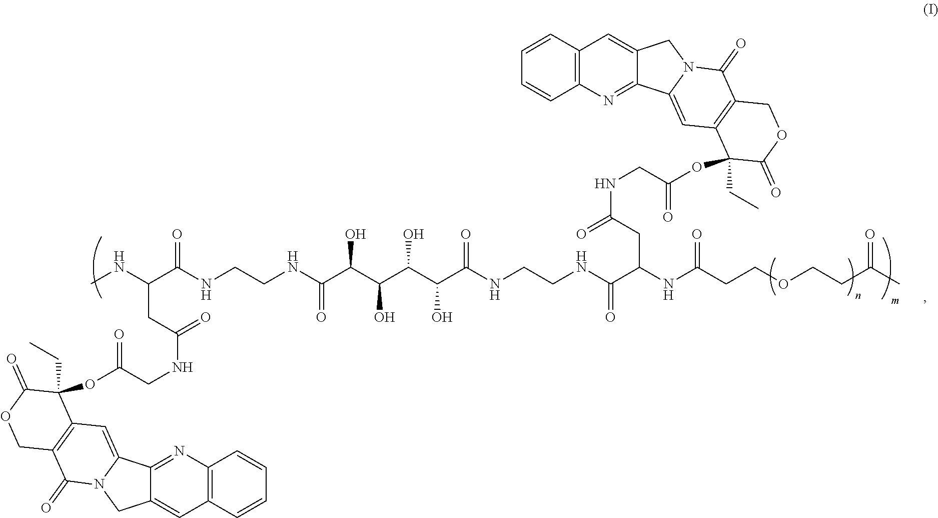

where n is the number of ethylene glycol repeating units, which is in the range of 20 to 200 units and m is the number of repeating units of MAP-Gly-CPT, which is in the range of 5 to 200.

In preferred aspects, the MAP-Gly-CPT compound is DAN-222, which has the structure of Formula I, wherein the number of repeating ethylene glycol repeating units (n) averages a number in a range from about 75 to 85. In preferred aspects, in the formulations and compositions of the invention n averages about 79. In certain aspects, the number of repeating units of MAP-Gly-CPT (m) averages 16±4.

Camptothecin (CPT), the active ingredient in DAN-222, is a naturally occurring, pentacyclic quinolone alkaloid isolated from the bark of Camptotheca acuminata. CPT is a topoisomerase I inhibitor that acts by forming a ternary complex with topoisomerase I and DNA during replication and transcription in tumor cells, which blocks the re-ligation of DNA, resulting in torsional tension downstream of the transcription fork or replication fork that causes DNA strand breaks.

Unfortunately, while CPT is known for its anti-tumor activity, including over a wide spectrum of human cancers, it has poor water solubility, low plasma stability, and dose-limiting toxicity.

However, as discovered by the present inventors, DAN-222 is more efficacious than other topoisomerase I inhibitors, including free CPT, due to its accumulation in tumors through an enhanced permeability and retention (EPR) effect, which leads to a greater and prolonged topoisomerase I inhibition.

In certain aspects, the present invention provides aqueous formulations comprising nanoparticles made of a mucic acid polymer (MAP) camptothecin (CPT) conjugate compound of Formula (I):

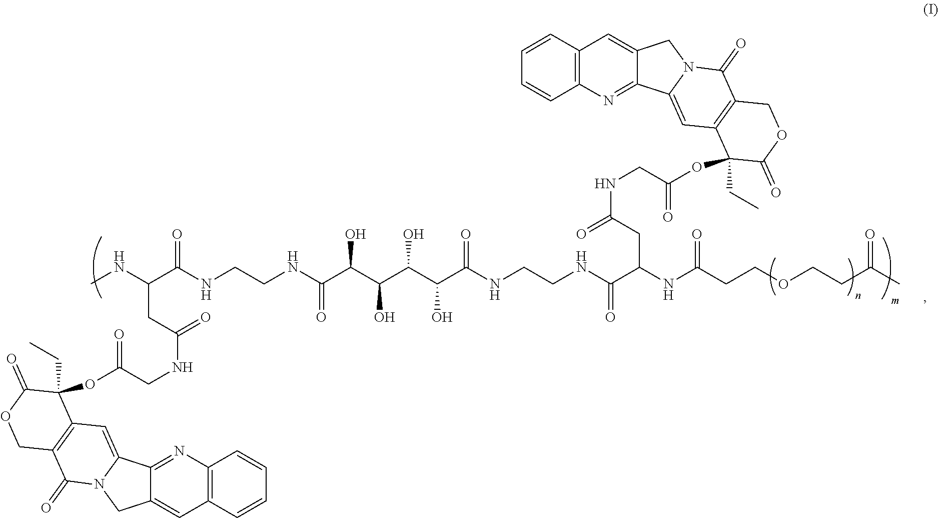

wherein n is a number in a range from 20 to 200; and
wherein m is a number in a range from 5 to 200.

In certain aspects, n is a number in a range from about 75 to 85. In certain formulations, m is a number in a range from about 12 to 20.

In certain formulations of the invention, the compound of Formula (I) has a molecular weight of about 75,000 Da. In preferred aspects, nanoparticles of the formulation comprise on average two strands comprising the compound of Formula (I) and have an average molecular weight of about 150,000 Da. The nanoparticles of the formulation may have an average particle size of about 20 nm to 80 nm. Preferably, the nanoparticles have an average particle size of about 30 to 40 nm. In certain aspects, the formulation has a nanoparticle concentration of about 17-23 mg/mL.

Formulations of the invention include those having a total concentration of CPT of between 1.7 and 3.6 mg/mL. In preferred aspects, the formulation has a total concentration of CPT of about 2.6 mg/mL. In certain aspects, the formulation comprises a concentration of about 20 mg/mL of the compound of Formula (I).

In certain aspects, the formulation has a pH between pH 4 and pH 5. In certain aspects, the formulation has a pH between pH 4 and pH 4.6. In preferred aspects, the formulation has a pH of about pH 4.3.

A formulation of the invention may further include at least one buffer selected from, e.g., sodium succinate, sodium citrate, sodium acetate, phosphoric acid, histidine-HCL, and sodium phosphate. In preferred aspects, the buffer is a sodium acetate buffer. The formulation may further include at least one tonicity modifier selected from, e.g., KCl, NaCl, Proline, Arginine-HCl, sucrose, and glycine. Preferably, the at least one tonicity modifier is NaCl.

In preferred aspects, the formulation comprises about 20 mg/mL of the compound of Formula (I), about 20 mM sodium acetate, about 200 mM NaCl, and has a pH of 4.3±0.3.

The present invention also provides processes for producing a formulation comprising the compound of Formula (I). In certain embodiments, a process for producing a formulation of a compound of Formula (I) comprises: (i) performing a derivatization of camptothecin (CPT) to yield Gly-CPT as the trifluoracetic acid (TFA) salt; (ii) synthesizing a parent mucic acid polymer (MAP); (iii) covalently attaching the Gly-CPT to the parent MAP to yield solid, amorphous MAP-Gly-CPT; and (iv) preparing an aqueous formulation using the amorphous MAP-Gly-CPT thereby producing an aqueous formulation of nanoparticles comprising the compound of Formula (I).

In certain aspects, step (i) of the process includes dissolving CPT, N-(tert-butoxycarbonyl)-Gly-OH (Boc-Gly), and 4-dimethylaminopyridine (DMAP) in methylene chloride (DCM) to form a reaction mixture; and adding diisopropylcarbodiimide (DIC) to the reaction mixture thereby producing a solution comprising a Boc-Gly-CPT intermediate. In certain embodiments, the reaction is conducted at 25-30° C. for about six hours.

In certain methods of the invention, step (i) further includes removing between 90% and 99% of the DCM from the solution comprising the Boc-Gly-CPT intermediate. In preferred methods, the DCM is removed via vacuum distillation. Step (i) may further include precipitating the Boc-Gly-CPT intermediate using methanol. Such methods may also include washing the precipitated Boc-Gly-CPT intermediate using isopropyl alcohol (IPA).

In certain aspects, step (i) further comprises treating the Boc-Gly-CPT intermediate with TFA in DCM to yield the TFA salt of Gly-CPT. In certain embodiments, treatment with TFA is conducted at 25-30° C. for about two hours.

In preferred methods for producing the formulation, step (ii) includes: charging a reactor with (succinimidyl propionate) 2-PEG3500 (diSPA-PEG3500) and a mucic acid monomer (MAM) neutral species of Formula (II):

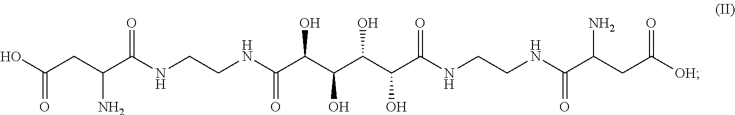 [SMILES: NC(CC(=O)O)C(=O)NCCNC(=O)[C@@H](O)[C@H](O)[C@H](O)[C@@H](O)C(=O)NCCNC(=O)C(N)CC(=O)O]

adding dimethyl sulfoxide (DMSO) to the reactor; initiating the reaction by adding TFA to the reactor; and adding DIPEA to the reaction, thereby yielding the parent MAP. In certain embodiments, the reaction is conducted at 21-23° C. for about three hours.

In certain aspects, the comonomer ratio of DIPEA: MAM used in step (ii) is at least about 2.5. Preferably, the comonomer ratio is at least 2.6.

In certain aspects, step (ii) further includes precipitating the parent MAP from the DMSO via addition of at least one anti-solvent. An exemplary anti-solvent is isopropyl alcohol (IPA). In preferred embodiments, the precipitating step includes a filtration step under an inert atmosphere, such as, for example, nitrogen or argon.

In certain methods of the invention, step (iii) includes: adding the parent MAP and DMSO to a reaction vessel; adding the Gly-CPT (TFA salt) and (7-azabenzotriazol-1-yloxy)tripyrrolidinophosphonium hexafluorophosphate (PyAOP) to the reaction vessel; and adding DIPEA to the reaction vessel, thereby producing a solution that comprises MAP-Gly-CPT. In certain embodiments, the reaction is conducted at 19-25° C. for 16-22 hours.

Step (iii) may further include purifying amorphous MAP-Gly-CPT from the solution using tangential flow filtration (TFF). Preferably, prior to the tangential flow filtration, the solution is diluted with pH 3 water and the tangential flow filtration uses polyethersulfone (PESU) TFF filters. In additional embodiments, membranes made of regenerated cellulose are used for TFF.

In certain methods of the invention, step (iv) includes: adding water with a pH of around pH 4 to the amorphous MAP-Gly-CPT to produce a nanoparticle (NP) solution; and adding a formulation buffer to the NP solution.

In certain aspects, the formulation buffer comprises sodium acetate. Preferably, the formulation buffer further comprises NaCl and has a pH of around pH 4.16.

In certain methods, after the step of adding the water with a pH of around pH 4, the NP solution is filtered. In certain embodiments, after the step of adding the formulation buffer to the NP solution, the NP solution is concentrated by ultrafiltration.

The present disclosure also provides methods for treating cancer in a subject using DAN-222 formulations as described herein. An exemplary method includes providing to a subject having cancer at least one dose of a composition comprising a compound of Formula (I):

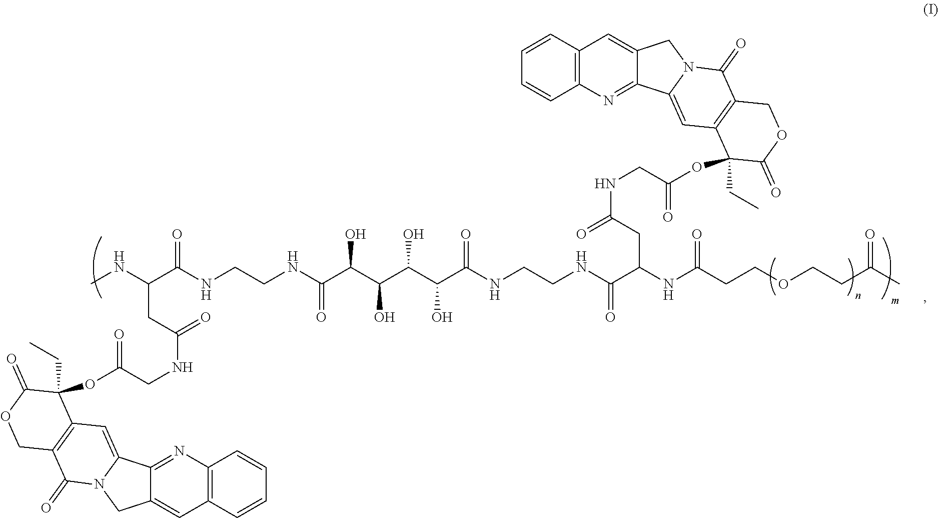

wherein n is a number in a range from 20 to 200; and wherein m is a number in a range from 5 to 200. In preferred methods: n is a number in a range from about 75 to 85; m is a number in a range from about 12 to 20; and the composition has a pH between pH 4 and pH 4.6.

Preferably, the composition is provided in solution for infusion.

In preferred methods, the composition comprises between 2 mg/mL and 3 mg/ml of camptothecin (CPT). In certain embodiments, the dose of the composition comprises between 2 mg/m² and 16 mg/m² of the compound of Formula (I). An exemplary dose of the composition comprises about 2 mg/m², about 4 mg/m², about 6 mg/m², about 8 mg/m², about 10 mg/m², about 12 mg/m², about 14 mg/m², or about 20 mg/m² of the compound of Formula (I).

In certain methods, a plurality of doses is provided to a subject during a treatment cycle. Preferably, the doses are provided as weekly doses, i.e., one dose provided every seven days.

Exemplary methods of treatment further include providing at least one dose of a poly (ADP-ribose) polymerase (PARP) inhibitor to the subject. The PARP inhibitor may include, for example, one of olaparib, rucaparib, niraparib, and talazoparib. Preferably, the PARP inhibitor is niraparib. Preferably, the subject receives a dose of niraparib daily during the treatment cycle. In exemplary methods, each dose of niraparib is a 100 mg dose.

In exemplary methods of the invention, the cancer is one or more cancers of the breast, ovary, brain, lung, testicle, head, neck, esophagus, lymphoma, central nervous system, peripheral nervous system, bladder, stomach, pancreas, liver, oral mucosa, colorectal, anus, kidney, bladder, uroepithelium, prostate, endometrium, uterus, fallopian tube, mesothelioma, melanoma, myeloma, leukemia, and Kaposi's sarcoma.

In preferred methods, the cancer is a breast cancer. The breast cancer may include, for example, a breast cancer that is a homologous recombination repair deficiency (HRD)-positive or HRD-negative breast cancer.

### BRIEF DESCRIPTION OF THE DRAWINGS

FIG. 1 shows the formation of a DAN-222 engineered nanoparticle.

FIG. 2 provides the calculated median tumor volumes (MTV) as a function over time presented by various treatment groups in an HRD-positive (ER-positive, PR-positive, HER2-positive) breast ductal carcinoma (BT-474 model). Treatment groups are:
vehicle control (circles, top line)
camptothecin (3 mg/kg) iv once weekly for 3 weeks (diamonds, next lower line)
irinotecan (100 mg/kg) iv once weekly for 3 weeks (triangles, next lower line)
DAN-222 (3 mg/kg) iv once weekly for 3 weeks (squares, lowest line).

FIG. 3 provides a Kaplan Meier plot of the time to endpoint (TTE), for animals in each of the treatment groups described in FIG. 2. Endpoint is a tumor volume of 2,000 mm3. Treatment groups are:
vehicle control (circles, first line that descends from 100%)
camptothecin (3 mg/kg) iv once weekly for 3 weeks (diamonds, second line that descends from 100%)
irinotecan (100 mg/kg) iv once weekly for 3 weeks (triangles, third line that descends from 100%)
DAN-222 (3 mg/kg) iv once weekly for 3 weeks (square, line that does not descend from 100%).

FIG. 4 shows median tumor volume measurements, over time, for various treatment groups in a HRD-positive, triple negative (ER-negative, PR-negative, HER2-negative) breast adenocarcinoma (MDA-MB-436) model. Treatment groups are, in order of the location of the right-most data point, from top to bottom:
Vehicle (dark circles)
25 mg/kg oral niraparib once daily for 28 days (light circles)
0.3 mg/kg intravenous DAN-222 once weekly for four weeks (light squares)
1 mg/kg intravenous DAN-222 once weekly for four weeks (dark squares)
0.3 mg/kg intravenous DAN-222 once weekly for four weeks plus 25 mg/kg oral niraparib once daily for 28 days (light triangles)
1 mg/kg intravenous_DAN-222 once weekly for four weeks plus 25 mg/kg oral niraparib once daily for 28 days (light triangles)
3 mg/kg intravenous DAN-222 once weekly for four weeks (dark squares)
3 mg/kg intravenous DAN-222 once weekly for four weeks plus 25 mg/kg oral niraparib once daily for 28 days (dark triangles).

FIG. 5 shows median tumor volume measurements, over time, after DAN-222 administration in HRD-positive (ER-negative, PR-negative, HER2-negative [triple negative]) breast adenocarcinoma (MDA-MB-436 model). Treatment groups are, in order of the location of the right-most data point, from top to bottom:
Vehicle (dark circles)
40 mg/kg oral niraparib once daily for 28 days (light circles)
0.3 mg/kg intravenous DAN-222 once weekly for four weeks (light squares)
1 mg/kg intravenous DAN-222 once weekly for four weeks (dark squares)
0.3 mg/kg intravenous DAN-222 once weekly for four weeks plus 40 mg/kg oral niraparib once daily for 28 days (light triangles)
3 mg/kg intravenous DAN-222 once weekly for four weeks (dark squares)
1 mg/kg intravenous DAN-222 once weekly for four weeks plus 40 mg/kg oral niraparib once daily for 28 days (dark triangles).

FIG. 6 shows Median Tumor Volume over time in a OVCAR3 Human Ovarian Cancer Xenograft Model with DAN-222 and high-dose Niraparib. Treatment groups, are in order of the location of the right-most data point, from top to bottom:
Vehicle (dark circles)
50 mg/kg oral niraparib once daily for 28 days (light circles)
1 mg/kg intravenous DAN-222 once weekly for four weeks (squares)
1 mg/kg intravenous DAN-222 once weekly for four weeks plus 50 mg/kg oral niraparib once daily for 28 days (triangles)
3 mg/kg intravenous DAN-222 once weekly for four weeks (squares)

FIG. 7 shows concentrations of CPT in plasma of male rats after intravenous dosing of DAN-222 on Days 1 and 22.

FIG. 7A (left side of figure) shows plasma levels of free CPT after intravenous administration of DAN-222 at three different doses: 1.1 mg/kg (triangles), 3.2 mg/kg (squares) and 9.6 mg/kg (circles). The upper panel shows free CPT levels after dosing at day 1. The lower panel shows free CPT levels after a repeat dose at day 22.

FIG. 7B (right side of figure) shows plasma levels of total CPT after intravenous administration of DAN-222 at three different doses: 1.1 mg/kg (triangles), 3.2 mg/kg (squares) and 9.6 mg/kg (circles). The upper panel shows total CPT levels after dosing at day 1. The lower panel shows total CPT levels after a repeat dose at day 22.

FIG. 8 shows stability of DAN-222 in Rat, Dog, and Human plasma at physiological pH over time. The three curves, in order of the location of the right-most data point, show results for dog, rat and human, respectively.

FIG. 9A shows biodistribution of DAN-222 in different tissues of JIMT-1 xenograft mice. Darker bars show results for total CPT; lighter bars show results for free CPT.

FIG. 9B shows biodistribution of DAN-222 in JIMT-1 xenograft mice over time. The left-most set of six bars shows amounts of total CPT in plasma; the center set of six bars shows amounts of total CPT in skeletal muscle; and the right-most set of six bars shows amounts of total CPT in tumor tissue.

FIG. 10 provides a summary of DAN-222 biodistribution studies performed.

FIG. 11 provides stability data for DAN-222 formulations as % free CPT and Avg. Particle Size trends over 14 days at 2-8° C.

FIG. 12 provides stability data for DAN-222 formulations as % free CPT and Avg. Particle Size trends over 14 days at 20-25° C.

FIG. 13 provides stability data for DAN-222 formulations as % free CPT and Avg. Particle Size trends over 14 days at 40° C.

FIG. 14 provides stability data for DAN-222 formulations as % free CPT and Avg. Particle Size over 24 hours of agitation.

FIG. 15 provides stability data for DAN-222 formulations as % free CPT and Avg. Particle Size trends over six freeze/thaw (F/T) cycles.

FIG. 16 provides stability data for DAN-222 formulations as % free CPT and Avg. Particle Size trends over 24 hours of 0.02% H₂O₂ Stress.

FIG. 17 provides stability data for DAN-222 formulations as % free CPT and Avg. Particle Size trends over 24 hours of 0.05% H₂O₂ Stress.

FIG. 18A provides stability data for DAN-222 formulations in acetate buffer as % free CPT and Avg. Particle Size trends over one month at 2-8° C.

FIG. 18B provides stability data for DAN-222 formulations in histidine buffer as % free CPT and Avg. Particle Size trends over one month at 2-8° C.

FIG. 19 provide stability data for DAN-222 formulations as % free CPT and Avg.

Particle Size trends over 4 months at 2-8° C.

FIG. 20A provides stability data for DAN-222 formulations in acetate buffer as % free CPT and Avg. Particle Size trends over one month at 20-25° C.

FIG. 20B provides stability data for DAN-222 formulations in histidine buffer as % free CPT and Avg. Particle Size trends over one month at 20-25° C.

FIG. 21A provides stability data for DAN-222 formulations in acetate buffer as % free

CPT and Avg. Particle Size trends over 7 days at 40° C.

FIG. 21B provides stability data for DAN-222 formulations in histidine-buffer as % free CPT and Avg. Particle Size trends over 7 days at 40° C.

FIG. 22 provides stability data for DAN-222 formulations as % free CPT and Avg. Particle Size trends over 4 months at −20° C.

FIG. 23 provides stability data for DAN-222 formulations as % free CPT and Avg. Particle Size trends over 4 months at −80° C.

FIG. 24A provides stability data for DAN-222 formulations in acetate buffer as % free CPT and Avg. Particle Size over 24 hours of agitation.

FIG. 24B provides stability data for DAN-222 formulations in histidine buffer as % free

CPT and Avg. Particle Size over 24 hours of agitation.

FIG. 25A provides stability data for DAN-222 formulations in acetate buffer as % free CPT and Avg. Particle Size trends over six freeze/thaw (f/T) cycles.

FIG. 25B provides stability data for DAN-222 formulations in histidine buffer as % free CPT and Avg. Particle Size trends over six freeze/thaw (F/T) cycles.

FIG. 26A provides stability data for DAN-222 formulations in acetate buffer as % free CPT and Avg. Particle Size trends over 24 hours of 0.05% H₂O₂ Stress.

FIG. 26B provides stability data for DAN-222 formulations in histidine buffer as % free CPT and Avg. Particle Size trends over 24 hours of 0.05% H₂O₂ Stress.

FIG. 27 provides stability data for DAN-222 formulations as % free CPT and Avg.

Particle Size trends over one month at 2-8° C.

FIG. 28 provides stability data for DAN-222 formulations as % free CPT and Avg. Particle Size trends over 3 months at 2-8° C.

FIG. 29 provides stability data for DAN-222 formulations as % free CPT and Avg. Particle Size trends over one month at 20-25° C.

FIG. 30 provides stability data for DAN-222 formulations as % free CPT and Avg. Particle Size trends over 7 days at 40° C.

FIG. 31 provides stability data for DAN-222 formulations as % free CPT and Avg. Particle Size trends over 3 months at −20° C.

FIG. 32 provides stability data for DAN-222 formulations as % free CPT and Avg.

Particle Size trends over 3 months at −80° C.

FIG. 33 provides stability data for DAN-222 formulations as % free CPT and Avg. Particle Size over 24 hours of agitation. at 450 rpm.

FIG. 34 provides stability data for DAN-222 formulations as % free CPT and Avg. Particle Size trends over six freeze/thaw (F/T) cycles.

FIG. 35 provides a schematic for a process of producing a DAN-222 formulation of the invention.

FIG. 36 provides a scheme for producing Gly-CPT.

FIG. 37A provides the first part of a workflow used in producing a DAN-222 formulation of the invention.

FIG. 37B provides the second part of a workflow used in producing a DAN-222 formulation of the invention.

FIG. 38 shows a synthesis scheme for preparing a mucic acid polymer (MAP).

FIG. 39 provides a workflow used in producing a DAN-222 formulation of the invention.

FIG. 40 provides a synthetic scheme for preparing amorphous MAP-Gly-CPT.

FIG. 41A provides the first part of a summary flow chart of the MAP-Gly-CPT amorphous synthetic process, including control points.

FIG. 41B provides the second part of a summary flow chart of the MAP-Gly-CPT amorphous synthetic process, including control points.

FIG. 42 provides a scheme for preparing a DAN-222 solution/formulation.

FIG. 43A provides the first part of a summary flow chart of the DAN-222 FBDS process, including control points.

FIG. 43B provides the second part of a summary flow chart of the DAN-222 FBDS process, including control points.

FIG. 44 provides results showing effect of MAM: diSPA-PEG comonomer ratio on MAP molecular weight (MW). Circles indicate a 1.01:1 ratio; squares indicate a 1.03:1 ratio; and triangles indicate a 1.10:1 ratio.

FIG. 45 shows chemical structures of mucic acid monomer (MAM)diTFA salt and MAM neutral species.

FIG. 46 provides the results of an initial set of experiments (screen 1) carried out to study the combined effects of different comonomer ratios as well as equivalents of TFA and DIPEA on the kinetics of polymerization of mucic acid monomer (MAM) to mucic acid polymer (MAP). Results are shown as polymer molecular weight over time. Reaction conditions are shown in the Table below the graph. Circles indicate results obtained using reaction conditions shown in Column 1-A of the table. Squares indicate results obtained using reaction conditions shown in Column 1-B of the table. Triangles indicate results obtained using reaction conditions shown in Column 1-C of the table. Diamonds indicate results obtained using reaction conditions shown in Column 1-D of the table.

FIG. 47 provides results of a second set of experiments using different conditions for preparation of mucic acid polymer (MAP). Results are shown as polymer molecular weight over time. Reaction conditions are shown in the Table below the graph. Circles indicate results obtained using reaction conditions shown in Column 2-A of the table. Squares indicate results obtained using reaction conditions shown in Column 2-B of the table. Triangles indicate results obtained using reaction conditions shown in Column 2-C of the table. Diamonds indicate results obtained using reaction conditions shown in Column 2-D of the table.

FIG. 48 shows MAP molecular weight as a function of time for scaled-up MAP synthesis reactions. Three reactions were conducted at 5 g scale (triangles, squares and upper set of circles), and one reaction was conducted at 50 g scale (lower set of circles).

FIG. 49 shows percent drug loading and grafting efficiency for synthesis of MAP-gly-CPT conjugate using PyAOP as a coupling reagent, as a function of CPT equivalents. Letters inside the circles refer to columns in Table 9.

FIG. 50 provides a summary of manufacturing differences between lots of DAN-222 formulations manufactured using the methods disclosed herein.

FIG. 51 provides compatibility results for DAN-222 IV bag administration in the low dose (0.022 mg/mL) formulation.

FIG. 52 provides compatibility results for DAN-222 IV bag administration in the high dose (0.400 mg/mL) formulation.

FIG. 53 provides the scheme for a Dose-Escalation Study of the Safety and Pharmacology of DAN-222 in Subjects with Metastatic Breast Cancer.

### DETAILED DESCRIPTION

The present invention provides methods, compositions, and uses of camptothecin (CPT) conjugated nanoparticles. In particular, the present invention is directed to compositions, formulations, and methods of use that include the compositions comprising DAN-222, which is a polymer-small molecule conjugate, formulated and assembled as engineered nanoparticles (NPs). The polymer is a linear, modified mucic acid-polyethylene glycol (PEG) copolymer. The small molecule active pharmaceutical ingredient is camptothecin (CPT), a topoisomerase I inhibitor. The CPT is conjugated with the polymer via a glycyl linkage. As a polymeric construct DAN-222 consists of multiple copies of the main repeat unit constructed as a linear polymer. The repeat unit for this polymer consists of a modified mucic acid bonded to a nominal 3,500 Da linear PEG derivative. The chemical structure of the DAN-222 MAP-Gly-CPT polymer-drug conjugate repeat unit is provided in Formula (I):

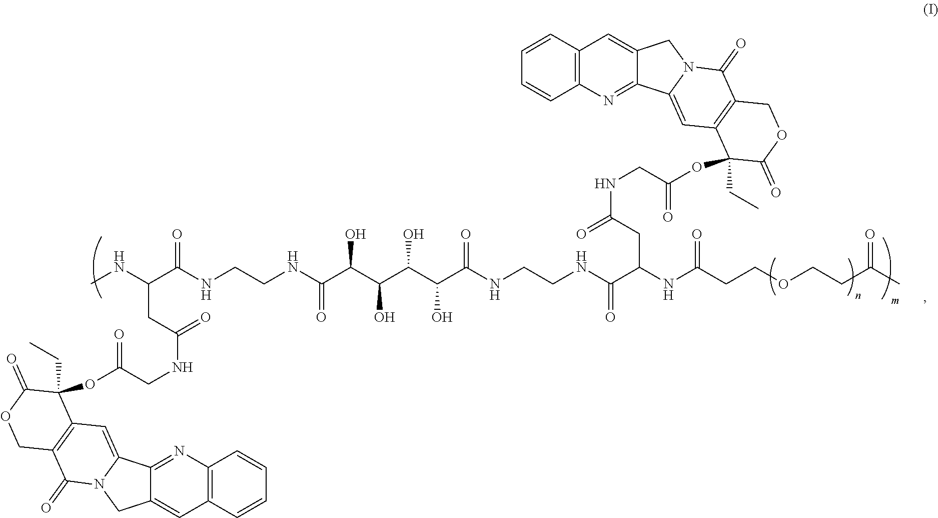

where n is the number of ethylene glycol repeating units (which on average n=79 for 3,500 Da PEG) and m is the number of repeating units of the MAP-Gly-CPT polymer-drug conjugate (which on average m=16±4 for a MAP parent polymer).

Formula (I) represents the polymer-drug conjugate (MAP-Gly-CPT) of DAN-222. The polymer repeat unit consists of a modified mucic acid moiety bonded to a bis-propanoyl PEG unit. Each PEG unit has a nominal molecular weight of 3,500 Da ((C₂H₄O)₇₉). On average, each polymer strand consists of 16 repeats of this PEG-MAP unit, each of which can be conjugated with up to two Gly-CPT molecules. When Gly-CPT is conjugated to MAP, the reaction is performed so that about 90% of available sites are reacted.

The average molecular weight of MAP parent polymer consisting of 16 repeat units is approximately 65,000 Da. The target molecular weight range for this material is 50,000-80,000 Da. The molecular weight of a 65,000 Da MAP polymer at 90% loading of CPT is approximately 75,000 Da.

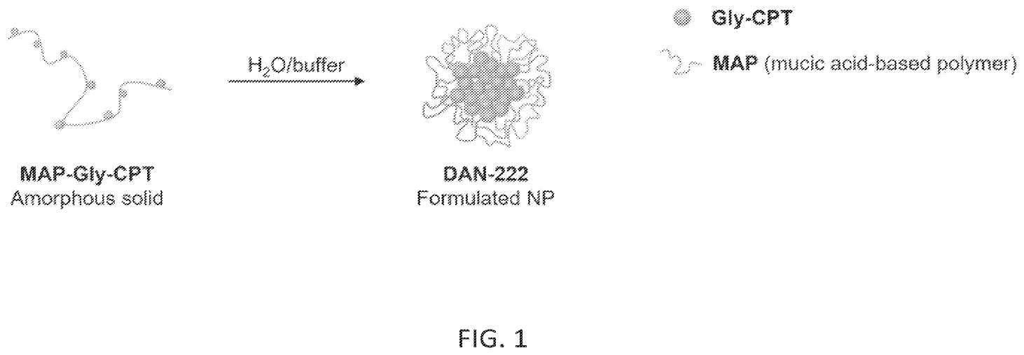

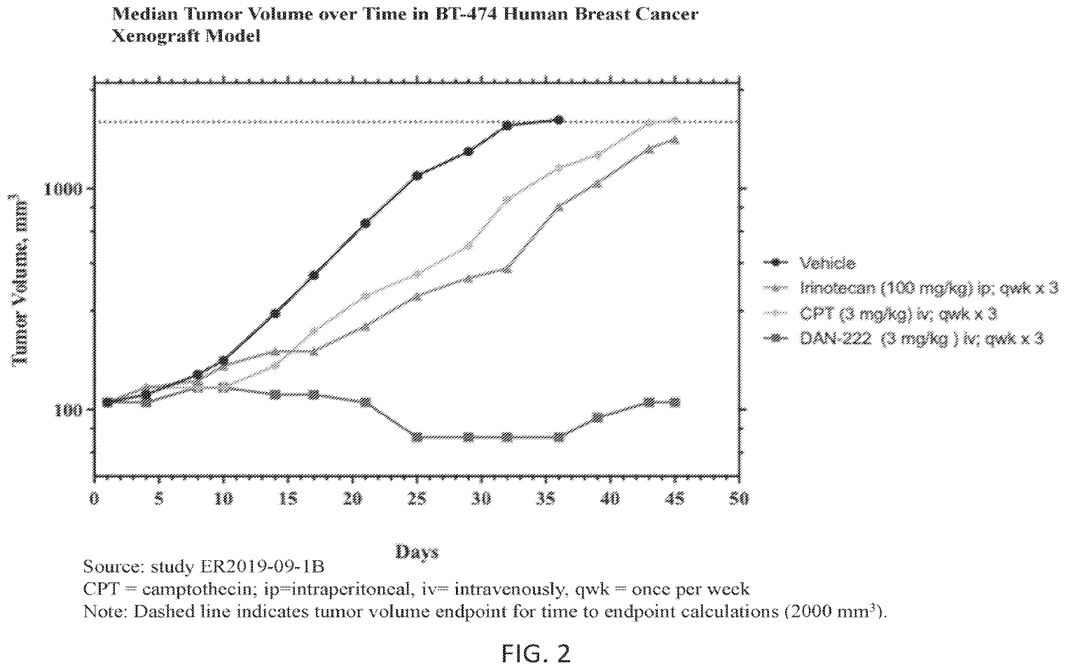

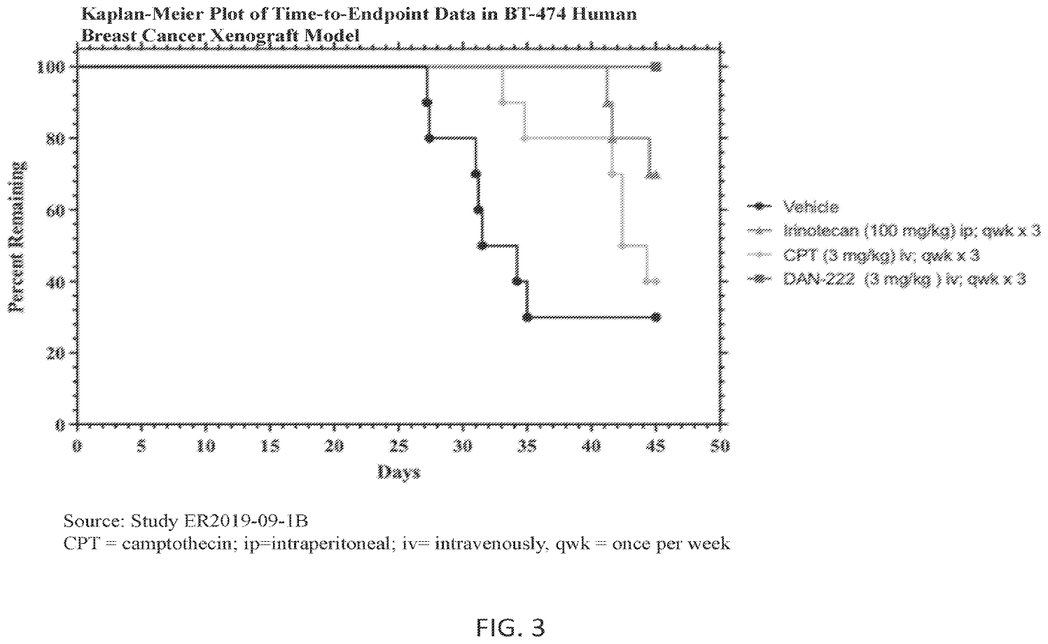

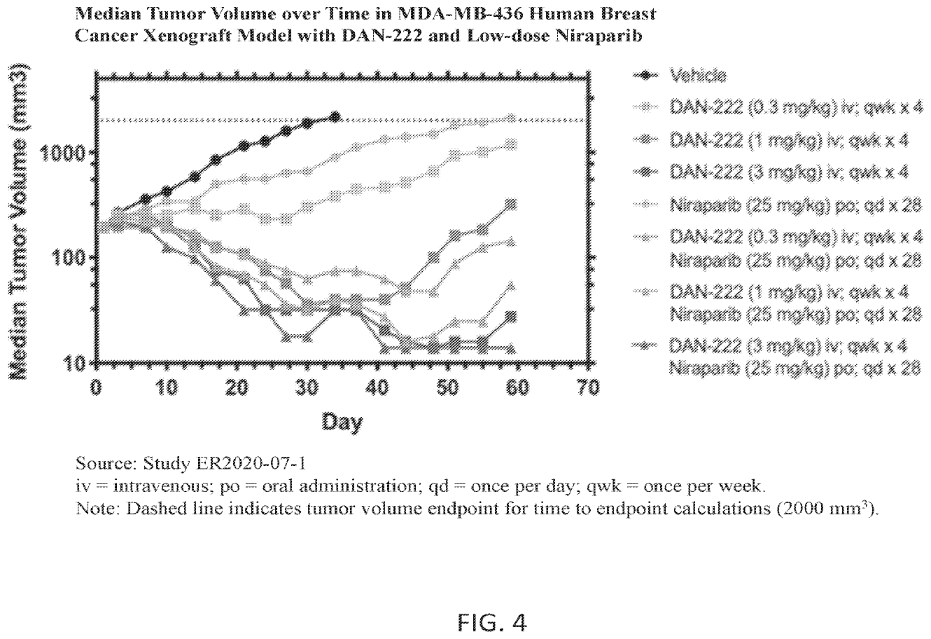

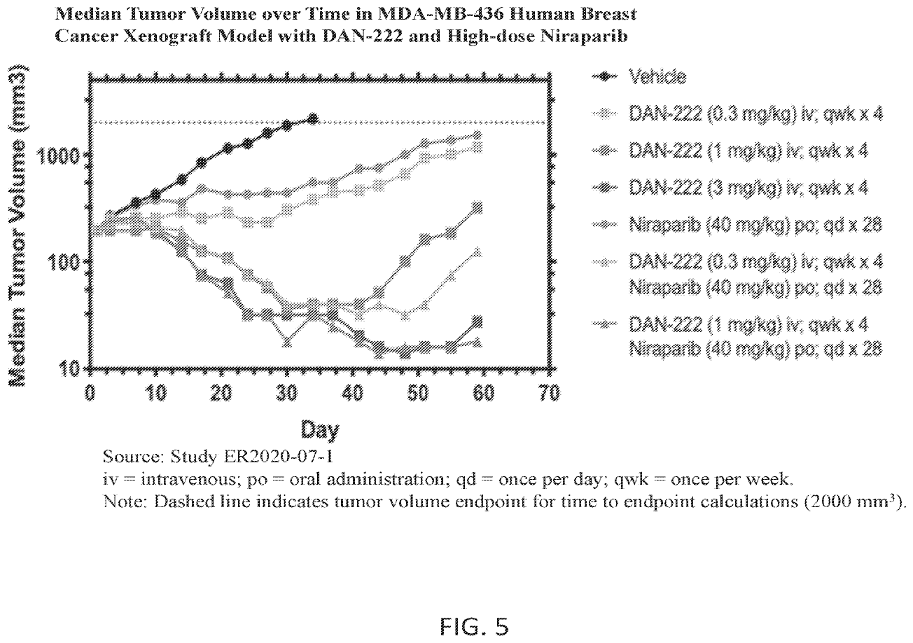

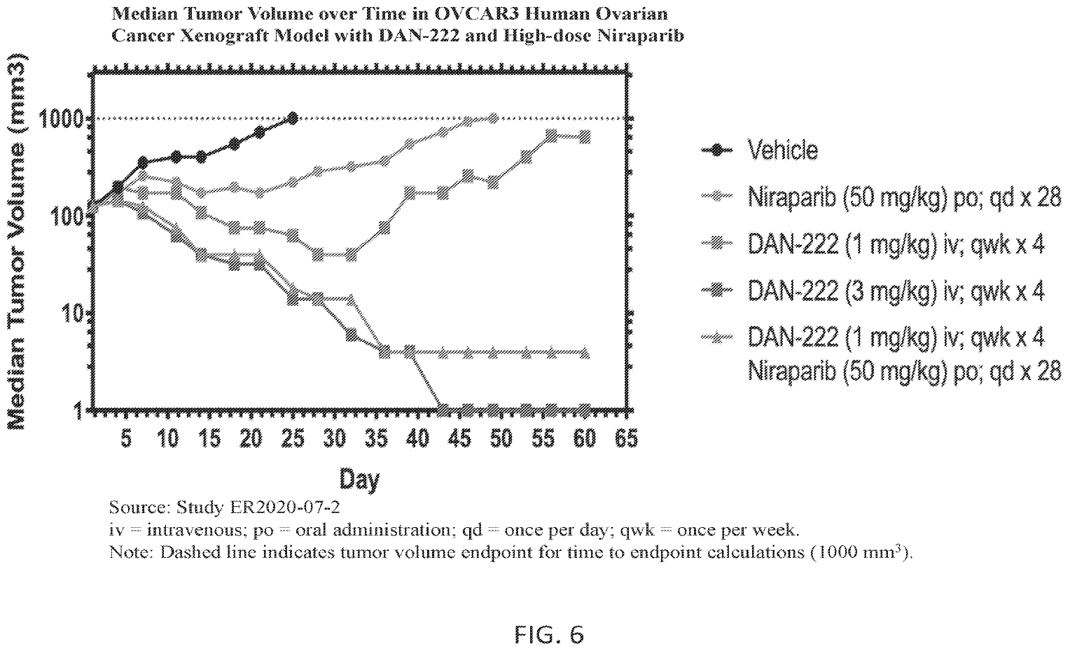

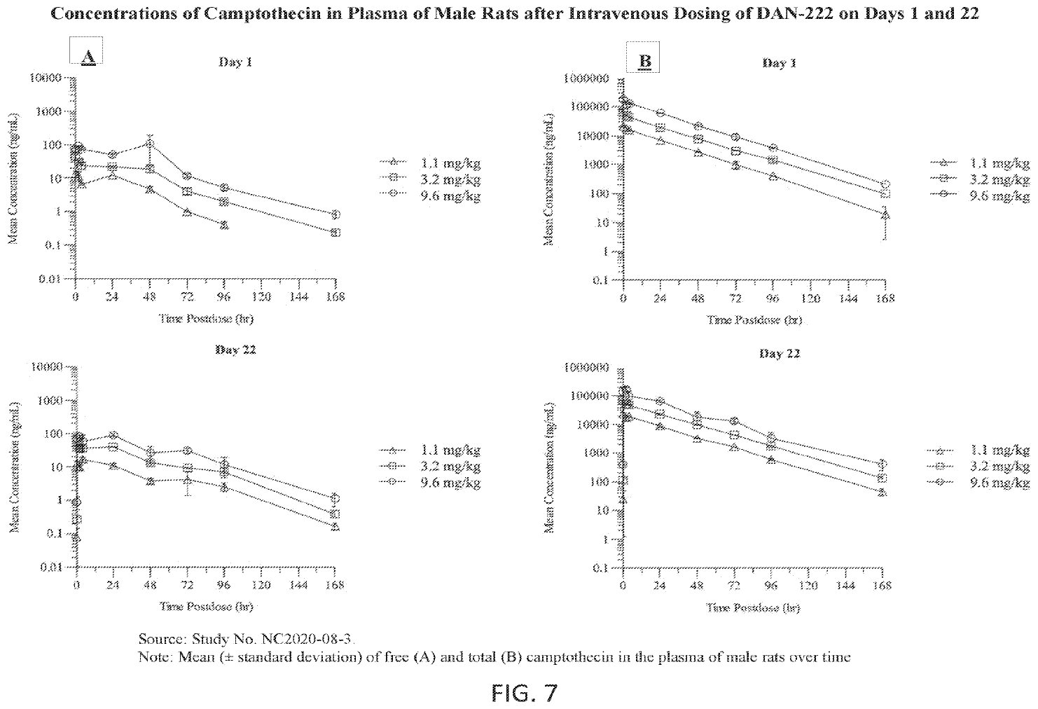

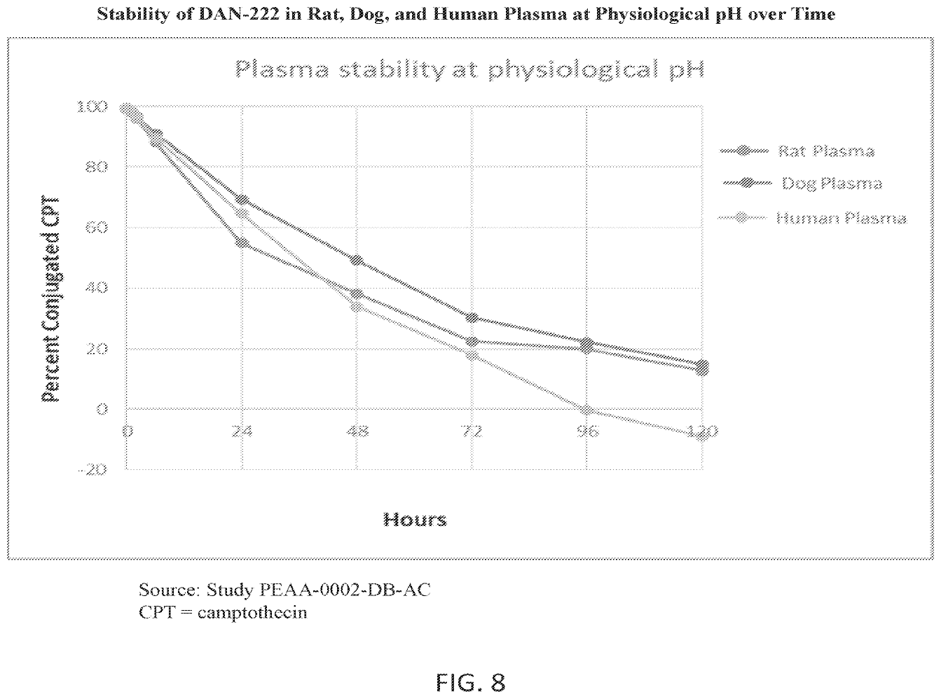

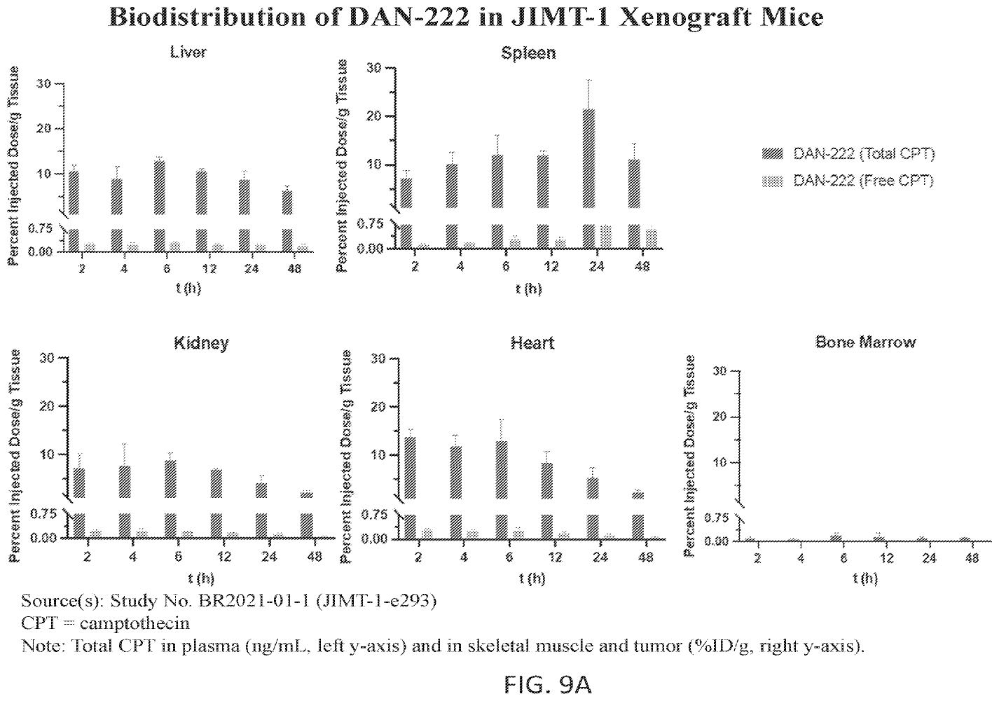

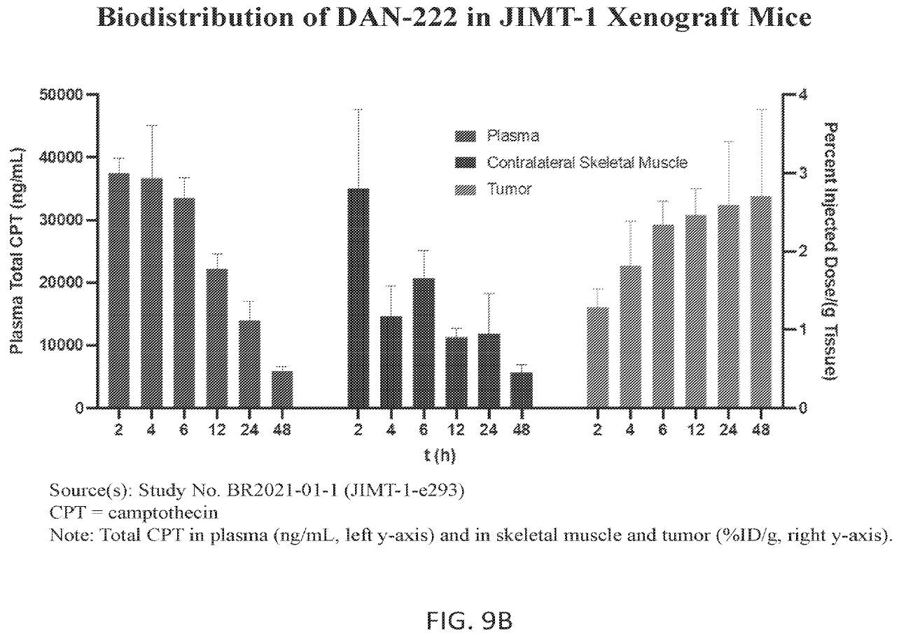

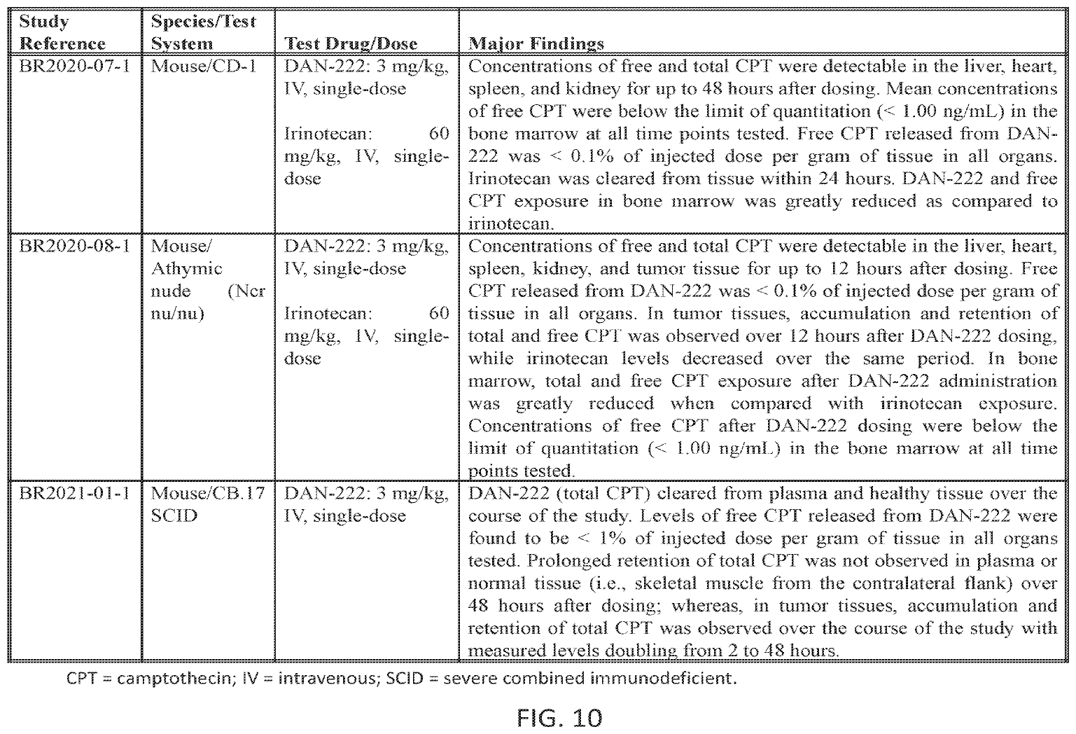

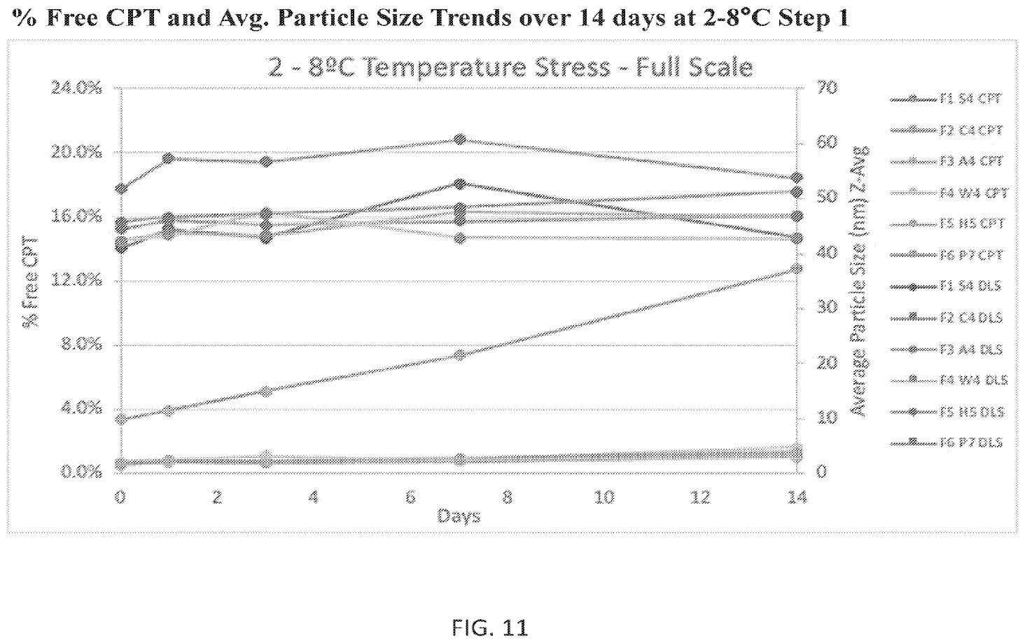

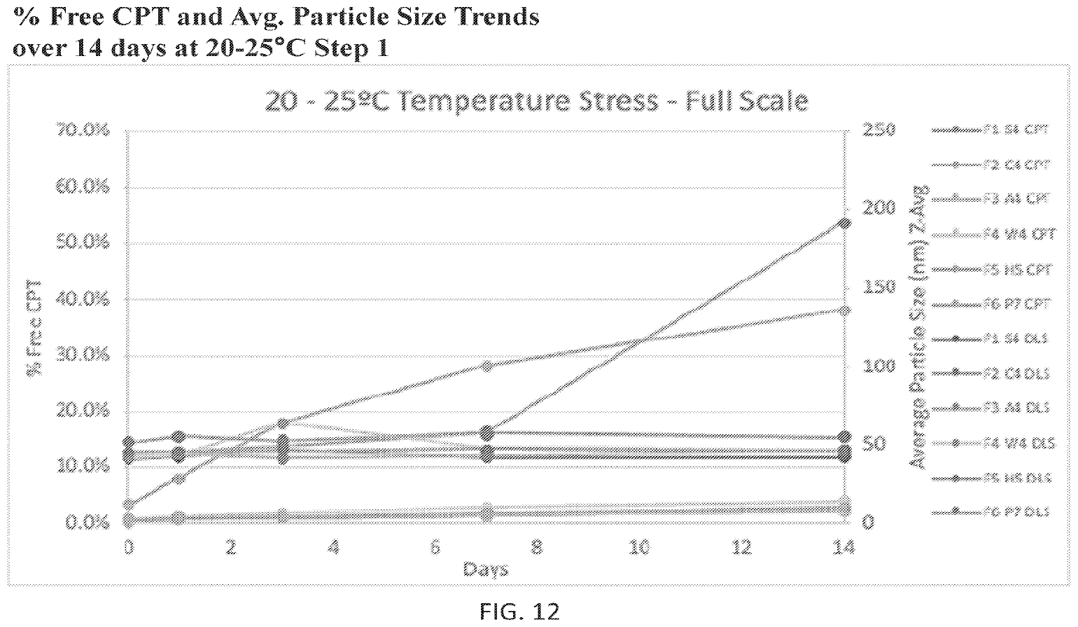

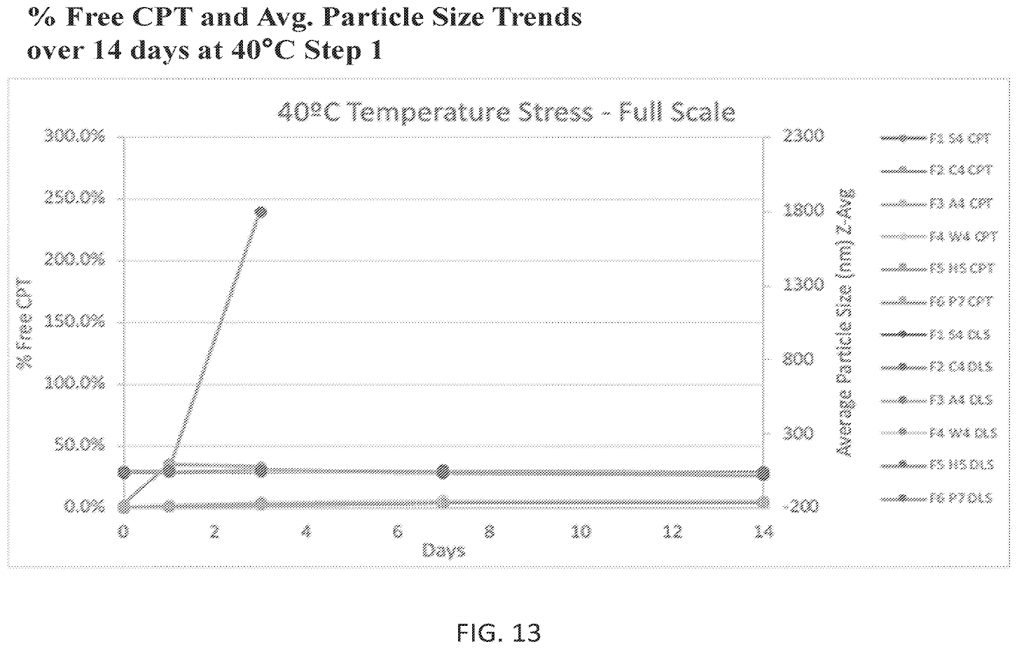

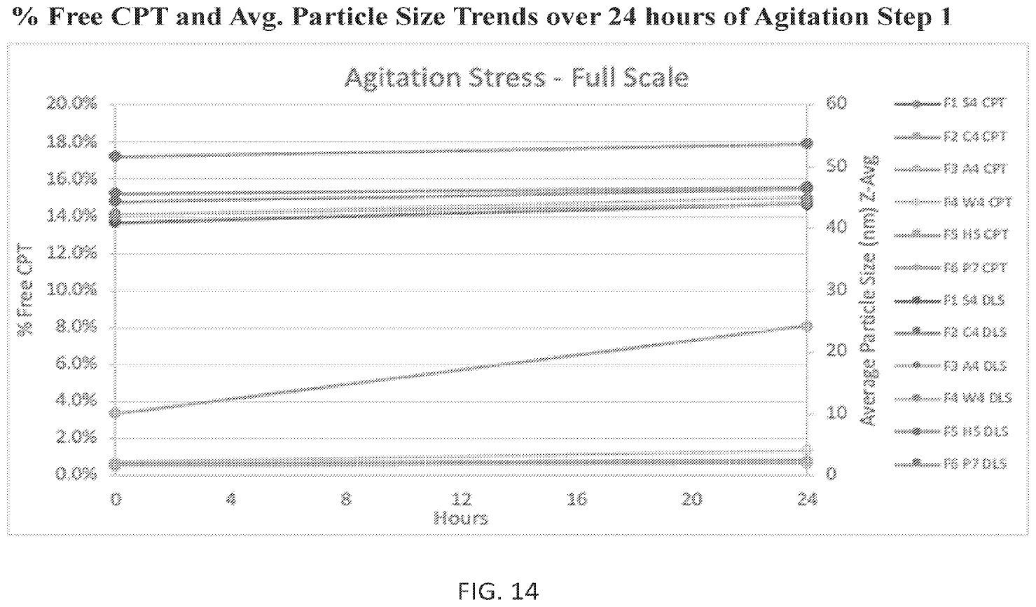

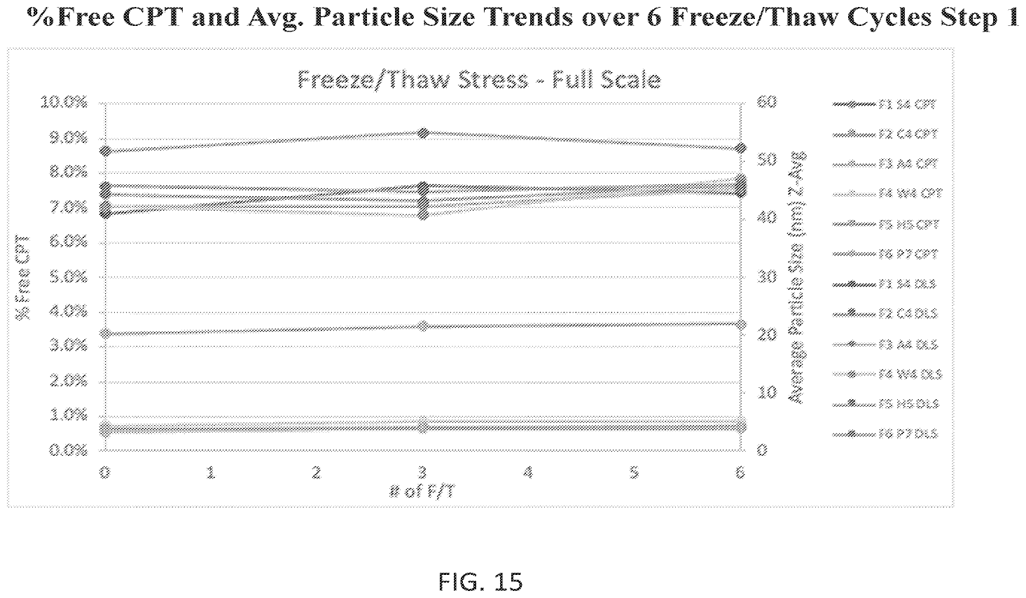

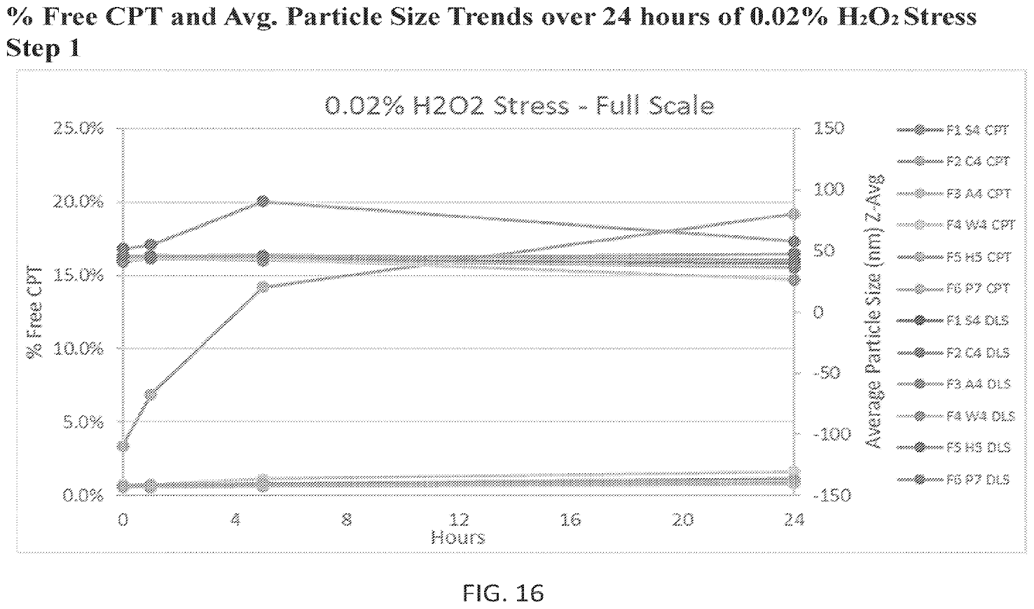

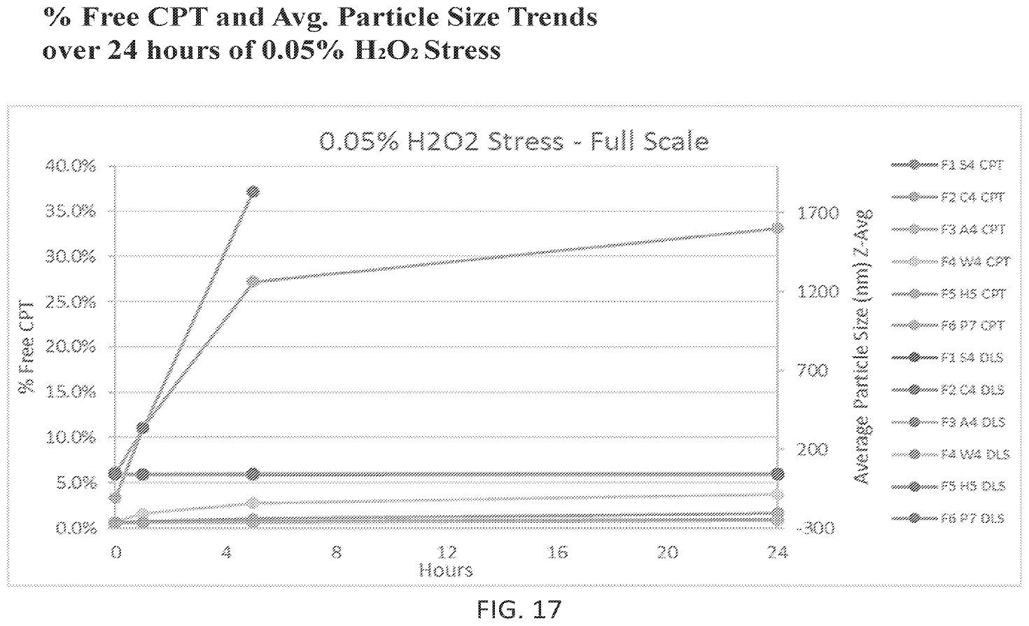

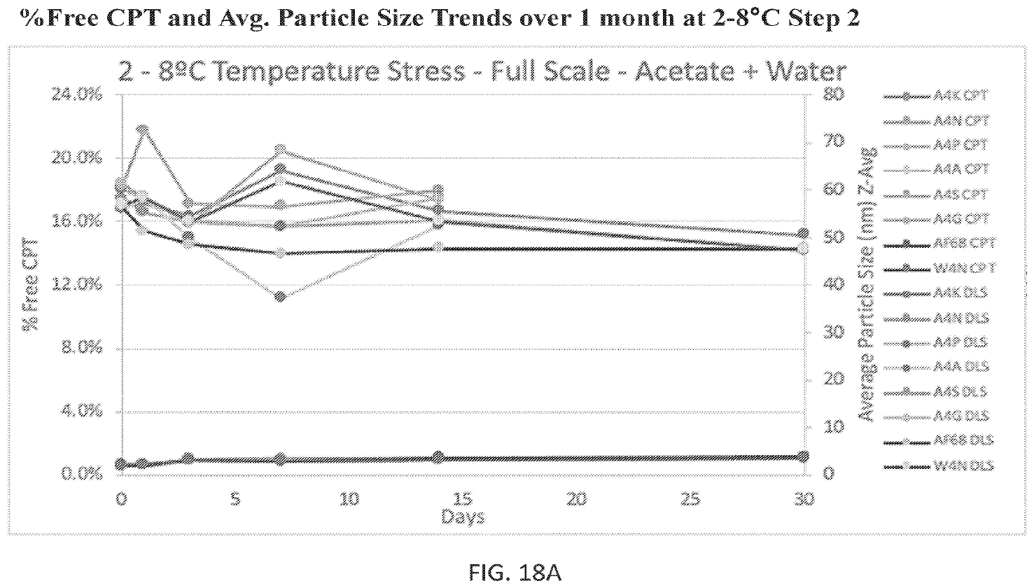

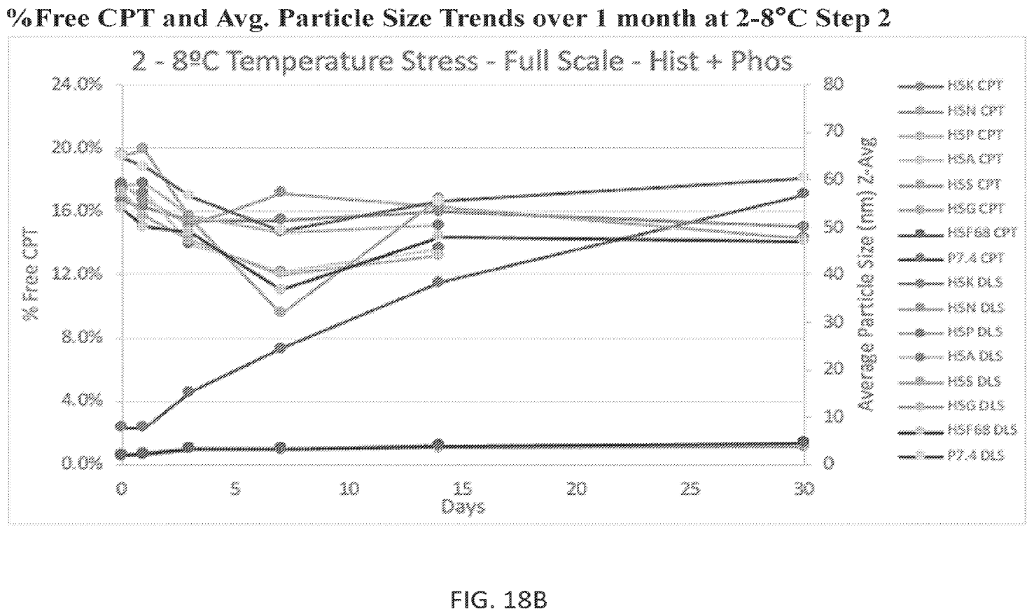

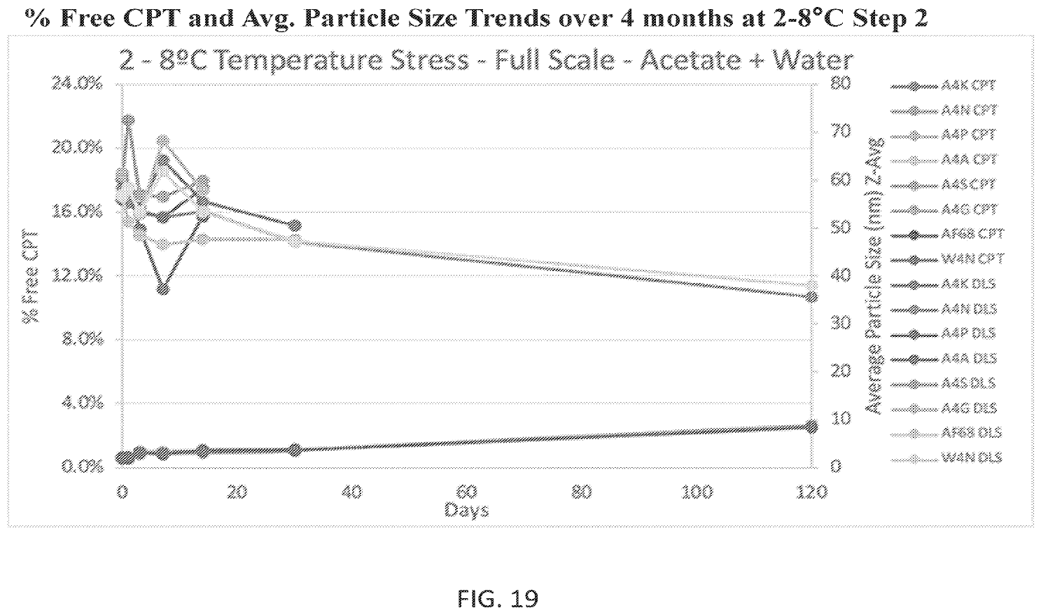

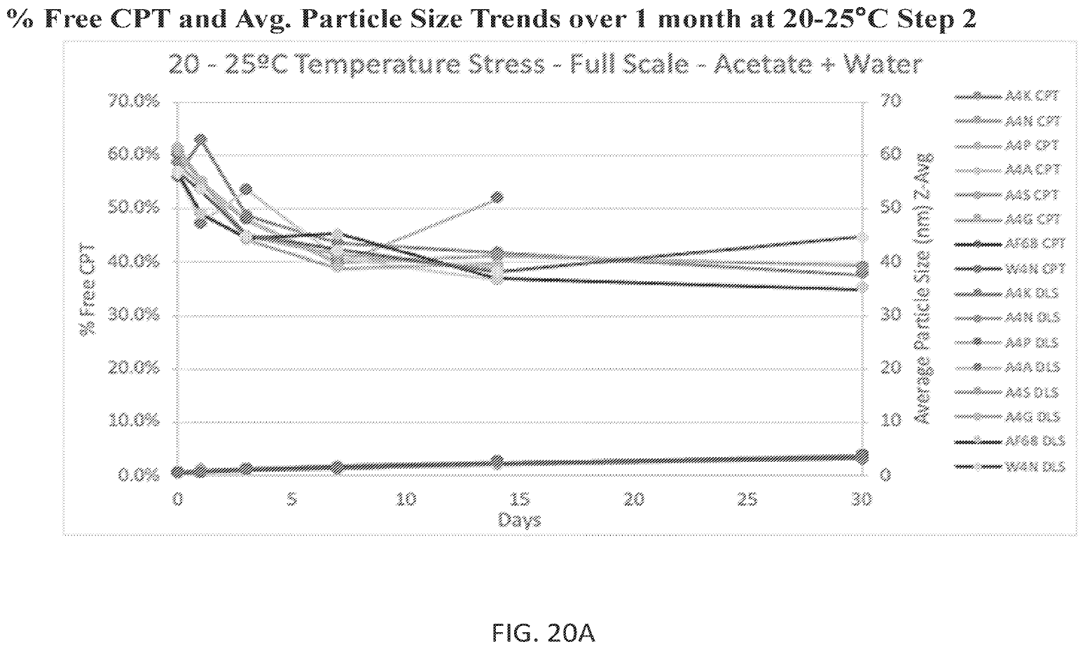

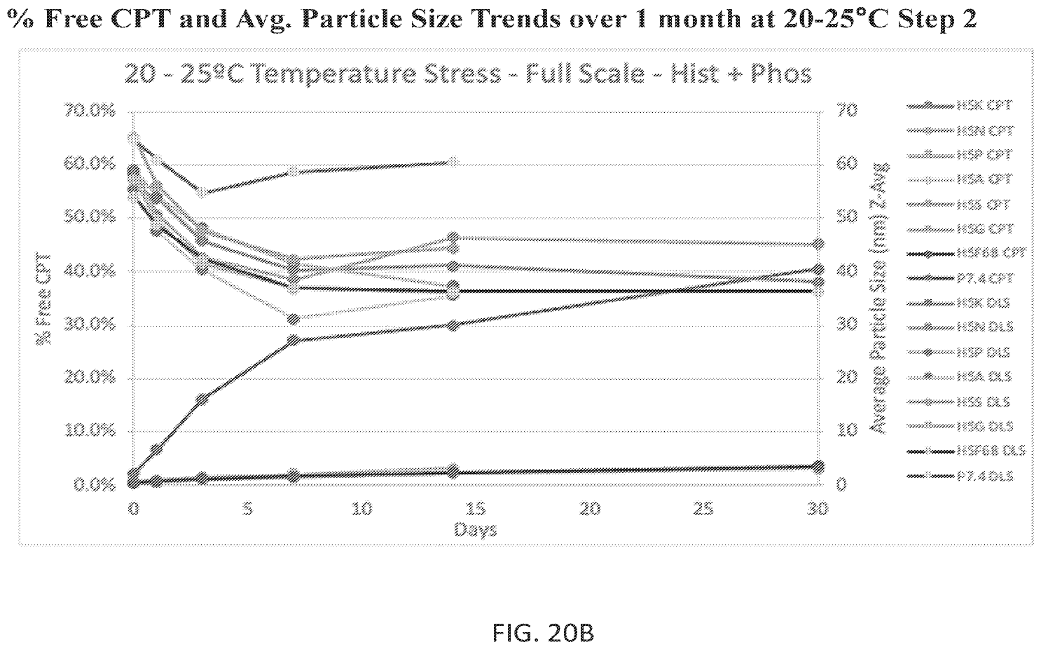

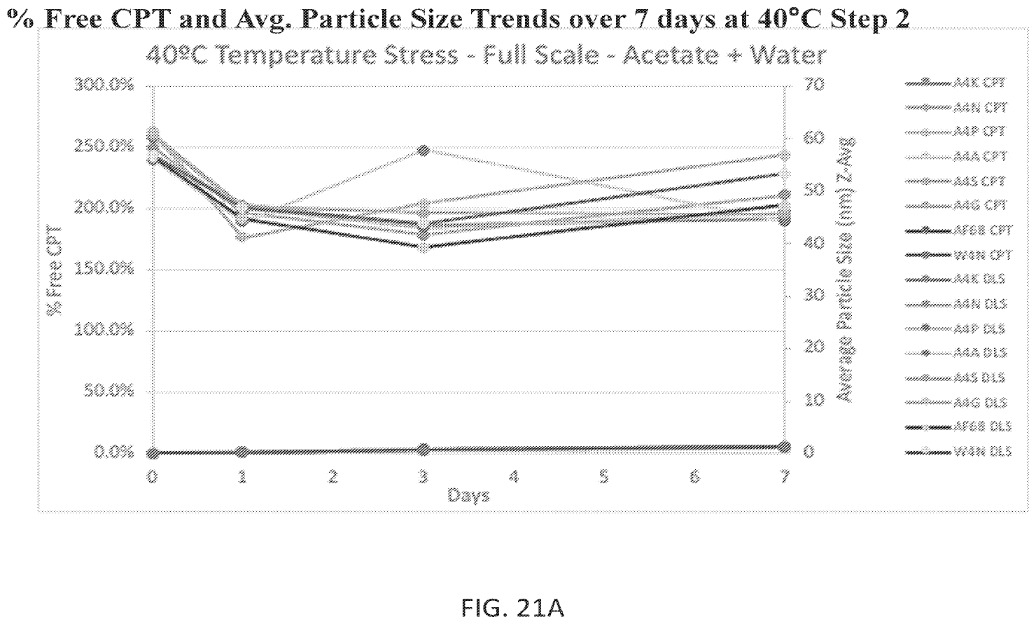

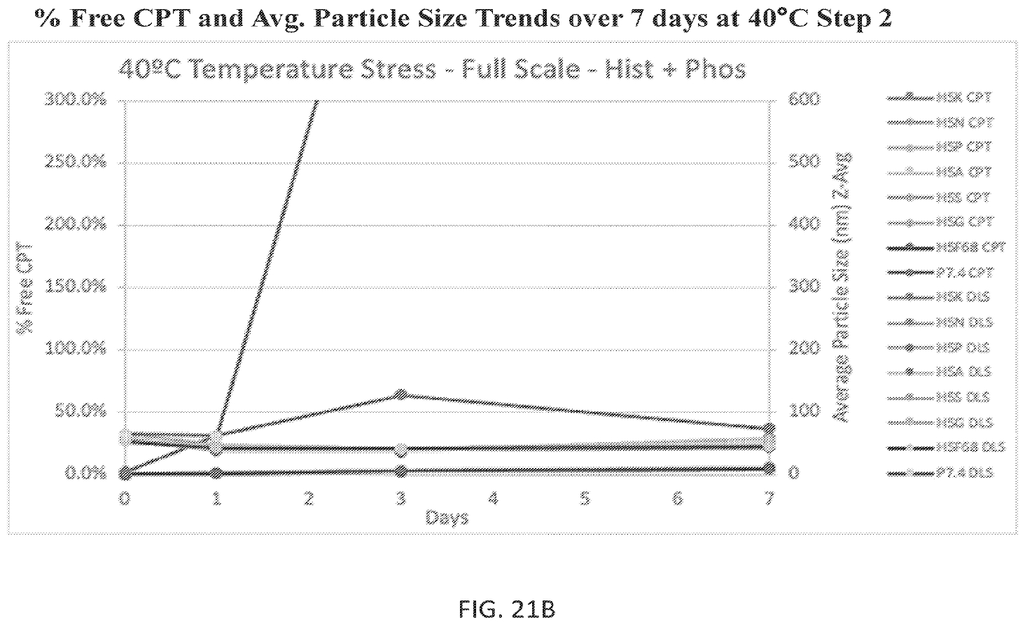

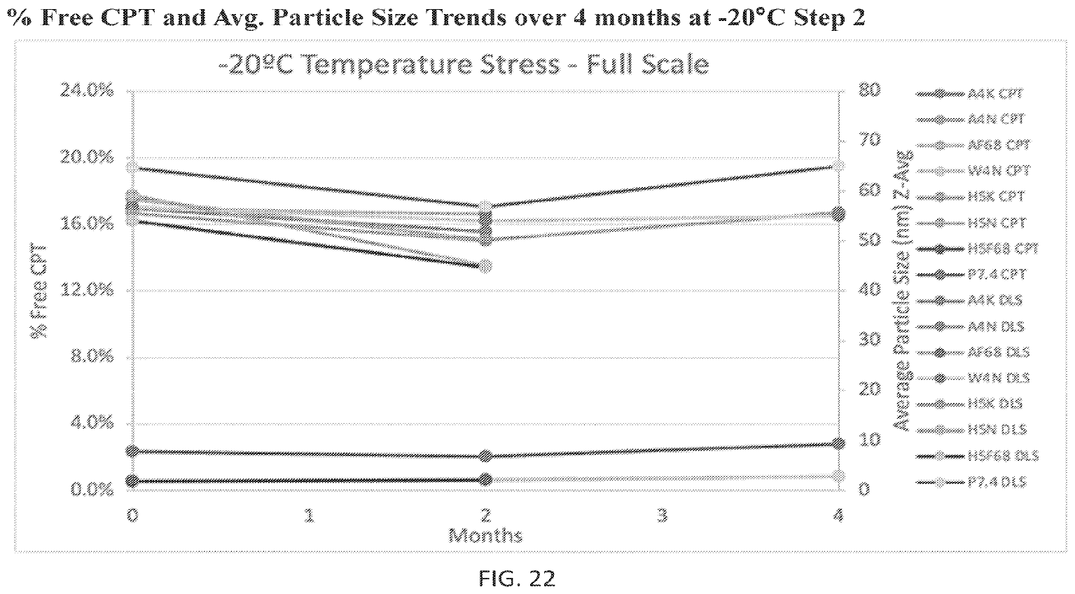

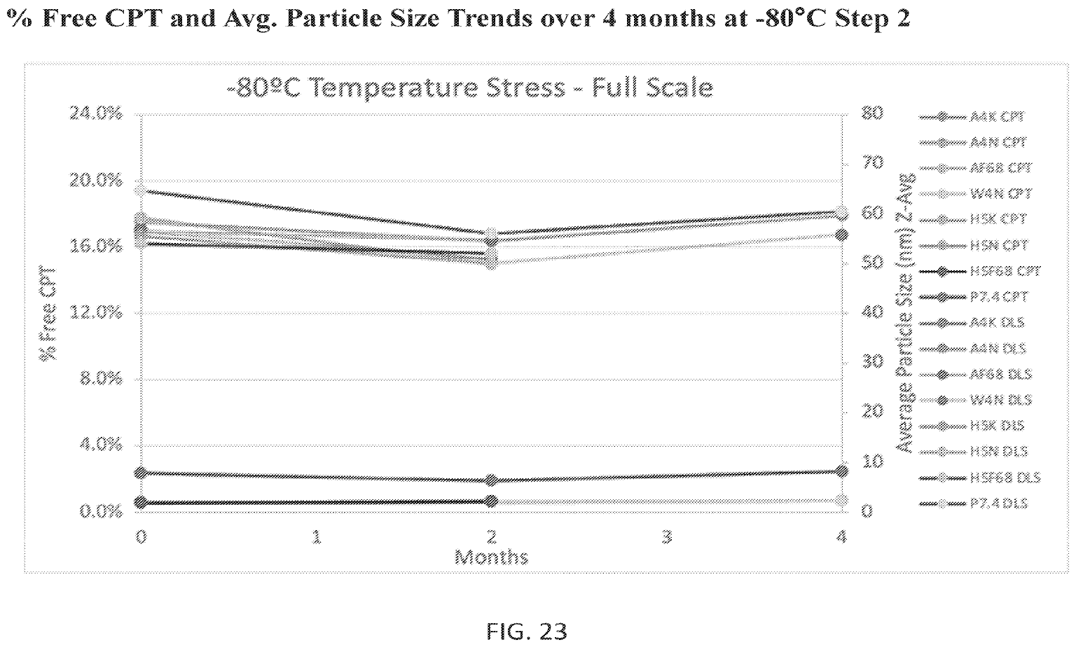

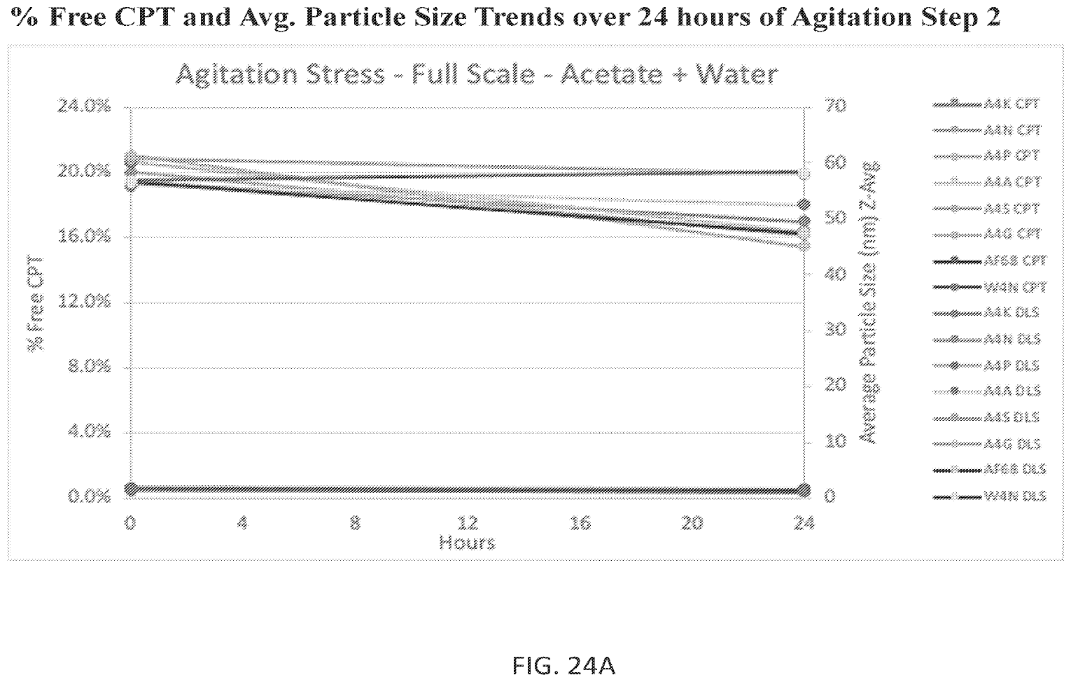

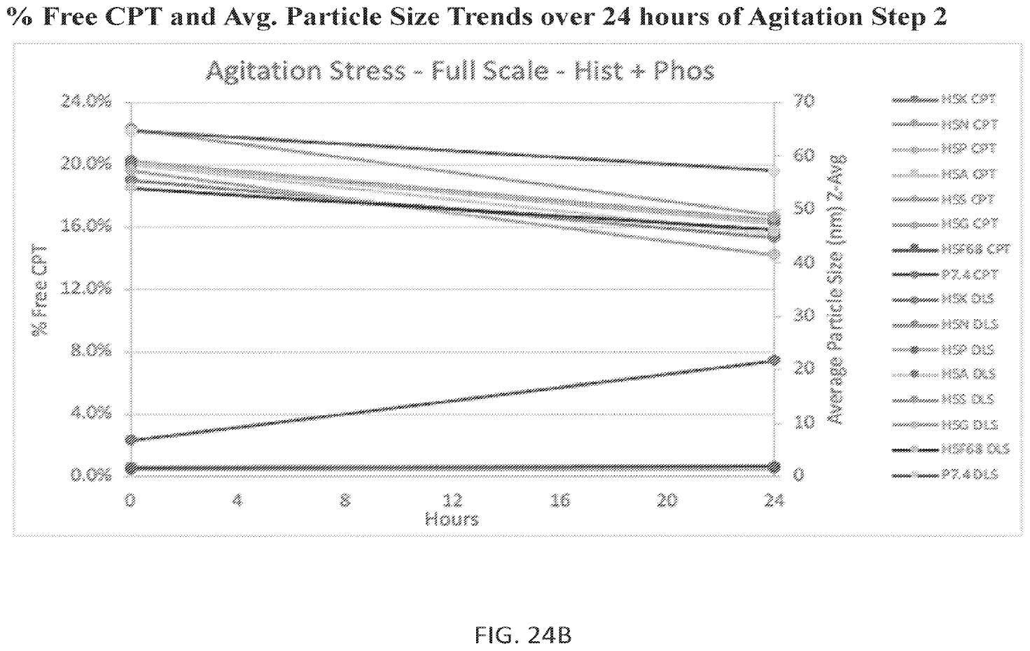

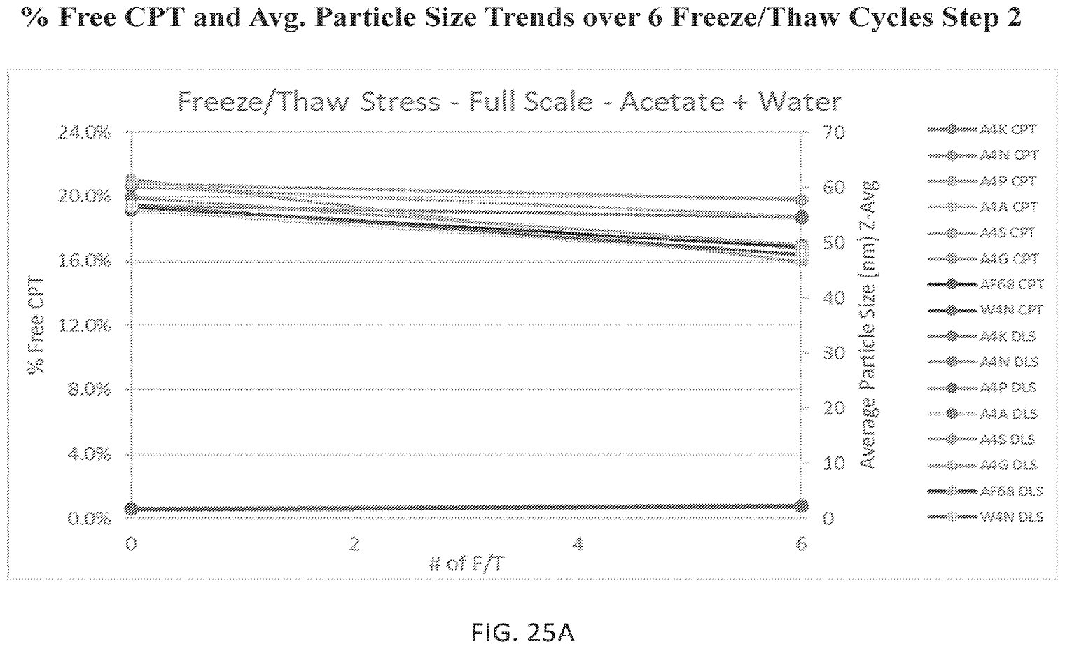

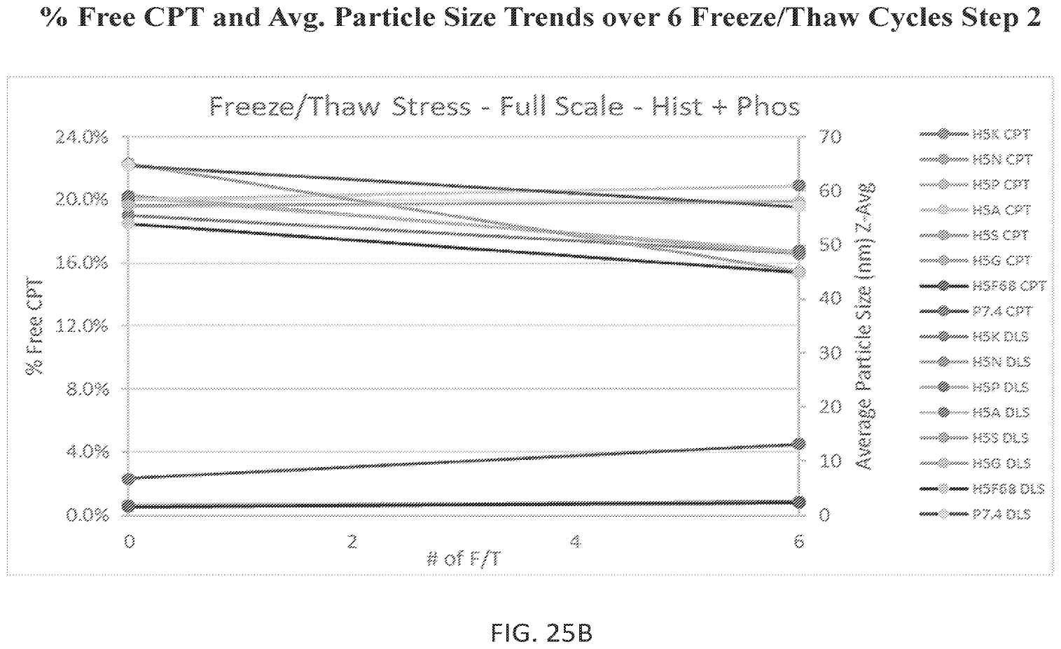

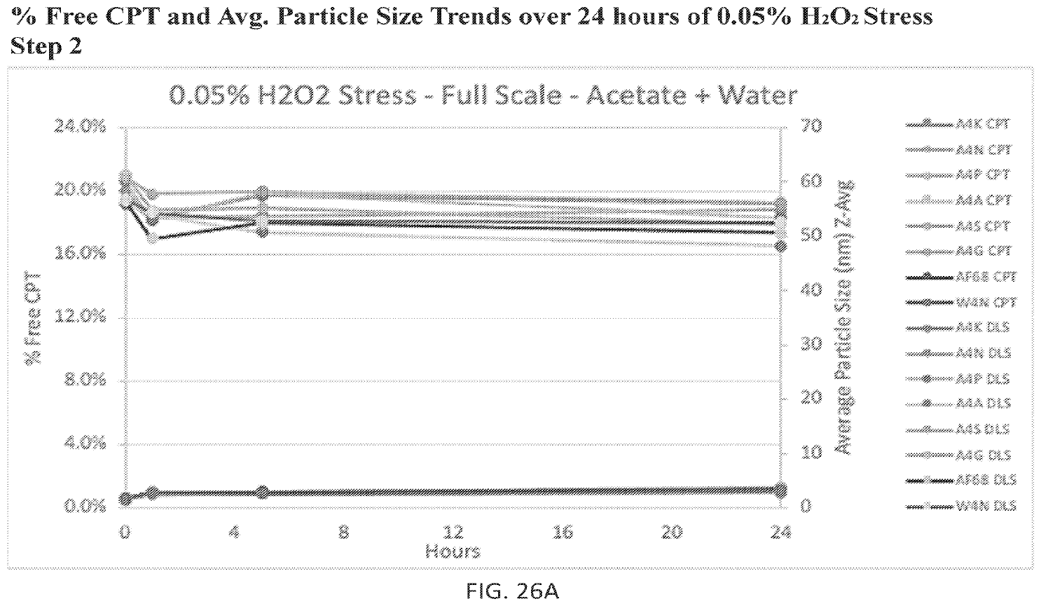

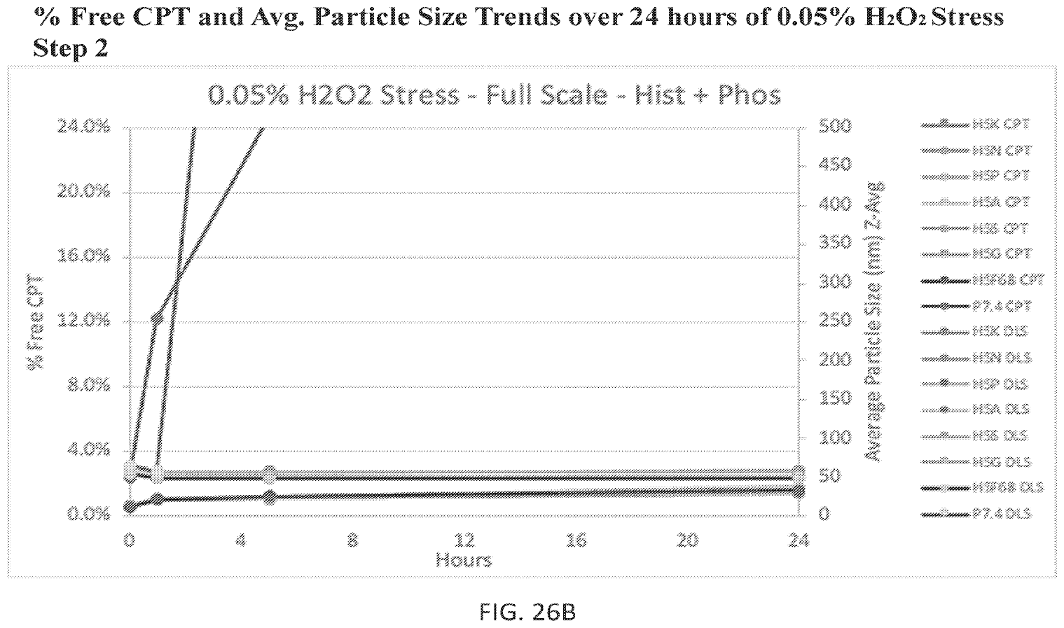

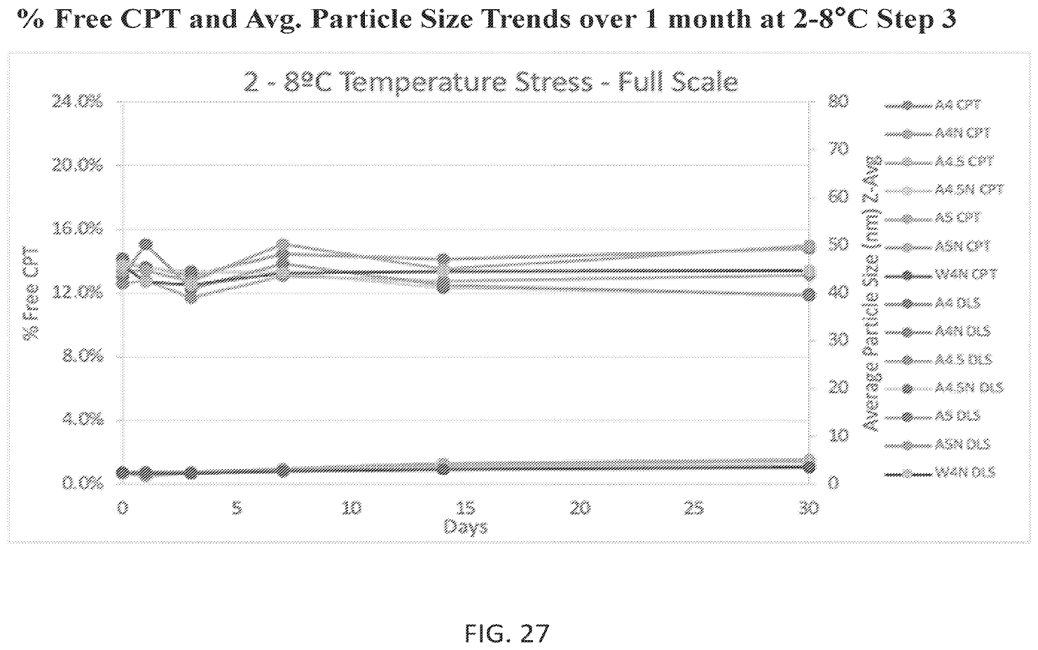

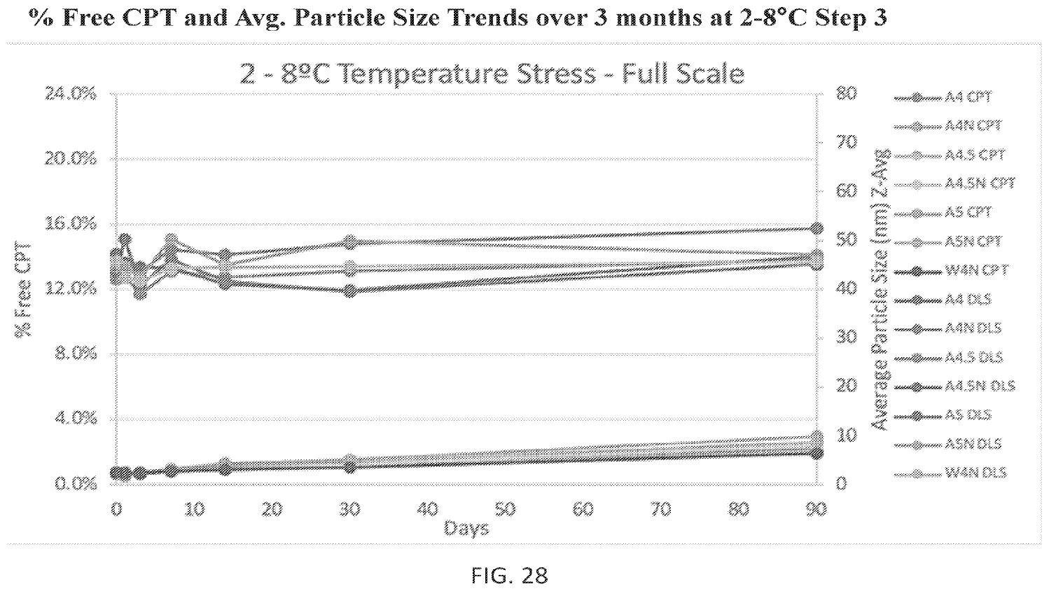

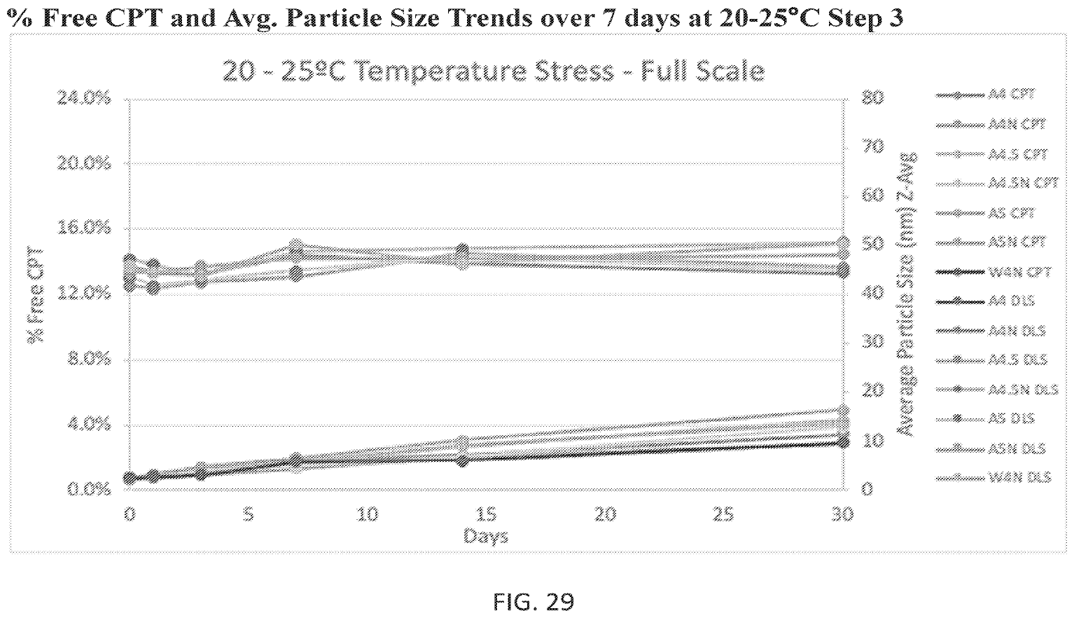

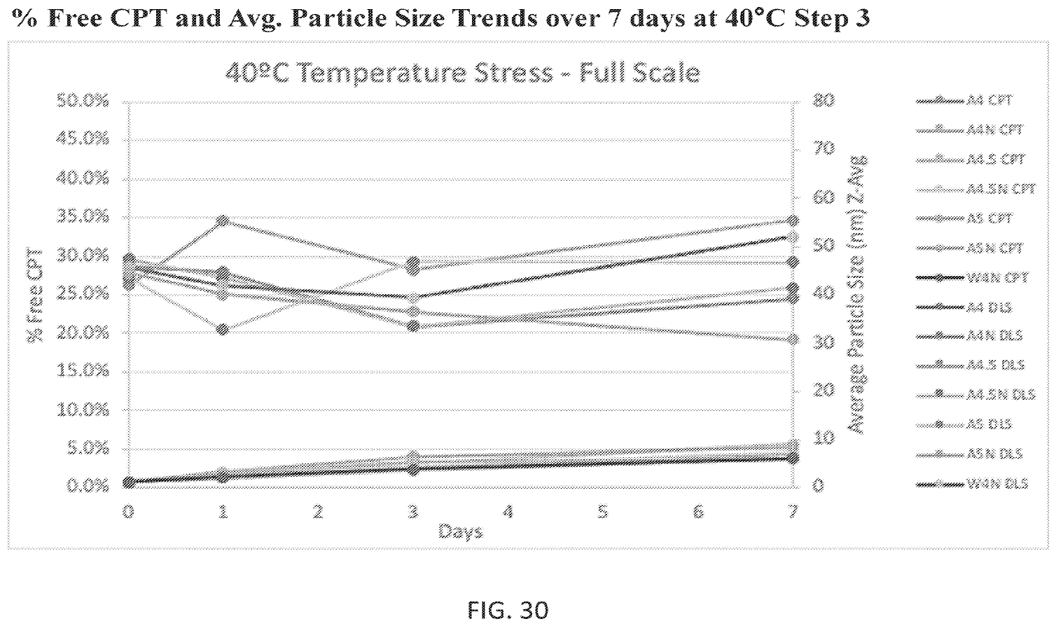

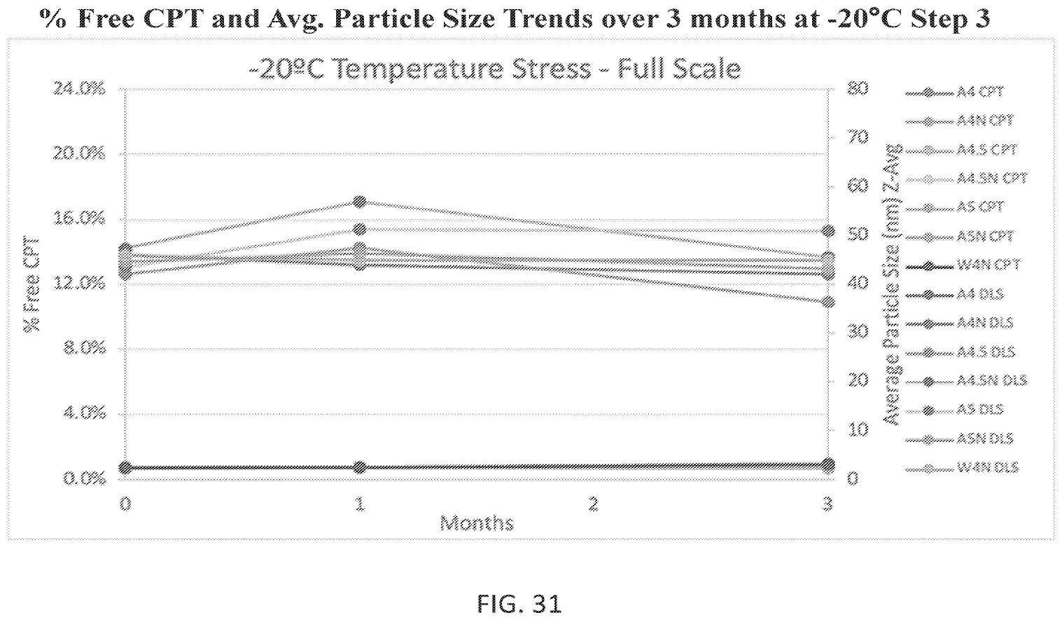

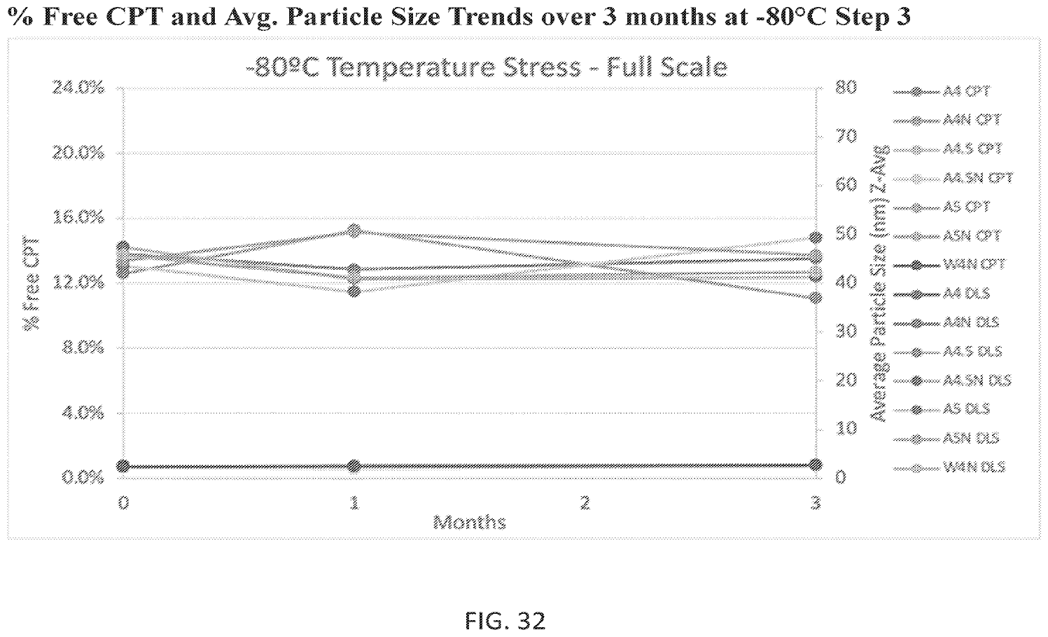

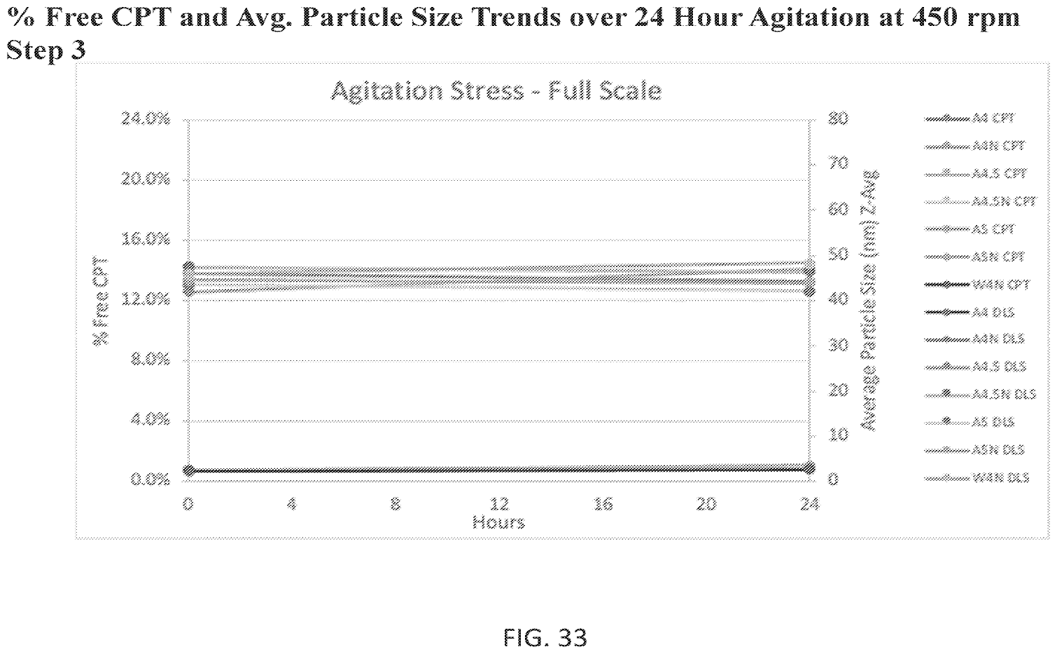

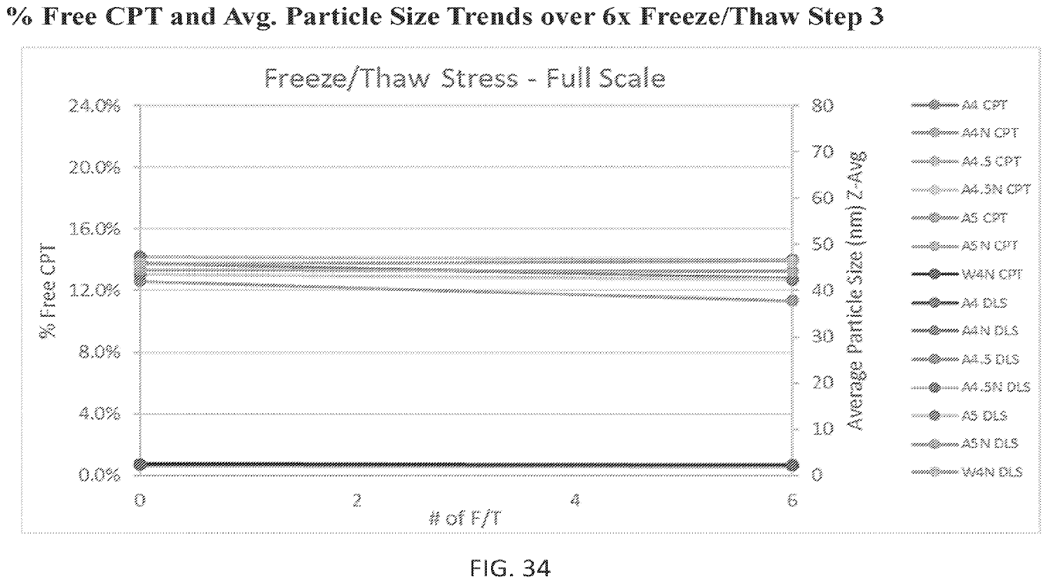

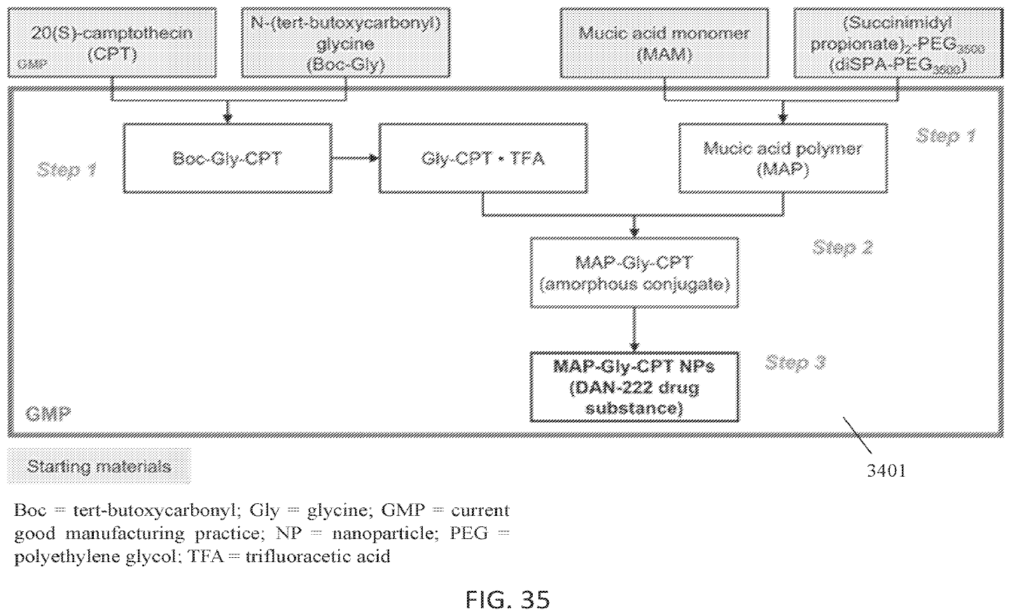

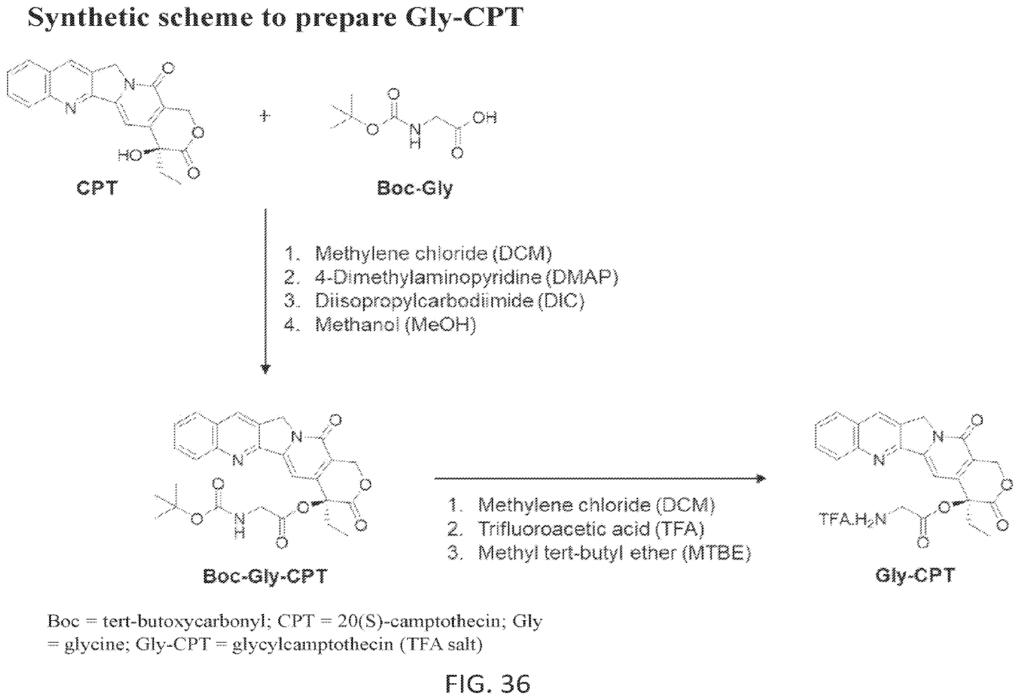

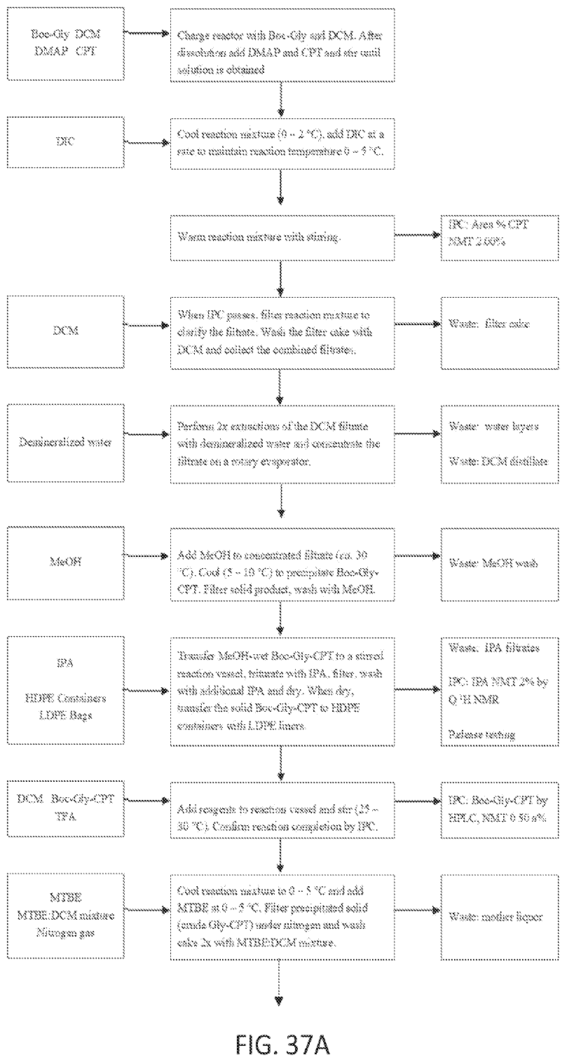

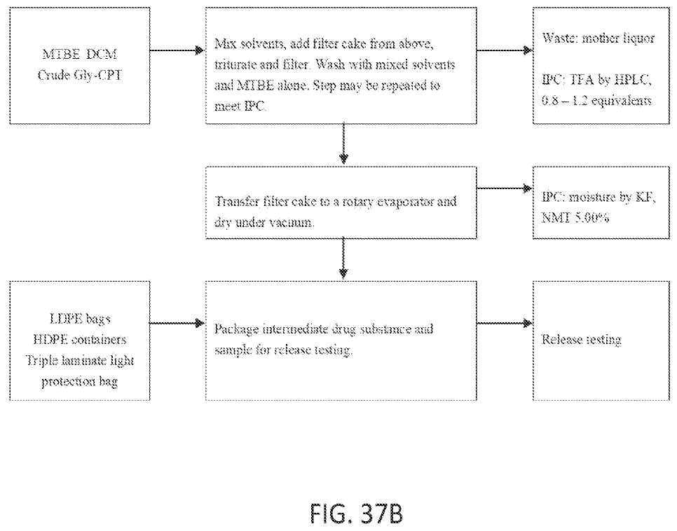

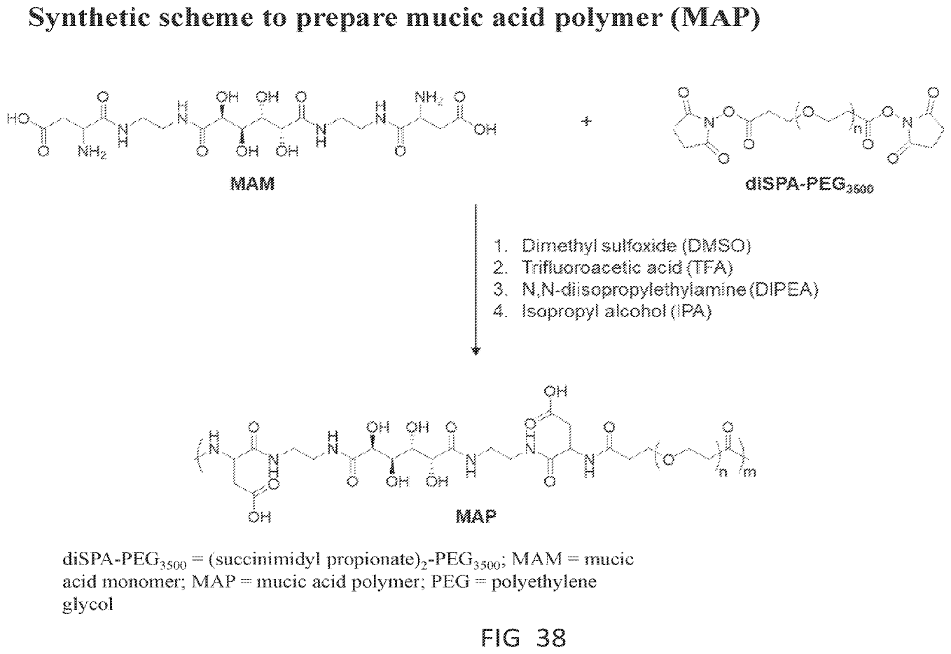

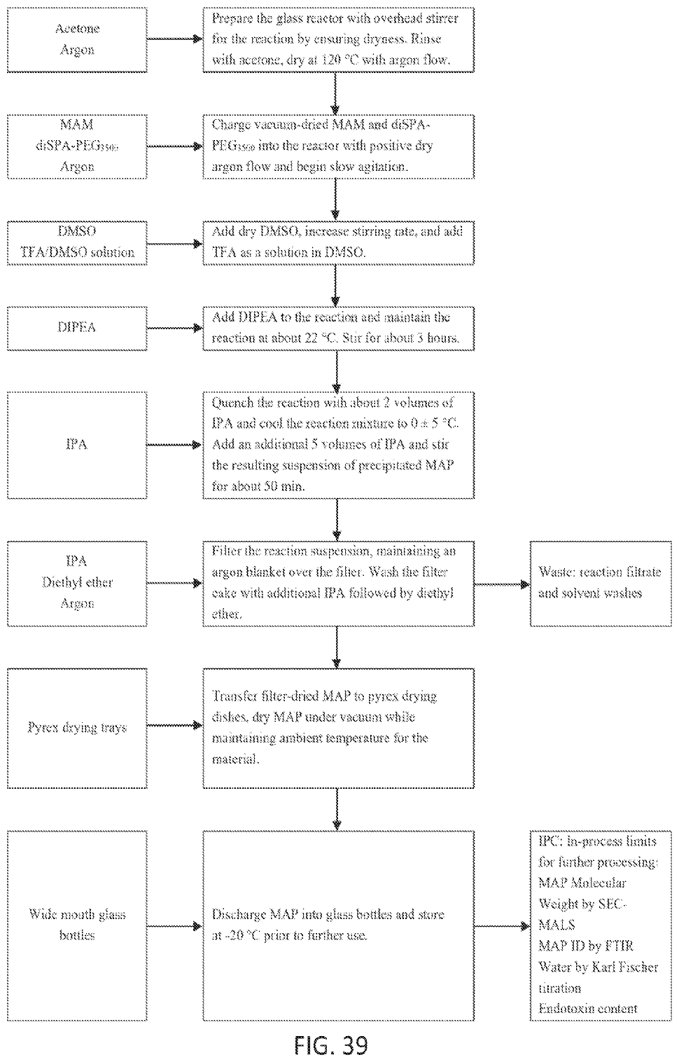

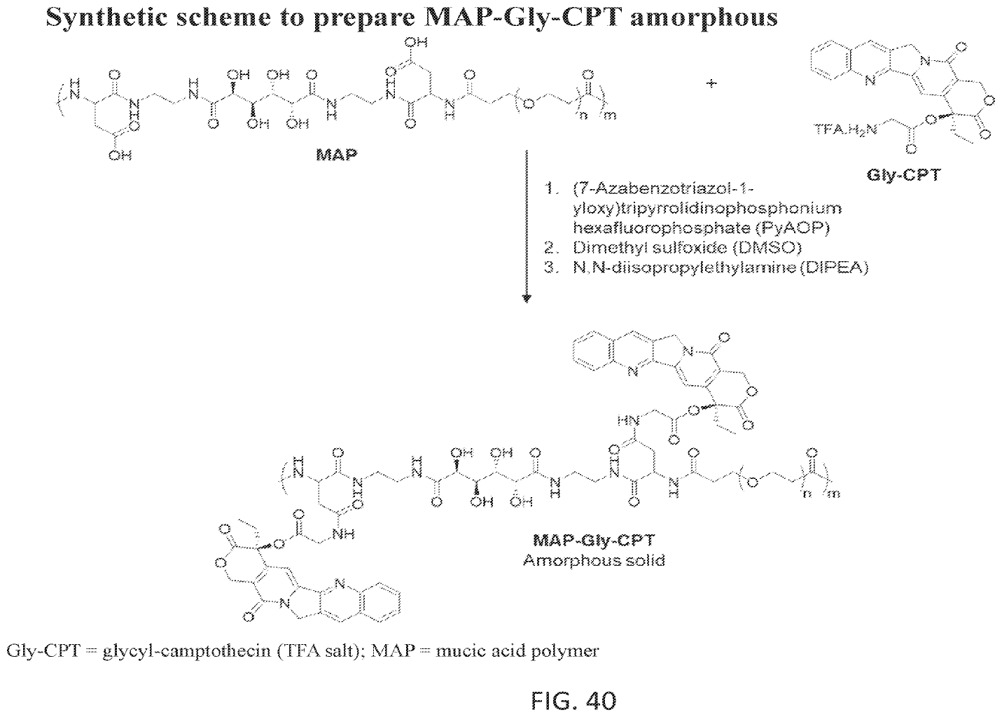

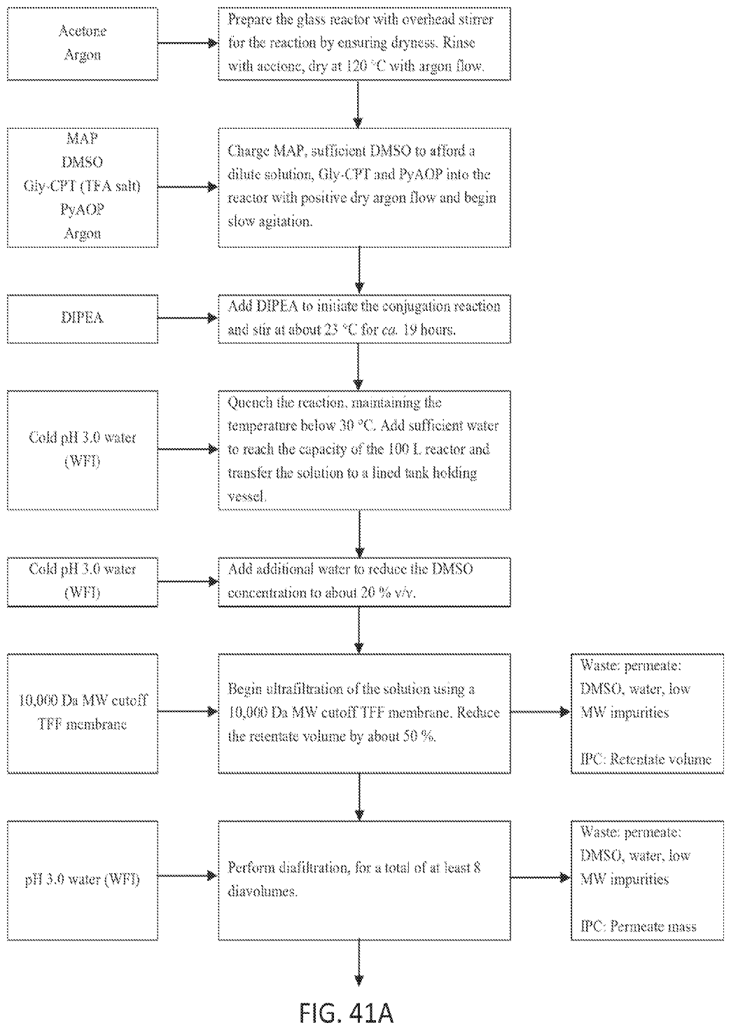

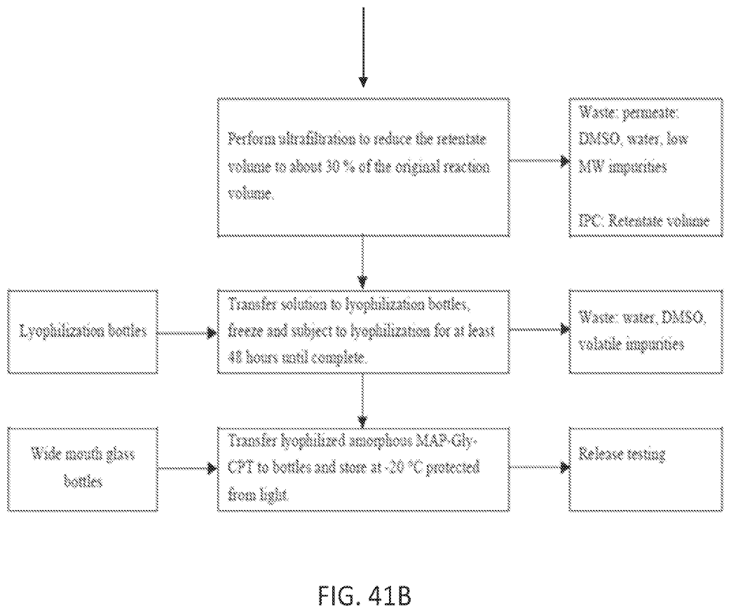

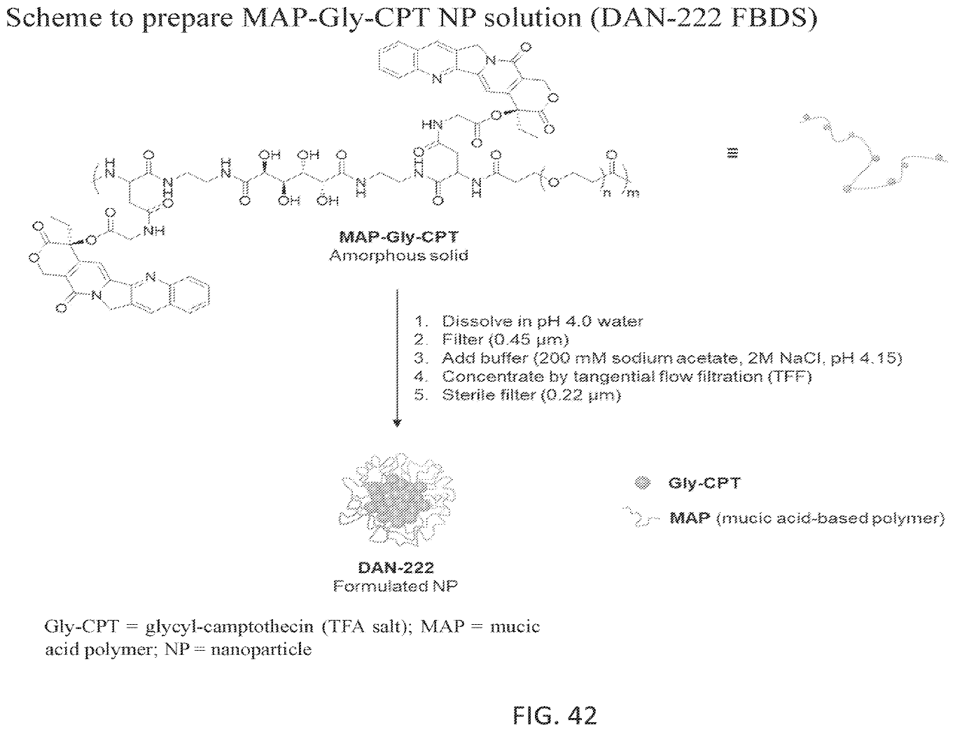

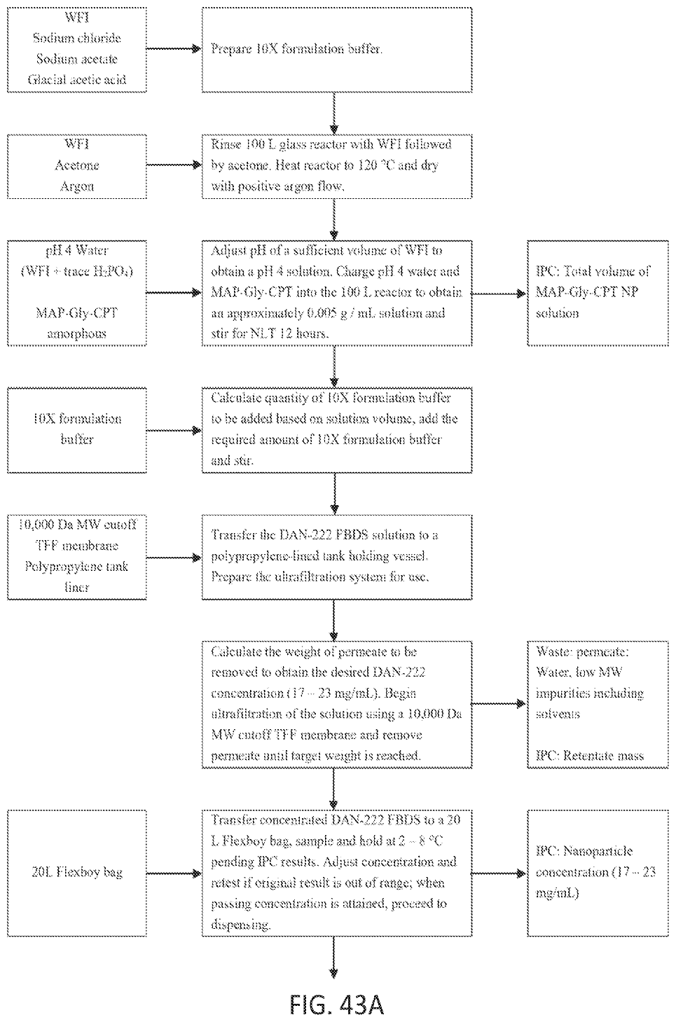

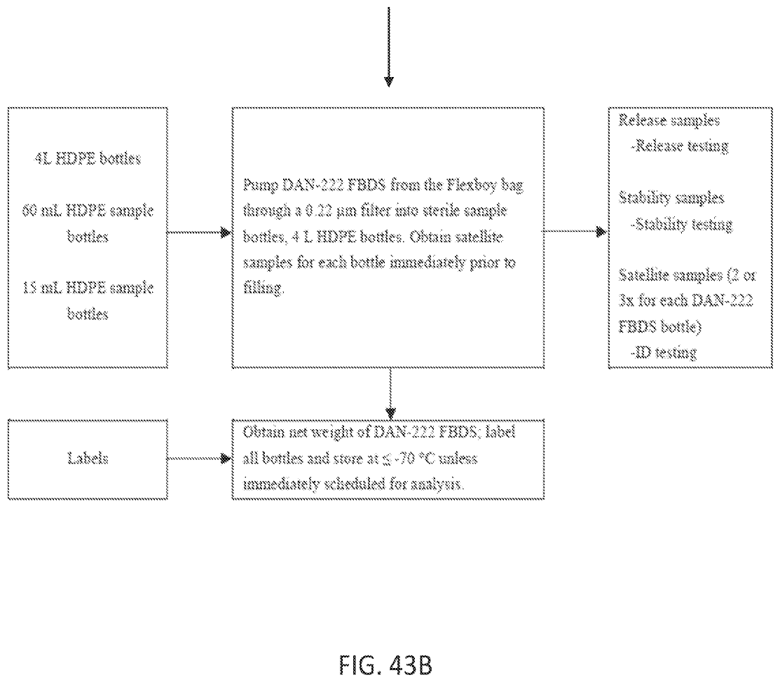

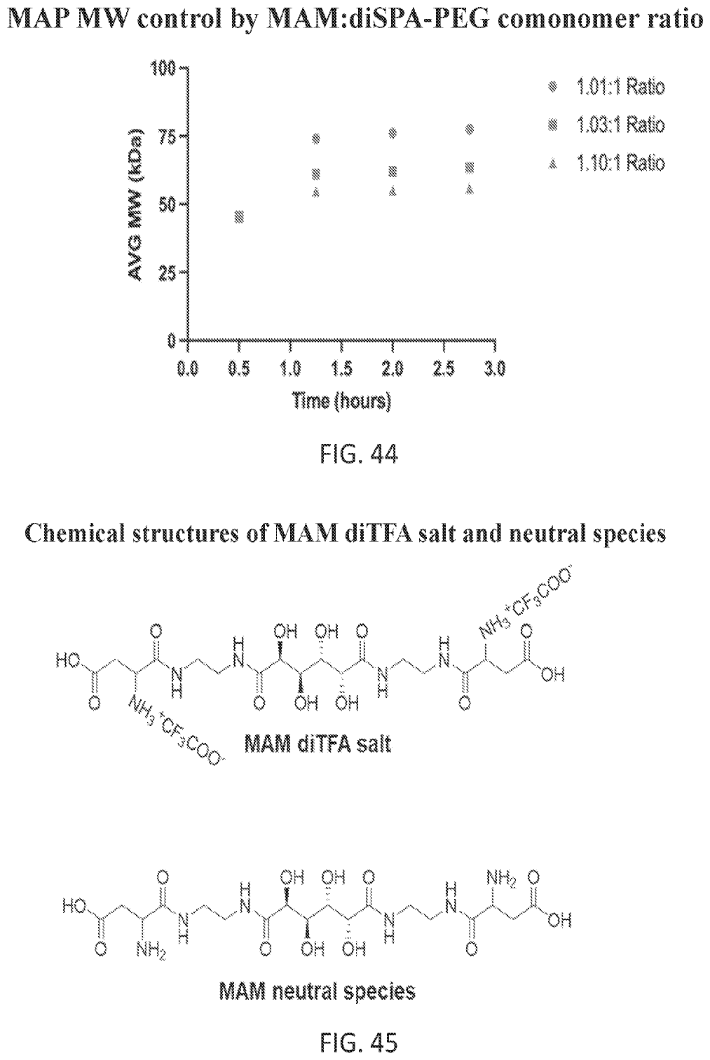

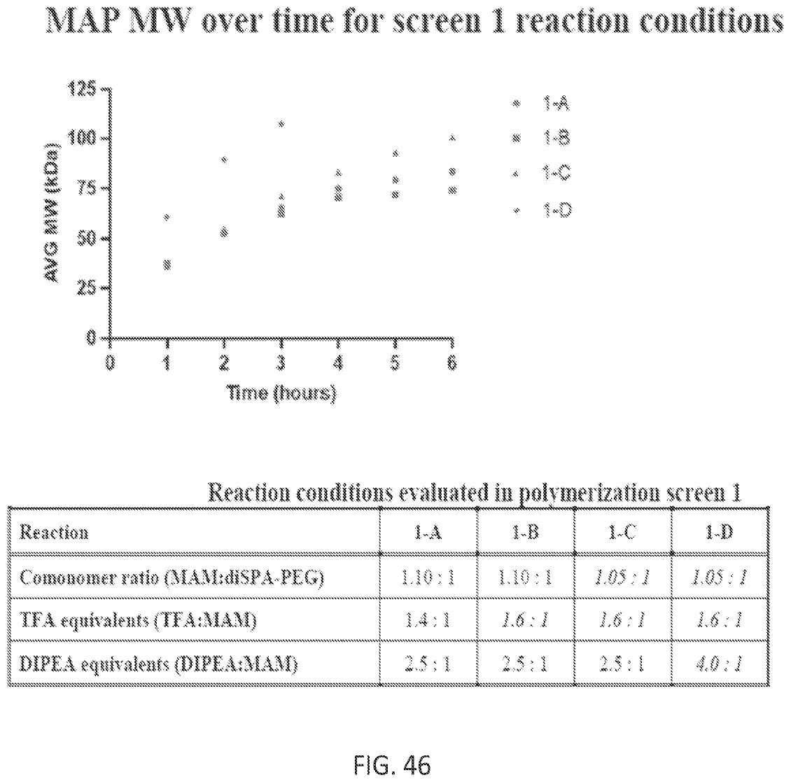

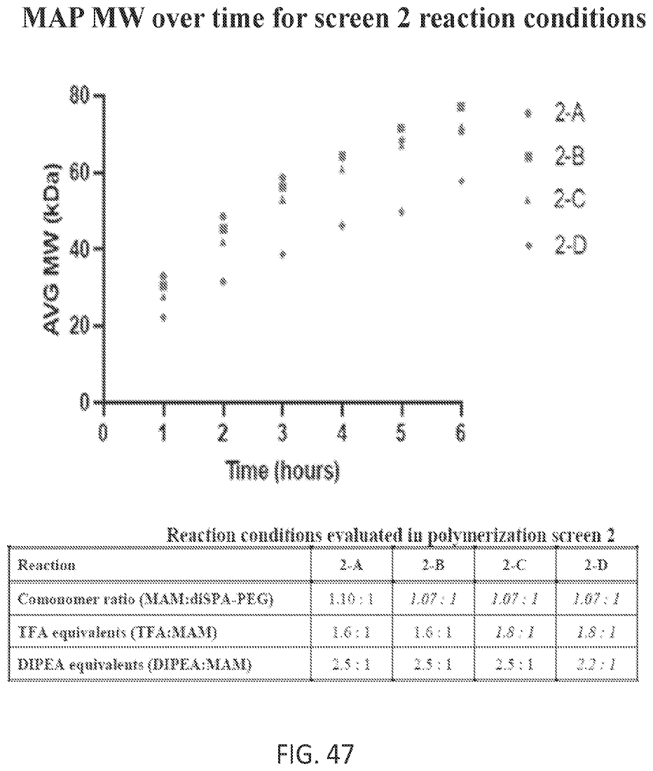

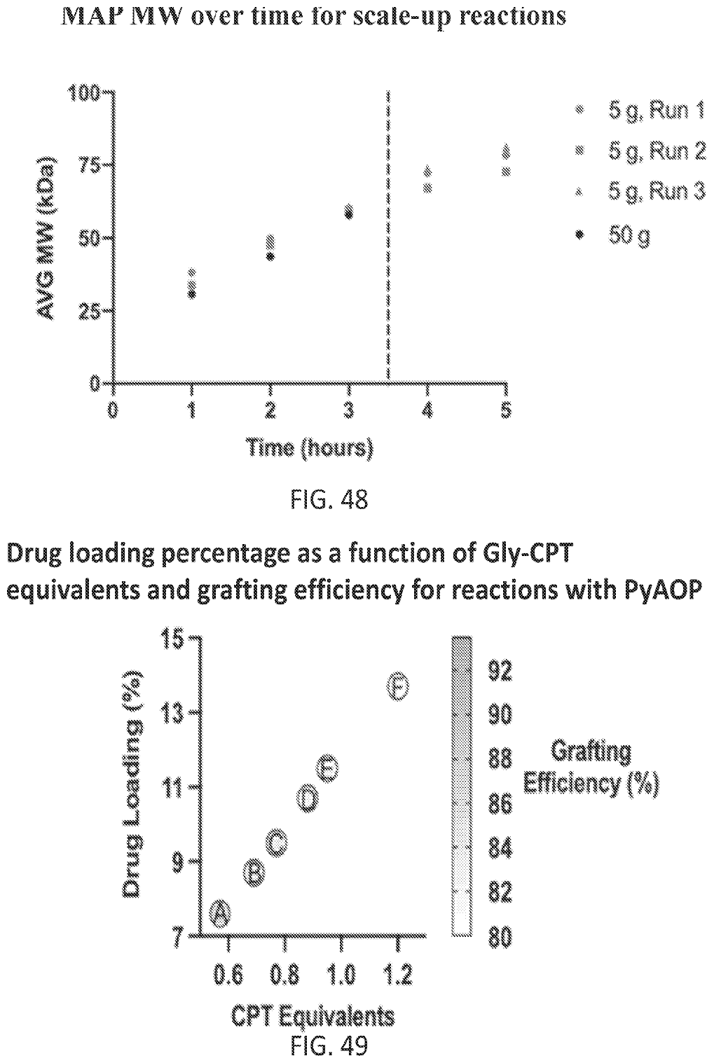

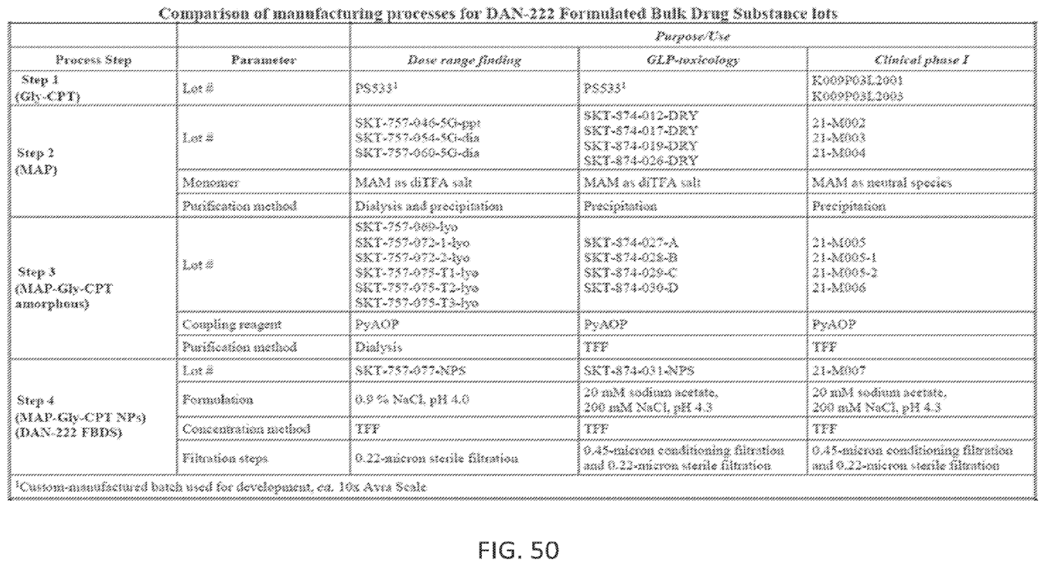

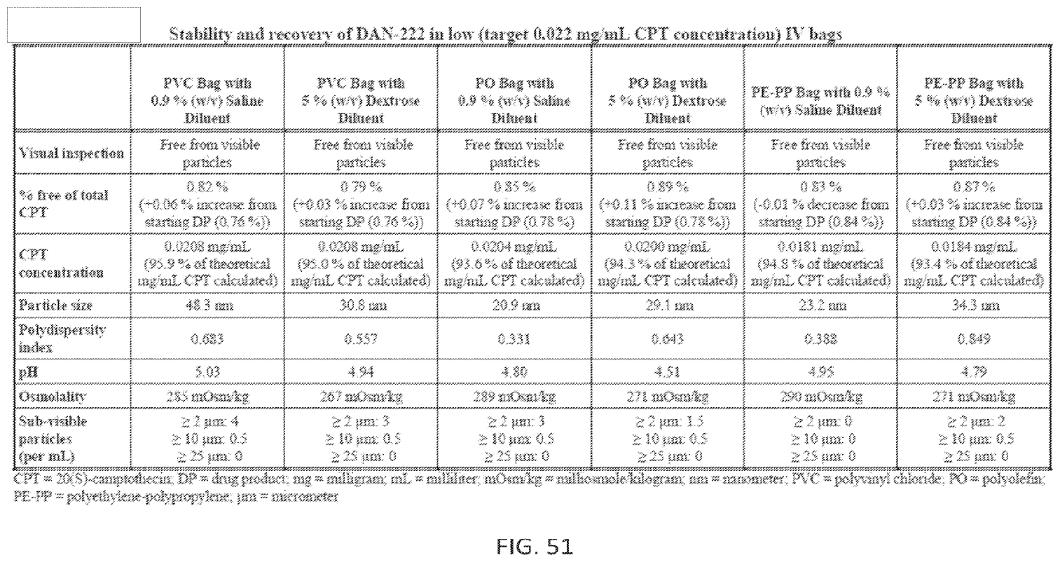

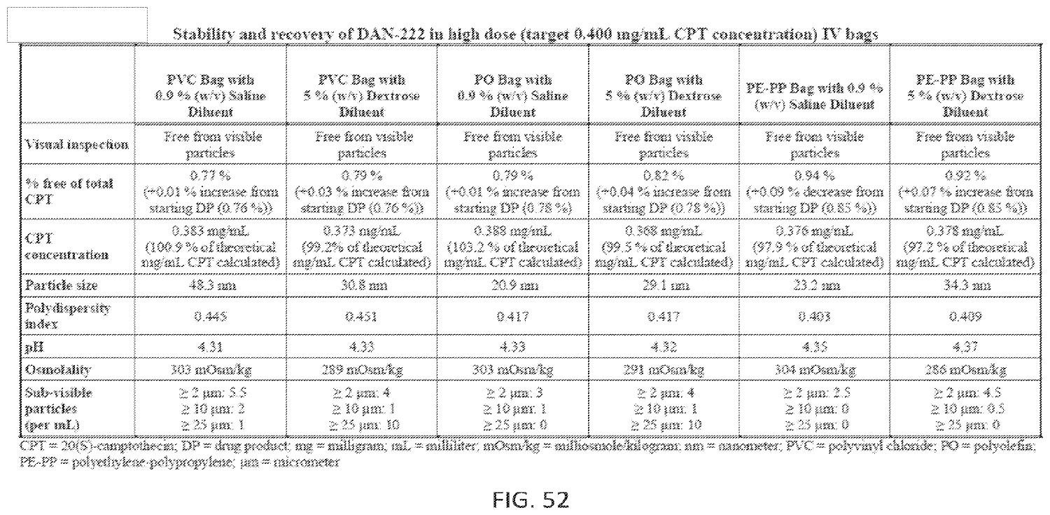

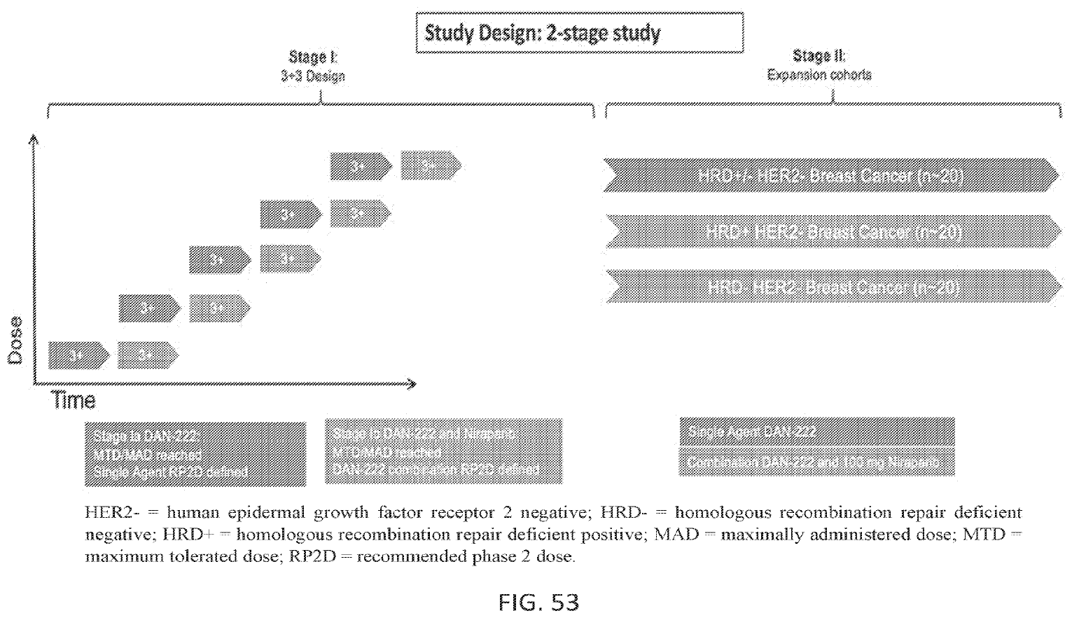

As shown in FIG. 1, when formulated in an aqueous media, DAN-222 self-assembles into NPs. When formulated as NPs, each NP is expected to consist of approximately two MAP-Gly-CPT strands. Consequently, an average DAN-222 NP will have a molecular weight of about 150,000 Da.

The inability to accurately repair complex DNA damage and resolve DNA replication stress leads to genomic instability and contributes to cancer etiology. For example, defects in homologous recombination (HR) repair are observed in various cancers. Breast cancer gene type 1 (BRCA1) and breast cancer gene type 2 (BRCA2) have long been known to encode proteins that play a key role in HR repair and mutations in one or both of these genes place patients at greater risk for development of breast, ovarian, prostate, melanoma, and pancreatic cancers.

Concurrently, this same inability to repair also makes cancer cells more vulnerable to DNA-damaging therapeutic agents. For example, tumor cells with defective HR repair show increased sensitivity to chemotherapeutic agents, which suggests that HR-proficient tumor cells might be sensitized to chemotherapeutics if HR repair could be therapeutically inactivated. Topoisomerase inhibitors, such as CPT, damage DNA by binding to topoisomerase I which prevents the unwinding of DNA required for replication, resulting in a stalled replication fork. This can prevent cancer cells for replicating and proliferating in a subject.

Camptothecin (CPT), the active ingredient in DAN-222, is a naturally occurring, pentacyclic quinolone alkaloid isolated from the bark of Camptotheca acuminata. CPT is a topoisomerase I inhibitor that acts by forming a ternary complex with topoisomerase I and DNA during replication and transcription in tumor cells, which blocks the re-ligation of DNA, resulting in torsional tension downstream of RNA polymerase to cause DNA strand breaks.

Treatment of many brain disorders such as brain cancer and metastases to the brain requires that therapeutic molecules be delivered to the brain. Direct delivery of therapeutics to the brain poses severe risks to the subject (e.g., breaching the skull), and cannot be feasibly carried out on a continuing basis, as is required for most chemotherapeutic treatments. However, systemic delivery (e.g., via the bloodstream) will not efficiently deliver molecules to the brain, because of the existence of the blood-brain barrier (BBB); a tightly-joined layer of endothelial cells lining the blood vessels of the brain. A similar permeation barrier, known as the blood-tumor barrier (BTB) exists in certain solid tumors.

Cancers of the breast frequently metastasize to the brain and these brain metastases could be treated with chemotherapeutic molecules used for treatment of breast cancer, if the therapeutic could be delivered to the brain (across the BBB) or a tumor (across the BTB) in sufficient concentrations. One such chemotherapeutic is CPT.

Unfortunately, while CPT is known for its anti-tumor activity over a wide spectrum of human cancers, it has poor water solubility, low plasma stability, and dose-limiting toxicity. Moreover, as described, the BBB and/or BTB may inhibit the ability of cells to take up CPT.

However, as discovered by the present inventors, DAN-222 is more efficacious than other topoisomerase I inhibitors, including free CPT, due to its accumulation in tumors through an enhanced permeability and retention (EPR) effect, which leads to a greater and prolonged topoisomerase I inhibition. The EPR effect relies on the differences in vasculature between healthy tissues and tumors. The DAN-222 nanoparticles encapsulate the CPT and are able to pass through the leaky walls of tumor neovasculature, while concurrently not passing through the tighter walls of healthy blood vessels. Moreover, encapsulation in the DAN-222 nanoparticles protects the CPT from conversion of its active lactone form to the inactive carboxylate form in the serum, thereby preventing rapid blood clearance of NP-encapsulated CPT.

For example, as described herein, the anti-tumor activity of DAN-222 was evaluated in several tumor xenograft studies, including cell lines from both breast (BT474, MDA-MB-23, MBA-MB-436) and ovarian (OVCAR3) tumors. DAN-222 had significantly greater efficacy and sustained tumor inhibition as compared to other topoisomerase I inhibitors, including free CPT and irinotecan (an FDA approved topoisomerase I inhibitor for cancer treatment), in all tumor xenograft models studied. The biodistribution of DAN-222 compared to irinotecan was evaluated in wild-type mice demonstrating a significantly lower exposure in bone marrow of DAN-222 relative to irinotecan.

Moreover, not only does DAN-222 show efficacy, but it is also designed to reduce or eliminate unwanted risks and side-effects associated with free CPT. The DAN-222 construct is designed to limit the acute toxicity of CPT by attaching it to the nanoparticle polymer, thereby controlling the release of the active CPT moiety. Release of free CPT is caused by hydrolysis, its release can be controlled in part by the nanoparticle construct, and also by using the DAN-222 formulations described herein.

The present invention also includes compositions and methods that combine DAN-222 with a poly (ADP-ribose) polymerase (PARP) inhibitor, such as niraparib. Recently PARPs have emerged as a new target in cancer treatment. PARP inhibitors exploit genomic instability as well as deficiencies in DNA repair pathways. Four PARP inhibitors, olaparib, rucaparib, niraparib, and talazoparib, are currently approved by the United States Food and Drug Administration

(FDA). Of these, olaparib and talazoparib are approved for HER2-negative locally advanced or mBC with germline BRCA1/2 mutations. Currently, PARP inhibitors are considered the most important therapeutic drugs for the BRCA mutations seen in TNBC and BRCA-mutated estrogen receptor-positive breast cancers.

As explained, topoisomerase inhibitors, such as CPT, damage DNA by binding to topoisomerase I and preventing the unwinding of DNA required for replication. This results in a stalled replication fork that can be repaired by poly (ADP-ribose) polymerase (PARP). PARP inhibitors block PARP enzyme DNA repair activity and have been shown to induce apoptosis in tumor cells. Thus, in addition to direct tumor killing, inhibition of PARP also increases the cytotoxicity of chemotherapeutic drugs and DNA-damaging agents such as topoisomerase inhibitors, such as CPT.

In certain aspects, the present invention includes formulations useful for treating cancer, cancer metastases and/or disorders of the brain and central nervous system. In certain aspects, the cancer is selected from breast cancer, lung cancer, colon cancer, ovarian cancer, melanoma, bladder cancer, kidney cancer, liver cancer, salivary gland cancer, stomach cancer, nerve Glioma, thyroid cancer, thymic cancer, epithelial cancer, head and neck cancer, stomach cancer and pancreatic cancer. In preferred aspects, the cancer is breast cancer or ovarian cancer. In certain aspects, the cancer is HER2-negative metastatic breast cancer (mBC). In certain aspects, the HER2-negative mBC is a homologous recombination deficient (HRD) positive or negative HER2-negative mBC. In additional aspects, the cancer is a brain metastasis of a breast tumor.

### Abbreviations and Definitions

Reference to alcohols, aldehydes, amines, carboxylic acids, ketones, or other similarly reactive functional groups also includes their protected analogs. For example, reference to hydroxy or alcohol also includes those substituents wherein the hydroxy is protected by acetyl (Ac), benzoyl (Bz), benzyl (Bn, Bnl), b-Methoxyethoxymethyl ether (MEM), dimethoxytrityl, [bis-(4-methoxyphenyl)phenylmethyl] (DMT), methoxymethyl ether (MOM), methoxytrityl [(4-methoxyphenyl)diphenylmethyl, MMT), p-methoxybenzyl ether (PMB), methylthiomethyl ether, pivaloyl (Piv), tetrahydropyranyl (THP), tetrahydrofuran (THF), trityl (triphenylmethyl, Tr), silyl ether (most popular ones include trimethyl silyl (TMS), tert-butyldimethylsilyl (TBDMS), tri-iso-propylsilyloxymethyl (TOM), and triisopropyl silyl (TIPS) ethers), ethoxyethyl ethers (EE). Reference to amines also includes those substituents wherein the amine is protected by a BOC glycine, carbobenzyloxy (Cbz), p-methoxybenzyl carbonyl (Moz or MeOZ), tert-butyloxycarbonyl (BOC), 9-fluorenylmethyloxycarbonyl (FMOC), acetyl (Ac), benzoyl (Bz), benzyl (Bn), carbamate, p-methoxybenzyl (PMB), 3,4-dimethoxybenzyl (DMPM), p-methoxyphenyl (PMP), tosyl (Ts) group, or sulfonamide (Nosyl & Nps) group. Reference to substituents containing a carbonyl group also includes those substituents wherein the carbonyl is protected by an acetal or ketal, acylal, or diathane group. Reference to substituents containing a carboxylic acid or carboxylate group also includes those substituents wherein the carboxylic acid or carboxylate group is protected by its methyl ester, benzyl ester, tert-butyl ester, an ester of 2,6-disubstituted phenol (e.g., 2,6-dimethylphenol, 2,6-diisopropylphenol, 2,6-di-tert-butylphenol), a silyl ester, an orthoester, or an oxazoline

### ABBREVIATIONS

ASCO American Society of Clinical Oncology
AUCO-last area under the curve from time 0 to the last measurable concentration
BOR best overall response
BRCA breast cancer gene
BRCA1 breast cancer gene type 1
BRCA2 breast cancer gene type 2
CAP College of American Pathologists
CBR clinical benefit rate
CI confidence interval
CL clearance
CL/F clearance after oral administration
Cmax maximum observed plasma concentration
Cmin minimum observed plasma concentration
CPT camptothecin
CR complete response
CRA Clinical Research Associate
CT computed tomography
CTC circulating tumor count
CYP3A4 cytochrome P450 3A4
DAN-222 a mucic acid polymer nanoparticle that contains 20 (S)-camptothecin
DAS dose-limiting toxicity evaluable analysis set
DCR disease control rate
DLT dose-limiting toxicities
DOR duration of response
EAS evaluable analysis set
ECG electrocardiogram
ECOG Eastern Cooperative Oncology Group
eCRF electronic case report form
EDC electronic data capture
EOT End-of-Treatment
EPR enhanced permeability and retention
FAS full analysis set
FDA Food and Drug Administration
GCP Good Clinical Practice
G-CSF granulocyte colony-stimulating factor
HER2 human epidermal growth factor receptor 2
HNSTD highest non-severely toxic dose level
HR homologous recombination
HRD homologous recombination repair deficient
ICF informed consent form
ICH International Council for Harmonisation
ILD interstitial lung disease
IRB/IEC Institutional Review Board/Independent Ethics Committee
IV intravenous
MAD maximally administered dose
mBC metastatic breast cancer
MDS/AML myelodysplastic syndrome/acute myeloid leukemia
MedDRA Medical Dictionary for Regulatory Activities
MRI magnetic resonance imaging
MTD maximum tolerated dose
NCI CTCAE National Cancer Institute Common Terminology Criteria for Adverse Events
NP nanoparticle
ORR objective response rate
PARP poly (ADP-ribose) polymerase
PD pharmacodynamic
PEG polyethylene glycol
PFS progression-free survival
PK pharmacokinetic(s)
PPAS per-protocol analysis set
PR partial response
PRES posterior reversible encephalopathy syndrome
QD every day
QW every week
RECIST v1.1 Response Evaluation Criteria in Solid Tumors, Version 1.1
RP2D recommended phase 2 dose
SAE serious adverse event
SAS safety analysis set
SAP statistical analysis plan
SD stable disease
SRT Safety Review Team
t1/2 terminal elimination half-life
TEAE treatment-emergent adverse event
TNBC triple negative breast cancer
ULN upper limit of normal
V volume of distribution
Vz/F volume of distribution after oral administration

When a value is expressed as an approximation by use of the descriptor “about,” it will be understood that the particular value forms another embodiment. In general, use of the term “about” indicates approximations that can vary depending on the desired properties sought to be obtained by the disclosed subject matter and is to be interpreted in the specific context in which it is used, based on its function. The person skilled in the art will be able to interpret this as a matter of routine. In some cases, the number of significant figures used for a particular value may be one non-limiting method of determining the extent of the word “about.” In other cases, the gradations used in a series of values may be used to determine the intended range available to the term “about” for each value. Where present, all ranges are inclusive and combinable. That is, references to values stated in ranges include every value within that range.

It is to be appreciated that certain features of the disclosure which are, for clarity, described herein in the context of separate embodiments, may also be provided in combination in a single embodiment. That is, unless obviously incompatible or specifically excluded, each individual embodiment is deemed to be combinable with any other embodiment s) and such a combination is considered to be another embodiment. Conversely, various features of the disclosure that are, for brevity, described in the context of a single embodiment, may also be provided separately or in any sub-combination. Finally, while an embodiment may be described as part of a series of steps or part of a more general structure, each said step may also be considered an independent embodiment in itself, combinable with others.

As used herein, a “pure isomeric” compound or “isomerically pure” compound is substantially free of other isomers of the compound. The term “pure isomeric” compound or “isomerically pure” denotes that the compound comprises at least 95% by weight, at least 96% by weight, at least 97% by weight, at least 98% by weight, at least 99% by weight, at least 99.5% by weight, at least 99.6% by weight, at least 99.7% by weight, at least 99.8% by weight, or at least 99.9% by weight of the compound with the specified structure. In certain embodiments, the weights are based upon total weight of all isomers of the compound.

As used herein, a “pure stereoisomeric” compound or “stereoisomerically pure” compound is substantially free of other stereoisomers of the compound. Thus, the composition is substantially free of isomers that differ at any chiral center. If the compound has multiple chiral centers, a substantial majority of the composition contains compounds having identical stereochemistry at all of the chiral centers. The term “pure stereoisomeric” compound or “stereoisomerically pure” denotes that the compound comprises at least 95% by weight, at least 96% by weight, at least 97% by weight, at least 98% by weight, at least 99% by weight, at least 99.5% by weight, at least 99.6% by weight, at least 99.7% by weight, at least 99.8% by weight, or at least 99.9% by weight of the compound with the specified stereochemistry. In certain embodiments, the weights are based upon total weight of all stereoisomers of the compound.

As used herein, a pure enantiomeric compound is substantially free from other enantiomers or stereoisomers of the compound (i.e., in enantiomeric excess). In other words, an “S” form of the compound is substantially free from the “R” form of the compound and is, thus, in enantiomeric excess of the “R” form. The term “enantiomerically pure” or “pure enantiomer” denotes that the compound comprises at least 95% by weight, at least 96% by weight, at least 97% by weight, at least 98% by weight, at least 99% by weight, at least 99.5% by weight, at least 99.6% by weight, at least 99.7% by weight, at least 99.8% by weight, or at least 99.9% by weight of the enantiomer. In certain embodiments, the weights are based upon total weight of all enantiomers or stereoisomers of the compound.

Compounds described herein may also comprise one or more isotopic substitutions. For example, H may be in any isotopic form, including ¹H, ²H (D or deuterium), and ³H (T or tritium); C may be in any isotopic form, including ¹²C, ¹³C, and ¹⁴C; N may be any isotopic form, including ¹⁴N and ¹⁵N; O may be in any isotopic form, including 160 and 180; and the like.

The articles “a” and “an” may be used herein to refer to one or to more than one (i.e., at least one) of the grammatical objects of the article. By way of example “an analogue” means one analogue or more than one analogue.

“Pharmaceutically acceptable” means approved or approvable by a regulatory agency of the Federal or a state government or the corresponding agency in countries other than the United States, or that is listed in the U.S. Pharmacopoeia or other generally recognized pharmacopoeia for use in animals, and more particularly, in humans.

“Pharmaceutically acceptable salt” refers to a salt of a compound of the invention that is pharmaceutically acceptable and that possesses the desired pharmacological activity of the parent compound. In particular, such salts are non-toxic and may be inorganic or organic acid addition salts and base addition salts. Specifically, such salts include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or (2) salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, N methylglucamine and the like. Salts further include, by way of example only, sodium, potassium, calcium, magnesium, ammonium, tetraalkylammonium, and the like; and when the compound contains a basic functionality, salts of non-toxic organic or inorganic acids, such as hydrochloride, hydrobromide, tartrate, mesylate, acetate, maleate, oxalate, and the like. The term “pharmaceutically acceptable cation” refers to an acceptable cationic counter-ion of an acidic functional group. Such cations are exemplified by sodium, potassium, calcium, magnesium, ammonium, tetraalkylammonium cations, and the like. See, e.g., Berge, et al., J. Pharm. Sci. (1977) 66 (1): 1-79.

“Solvate” refers to forms of the compound that are associated with a solvent or water (also referred to as “hydrate”), usually by a solvolysis reaction. This physical association includes hydrogen bonding. Conventional solvents include water, ethanol, acetic acid, and the like. The compounds of the invention may be prepared e.g., in crystalline form and may be solvated or hydrated. Suitable solvates include pharmaceutically acceptable solvates, such as hydrates, and further include both stoichiometric solvates and non-stoichiometric solvates. In certain instances, the solvate will be capable of isolation, for example when one or more solvent molecules are incorporated in the crystal lattice of the crystalline solid. “Solvate” encompasses both solution-phase and isolable solvates.

The procedures described herein include standard solvents and catalysts as are known in the art. Although particular compounds (e.g., acids, bases, coupling agents) are recited in the disclosure, it is clear to one of skill in the art that different reagents can be used.

Exemplary solvents include but are not limited to chlorinated solvents (e.g., dichloromethane, chloroform, 1,2-dichloroethane), ethers (e.g., diethyl ether, tert-butyl methyl ether, cyclopentyl methyl ether, diglyme, 1,4-dioxane, 2-methyltetrahydrofuran), alcohols (e.g., methanol, ethanol, isopropanol, tert-butanol), alkanes (e.g., pentane, hexanes, heptanes), glycols (e.g., ethylene glycol, polyethylene glycol), polar aprotic solvents (e.g., dimethylacetamide, acetonitrile, dimethyl sulfoxide, dimethyl formamide, acetone, N-methyl-2-pyrrolidone,), and polar protic solvents (e.g., water, ethanol, acetic acid, propionic acid).

Exemplary acids include but are not limited to mineral acids (e.g., hydrochloric acid, nitric acid, phosphoric acid, sulfuric acid) and organic acids (e.g., acetic acid, malonic acid, methanesulfonic acid, propionic acid, thioacetic acid, p-toluenesulfonic acid, tribromoacetic acid, trichloroacetic acid, trifluoroacetic acid).

Exemplary bases include but are not limited to amino bases (e.g. 1,4-diazabicyclo[2.2.2]octane, diethylamine, triethylamine, N,N-diisopropylethylamine, lithium amide, lithium bis(trimethylsilyl)amide, morpholine, piperidine), alkoxides (e.g., barium tert-butoxide, lithium tert-butoxide, sodium methoxide), hydroxides (e.g., tetrabutylammonium hydroxide, sodium hydroxide, potassium hydroxide), organometallic bases (e.g., n-butyllithium, tert-butyllithium, butyl magnesium chloride), pyridines (e.g., 4-dimethylaminopyridine, 2,6-lutidine, pyridine), carbonates (e.g., lithium carbonate, sodium carbonate, magnesium carbonate, potassium carbonate), and hydrides (e.g., sodium hydride, calcium hydride, potassium hydride).

Exemplary coupling agents include but are not limited to carbodiimide reagents (e.g., N,N′-diisopropylcarbodiimide, 1-(3-dimethylaminopropyl)-3-ethylcarbodiimide hydrochloride, N,N′-dicyclopentylcarbodiimide), additives for carbodiimide reagents (e.g., 1-hydroxy-7-azabenzotriazole, 6-chloro-1-hydroxy benzotriazole, N-hydroxysuccinimide, 1-hydroxy-2-pyridinone, 6-chloro-N-hydroxy-2-phenylbenzimidazole, ethyl 2-cyano-2-(hydroxyimino)acetate), anhydride based or forming reagents (e.g., ditertbutyl carbonate, acetic anhydride, 2-ethoxy-1-ethoxy carbonyl-1,2-dihydroquinoline, ethylchloroformate), acylazoles (e.g., carbonyl diimidazole), acid halide generating reagents (e.g., thionyl chloride, phosgene, cyanuric chloride, benzyltriphenylphosphonium dihydrogen trifluoride), phosphonium salt coupling reagents (e.g., benzotriazol-1-yloxytris (diemthylamino)phosphonium hexafluorophosphate), tetramethyl aminium reagents (e.g., 2-(2-oxo-1 (2H)-pyridyl-1,1,3,3-tetramethyluronium tetrafluorob orate), aminium reagents (e.g., 2-chloro-1,3-dimethylimidazolidinium hexafluorophosphate), oxyma uranium salts (e.g., 1-((1-cyano-2-ethoxy-2-oxoethylideneaminooxy) (morpholino)methylene) pyrrolidinium hexafluorophosphate), antimonate uranium salts (e.g., benzotriazol-1-yloxuy-N,N-dimethyl-methaniminium hexachloroantimonate), organophosphorus reagents (e.g., diethylcyanophosphonate, 1-oxo-chlorophospholane, 2-propanephosphonic acid anhydride), triazine based reagents (e.g., 2-chloro-4,6-dimethoxy-1,3,5-triazine), organosulfur reagents (e.g., pentafluorophenyl-4-nitrobenzenesulfonate), pyridinium reagents (e.g., 2-chloro-1-methylpyridinium iodide), polymer bound reagents (e.g., polymer supported N-ethoxycarbonyl-2-ethoxy-1,2-dihydroquinoline).

As used herein, the term “isotopic variant” refers to a compound that contains unnatural proportions of isotopes at one or more of the atoms that constitute such compound. For example, an “isotopic variant” of a compound can contain one or more non-radioactive isotopes, such as for example, deuterium (²H or D), carbon-13 (¹³C), nitrogen-15 (¹⁵N), or the like. It will be understood that, in a compound where such isotopic substitution is made, the following atoms, where present, may vary, so that for example, any hydrogen may be 2H/D, any carbon may be ¹³C, or any nitrogen may be ¹⁵N, and that the presence and placement of such atoms may be determined within the skill of the art. Likewise, the invention may include the preparation of isotopic variants with radioisotopes, in the instance for example, where the resulting compounds may be used for drug and/or substrate tissue distribution studies. The radioactive isotopes tritium, i.e., 3H, and carbon-14, i.e., ¹⁴C, are particularly useful for this purpose in view of their ease of incorporation and ready means of detection. Further, compounds may be prepared that are substituted with positron emitting isotopes, such as ¹¹C, ¹⁸F, ¹⁵O, and ¹³N, and would be useful in Positron Emission Topography (PET) studies for examining substrate receptor occupancy. All isotopic variants of the compounds provided herein, radioactive or not, are intended to be encompassed within the scope of the invention.

“Stereoisomers”: It is also to be understood that compounds that have the same molecular formula but differ in the nature or sequence of bonding of their atoms or the arrangement of their atoms in space are termed “isomers.” Isomers that differ in the arrangement of their atoms in space are termed “stereoisomers.” Stereoisomers that are not mirror images of one another are termed “diastereomers”, and those that are non-superimposable mirror images of each other are termed “enantiomers.” When a compound has an asymmetric center, for example, and an atom, such as a carbon atom, is bonded to four different groups, a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+) or (−)-isomers respectively). A chiral compound can exist as either an individual enantiomer or as a mixture of both enantiomers. A mixture containing equal proportions of the enantiomers is called a “racemic mixture”.

“Tautomers” refer to compounds that are interchangeable forms of a particular compound structure, and that vary in the displacement of hydrogen atoms and electrons. Thus, two structures may be in equilibrium through the movement of n electrons and an atom (usually H).

For example, enols and ketones are tautomers because they are rapidly interconverted by treatment with either acid or base. Another example of tautomerism is the aci- and nitro-forms of phenylnitromethane, that are likewise formed by treatment with acid or base. Tautomeric forms may be relevant to the attainment of the optimal chemical reactivity and biological activity of a compound of interest.

A “subject” to which administration is contemplated includes, but is not limited to, a human (i.e., a male or female of any age group, e.g., a pediatric subject (e.g., infant, child, adolescent) or adult subject (e.g., young adult, middle-aged adult or senior adult)) and/or a non-human animal, e.g., a mammal such as primates (e.g., cynomolgus monkeys, rhesus monkeys), cattle, pigs, horses, sheep, goats, rodents, cats, and/or dogs. In certain embodiments, the subject is a human. In certain embodiments, the subject is a non-human animal.

Disease, disorder, and condition are used interchangeably herein.

As used herein, and unless otherwise specified, the terms “treat,” “treating” and “treatment” contemplate an action that occurs while a subject is suffering from the specified disease, disorder or condition, which reduces the severity of the disease, disorder or condition, or retards or slows the progression of the disease, disorder or condition (“therapeutic treatment”), and also contemplates an action that occurs before a subject begins to suffer from the specified disease, disorder or condition (“prophylactic treatment”).

In general, the “effective amount” of a compound refers to an amount sufficient to elicit the desired biological response, e.g., to treat a cancer. As will be appreciated by those of ordinary skill in this art, the effective amount of a compound, composition, or formulation of the invention may vary depending on such factors as the desired biological endpoint, the pharmacokinetics of the compound, the disease being treated, the mode of administration, and the age, weight, health, and condition of the subject. An effective amount encompasses therapeutic and prophylactic treatment.

As used herein, and unless otherwise specified, a “therapeutically effective amount” of a compound is an amount sufficient to provide a therapeutic benefit in the treatment of a disease, disorder, or condition, or to delay or minimize one or more symptoms associated with the disease, disorder, or condition. A therapeutically effective amount of a compound means an amount of therapeutic agent, alone or in combination with other therapies, which provides a therapeutic benefit in the treatment of the disease, disorder, or condition. The term “therapeutically effective amount” can encompass an amount that improves overall therapy, reduces or avoids symptoms or causes of disease or condition, or enhances the therapeutic efficacy of another therapeutic agent, i.e., to produce a reduction in the amount and/or severity of the symptoms associated with that disorder. For example, in the case of a cancer metastasis to the brain, administration of a therapeutically effective amount of the nanoparticles or a formulation of nanoparticles, as described herein, results in reduction and/or reversal of the symptoms of metastasis; e.g., regression of the metastatic tumor. Therapeutically effective amounts for treatment of brain metastases vary with the type and extent of brain damage, and can also vary depending on the overall condition of the subject.

As used herein, and unless otherwise specified, a “prophylactically effective amount” of a compound is an amount sufficient to prevent a disease, disorder, or condition, or one or more symptoms associated with the disease, disorder, or condition, or prevent its recurrence. A prophylactically effective amount of a compound means an amount of a therapeutic agent, alone or in combination with other agents, which provides a prophylactic benefit in the prevention of the disease, disorder, or condition. The term “prophylactically effective amount” can encompass an amount that improves overall prophylaxis or enhances the prophylactic efficacy of another prophylactic agent.

Aqueous suspensions may contain the compounds in admixture with excipients suitable for the manufacture of aqueous suspensions. Such excipients can be suspending agents, for example sodium carboxymethylcellulose, methylcellulose, hydroxypropylmethylcellulose, sodium alginate, polyvinylpyrrolidone, gum tragacanth and gum acacia; dispersing or wetting agents such as a naturally occurring phosphatide, for example lecithin, or condensation products of an alkylene oxide with fatty acids, for example, polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such a polyoxyethylene with partial esters derived from fatty acids and hexitol anhydrides, for example polyoxyethylene sorbitan monooleate. The aqueous suspensions may also contain one or more preservatives, for example ethyl, or n-propyl p-hydroxybenzoate, one or more coloring agents, one or more flavoring agents, and one or more sweetening agents, such as sucrose or saccharin.

### DAN-222 Formulations

The chemical structure of the DAN-222 MAP-Gly-CPT polymer-drug conjugate repeat unit is provided in Formula (I):

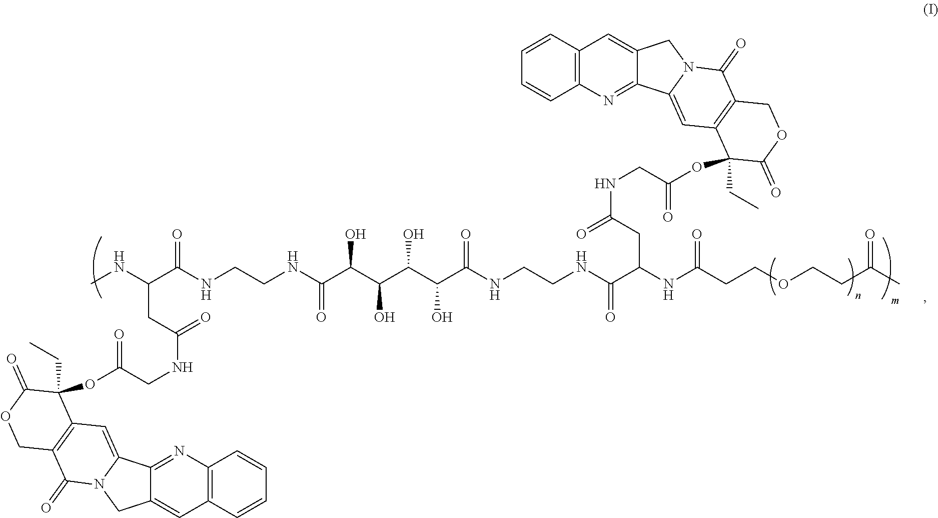

Formula (I) represents the polymer-drug conjugate (MAP-Gly-CPT) of DAN-222. The polymer repeat unit consists of a modified mucic acid moiety bonded to a bis-propanoyl PEG unit. As shown in FIG. 1, when formulated in an aqueous media, DAN-222 self-assembles into nanoparticles (NPs). When formulated as NPs, each NP is expected to consist of approximately two MAP-Gly-CPT strands.

In Formula (I), (n) is the number of ethylene glycol repeating units. In certain aspects, in an exemplary formulation of the invention, (n) is a number in a range between 5 and 200. Preferably, in formulations of the invention, on average (n) is about 79 for MAP-Gly-CPT molecules of Formula (I) in the formulation.

In Formula (I), (m) is the number of repeating units of MAP-Gly-CPT in a strand of the polymer-drug conjugate. In certain aspects, (m) is a number between 20 and 200. In preferred formulations of the invention, on average, each polymer strand consists of 16±4 repeats of this PEG-MAP unit, each of which can be conjugated with up to two Gly-CPT molecules.

When Gly-CPT is conjugated to MAP, the reaction is performed so that conjugation occurs at between about 75% and 100% of sites on the MAP that are available for reaction with Gly-CPT. In certain aspects, about 75%, 80%, 85%, 90%, 95%, 98%, 99%, or 100% of available sites are reacted and conjugated to CPT. In preferred aspects, at least about 90% of available sites are reacted and conjugated to CPT.

As explained, when formulated in an aqueous formulation, DAN-222 self-assembles into nanoparticles (NPs).

In certain aspects, in formulations of the invention, the size of the nanoparticles ranges from about 20 to about 80 nm in diameter. Exemplary nanoparticle diameters are about: 15 nm, 16 nm, 17 nm, 18 nm, 19, nm, 20 nm, 21 nm, 22 nm, 23 nm, 24 nm, 25 nm, 26 nm, 27 nm, 28 nm, 29 nm, 30 nm, 31 nm, 32 nm, 33 nm, 34 nm, 35 nm, 36 nm, 37 nm, 38 nm, 39 nm, 40 nm, 45 nm, 50 nm, 55 nm, 60 nm, 65 nm, 70 nm, 75 nm, or 80 nm. In preferred aspects, the size of the nanoparticles ranges from about 30 nm to about 40 nm in diameter.

In certain aspects embodiments, values of (m) and (n) are chosen such that, after assembly of DAN-222 strand(s) into a nanoparticle, the size of the nanoparticle is in a range of from 10 to 900 nm or any integral values therebetween, e.g., from 20 nm to 80 nm. In certain aspects, the formulations of the invention include a DAN-222 nanoparticle concentration of between 5 mg/mL and 40 mg/mL, of between 10 mg/mL and 30 mg/mL or between 15 mg/mL and 25 mg/mL. In preferred aspects, the nanoparticle concentration is between 17 mg/mL and 23 mg/mL.

In certain aspects, the formulations of the invention include DAN-222 nanoparticles having a polydispersity index (PDI) less than or equal to 0.9. In certain aspects, the nanoparticles have a PDI less than or equal to 0.8, 0.7, 0.6, 0.5, 0.4, 0.3, 0.2, or 0.1.

In certain aspects, the formulations of the invention include a total concentration of CPT between 0.5 mg/ml and 10 mg/ml, between 1 mg/ml and 9 mg/ml, between 2 mg/ml and 8 mg/ml, between 3 mg/ml and 7 mg/ml, between 4 mg/ml and 6 mg/ml, between 1 mg/ml and 4 mg/ml, between 1.5 mg/ml and 3.8 mg/ml, between 2 mg/ml and 3 mg/ml, between 2.5 mg/ml and 3.0 mg/ml, or between 1.7 mg/ml and 3.6 mg/ml. In preferred aspects, the formulations of the invention include a total CPT concentration of about 2.6 mg/ml.

In certain aspects, the formulation comprises a concentration of between 5 mg/ml and 100 mg/ml, between 4 mg/ml and 77 mg/ml, between 10 mg/ml and 90 mg/ml, between 10 mg/ml and 80 mg/ml, between 15 mg/ml and 50 mg/ml, between 15 mg/ml and 40 mg/ml, between 15 mg/ml and 30 mg/ml, between 15 mg/ml and 25 mg/ml, between 13 mg/ml and 28 mg/ml, or about 20 mg/ml of the compound of Formula (I).

In certain aspects, an exemplary formulation of the invention has a pH of between 3 and 6. In additional embodiments, the formulation has a pH of between pH 3.5 and pH 5.5, or between pH 4 and pH 5. In certain aspects, the formulation has a pH between pH 4 and pH 4.6. In preferred aspects, the formulation has a pH of about pH 4.3. The present Inventors discovered that formulations having a lower pH, e.g., a pH between pH 3 and pH 5, showed less CPT loss during storage, with CPT stability increasing with lower pH value of the formulation. However, formulations at a pH less than pH 4 may cause patient discomfort.

A formulation of the invention may further include at least one buffer selected from sodium succinate, sodium citrate, sodium acetate, phosphoric acid, histidine-HCL, and sodium phosphate. In preferred aspects, the buffer is a sodium acetate buffer. The formulation may further include at least one tonicity modifier selected from KCl, NaCl, Proline, Arginine-HCl, sucrose, and glycine. Preferably, the at least one tonicity modifier is NaCl.

In preferred aspects, the formulation comprises about 20 mg/mL of the compound of Formula (I), about 20 mM sodium acetate, about 200 mM NaCl, and has a pH of 4.3±0.3.

The zeta potential is a measure of surface charge. In certain DAN-222 formulations of the invention, the zeta potential is from −5 mV to +5 mV. In preferred aspects, DAN-222 formulations of the invention exhibit zeta potentials close to neutral.

In certain aspects, the osmolality of the DAN-222 formulations of the invention is between 300 mOsm/kg and 600 mOsm/kg. In preferred aspects, the osmolality of a DAN-222 formulations is between about 336 and about 504 mOsm/kg. In certain aspects, this range is representative of the osmolality of the formulation buffer, as the DAN-222 molecule has little effect on the osmolality result.

The molecular weight of a DAN-222 nanoparticle in an aqueous formulation as described herein may be determined by gel permeation chromatography (GPC) using PEO standards with nominal molecular weights of 100 kDa and 200 kDa. The molecular weight of the DAN-222 conjugate is calculated from the molecular weight of the polymer (i.e., MAP) used in the synthesis of the conjugate, adjusted for drug (CPT) loading as follows:

$${MW}_{conjugate} = {MW}_{polymer} / ( 1 - T ) ,$$

where MW_{polymer} is the molecular weight of the MAP used in the synthesis of the DAN-222 conjugate, and Tis the fractional loading of the therapeutic agent (CPT), expressed as a decimal fraction.

In preferred formulations of the invention, the average molecular weight of MAP parent polymer consisting of, on average, 16 repeat units is approximately 65,000 Da. The target molecular weight range for this material is 50,000-80,000 Da. The molecular weight of a 65,000 Da MAP polymer at 90% CPT loading is approximately 75,000 Da. In certain embodiments, the molecular weight of a MAP-Gly-CPT molecule is between 30 kDa and 100 kDa, between 40 kDa and 90 kDa, between 50 kDa and 80 kDa or between 60 kDa and 70 kDa. In preferred aspects, nanoparticles of the formulation comprise on average two strands comprising the compound of Formula (I) and have an average molecular weight of about 150,000 Da. In additional embodiments, nanoparticles comprise 1, 2, 3, 4, 5 or 6 strands of the compound of Formula (I).

Strands per particle (SpP), as used herein, is the number of mucic acid polymer (“MAP”) therapeutic agent (CPT) conjugate molecules present in a particle or nanoparticle. For purposes of determining SpP, a particle or nanoparticle is an entity having at least one MAP-Gly-CPT strand/molecule which, at the concentration suitable for administration to humans, behaves as a single unit in any aqueous solution, e.g., water at neutral pH, PBS at pH 7.4, or any formulation in which it will be administered to patients. For purposes of calculating strands per particle, a MAP-Gly-CPT molecule is a single MAP-Gly-CPT conjugate strand. In preferred formulations of the invention, the DAN-222 nanoparticles include, on average, two strands of the MAP-Gly-CPT conjugate.

Methods disclosed herein provide for evaluating a nanoparticle wherein said particles comprise one or more MAP-Gly-CPT molecules. Generally, the method requires providing a sample comprising a plurality of said particles and determining an average value for the number of MAP-Gly-CPT molecules per particle in the sample, to thereby evaluate a preparation of particles. The value for a particle sample will be a function of values obtained for a plurality of particles.

As discussed above, SpP is defined as the number of MAP-Gly-CPT molecules that self-assemble into a particle or nanoparticle, thus SpP= [MAP-Gly-CPT molecules]/P (or NP), where [MAP-Gly-CPT molecules] is the number of MAP-Gly-CPT strands/molecules, and P (or NP) is a single particle (or nanoparticle).

In certain embodiments, the method further comprises comparing the determined value with a reference value. The comparison can be conducted in a number of ways. By way of example, in response to a comparison or determination made in the method, a decision or step is taken, e.g., a production parameter in a process for making a particle is altered, the sample is classified, selected, accepted or discarded, released or withheld, processed into a drug product, shipped, moved to a different location, formulated, e.g., formulated with another substance, e.g., an excipient, labeled, packaged, released into commerce, or sold or offered for sale. For example, based on the result of the determination, or upon comparison to a reference standard, the batch from which the sample is taken can be processed, e.g., as just described.

To calculate the number of MAP-Gly-CPT strands per particle, the size of the particle is determined, e.g., by molecular weight by light scattering of self-assembled particles, and the size of individual polymers, e.g., by molecular weight by light scattering of individual polymers, and the loading of CPT, e.g., by mass %. With these values, SpP may be calculated as follows:

SpP=MW_{particle}/(MW_{conjugate}),

where MW particle is the molecular weight of the particle, and MW_{conjugate} is the molecular weight of a MAP-Gly-CPT molecule which is calculated as follows:

$${MW}_{conjugate} = {MW}_{polymer} / ( 1 - T \% ) ,$$

where MW polymer is the molecular weight of the MAP, and T % is the percent loading of therapeutic agent (CPT) expressed as a decimal, e.g. 10% loading results in T %=0.1.

Polymer molecular weight distribution and particle dispersity: MAPs are synthesized such that they have a range of molecular weights. Molecules of varied molecular weight provide varying contributions to particle diameter and the strands per particle.

Particle shape: Particle shape is assumed to be approximately spherical. Self-assembly is assumed to be driven by the hydrophobic region created by the therapeutic agents of the MAP-Gly-CPT molecule.

### Methods for Producing DAN-222 Formulations

The present invention provides methods for producing DAN-222 formulations, as described herein. The drug substance, DAN-222, consists of 20 (S)-camptothecin (CPT) covalently bound to a mucic acid-based polymer carrier via a glycyl linkage (MAP-Gly-CPT). During manufacture, MAP-Gly-CPT is isolated as an amorphous solid. In aqueous solution, the MAP-Gly-CPT polymer-drug conjugate self-assembles into nanoparticles (NPs).

In certain aspects, an exemplary method for manufacturing a DAN-222 formulation includes four main steps: (i) derivatization of camptothecin (CPT) to yield Gly-CPT (as the trifluoracetic acid (TFA) salt); (ii) synthesis of the parent mucic acid polymer (MAP); (iii) covalent attachment of derivatized camptothecin to the parent polymer to yield solid, amorphous MAP-Gly-CPT polymer-drug conjugate; and (iv) aqueous formulation of MAP-Gly-CPT to form nanoparticles (NPs).

Examples 5-8 provide details and data pertaining to exemplary methods for manufacturing DAN-222 and DAN-222 formulations of the invention.

FIG. 35 provides a scheme for an exemplary method of DAN-222 manufacture. Starting materials are indicated in dark boxes and manufacturing steps performed under GMP conditions for the Phase 1 clinical trial drug substance are indicated with text or are surrounded by box 3401.

Briefly, in an exemplary method, in step (i), the production of the derivatized CPT is a two-step process whereby the hydroxyl group of CPT is esterified with a tert-butoxycarbonyl-protected glycine linker (Boc-Gly), followed by the removal of the Boc protection group and isolation to yield glycine-linked CPT (Gly-CPT) as a trifluoroacetic acid (TFA) salt.

As described in Example 7, the present inventors discovered several improvements in methods for producing Gly-CPT as a trifluoroacetic acid (TFA) salt.

For the Boc-Gly-CPT synthesis step, relative to prior methods, the reaction was found to proceed more smoothly when run under more dilute conditions at a higher reaction temperature. Further, a new aqueous extraction step was added to improve removal of process impurities. In earlier syntheses, DCM was removed to afford a crude solid. In contrast, the present inventors employed vacuum distillation to remove only 95% of the DCM volume and a significantly lower proportion of methanol added for final precipitation at a higher temperature. An additional wash with isopropyl alcohol (IPA) was found to improve performance of the isolation.

For deprotection and isolation of Gly-CPT, for the subsequent precipitation step, a significantly lower volume of methyl tert-butyl ether (MTBE) was used, and subsequent washes of the filter cake with an MTBE: DCM mixture were performed with a lower ratio of MTBE: DCM.

In an exemplary method for producing a DAN-222 formulation, in step (ii), the production of the parent polymer, mucic acid polymer (MAP), is a step-growth polymerization of the two comonomers, MAM and diSPA-PEG, a linear PEG molecule with succinimide-activated propionyl esters at each end. The comonomers are dissolved in dimethyl sulfoxide (DMSO), and the reaction is initiated by the addition of N,N-diisopropylethylamine (DIPEA), triggering the amidation of free amines at each end of the MAM monomer. The reaction is quenched with water when the MAP molecular weight (MW) is expected to be at or near the target value, and the MAP is isolated by dialysis against DMSO and water, followed by lyophilization.

As described in Example 7, the present inventors discovered several improvements in methods for producing the necessary MAP for use in preparing DAN-222.

The process of step (ii) aims to control the reaction kinetics and achieve a near plateau in polymer MW over time. While conditions for MAP production can be identified for approximately linear growth of the MW with time, this strategy requires precise in-process monitoring of MW and reaction quenching. Alternatively, given that the polymerization has been shown to follow known models for polymer step-growth, a strategy was developed based on polymerization theory (the Modified Carothers's Equation and Flory-Shulz Distribution) that stalls the reaction over time. This strategy achieves a plateau in growth by utilizing a stoichiometric imbalance of the comonomers and slows the reaction as the MAP approaches the targeted MW. Example polymerizations for which the MW of the MAP at late time points is controlled by the stoichiometric ratio of MAM to diSPA-PEG in the reaction mixture are shown in FIGS. 44 and 46-48.

The present Inventors found that an increase in the ratio of MAM: diSPA-PEG results in production of a lower MW polymer. Thus, it was desired that monomer starting materials be prepared with a high degree of functionalization as specified herein, and that the reaction be performed under anhydrous conditions to retain stability of the activated SPA groups on diSPA-PEG to protect against early termination of the amidation reaction, allowing for a well-controlled stoichiometric ratio of the comonomers at or near the target.

Over the course of the Step (ii) development, several modifications were made to the process to make it more robust, scalable, safer, and/or to lower impurity levels. These modifications included: (i) using MAM as the diTFA salt versus the neutral species; (ii) MAM, diSPA-PEG, TFA, and DIPEA stoichiometry; and (iii) using dialysis versus precipitation for purification.

To reduce potential variability, it was decided to isolate MAM as the neutral species (lacking the TFA counterion) and allow for a more precise addition of TFA as a subsequent reaction step prior to polymerization.

Development of the MAM modification occurred as a result of reactions carried out using MAM isolated as the diTFA salt or as the neutral species (isolated with only a trace of TFA) to study the effect of the presence or absence of TFA in the monomer starting material on the reaction behavior. While the TFA group does not participate in the actual polymerization reaction, it was found to significantly impact the solubility of MAM in DMSO, and hence the reaction kinetics. Unlike the diTFA salt, which is generally soluble in DMSO, the neutral species was found to be minimally soluble in DMSO. Despite its poor solubility, investigation of the polymerization of the neutral species with diSPA-PEG showed the reaction to proceed without addition of TFA, though with generally linear kinetics regardless of the comonomer ratio. In contrast, polymerization of the neutral species with diSPA-PEG whereby approximately 1-2 eq. TFA was added to the reaction prior to initiation with DIPEA led to a recovery of the desired plateau behavior.

Based on the results obtained from the initial experiments, it was decided to pursue development of reaction conditions with MAM isolated as the neutral species as well as subsequent addition of TFA that would allow for plateau of the polymer MW at or near the target.

Further, the present Inventors performed a series of experiments to study the combined effects of different comonomer ratios as well as equivalents of TFA and DIPEA on the kinetics of the reaction in Step (ii). The results showed that a reduced amount of DIPEA (˜2.5 equivalents) led to decreased reaction kinetics, while a decrease in the MAM: diSPA-PEG ratio towards 1:1 led to increased MW of the MAP product. An increase in TFA concentration led to an improved, logarithmic, plateau in MW value; rather than a linear plateau. The combined results of the initial screen suggested that the comonomer ratio, equivalents of TFA and DIPEA, and reaction time were sufficient handles to achieve the targeted degree of MAP polymerization.

As described in Example 7, conditions were identified for polymerization that allowed for plateau of the MW and provided MAP with MW in the targeted range of 50,000-80,000 Da over a broad window of time. A modest increase in DIPEA (2.6 vs 2.5 equivalents) was evaluated for scale-up reactions and provided similar kinetics, compared to those observed in small-scale experiments.

To address aspects of scaling up MAP production, step (ii) may include a step of precipitating the MAP from DMSO using IPA under an inert atmosphere such as argon or nitrogen. As described in Example 7, using this procedure for isolating MAP results in a product with low levels of impurities that is a dry or reasonably dry solid, which is easily recovered.

Other exemplary methods for producing MAP include those as provided in U.S. Patent Pub. No. US2021/0170049A1, which is hereby incorporated by reference.

In an exemplary method for producing a DAN-222 formulation, in step (iii), the Synthesis of MAP-Gly-CPT Amorphous Polymer-Drug Conjugate includes using EDC/NHS coupling chemistry. However, as described in Example 7 herein, to reduce the excess in starting materials and reagents in the reaction, particularly that of the Gly-CPT, the use of the more active (7-azabenzotriazol-1-yloxy)tripyrrolidinophosphonium hexafluorophosphate (PyAOP) coupling reagent to prepare MAP-Gly-CPT is preferred.

To minimize the process risk of forming high molecular weight species (HMWS), high drug loading levels are used to minimize the number of unreacted carboxylic acid groups on the MAM. Additionally, performing the reaction at increased dilution significantly reduces the likelihood of polymer chain end groups participating in the reaction. As described in Example 7, development reactions were executed with increased dilution on both 2.5 and 4 g scale with 1.0, 1.1, and 1.2 equivalents of Gly-CPT. With the modified reaction conditions, all reactions proceeded well and gave no indication of formation of HMWS.

In an exemplary method for producing a DAN-222 formulation, in step (iv), the preparation of MAP-Gly-CPT NPs requires dissolution of the amorphous polymer drug conjugate in an aqueous medium, whereby the hydrophobic nature of the CPT drives self-assembly of the conjugate into NPs. The initially developed process included dissolution of the MAP-Gly-CPT amorphous material in pH 4.0 water, addition of concentrated saline (9% NaCl, pH 4.0), concentration using a centrifugal ultrafiltration device, and 0.22-micron sterile filtration.

Over the course of the Step (iv) development, several modifications were made to the process to make it more robust, scalable, safer, and/or to lower impurity levels, including: (i) a concentrated formulation buffer change; (ii) use of centrifugal ultrafiltration versus TFF concentration; and (iii) a conditioning filtration step.

While the MAP-Gly-CPT NPs demonstrate good stability in unbuffered saline solution (0.9% NaCl, pH 4.0), the nanoparticles are pH-sensitive; and a robust drug product formulation requires a buffer system to protect against pH drift and degradation. Thus, in preferred aspects, rather than addition of concentrated saline, addition of a concentrated formulation buffer (e.g., 200 mM sodium acetate, 2 M NaCl, pH 4.16) is used. An exemplary resulting formulation using such a buffer includes 20 mM sodium acetate, 200 mM NaCl, pH 4.3±0.3. As described in Example 7, when using such a buffer to produce DAN-222 formulations, no impact on particle properties (e.g., size, zeta potential, filterability) was observed and the product showed ample stability.

In certain aspects step (iv) includes a concentration step. The concentration process may include centrifugal ultrafiltration. In preferred aspects, the concentration process includes TFF ultrafiltration. Scale-up versions of centrifugal ultrafiltration require longer processing times and generally cannot be performed at scale when compared with TFF ultrafiltration.

In certain aspects, step (iv) includes a single final filtration step. In preferred aspects, step (iv) includes an intermediate conditioning filtration step, which may allow seamless and de-risked final sterile filtration. During development production of DAN-222 formulations of the invention, it was observed that the final sterile filtration process was often difficult, and multiple sterile filters would typically be needed to complete filtration.

To mitigate this process risk, the MAP-Gly-CPT amorphous material may be dissolved into the pH 4.0 water and filtered to condition the material and remove some product-related particulates. Upon subsequent addition of the concentrated formulation buffer, and performance of the TFF steps to formulate and concentrate the FBDS, the final 0.22-micron sterile filtration can proceed without risk of extended filtration times.

### Methods of Treatment, Formulations, Kits, and Routes of Administration

Therapeutic compositions and formulation comprising DAN-222, as disclosed herein, are also provided. Such formulations typically comprise the nanoparticles and a pharmaceutically acceptable carrier. Supplementary active compounds can also be incorporated into such formulations.

In certain aspects, the present invention includes formulations useful for treating, cancer, cancer metastases and/or disorders of the brain and central nervous system. In certain aspects, the cancer is selected from breast cancer, lung cancer, colon cancer, ovarian cancer, melanoma, bladder cancer, kidney cancer, liver cancer, salivary gland cancer, stomach cancer, nerve Glioma, thyroid cancer, thymic cancer, epithelial cancer, head and neck cancer, stomach cancer and pancreatic cancer.

In preferred aspects, the cancer is breast cancer or ovarian cancer. In certain aspects, the cancer is HER2-negative metastatic breast cancer (mBC). In certain aspects, the HER2-negative mBC is a homologous recombination deficient (HRD) positive or negative HER2-negative mBC. The inability to accurately repair complex DNA damage and resolve DNA replication stress leads to genomic instability and contributes to cancer etiology. For example, defects in homologous recombination (HR) repair are observed in various cancers. Breast cancer gene type 1 (BRCA1) and breast cancer gene type 2 (BRCA2) have long been known to encode proteins that play a key role in HR repair and mutations in one or both of these genes place patients at greater risk for development of breast, ovarian, prostate, melanoma, and pancreatic cancers.

Concurrently, this same inability to repair also makes cancer cells more vulnerable to DNA-damaging therapeutic agents. For example, tumor cells with defective HR repair show increased sensitivity to chemotherapeutic agents, which suggests that HR-proficient tumor cells might be sensitized to chemotherapeutics if HR repair could be therapeutically inactivated. Topoisomerase inhibitors, such as CPT, damage DNA by binding to topoisomerase I which prevents the unwinding of DNA required for replication, resulting in a stalled replication fork. This can prevent cancer cells from replicating and proliferating in a subject.

Camptothecin (CPT), the active ingredient in DAN-222, is a naturally occurring, pentacyclic quinolone alkaloid isolated from the bark of Camptotheca acuminata. CPT is a topoisomerase I inhibitor that acts by forming a ternary complex with topoisomerase I and DNA during replication and transcription in tumor cells, which blocks the re-ligation of DNA, resulting in torsional tension downstream of RNA polymerase which causes DNA strand breaks.

Treatment of many brain disorders such as brain cancer and metastases to the brain requires that therapeutic molecules be delivered to the brain. Direct delivery of therapeutics to the brain poses severe risks to the subject (e.g., breaching the skull), and cannot be feasibly carried out on a continuing basis, as is required for most chemotherapeutic treatments. However, systemic delivery (e.g., via the bloodstream) will not efficiently deliver molecules to the brain, because of the existence of the blood-brain barrier (BBB); a tightly-joined layer of endothelial cells lining the blood vessels of the brain. A similar permeation barrier, known as the blood-tumor barrier (BTB) exists in certain solid tumors.

Cancers of the breast frequently metastasize to the brain and these brain metastases could be treated with chemotherapeutic molecules used for treatment of breast cancer, if the therapeutic could be delivered to the brain (via the BBB) or a tumor (via the BTB) in sufficient concentrations. One such chemotherapeutic is CPT.

Unfortunately, while CPT is known for its anti-tumor activity, including over a wide spectrum of human cancers, it has poor water solubility, low plasma stability, and dose-limiting toxicity. Moreover, as described, the BBB and/or BTB may inhibit the ability of cells to uptake CPT.

However, as discovered by the present inventors, DAN-222 is more efficacious than other topoisomerase I inhibitors, including free CPT, due to its accumulation in tumors through an enhanced permeability and retention (EPR) effect, which leads to a greater and prolonged topoisomerase I inhibition. The EPR effect relies on the different vasculatures of healthy tissues versus tumors. The DAN-222 nanoparticles encapsulate the CPT and are able to pass through the leaky walls of tumor neovasculature, while concurrently not passing through the tighter walls of healthy blood vessels. Moreover, encapsulation in the DAN-222 nanoparticles provides protection for the CPT from conversion of CPT from its active lactone form to its inactive carboxyl form in the serum, which prevents rapid blood clearance and toxicity to non-malignant tissues.

In certain aspects, an exemplary method of the invention for treating cancer in a subject, includes providing to a subject having cancer at least one dose of a composition comprising a compound of Formula (I):

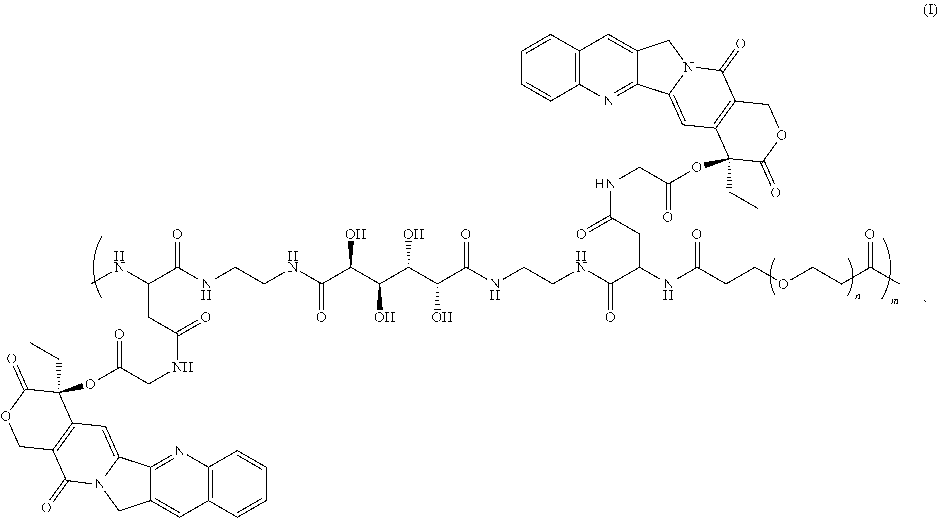

wherein n is a number in a range from 20 to 200; and
wherein m is a number in a range from 5 to 200.

In exemplary methods, n is a number in a range from about 75 to 85; m is a number in a range from about 12 to 20; and the composition has a pH between pH 4 and pH 4.6.

In preferred methods, the composition comprises between 2 mg/mL and 3 mg/ml of camptothecin (CPT). In preferred methods, the dose of the composition comprises between 2 mg/m² and 16 mg/m² of the compound of Formula (I). For example, the dose of the composition may include about 2 mg/m², about 4 mg/m², about 6 mg/m², about 8 mg/m², about 10 mg/m², about 12 mg/m², about 14 mg/m², or about 20 mg/m² of the compound of Formula (I). In certain aspects, the composition comprises about 2.6 mg/m² of the compound of Formula (I).

The DAN-222 formulations and compositions described herein can be administered to a subject by any suitable route, including, but not limited to, inhalation, topically, nasally, orally, parenterally (e.g., intravenously, intraperitoneally, intravesically or intrathecally) or rectally; in a vehicle comprising one or more pharmaceutically acceptable carriers, the proportion of which is determined by the solubility and chemical nature of the compound, chosen route of administration and standard practice. Administration of DAN-222, as described herein, can be carried out using any method known in the art. For example, administration may be transdermal, parenteral, intravenous, intra-arterial, subcutaneous, intramuscular, intracranial, intraorbital, ophthalmic, intraventricular, intracapsular, intraspinal, intracisternal, intraperitoneal, intracerebroventricular, intrathecal, intranasal, by aerosol, by suppositories, or by oral administration. A pharmaceutical composition of the DAN-222 described herein can be for administration by injection, or for oral, pulmonary, nasal, transdermal, or ocular administration.

In preferred aspects, the composition comprising a compound of Formula (I) is provided to a subject as a sterile solution for infusion.

Exemplary formulations may include, but are not limited to, those suitable for parenteral administration, e.g., intrapulmonary, intravenous, intra-arterial, intra-ocular, intra-cranial, sub-meningeal, or subcutaneous administration, including formulations encapsulated in micelles, liposomes or drug-release capsules (active agents incorporated within a biocompatible coating designed for slow-release); ingestible formulations; formulations for topical use, such as eye drops, creams, ointments and gels; and other formulations such as inhalants, aerosols and sprays. The dosage of the compositions of the disclosure will vary according to the extent and severity of the need for treatment, the activity of the administered composition, the general health of the subject, and other considerations well known to the skilled artisan.

In additional embodiments, the DAN-222 compositions described herein are delivered at or near a site of injury or metastasis. Such localized delivery allows for the delivery of the composition non-systemically, thereby reducing the body burden of the composition as compared to systemic delivery. Local delivery can be achieved, for example, by injection, or through the use of various medically implanted devices including, but not limited to, stents and catheters, or can be achieved by inhalation, phlebotomy, or surgery. Methods for coating, implanting, embedding, and otherwise attaching desired agents to medical devices such as stents and catheters are established in the art and contemplated herein.

The composition may be provided under any suitable dosing regimen. For example, the composition may be provided as a single dose or in multiple doses. Provision of multiple doses may be separated by intervals, such as 12 hours, 24 hours, 2 days, 3 days, 4 days, 5 days, 1 week, 2 weeks, 3 weeks, 4 weeks, or more. Multiple doses may be provided within a period of time. For example, multiple doses may be provided over a period of 1 day, 2 days, 3 days, 4 days, 5 days, 1 week, 2 weeks, 3 weeks, 4 weeks, or more. The compositions may be provided repeatedly for a specified duration. For example and without limitation, the compositions may be provided for 1 week, 2 weeks, 3 weeks, 4 weeks, 6 weeks, 8 weeks, 12 weeks, 3 months, 4 months, 5 months, 6 months, 8 months, 10 months, 12 months or more.

In preferred aspects, the composition comprising a compound of Formula (I) is provided to a subject as a sterile solution for infusion in once-weekly doses. In preferred aspects, the composition comprising a compound of Formula (I) is provided to a subject as a sterile solution for infusion in once-weekly doses over a four-week treatment cycle.

Exemplary methods of the invention include providing one or more doses of the composition comprising a compound of Formula (I) in conjunction with providing at least one dose of a poly (ADP-ribose) polymerase (PARP) inhibitor.

Exemplary PARP inhibitors used in the methods of the invention include, by way of example, olaparib, rucaparib, niraparib, and talazoparib. Preferably, the PARP inhibitor is niraparib.

In preferred aspects, a dose of niraparib is provided to the subject daily during a treatment cycle. In certain aspects, multiple doses of niraparib may be provided, separated by intervals such as 12 hours, 24 hours, 2 days, 3 days, 4 days, 5 days, 1 week, 2 weeks, 3 weeks, 4 weeks, or more. Multiple doses may be provided within a period of time. For example, multiple doses may be provided over a period of 1 day, 2 days, 3 days, 4 days, 5 days, 1 week, 2 weeks, 3 weeks, 4 weeks, or more. The niraparib may be provided repeatedly for a specified duration. For example, and without limitation, the niraparib may be provided for 1 week, 2 weeks, 3 weeks, 4 weeks, 6 weeks, 8 weeks, 12 weeks, 3 months, 4 months, 5 months, 6 months, 8 months, 10 months, 12 months or more.

In preferred aspects, the methods of the invention include providing each dose of niraparib as a dose between 25 mg and 200 mg dose. In preferred aspects, the dose is a dose of between about 50 mg and 100 mg. In preferred aspects, the dose is a 100 mg dose.

In certain methods, a subject is provided with a weekly dose of the composition comprising the compound of Formula (I) and a daily dose of niraparib during a treatment cycle.

In certain methods, the cancer is one or more cancers of the breast, ovary brain, lung, testicle, head, neck, esophagus, lymphoma, central nervous system, peripheral nervous system, bladder, stomach, pancreas, liver, oral mucosa, colorectal, anus, kidney, bladder, uroepithelium, prostate, endometrium, uterus, fallopian tube, mesothelioma, melanoma, myeloma, leukemia, and Kaposi's sarcoma.

In preferred aspects, the cancer is a breast cancer. In certain aspects, the breast cancer is a homologous recombination repair deficiency (HRD) positive or HRD-negative breast cancer.

The therapeutic compositions disclosed herein are useful for, inter alia, treating cancer, cancer metastases and disorders of the brain and central nervous system. Accordingly, a “therapeutically effective amount” of a composition comprising DAN-222 is any amount that reduces symptoms or, e.g., stimulates tumor regression. For example, dosage amounts of DAN-222 can vary from about 0.1-1.0 mg/kg body weight, or from about 0.5 to 2.0 mg/kg body weight or from about 1-5 mg/kg body weight or from about 1 mg/kg body weight to about 10 mg/kg body weight or more (or any integral value therebetween).

Doses may be provided to subjects with a frequency of administration of, e.g., hourly, twice per day, once per day, twice per week, once per week, twice per month, once per month, depending upon, e.g., body weight, route of administration, severity of disease, etc. Thus, a therapeutically effective amount can comprise a plurality of administrations of the same amount, or different amounts, of DAN-222. In certain embodiments, a single administration of DAN-222 is a therapeutically effective amount.

In certain embodiments, DAN-222 formulations are administered at a dosage of 1-20 mg DAN-222 (or any integral or decimal value therebetween) per square meter of the surface area of the body of the subject, as is typical for dosages of chemotherapeutics.

Various pharmaceutical compositions and techniques for their preparation and use are known to those of skill in the art in light of the present disclosure. For a detailed listing of suitable pharmacological compositions and techniques for their administration one may refer to texts such as Remington's Pharmaceutical Sciences, 17th ed. 1985; Brunton et al., “Goodman and Gilman's The Pharmacological Basis of Therapeutics,” McGraw-Hill, 2005; University of the Sciences in Philadelphia (eds.), “Remington: The Science and Practice of Pharmacy,” Lippincott Williams & Wilkins, 2005; and University of the Sciences in Philadelphia (eds.), “Remington: The Principles of Pharmacy Practice,” Lippincott Williams & Wilkins, 2008.

The DAN-222 nanoparticles and DAN-222 nanoparticle formulations described herein can be suspended in a physiologically compatible carrier for administration. As used herein, the term “physiologically compatible carrier” refers to a carrier that is compatible with DAN-222 and with any other ingredients of the formulation, and is not deleterious to the recipient thereof. Those of skill in the art are familiar with physiologically compatible carriers. Examples of suitable carriers include water (e.g., pH 4 water), phosphate-buffered saline, Hank's balanced salt solution +/− glucose (HBSS), and multiple electrolyte solutions such as, e.g., Plasma-Lyte™ A (Baxter).

The volume of a DAN-222 suspension administered to a subject will vary depending on the site of administration, treatment goal and number concentration of DAN-222 in solution. Typically, the amount of nanoparticles administered will be a therapeutically effective amount. As used herein, a “therapeutically effective amount” or “effective amount” refers to the amount of DAN-222 and/or another drug, such as a PARP inhibitor, which is required to effect treatment of the particular disorder; i.e., to produce a reduction in the amount and/or severity of the symptoms associated with that disorder. For example, in the case of a cancer metastasis, administration of a therapeutically effective amount of a DAN-222 formulation as described herein results in reduction and/or reversal of the symptoms of metastasis; e.g., regression of the metastatic tumor. Therapeutically effective amounts vary with the type and extent of damage caused by the cancer, and can also vary depending on the overall condition of the subject.

The disclosed therapeutic compositions and formulations can also include pharmaceutically acceptable materials, compositions or vehicle, such as a liquid or solid filler, diluent, excipient, solvent or encapsulating material, i.e., carriers. These carriers can, for example, stabilize the DAN-222 nanoparticles and/or facilitate the retention of the DAN-222 nanoparticles in the body. Each carrier is “acceptable” in the sense of being compatible with the other ingredients of the formulation and not injurious to the subject. Some examples of materials which can serve as pharmaceutically-acceptable carriers include: sugars, such as lactose, glucose and sucrose; starches, such as corn starch and potato starch; cellulose and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol and polyethylene glycol; polyols, such as glycerin, sorbitol and mannitol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations. Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Another aspect of the present disclosure relates to kits for carrying out the administration of the DAN-222 formulations described herein, optionally in combination with another therapeutic agent (e.g., a PARP inhibitor such as niraparib), to a subject. In one embodiment, a kit comprises a composition of DAN-222 formulated in a pharmaceutical carrier, suitable for administration, e.g., by injection or infusion.

The composition may be formulated a single daily dosage. The composition may be formulated for multiple daily doses, e.g., two, three, four, five, six or more daily doses.

The composition may be provided to the subject according to any dosing schedule. The composition may be provided once per day. The composition may be provided multiple times per day.

The methods of treating a subject include providing a composition of the invention, as described above, to the subject. Providing may include administering the composition to the subject. The composition may be administered by any suitable route or means, such as orally, intravenously, enterally, parenterally, dermally, buccally, topically (including transdermally), by injection, infusion, nasally, pulmonarily, and with or on an implantable medical device (e.g., stent or drug-eluting stent or balloon equivalents).

The composition may be provided as a single unit dosage. The composition may be provided as a divided dosage.

The composition may be provided under any suitable dosing regimen. For example, the composition may be provided as a single dose or in multiple doses. Multiple doses may be provided in provided separated by intervals, such as 12 hours, 24 hours, 2 days, 3 days, 4 days, 5 days, 1 week, 2 weeks, 3 weeks, 4 weeks, or more. Multiple doses may be provided within a period of time. For example, multiple doses may be provided over a period of 1 day, 2 days, 3 days, 4 days, 5 days, 1 week, 2 weeks, 3 weeks, 4 weeks, or more. The compositions may be provided repeatedly for a specified duration. For example, and without limitation, the compositions may be provided for 1 week, 2 weeks, 3 weeks, 4 weeks, 6 weeks, 8 weeks, 12 weeks, 3 months, 4 months, 5 months, 6 months, 8 months, 10 months, 12 months or more.

### EXAMPLES

Aspects of the invention are illustrated in the examples provided below.

### Example 1-Antitumor Activity of DAN-222 In Vivo

The anti-tumor activity of DAN-222 was evaluated in several tumor xenograft studies and included HRD-positive and HRD-negative cell lines from both breast (BT-474, MDA-MB-231, MBA-MB-436) and ovarian (OVCAR3) tumors.

Camptothecin, the active ingredient in DAN-222, is a topoisomerase I inhibitor that acts by forming a ternary complex with topoisomerase I and DNA during replication and transcription in tumor cells, which blocks the re-ligation of DNA, resulting in torsional tension downstream of the replication fork, or of the RNA polymerase transcription complex, to cause DNA strand breaks. The results of the tumor xenograft studies show that DAN-222 is likely to be more efficacious as a cancer treatment than other topoisomerase I inhibitors due to its tumor accumulation through the enhanced permeability and retention (EPR) effect leading to greater and more prolonged topoisomerase I inhibition.

As described below, the anti-tumor activity of DAN-222 was evaluated in several tumor xenograft studies and included HRD-positive and HRD-negative cell lines from both breast (BT-474, MDA-MB-231, MBA-MB-436) and ovarian (OVCAR3) tumors. DAN-222 provided significantly greater efficacy by sustained tumor inhibition as compared to free CPT and the CPT derivative irinotecan alone.

Further, the effects of DAN-222 treatment in combination with orally administered niraparib tosylate (niraparib) were evaluated in the HRD-positive MDA MB-436 and the HRD-negative OVCAR3 xenograft models, respectively. The combination of DAN-222 with a PARP inhibitor showed significantly increased tumor inhibition compared to either agent alone. The efficacy data from these studies, demonstrating a more potent anti-tumor response when compared with those of vehicle control or either agent alone, provide evidence of the in vivo complementary effectiveness of DAN 222 as an anti-tumor agent, as well as information on effective doses and dosing schedules.

### Study ER2019-09-1B: HRD-Positive (ER-Positive, PR-Positive, HER2-Positive) Breast Ductal Carcinoma (BT-474 Model)

In Study ER2019-09-1B, DAN-222 was evaluated as a single agent in the BT-474 murine xenograft model of human breast cancer.

FIG. 2 provides the calculated median tumor volume (MTV) as a function of time presented by treatment group. FIG. 3 provides a Kaplan Meier plot of the time to endpoint (TTE, 2,000 mm3) data, showing the percentage of animals in each group remaining on study over time. As described in FIGS. 2 and 3, the animals in the study were dosed with either DAN-222, unconjugated (free) CPT, or the CPT derivative irinotecan.

The results of the study provided in FIGS. 2 and 3, demonstrate that once-weekly IV dosing with DAN-222 (3 mg/kg) for three weeks resulted in sustained tumor growth inhibition throughout the treatment and post-treatment period in the BT-474 model animals treated with DAN-222 when compared with that of vehicle control, unconjugated CPT, or irinotecan.

### Study ER2020-07-1: HRD-Positive (ER-Negative, PR-Negative, HER2-Negative [Triple Negative]) Breast Adenocarcinoma (MDA-MB-436 Model)

In Study ER2020-07-1, the anti-tumor activity of a range of doses of DAN-222 (0.3-3 mg/kg) given weekly, for four weeks, was studied as a single agent and in combination with low dose niraparib (25 mg/kg/day) in an HRD-positive (MDA-MB-436) xenograft model. The anti-tumor activity of a range of doses of DAN-222 (0.3-3 mg/kg) given weekly, for four weeks, was also studied in combination with a higher dose of niraparib (50/40 mg/kg/day) in HRD-positive (MD-MB-436) xenograft models.

FIGS. 4 and 5 provide median tumor volume measurements, over time, for Study ER2020-07-1, presented by treatment group.

As shown in FIG. 4, DAN-222 demonstrated significant single agent activity in the MDA-MB-436 study with weekly treatment up to 3 mg/kg DAN-222 for 4 weeks which resulted in tumor growth inhibition or tumor regression. Specifically, DAN-222 (0.3, 1 and 3 mg/kg) induced 11 partial regression (0.3 mg/kg, n=1; 1 mg/kg, n=6; 3 mg/kg, n=4) and four complete regressions (3 mg/kg, n=4) with three tumor free survivors observed at the 3 mg/kg dose level.

As shown in FIGS. 4 and 5, combination of DAN-222 with either low-dose (25 mg/kg) or high-dose (50/40 mg/kg) niraparib resulted in a more potent anti-tumor response when compared to either agent alone. There continued to be a dose response of DAN-222 to complete regression when combined with either high or low dose niraparib.

Study ER2020-07-01 also revealed that the combination of DAN-222 (3 mg/kg) and high dose niraparib (50/40 mg/kg) was considered intolerable based on limited safety endpoints (i.e., mortality and body weights). The efficacy was not measured.

### Study ER2020-07-2: HRD-Negative (ER-Positive, PR-Positive) Ovarian Adenocarcinoma Cell Lines (OVCAR3 Model)

In Study ER2020-07-2, the effects of DAN-222 treatment in combination with orally administered niraparib tosylate (niraparib) were evaluated in the HRD-negative OVCAR3 xenograft model.

Combination treatment regimens consisting of weekly injections of up to 3 mg/kg DAN-222 with daily administration of high-dose niraparib (50 mg/kg) were evaluated, and the results are presented in FIG. 6 for low-dose niraparib. As shown in FIG. 6, the OVCAR3 study demonstrated the combination of DAN-222 with niraparib showed efficacy independent of HRD status. Further, DAN-222 given as a single agent at 3 mg/kg had a profound effect with complete response in all seven animals, all of which were tumor free survivors. In addition, the combination treatment with 1 mg/kg DAN 222 with high-dose (50 mg/kg) niraparib resulted in one partial response and six complete responses, including four tumor free survivors.

### Example 2-Safety Pharmacology Studies

Camptothecin exposure and toxicokinetic (TK) parameters were evaluated in rats and dogs after either a single dose or repeated dosing of DAN-222 in studies that were part of a toxicology program. The rat and dog were chosen as the pharmacologically relevant rodent and non-rodent species for evaluating the toxicology of DAN-222 and for selecting a safe starting dose for initial human investigations. Since DAN 222 is a conjugated product, toxicokinetic (TK) evaluations included assessments of both total (conjugated and unconjugated) and free (unconjugated) CPT.

For evaluating the nonclinical safety profile of DAN-222 in support of a clinical IV dosing regimen, where a dose is administered every week, and to establish a safe clinical starting dose for a Phase I trial, studies in the toxicology program consisted of dose-range finding and repeat-dose rat and beagle toxicity studies. With the exception of the dose-range finding studies in rats and beagles, all studies were conducted in compliance with the Food and Drug Administration's regulations for Good Laboratory Practice (GLP) for Nonclinical Laboratory Studies (21 CFR, § 58).

The effects of DAN-222 on vital organ functions (e.g., cardiovascular, respiratory, and central nervous system) were evaluated as endpoints repeat-dose general toxicology studies performed in rats and dogs.

### Rat Studies

In Study NC2020-08-1, acute single dose IV administration established the maximum tolerated dose (MTD) at 9 mg/kg treatment and doses above the MTD (18 mg/kg) resulted in mortality. In each case, decreased hematopoietic cell counts in the bone marrow was considered cause of death or debility. At doses up to the MTD, dose dependent effects on body weights were noted at 3 mg/kg and higher, and effects on food consumption correlating with mean body weight losses were observed at doses of at least 9 mg/kg.

In Study NC2020-08-3, a GLP 4-week IV repeat-dose toxicology study conducted in Sprague Dawley rats treated with 1 to 9.6 mg/kg DAN-222, there were no test article-related effects on activity, autonomic, excitability, neuromuscular, physiological, or sensorimotor observations in male animals tested in the Functional Observational Battery on Days 1, 8, and 22. Adverse treatment-related findings observed in both males and females at 9.6 mg/kg included lower body weights corresponding with reduced food consumption, as well as changes in bone marrow cytology associated with microscopic findings of hypocellularity in the bone marrow. DAN-222 treatment related hematology findings included decreased red cell mass and decreased white blood cell count at doses of 3.2 mg/kg and higher. Decreased lymphocyte counts were also observed 9.6 mg/kg. In males, testicular degeneration at doses of at least 3.2 mg/kg was observed, and correlated with lower mean testis and epididymis weights.

Based on the findings, the NOAEL was considered to be 3.2 mg/kg CPT for females and 1.1 mg/kg for males.

The severely toxic dose to 10% of animals (STD10) was not able to be determined based on the nature and reversibility of findings and a lack of DAN-222 related mortality. The STD10 is thus assumed to be >9.6 mg/kg.

### Dog Studies

In Study NC2020-08-2, acute single dose IV administration established the MTD at 1.5 mg/kg in dogs. Treatment with DAN-222 at doses above the MTD (2.6 mg/kg) resulted in animals being euthanized early due to declining physical condition attributed to gastrointestinal degeneration and atrophy. Clinical observations that were noted prior to early euthanasia included decreased activity, suspected dehydration, hunched posture, and coldness to the touch, and both animals experienced body weight losses during the initial post-dosing days. In animals treated up to the MTD, clinical observations related to gastrointestinal stress correlating with reduced food consumption and body weight losses were noted; however, increases in these endpoints during the study suggested a trend toward recovery.

In Study NC2020-08-4, a GLP 4-week repeat-dose IV toxicology study conducted in beagle dogs treated with 0.32 to 0.96 mg/kg DAN-222, all electrocardiogram (ECG) evaluations were qualitatively and quantitatively normal at all dose levels tested. No abnormalities in rhythm or waveform morphology were observed based on comparisons of pre-treatment and post-treatment ECG recordings. No treatment-related effects on heart rate, QRS duration, QT, or QTc interval were observed based on comparisons of Days 1 and 22 (pre-dose and 2 hours post-dose) and at the end of the recovery period.

Adverse findings associated with treatment included lower mean body weights, lower body weight gains, reduced food consumption, and clinical observations of decreased activity and hunched posture were seen in the high dose (0.96 mg/kg) group of males. Decreased cortical cellularity of the thymus correlating with decreased thymus weight was noted in the high dose (0.96 mg/kg) groups of males and females.

Dose dependent decreases were observed in all leukocyte parameters, red blood cell mass parameters, platelets, and reticulocytes, correlating with microscopic findings of decreased bone marrow cellularity and increased myeloid: erythroid (M: E) ratio and percentage of lymphocytes in the bone marrow in 0.64 mg/kg group females and 0.96 mg/kg group males. Perivascular mixed cell infiltrates were detected in the liver at the end of recovery in males and females at 0.96 mg/kg and were considered adverse.

The NOAEL was considered to be 0.32 mg/kg for females and 0.64 mg/kg for males. Since recovery of the decreased bone marrow cellularity and correlating effects on hematology and bone marrow cytology was generally observed, the HNSTD was considered to be 0.64 mg/kg in both sexes, which translates to 12.8 mg/m² in humans.

### Genetic Toxicology

A single GLP in vitro genotoxicity study evaluating the potential mutagenic activity of DAN-222 in a bacterial reverse mutation assay was conducted (Study T2020-08). No evidence of mutagenicity was observed at any dose level tested in this study (at 1.0, 5.0, 10, 50, 100, 500, 1000, or 5000 μg/plate).

### Local Tolerance

The tolerability of IV administration of the formulation intended for clinical investigation was evaluated as part of the toxicology studies in rats and dogs.

### Nonclinical Safety Summary of DAN-222

The studies described above formed part of a comprehensive pharmacology and toxicology program designed to evaluate the potential effectiveness and toxicity of DAN-222 in relevant models when administered using the clinical route of administration and a dosing schedule that mimics the intended clinical regimen. These completed studies provide information characterizing the toxicological properties of DAN-222 in a manner sufficient to understand the seriousness and reversibility of toxicities associated with its use and to inform the selection of a safe clinical starting dose. Additionally, these toxicology studies have provided information that will allow for monitoring for potential safety signals in the clinical setting.

In the acute dose range finding toxicology studies, maximum tolerated dose (MTD) was defined (9 mg/kg DAN-222 in rats; 1.5 mg/kg DAN-222 in dogs) and doses above the MTD resulted in mortality from bone marrow toxicity and decreased hematopoietic cell counts in rats, or early euthanasia due to gastrointestinal degeneration and atrophy in dogs.

In the Good Laboratory Practice (GLP) repeat-dose toxicology studies, animals were treated once per week for 4 weeks mimicking one dosing cycle of DAN-222 to support the weekly administration in the clinical study. In rats, reduced food consumption and lower body weights, bone marrow toxicity, and testicular degeneration in males were adverse effects observed with treatment, resulting in a no observable adverse effect level (NOAEL) of 3.2 mg/kg for females and 1.1 mg/kg for males (equivalent to 19.2 and 6.6 mg/m², respectively). In dogs, lower body weights and reduced food consumption, hematological changes, and bone marrow toxicity were adverse effects observed with treatment, resulting in a NOAEL of 0.32 mg/kg for females and 0.64 mg/kg for males (6.4 and 12.8 mg/m²) and a highest non-severely toxic dose (HNSTD) of 0.64 mg/kg (12.8 mg/m²) in both sexes.

The proposed first-in-human (FIH) dose of DAN-222, determined by the maximum recommended starting dose (MRSD), is based on the HNSTD determined in dogs, considered to be the most relevant animal species tested. In the 4-week toxicology study in dogs, the HNSTD was 0.64 mg/kg. The 0.64 mg/kg dose in dogs scales to a human equivalent dose of 12.8 mg/m². Therefore, in accordance with recommendations in ICH S9, the MRSD is approximately 2 mg/m², based on a 6-fold safety factor applied to the HNSTD.

### Example 3-PK Parameters of DAN-222

Exposure and other PK parameters of CPT have been evaluated in the animal species used for toxicology studies (i.e., rat and dog). The distribution of CPT after DAN-222 dosing in naïve mice and in murine tumor models was also evaluated. Since DAN 222 is a conjugated product, PK evaluations included assessments of both total (conjugated and unconjugated) and free (unconjugated) CPT. Additionally, the stability of DAN-222 was tested using in vitro methods in human, dog, and rat plasma.

Following once weekly IV injection of DAN-222 to rats, exposure (area under the plasma concentration-time curve from time zero to the time of last observation [AUCₜₗₐₛₜ]) to free and total CPT increased with dose level from 1.1 to 9.6 mg/kg DAN-222, on Days 1 and 22. The exposure to free CPT was low (approximately 0.1% to 0.2% of the exposure to total CPT). The terminal half-life for total CPT was approximately 15 to 21 hours. Clearance and volume of distribution ranged from 14.9 to 24.2 mL/hr/kg and from 404 to 647 mL, respectively. This data suggests clearance was low relative to hepatic blood flow (0.4%), and volume of distribution indicated minimal distribution into tissues.

In a similar manner, the PK of DAN-222 was characterized in beagle dogs, following repeat dose weekly administration at dose levels of 0.32, 0.64, and 0.96 mg/kg DAN-222. Exposure to total CPT and free CPT, in terms of AUCₜₗₐₛₜ, increased with dose level in an approximately dose proportional manner. Clearance and volume of distribution ranged from 16.4 to 18.7 mL/hr/kg and from 563 to 649 mL/kg, respectively, on Days 1 and 22. Terminal half-life for total CPT was approximately 23.2 to 26 hours. Total CPT exposure increased in a dose proportional manner. There was no accumulation of total CPT or free CPT.

Calculated mean concentrations of free and total CPT in the plasma were similar between male and female rats. FIGS. 7A and 7B provide representative graphs of mean concentrations of free and total plasma CPT in male rats following IV administration of DAN-222 on Days 1 and 22.

The stability of DAN-222 in plasma from rat, dog, and human, based on the half-life of conjugated CPT in each matrix, was evaluated using in vitro methods. FIG. 8 provides the results of these studies, which shows the stability of DAN-222, presented as percent conjugated CPT over time in rat, dog, and human plasma.

As shown in FIG. 8, in rat and dog plasma, the half-life of DAN-222 was 35.3 and 45.5 hours, respectively. When compared with that in human plasma, in which the stability of DAN-222 was calculated as 35.7 hours, these results indicate that DAN-222 is sufficiently stable in rat and dog plasma, and the in vivo performance of the compound in the animals is predicted to be similar to that in humans, which supports the relevance of the selected species as models in the toxicology studies.

In the single-dose studies conducted in rats or dogs, systemic exposure to free and total CPT increased with dose level in both males and females, and exposure to free CPT represented only a small percentage (i.e., <1%) of the total CPT exposure. In the pivotal toxicology studies conducted in rats and dogs, animals received four weekly IV injections of DAN-222 (rats ranging from 1.1 to 9.6 mg/kg, and dogs ranging from 0.32 to 0.96 mg/kg) with TK evaluations after the first and the fourth doses. Terminal half-life of total and free CPT, respectively, ranged from 15 to 21 hours and 14 to 26 hours in rats, and from 23 to 26 hours and 24 to 42 hours in dogs across evaluation days. In general, increases in systemic exposure to total and free CPT with increasing dose level occurred in an approximately dose-proportional manner. Again, systemic exposure to free CPT represented only a small percentage of the total CPT exposure; in terms of AUCₜₗₐₛₜ, free CPT exposure accounted for approximately 0.1% to 0.2%, or <0.1%, of total CPT exposure in rats or dogs, respectively. Additionally, exposure was similar between males and females of both species, and no significant accumulation of total or free CPT after repeated dosing of DAN-222 was observed in any of the dosing groups evaluated.

The biodistribution of CPT to organs of interest was evaluated after a single dose of DAN-222 in female mice, including one study in naïve CD-1 mice and two studies in immunodeficient mice bearing human breast cancer xenografts. FIGS. 9A-9B provide the results of these studies and show the biodistribution of free and total CPT from 2 to 48 hours in liver, spleen, kidney, heart, and bone marrow (FIG. 9A), as well as total CPT levels in plasma and in skeletal muscle and tumor tissue (FIG. 9B), of tumor-bearing mice.

FIG. 10 provides a table summarizing the mouse biodistribution studies and their results.

These biodistribution studies conducted in the naïve and tumor-bearing mice demonstrated limited exposure to free CPT in healthy tissues (e.g., liver, heart, spleen, and kidney); and only low levels of total CPT, contained in the NPs but not released as free CPT, were quantifiable in bone marrow. In both naïve and tumor-bearing mice, the biodistribution of CPT after DAN-222 dosing demonstrated reduced exposure in bone marrow when compared to that of irinotecan. In tumor-bearing mice, increasing accumulation of CPT in the tumor from 2 to 48 hours was observed after a single dose of DAN-222, while levels decreased in plasma, liver, spleen, heart, and skeletal muscle from the contralateral flank over the same period of time.

Overall, free and total CPT were detected in most organs tested (e.g., liver, heart, spleen, and kidney); however, when compared with that of irinotecan, biodistribution to bone marrow was significantly reduced with DAN-222 administration. Levels of free CPT were less than 1% of the injected dose per gram of tissue in the organs tested. In tumor-bearing mice, accumulation of CPT was observed in tumor tissue, but not in normal tissues, demonstrating the EPR effect. Overall, the data from studies characterizing PK parameters and distribution of CPT after DAN-222 administration support the proposed clinical dosing schedule.

### Example 4-Preferred Formulation Development

Three iterative, experimental steps were performed as part of a Preliminary Formulation Development Screening to determine a preferred clinical formulation of DAN-222. This screening was designed to determine the initial indications of material stability and critical quality attributes related to DAN-222. Throughout the three formulation development steps, described below, many critical quality attributes (CQAs) were assessed to determine a preferred formulation of DAN-222. After each step, the highest performing formulations were selected for evaluation in the subsequent step. In this way, a preferred formulation of DAN-222 was determined.

The study was split into three steps performed under accelerated stability conditions: (1) univariate buffer and pH selection; (2) addition of critical excipients to top two buffers from step 1; and (3) top buffer/excipient from step 2 at a range of pH values.

As explained, DAN-222 is a nanoparticle comprised of strands of MAP-Gly-CPT (i.e., camptothecin conjugated to a hydrophilic copolymer). MAP-Gly-CPT consists of a 50-80 kDa mucic acid-polyethylene glycol (PEG) copolymer (MAP) conjugated to multiple CPT molecules via glycine linkers. When dissolved in aqueous media, the MAP-Gly-CPT polymer strands spontaneously assemble as nanoparticles with hydrophobic CPT in the core surrounded by hydrophilic MAP. On average, the nanoparticles have a mass of about 180 kDa and are 40-60 nm in diameter (see FIG. 1). The target CPT loading of formulated nanoparticles is 10-12 wt % with an average of two MAP-Gly-CPT strands per nanoparticle.

The physicochemical characteristics of DAN-222 drug substance necessitate its delivery as a parenteral solution. Consequently, pharmaceutical development focused on the creation of a drug product formulation for intravenous infusion. The formulation must meet several criteria: sterility; acceptable stability during bulk drug substance and drug product manufacture; acceptable stability over long-term storage; and acceptable stability during handling and administration. As an aqueous solution is required for formation of DAN-222 nanoparticles, formulation design focused on identification of excipients that provide both isotonicity as well as stability within a desired pH range (e.g., buffered pH). Intentionally, no unusual or novel excipients were evaluated. All excipients are available in compendial grades and are listed in the FDA inactive ingredients database.

The principal stability concern of DAN-222 formulated bulk drug substance (FBDS) and DAN-222 sterile solution for infusion is the release of free CPT from the nanoparticle. This measurement (free CPT as a percentage of total CPT) constitutes a key purity assay for DAN-222 and is the clearest indicator of product stability. The DAN-222 construct is designed to limit the acute toxicity of CPT by attaching it to the nanoparticle polymer, thereby controlling the release of this active moiety. Release of free CPT is caused by hydrolysis, and can be controlled with an optimized formulation, avoiding elevated temperatures in manufacturing and fill/finish, and keeping the drug substance and drug product frozen during long-term storage. As shown from the results of these studies, release of free CPT from DAN-222 was controlled with an optimized formulation, minimizing elevated temperatures in manufacturing and fill/finish, and keeping the drug substance (DS) and drug product (DP) frozen or assessing feasibility of lyophilization.

Nanoparticle size and polydispersity (PDI) is another avenue that has potential to impact pharmacokinetics and in vivo activity of the product. Moreover, solution properties, including pH, viscosity, solubility, and particle formation are all critical to control to ensure a safe and effective product that can be manufactured and administered. Nanoparticle size, polydispersity, and solution properties were evaluated for a number of different formulations exposed to varied conditions as part of the three studies.

### Screening Methods and Materials

### DAN-222 Active Pharmaceutical Ingredient

The active pharmaceutical ingredient (API) examined in these studies was DAN-222 manufactured under development conditions by Abzena, Bristol, PA. The material used for Step 1 and Step 3 of the screening was batch ABZ-2583/SKT-757-010-LYO/45 kDa. The material used for Step 2 was batch SKT-757-009-Lyo/53 kDa/11.79% CPT.

### Formulation Parameters

In each stability study, the formulations had fixed parameters of a buffer concentration of 20 mM, a fill volume of 0.5 mL, and an API concentration of 10 mg/mL.

Tables 1-3 provide the various formulations investigated at Steps 1-3 of the Preliminary Formulation Development Screening. The formulation codes are used when discussing the results obtained from the three steps of the screening.

TABLE 1
Formulation Matrix for Accelerated Stability Study Step 1

| Formulation | Formulation | | | API |
|---|---|---|---|---|
| No. | Code | Buffer (20 mM) | pH | (mg/mL) |
| 1 | S4 | Sodium succinate | 4.0 | 10 |
| 2 | C4 | Sodium citrate | 4.0 | |
| 3 | A4 | Sodium acetate | 4.0 | |
| 4 | W4 | Water + phosphoric acid | 4.0 | |
| 5 | H5 | Histidine-HCl | 5.0 | |
| 6 | P7.4 | Sodium phosphate | 7.4 | |

TABLE 2
Formulation Matrix for Accelerated Stability Study Step 2

| Formulation | | | Tonicity | | API | |
|---|---|---|---|---|---|---|
| Code | Buffer (20 mM) | pH | Modifier | | (mg/mL) | Surfactant |
| A4K | Sodium acetate | 4.0 | 200 | mM KCl | 10 | — |
| A4N | | | 200 | mM NaCl | | — |
| A4P | | | 200 | mM Proline | | — |
| A4A | | | 200 | mM Arginine-HCl | | — |
| A4S | | | 200 | mM Sucrose | | — |
| A4G | | | 200 | mM Glycine | | — |
| A4F68 | | | — | | | 0.05% F-68 |
| W4N | Water + phosphoric acid | 4.0 | 0.9% | NaCl | | — |
| H5K | Histidine | 5.0 | 200 | mM KCl | | — |
| H5N | | | 200 | mM NaCl | | — |
| H5P | | | 200 | mM Proline | | — |
| H5A | | | 200 | mM Arginine-HCl | | — |
| H5S | | | 200 | mM Sucrose | | — |
| H5G | | | 200 | mM Glycine | | — |

| | | | | |
|---|---|---|---|---|
| H5F68 | | | — | 0.05% F-68 |
| P7.4 | Sodium phosphate | 7.4 | — | — |

TABLE 3
Formulation Matrix for Accelerated Stability Study Step 3

| Formulation | | | | | API |
|---|---|---|---|---|---|
| Code | Buffer (20 mM) | pH | Excipient | | (mg/mL) |
| A4 | Sodium acetate | 4 | — | | 10 |
| A4N | | | 200 | mM NaCl | |
| A4.5 | | 4.5 | — | | |
| A4.5N | | | 200 | mM NaCl | |
| A5 | | 5 | — | | |
| A5N | | | 200 | mM NaCl | |
| W4N | Water + phosphoric | 4 | 0.9% | NaCl | |
| | acid | | | | |

In all three stages of the initial formulation development study, each formulation described above was subjected to various stress conditions: temperature; agitation; freeze-thaw; and forced oxidation. These conditions were designed to assess key manufacturing process risk factors as well as various temperature risk factors to product stability that may impact DAN-222 critical quality attributes.

Solution properties, including pH and visible particle formation, are also critical for the product. Through these formulation studies, it was found that pH is the most critical formulation property for nanoparticle purity. At around neutral pH and above, the release rate of CPT from the nanoparticle increased. At lower pH (e.g., pH 4-5), a significantly lower rate of CPT loss from the nanoparticle was observed. At a pH lower than 4 (i.e., pH 3), the release rate of CPT was slower than it was at pH 4. However, below pH 4, there are concerns of pain upon infusion. A pH of 4.3 was selected to provide a low release rate of CPT at both frozen and refrigerated (2-8° C.) temperatures as well as to reduce the risk of patient discomfort during infusion of this moderately acidic solution. Furthermore, it was determined that visible particle formation in solution occurs when the amount of free CPT reaches a critical level under stress conditions (roughly 30% of total CPT.)

Nanoparticle size and polydispersity index (PDI) are additional product quality attributes that have potential to impact in-vivo behavior of the product. Particle size and polydispersity can also give evidence of aggregation, which can lead to poor product performance and potential safety issues for the patient.

### Accelerated Stability Studies

Solid MAP-Gly-CPT conjugate (amorphous DAN-222 DS) was reconstituted in bulk with Milli-Q (MQ) water adjusted to pH 4 with phosphoric acid and stirred vigorously, but without formation of bubbles, for approximately 12 hours at ambient temperature.

Steps 1 and 3 utilized conjugate material from Lot SKT-757-010-LYO and Step 2 utilized conjugate material from Lot SKT-757009-Lyo. For Step 3, following reconstitution, an aliquot of bulk MAP-Gly-CPT in pH 4 MQ water was pulled for concentration measurement by absorbance (pre-0.45 μm filtration measurement) via SOP-0001-CPTRP. Following reconstitution, the solution was filtered using a 0.45 μm vacuum filter to clarify. Following filtration, the bulk formulated MAP-Gly-CPT in pH 4 water was split into appropriate portions, according to Tables 1, 2, and 3. Concentrated (10×) stocks of either buffer alone (Steps 1/3) or combination of buffer/excipient/surfactant (Steps 2/3) were slowly added into the pH 4 aqueous solutions with gentle stirring, at a 1/10 ratio to achieve target formulations at circa 10 mg/mL. For Step 3, following filtration and 10× buffer/excipient spiking, an aliquot of each formulated MAP-Gly-CPT portion was pulled for concentration measurement by absorbance (post-0.45 μm filtration/spiking measurement) via SOP-0001-CPTRP. All concentration measurements by absorbance were performed along with time zero measurements. No adjustments were made to sample concentrations following measurements.

After all formulation preparations were completed, formulations were sterile-filtered in an aseptic biological safety cabinet (BSC) using 0.22 μm syringe barrel filters and filled into the final container closures at 0.5 mL fill volumes. A 100 μL aliquot (Step 2) and 1 mL aliquot (Step 3) of each formulation was taken and lyophilized overnight to perform concentration analysis by weight of solids. After filling, the containers were sealed before removal from the BSC, capped/crimped and labeled. Samples were stored at the designated temperatures or exposed to acute stress conditions, such as agitation, freeze/thaw, and forced oxidation. Time zero analyses were performed.

Samples in the agitation studies were agitated on an orbital shaker at 450 rpm for 6 hours and 24 hours. An identical set of samples was also incubated at room temperature without agitation for comparison (Step 1). The one-day 25° C. samples were used as the static control for Steps 2 and 3. The forced oxidation samples were exposed to different concentrations of H₂O₂, according to Table 4, and immediately analyzed following 1 hour, 5 hours, and 24 hours (Steps 1 and 2). The freeze/thaw samples were exposed to consecutive cycles of uncontrolled freezing and thawing from −80° C. to ambient temperature and sampled at 3× F/T and 6× F/T (Step 1) or 6× F/T (Steps 2/3).

The accelerated stability study included the incubation of formulation candidates at frozen (−80° C. and −20° C.), refrigerated (2-8° C.), stressed (20-25° C.), and accelerated (40° C.) storage temperatures. The storage temperature portion of the study was performed over a 1-week period. Additional extended timepoints were added pending availability of additional sample volume following initial screening. Any additional timepoints were removed from the original vial and were not sterile.

Table 4 summarizes the acute stress conditions that were used for formulation stability evaluation.

TABLE 4
Summary of Accelerated Stability Stress Conditions

| Stress | Conditions | Time Point(s) |
|---|---|---|
| Temperature | −80° C. and −20° C. | 1 month, 3 months; 1 year time point |
| | 2-8° C.; 20-25° C.; 40° C. | T = 0, 1 day, 3 day, 1 week; extended |
| | | timepoints |
| Agitation | Vortex | Steps 1/2: 6 hr, 24 hr Step |
| | (450 rpm at ambient | 3: 24 hr |
| | temperature) | |
| Freeze-thaw | Uncontrolled | Step 1: 2 intervals of 3 consecutive |
| | (−80° C. to ambient | cycles (3x and 6x) |
| | temperature) | Steps 2/3: 1 interval of 6 consecutive |
| | | cycles (6x) |
| Forced | Step 1: 0.02% and 0.05% | Steps 1/2: 1 hr, 5 hr, 24 hr |
| Oxidation | H₂O₂ | |
| | Step 2: 0.05% H₂O₂ | |

Table 5 summarizes the analytical methods used in the screening.

TABLE 5
Analytical Methods used in Accelerated Stability Study

| | | | | Temperature Stress Time Points | | | | | |
|---|---|---|---|---|---|---|---|---|---|
| | Acute Stress | | | 2-8° C.; 20-25° C.; 40° C. | | | | −80° C. | |
| Analytical | | Freeze/ | Forced | | 1 | 3 | 7 | and −20° C. | |
| Methods | Agitation | Thaw | Ox | T = 0 | day | day | day | 1 mo | 3 mo |
| Visual | X | X | | X | X | X | X | X | X |
| pH | X | X | | X | X | X | X | X | X |
| Osmolality | X | X | | X | X | X | X | X | X |
| RP-HPLC | X | X | X | X | X | X | X | X | X |
| DLS | X | X | X | X | X | X | X | X | X |
| GPC | X | X | | X | X | X | X | X | X |

Visual inspection was performed under a white light source against black and white backgrounds. pH analysis was performed with an Orion 2 Star® pH Meter, calibrated with three pH standard solutions (pH 4, 7, and 10) with a calibration slope of 95% or higher. Samples were not temperature adjusted and were allowed to equilibrate to ambient temperature before being measured. Osmolality was measured using a Model 2020 Freezing Point Osmometer (Advanced Instruments).

For the RP-HPLC assays, free CPT samples were treated with HCl to convert the carboxylate CPT form to the lactone form. If CPT is not converted to the lactone form, it elutes earlier (around 2 to 4 minutes) along with polymer-bound CPT. The free CPT peak between 5.6 to 7.0 minutes increases in area as more CPT is lost from the polymer by hydrolysis. Total CPT samples were first treated with NaOH to release CPT from the parent polymer followed by HCl to convert the carboxylate CPT form to the lactone form. The amounts of these species were measured by monitoring peak absorbance at 370 nm.

Dynamic Light Scattering (DLS) was used for characterization of particle size based on the scattering of visible light resulting from the difference in refractive index between dispersed colloids and dispersion medium. DLS measures the fluctuations in light scattered by vibrating particles suspended in a liquid as a function of time. Brownian motion, which is caused by collision with solvent molecules of the liquid, causes the vibration.

### Step 1 Results

In Accelerated Stability Study Step 1, specific buffer and pH impacts on DAN-222 performance were assessed. As historical development data have shown that DAN-222 exhibits enhanced stability at lower pH, low pH buffer systems, including succinate, citrate, acetate, and histidine, were assessed. Slightly basic (pH 7.4) phosphate buffer was included in the assessment as a control (P7.4 in Tables 1 and 2).

### Visual Inspection Results

At time zero, as well as up to 7 days at 2-8° C. and 20-25° C., all formulations (listed in Table 1) were free from visible particles (FFVP). Following 3 days at 40° C., the control (P7.4) had visible particles present (PP). After 7 days at 40° C., formulation W4 and the control (P7.4) both had visible particle present, formulation H5 was practically free from visible particles (PFP), and all other formulations were free from visible particles. After 3 months at both −20° C. and −80° C., all formulations were free of visible particles.

Following 24 hours of agitation at 450 rpm, all formulations were free from visible particles (FFVP). After 6 consecutive, uncontrolled rounds of freezing and thawing from −80° C. to ambient temperature, all formulations were free from visible particles, but the control (P7.4) did form a gel like matrix until it reached room temperature when it returned to a liquid state. After 24 hours of exposure to either 0.02% or 0.05% peroxide, all formulations except the control (P7.4) were free from visible particles. The control (P7.4) had visible particles present (PP).

### pH and Osmolality Results

At time zero, the pH and osmolality of all formulations (listed in Table 1), with exception of formulation W4, were within target range. W4 experienced an increase in pH of 1.4 units following filtration using a PALL Acrodisc® 25 mm w/0.2 μm Supor® syringe filter (PALL Cat No. 4612). All osmolality values were within target range to theoretical values. After 7 days at 2-8° C., all formulations displayed pH and osmolality values similar to those observed at time zero. Following 7 days at 20-25° C., all formulations displayed pH and osmolality values comparable to those observed at time zero. After 7 days at 40° C., all formulations, except H5, displayed pH and osmolality values similar to those observed at time zero. H5 displayed a pH decrease of −1.1 pH units after 7 days at 40° C. After 3 months at −20° C. and −80° C., all formulations, except W4, displayed pH values similar to those observed at time zero. W4 exhibited a 1.0 unit pH increase at both temperatures, emphasizing the importance of adding a buffer to maintain pH. All formulations displayed osmolality values similar to those observed at time zero.

Following 24 hours of agitation at 450 rpm, all formulations displayed pH and osmolality values similar to those observed at time zero and to the static controls. After 3 consecutive uncontrolled freezing and thawing cycles from −80° C. to room temperature, all formulations displayed pH and osmolality values comparable to those observed at time zero. pH and osmolality data were not collected for six consecutive uncontrolled freezing and thawing cycles from −80° C. to room temperature.

Following addition of 0.02% peroxide and subsequent monitoring over 24 hours, all formulations displayed pH and osmolality values similar to those observed at time zero. pH and osmolality data were not collected at the 5-hour timepoint. After addition of 0.05% peroxide and subsequent monitoring over 24 hours, all formulations displayed pH and osmolality values comparable to those observed at time zero. pH and osmolality data were not collected at the 5-hour timepoint.

### RP-HPLC and DLS Results

At time zero, all formulations (listed in Table 1), except the control (P7.4), exhibited low percent free CPT. The phosphate control exhibited a larger increase of free CPT at time zero (+3.37%). All formulations displayed particle sizes in the range of 41.0 to 51.7 nm and similar PDI, ranging 0.293 to 0.438.

As shown in FIG. 11, following 14 days at 2-8° C., formulations S4, C4, and A4 at pH 4 displayed relatively small changes in release of free CPT compared to that observed at time zero (+0.51% to +0.58%). Formulations W4 and H5 displayed a slightly higher release of free CPT compared to time zero, ranging from +0.73% to +0.93%. The control formulation (P7.4) displayed a larger burst release of CPT following 14 days at 2-8° C., increasing 9.39% compared to time zero. Following 14 days at 2-8° C., all low pH formulations showed no significant change in particle size and PDI compared to time zero, with the exception of formulation S4 at 7 days. The control formulation (P7.4) showed a slight increase in particle size (+9 nm) compared to time zero.

As shown in FIG. 12, following 14 days at 20-25° C., formulations S4, C4, and A4 at pH 4 displayed a slight increase in release of free CPT compared to that observed at time zero (+1.60% to +1.76%). Formulations W4 and H5 displayed a slightly higher release of free CPT compared to that observed at time zero, ranging+2.34% to +3.30%. The control formulation (P7.4) displayed significant change in release of CPT following 14 days at 20-25° C., increasing 34.7% compared to that observed at time zero.

As shown in FIG. 12, following 14 days at 20-25° C., the low pH formulations showed little change in particle size and PDI compared to those observed at time zero. The citrate containing formulation displayed a slight increase in particle size after 7 and 14 days (+10.3 to +13.9 nm). The control formulation (P7.4) showed a significant increase in particle size (+140.1 nm) compared to time zero.

As shown in FIG. 13, following 14 days at 40° C., the low pH formulations displayed an increase in release of free CPT (+3.73% to +4.43%) compared to that observed at time zero, showing a more significant change from time zero, but significantly less release than high pH formulations. Formulations W4 and H5 displayed a slightly higher release of free CPT compared to time zero, ranging+4.16% to +5.23%. The control formulation (P7.4) displayed significant increase in release of CPT following 3 days at 40° C. (+32.47%) compared to time zero. The control formulation was not analyzed at later timepoints due to the presence of heavy particulate matter.

Following 3 days at 40° C., most formulations showed significant particle aggregation. While data suggests particle aggregation (+17.4 nm compared to time zero), the low pH formulations did not have any visible particles present after 14 days at 40° C. The control formulation (P7.4) showed significant particle aggregation after 3 days at 40° C. (+1476.3 nm) compared to that observed at time zero.

Following 3 months at −20° C., all formulations displayed little significant change in release of free CPT compared to that observed at time zero (+0.17% to +0.24%) and compared to 1 day at 2-8° C. (+0.0% to +0.07%).

Following 3 months at −20° C., all formulations showed no significant change in particle size and PDI compared to that observed at time zero.

Following 3 months at −80° C., all formulations displayed little significant change in release of free CPT compared to that observed at time zero (+0.14% to +0.21%) and compared to 1 day at 2-8° C. (+0.06%).

Following 3 months at −80° C., all formulations showed no significant change in particle size and PDI compared to those observed at time zero.

As shown in FIG. 14, following 24 hours of agitation at 450 rpm, all formulations, with the exception of control formulation P7.4, showed no significant change in release of free CPT compared to static controls. The control formulation (P7.4) displayed a slight increase in release of free CPT (+1.56%) compared to the static control.

Following 24 hours of agitation at 450 rpm, all formulations showed no significant change in particle size, when compared to the static control, and no visible particles were present.

Following 6 consecutive, uncontrolled rounds of freezing and thawing from −80° C. to room temperature, all formulations displayed free CPT percentages similar to those observed at time zero (+0.10% to +0.39%). There was no significant difference between 3 or 6 consecutive freezing and thawing cycles.

As shown in FIG. 15, following 6 consecutive, uncontrolled rounds of freezing and thawing from −80° C. to room temperature, all formulations showed no significant change in particle size, when compared to that observed at time zero. The high pH control formulation did exhibit gelation following thaw from freezing until the material reached room temperature.

As shown in FIG. 16, after 24 hours following addition of 0.02% peroxide, all low pH formulations showed little change in release of CPT compared to that observed at time zero (+0.28% to +0.32%). The significant difference in CPT release shown between W4 and H5, +0.95% vs+0.53%, suggests protective effects for buffer vs no buffer. The control formulation at high pH displayed a significant increase in release of free CPT (+16.58%) compared to that observed at time zero. After 24 hours following addition of 0.02% peroxide, all formulations, with exception of control formulation P7.4, showed little change in particle size compared to that observed at time zero. Particle size averages confirm that both physical and chemical degradation resulting from oxidation are correlated with increasing pH. The high pH control formulation displayed significant visible particle formation after the first hour following peroxide addition.

As shown in FIG. 17, after 24 hours following addition of 0.05% peroxide, all low pH formulations showed little change in release of CPT compared to that observed at time zero (+0.34% to +0.40%). The significant difference in CPT release shown between W4 and H5, +3.05% vs+1.05% increase, suggests protective effects for buffer vs no buffer. The control formulation at high pH displayed a significant increase in release of free CPT (+29.76%) compared to that observed at time zero. After 24 hours following addition of 0.05% peroxide, all formulations, with exception of control formulation P7.4, showed little change in particle size compared to that observed at time zero. Particle size averages confirm that both physical and chemical degradation resulting from oxidation are correlated with increasing pH. The high pH control formulation displayed significant visible particle formation and significant increase in particle size (+282.4 nm) after the first hour following peroxide addition.

### Step 2 Results

In Accelerated Stability Study Step 2, the two best performing buffer systems from Step 1 were chosen and used to assess specific excipient impacts on DAN-222 product performance. Common compendial excipients such as sugars, salts, amino acids, and surfactants were screened.

### Visual Inspection Results

At time zero, as well as up to 1 month at 2-8° C. and 7 days at 20-25° C., all formulations were free from visible particles (FFVP). The control (P7.4) developed visible particles (PP) after 14 days at 20-25° C., while all other formulations were free from visible particles for up to 1 month. Following 7 days at 40° C., all formulations, with exception of the control (P7.4), were free from visible particles. The control (P7.4) had visible particles present following 1 day at 40° C. All formulations tested were free from visible particles for up to 2 months at −20° C. and −80° C.

Following 24 hours of agitation at 450 rpm, 6 consecutive, uncontrolled rounds of freezing and thawing from −80° C., and 24 hours of exposure to 0.05% peroxide, all formulations were free from visible particles (FFVP).

### pH and Osmolality Results

At time zero, all formulations exhibited pH values within +0.38 pH units from target and osmolality values within +87 mOsm/kg from theoretical buffer osmolality. Following 1 month at 2-8° C., all formulations displayed similar pH values, ranging+0.17 pH units, and osmolality values, ranging #23 mOsm/kg, compared to these values at time zero. Following 1 month at 20-25° C., all formulations displayed similar pH values, ranging+0.19 pH units, and osmolality values, ranging+25 mOsm/kg, compared to the values observed at time zero. Following 7 days at 40° C., all formulations displayed a slight decrease in pH compared to time zero. Acetate formulations exhibited a decrease ranging+0.26 pH units and histidine formulations exhibited a decrease ranging+1.09 pH units. All formulations exhibited osmolality values similar to those observed at time zero, ranging+24 mOsm/kg.

Following 2 months at −20° C. and −80° C., all formulations displayed similar pH values, ranging+0.09 pH units, and osmolality values, ranging+19 mOsm/kg, compared to those observed at time zero. Following 24 hours of agitation at 450 rpm, all formulations displayed similar pH values, ranging±0.17 pH units, and osmolality values, ranging±24 mOsm/kg, compared to those observed at time zero and to those observed with the static control. Following 6 consecutive, uncontrolled freezing and thawing cycles from −80° C. to room temperature, all formulations displayed similar pH values, ranging±0.18 pH units, and osmolality values, ranging ±13 mOsm/kg, compared to those observed at time zero.

Twenty-four hours after addition of 0.05% peroxide, all formulations displayed similar pH values, ranging±0.18 pH units, and osmolality values, ranging±24 mOsm/kg, compared to those observed at time zero.

### RP-HPLC and DLS Results

At time zero, all formulations, except the control (P7.4), exhibited low percent free CPT values, ranging 0.54% to 0.67%. The phosphate control exhibited a larger amount of free CPT at time zero (2.35%). All formulations displayed particle sizes in the range of 54.0 nm to 65.1 nm.

Following 14 days at 2-8° C., all acetate and histidine formulations displayed a small change of free CPT compared to time zero (+0.31% to +0.47% vs+0.43% to 0.62%). The control (P7.4) displayed a larger burst release of CPT following 1 month at 2-8° C. (+9.13%) compared to the amount released at time zero.

As shown in FIGS. 18A-18B, following 1 month at 2-8° C., the select low pH formulations tested displayed little significant change of free CPT compared to time zero (+0.44% to +0.81%) in both acetate (FIG. 18A) and histidine (FIG. 18B) buffers. The control (P7.4) displayed a large burst release of CPT following 1 month at 2-8° C. (+14.72%) compared to the amount released at time zero. All formulations showed no significant change in particle size and PDI compared to time zero. Most formulations displayed a slight decrease in particle size.

As shown in FIG. 19, following 4 months at 2-8° C., A4N and W4N both displayed an increase in free CPT (+1.96% to +2.09%) as well as a decrease in particle size (−18.7 nm to −22.6 nm) compared to the values observed at time zero.

As shown in FIGS. 20A-20B, following 7 days at 20-25° C., all acetate and water formulations displayed an increase of +0.86% to +1.14% free CPT compared to time zero, while after 1 month at 20-25° C., the select acetate and water formulations tested exhibited a +2.54% to +3.18% increase in release of free CPT (FIG. 20A). The histidine formulations displayed an increase of +1.00% to +1.45% free CPT after 7 days at 20-25° C. compared to the amount of free CPT at time zero, while the select histidine formulations tested after 1 month at 20-25° C. displayed a +2.59% to +3.12% increase of free CPT compared to the amount of free CPT at time zero (FIG. 20B). The control formulation (P7.4) displayed a significant change in release of CPT following 7 days (+24.71%) and 1 month (+38.14%) at 20-25° C. Following 14 days at 20-25° C., the acetate, water, and histidine containing formulations showed a slight to moderate decrease in particle size (−4.2 to −22.8 nm) compared to time zero. Those select formulations tested after 1 month at 20-25° C. continued the trend of displaying smaller particle size compared to time zero (−14.0 to −21.6 nm). The control formulation (P7.4) displayed little to no change in particle size after 14 days at 20-25° C. compared to time zero.

As shown in FIGS. 21A-21B, following 7 days at 40° C., all low pH formulations displayed a large increase in release of free CPT compared to the amount of free CPT present at time zero (+4.05% to +5.74%). Results for acetate buffer are shown in FIG. 21A; results for histidine buffer are shown in FIG. 21B. The control formulation (P7.4) displayed a significant change in release of CPT (+34.78%) following 7 days at 40° C. Following 7 days at 40° C., the acetate, water, and histidine containing formulations showed a slight decrease in particle size (−0.2 to −16.9 nm) compared to size observed at time zero. The control formulation (P7.4) displayed a significant increase in particle size (+990.3 nm) after 3 days at 40° C. compared to that observed at time zero and was not tested at further timepoints.

As shown in FIG. 22, following 2 months at −20° C., all formulations tested displayed little significant change in release of free CPT compared to the amount of free CPT present at time zero (+0.03% to +0.09%). The control formulation (P7.4) showed a slight decrease of free CPT, compared to time zero (−0.32%). Following 4 months at −20° C., the formulations tested showed a slight increase in release of free CPT compared to the amount of free CPT present at time zero (+0.27% to 0.44%). Following 2 and 4 months at −20° C., all formulations showed no significant change in particle size and PDI compared to time zero. Most formulations displayed a slight decrease in particle size.

As shown in FIG. 23, following 2 and 4 months at −80° C., all formulations tested displayed little significant change in release of free CPT compared to the amount of free CPT present at time zero (+0.01% to +0.08% and +0.11% to 0.15%, respectively). After 2 months, the control formulation (P7.4) showed a slight decrease of free CPT, compared to the amount of free CPT present at time zero (−0.43%). Following 2 and 4 months at −80° C., all formulations showed no significant change in particle size and PDI compared to time zero. Most formulations displayed a slight decrease in particle size.

As shown in FIGS. 24A-24B, following 24 hours of agitation at 450 rpm, all formulations, with exception of the control, showed no significant change in release of free CPT compared to the amount of free CPT present at time zero. The control formulation (P7.4) displayed a +5.11% increase of free CPT compared to time zero. Following 24 hours of agitation at 450 rpm, all formulations showed a slight decrease in particle size compared to time zero (−2.7 nm to −16.3 nm). Results for formulations in acetate buffer are shown in FIG. 24A; results for formulations in histidine buffer are shown in FIG. 24B.

As shown in FIGS. 25A-25B, following six consecutive uncontrolled rounds of freezing and thawing from −80° C. to room temperature, all formulations, with exception of the control, displayed similar free CPT percentages compared to the amount of free CPT present at time zero (+0.14% to +0.28%). The control formulation (P7.4) showed a +2.16% increase of free CPT compared to time zero. Following six consecutive cycles of uncontrolled freezing and thawing from −80° C. to room temperature, all formulations showed a trend towards slight decreases in particle size when compared to time zero (+2.7 to −20.0 nm). Results for formulations in acetate buffer are shown in FIG. 25A; results for formulations in histidine buffer are shown in FIG. 25B.

As shown in FIGS. 26A-26B, at twenty-four hours following addition of 0.05% peroxide, all acetate and water formulations showed little change in release of CPT compared to time zero (+0.30% to +0.72%) (FIG. 26A). The histidine formulations displayed a slightly higher increase in release of CPT compared to time zero (+0.72% to +1.18%) (FIG. 26B). The control formulation at high pH displayed a significant increase in release of free CPT (+47.64%) compared to time zero (FIG. 26B). Twenty-four hours after addition of 0.05% peroxide, all formulations, with exception of the control, showed little change in particle size compared to the size observed at time zero. The control formulation (P7.4) displayed a significant increase in particle size (+1304.3 nm) after 5 hours following peroxide addition (FIG. 26B).

### Step 3 Results

In Accelerated Stability Study Step 3, the top performing buffer system/excipient combination, from Step 2, was chosen to run a robustness examination at pH's 4.0, 4.5, and 5.0.

### Visual Inspection Results

At time zero, as well as up to 1 month at 2-8° C. and 20-25° C., all formulations were free from visible particles (FFVP). Following 1 day at 40° C., all formulations, with exception of those at pH 5, were free from visible particles. The pH 5 formulations had visible particles present (PP).

Following 1 month at −20° C. and −80° C., all formulations were free from visible particles.

Following 24 hours of agitation at 450 rpm and six consecutive, uncontrolled rounds of freezing and thawing from −80° C., all formulations were free from visible particles.

### pH and Osmolality Results

At time zero, all formulations exhibited pH values within +0.14 pH units of target and osmolality values within +34 mOsm/kg of theoretical buffer osmolality. Following 1 month at 2-8° C., all formulations, with exception of W4N, displayed similar pH values, ranging±0.22 pH units, and osmolality values, ranging±15 mOsm/kg, compared to those observed at time zero. W4N exhibited a 0.54 pH unit decrease compared to time zero.

Following 1 month at 20-25° C., all formulations displayed similar pH values, ranging±0.24 pH units, and osmolality values, ranging #16 mOsm/kg, compared to those observed at time zero. Following 7 days at 40° C., all formulations displayed similar pH values, ranging±0.09 pH units, and osmolality values, ranging±5 mOsm/kg, compared to those observed at time zero. Following 1 month at −20° C. and −80° C., all formulations displayed similar pH values, ranging #0.12 pH units, and osmolality values, ranging±10 mOsm/kg, compared to those observed at time zero.

Following 24 hours of agitation at 450 rpm, all formulations displayed similar pH values, ranging±0.04 pH units, and osmolality values, ranging±5 mOsm/kg, compared to those observed at time zero. Following 6 consecutive uncontrolled freezing and thawing cycles from −80° C. to room temperature, all formulations displayed similar pH values, ranging±0.12 pH units, and osmolality values, ranging±10 mOsm/kg, compared to those observed at time zero.

### RP-HPLC and DLS Results

At time zero, all formulations exhibited low percent free CPT values, ranging 0.65% to 0.74% and particle sizes in the range of 42.2 nm to 47.3 nm.

As shown in FIG. 27, following 1 month at 2-8° C., all low pH formulations displayed a slight increase of free CPT compared to the amount of free CPT present at time zero (+0.33% to +0.80%). Formulations containing buffer and salt displayed lower percent free CPT (avg. −0.12%) than their respective pH counterpart containing buffer only. In addition, the lower the formulation pH, the lower the percent free CPT. All formulations showed no significant change in particle size and PDI compared to those values observed at time zero.

As shown in FIG. 28, following 3 months at 2-8° C., all formulations displayed an increase of free CPT compared to the amount of free CPT present at time zero (+1.34% to +2.23%). Formulations containing buffer and salt displayed lower percent free CPT (avg. −0.28%) than their respective pH counterpart containing buffer only. In addition, the lower the formulation pH, the lower the percent free CPT. All formulations showed no significant change in particle size and PDI compared to those values observed at time zero (−1.0 nm to +6.45 nm).

As shown in FIG. 29, following 7 days at 20-25° C., all formulations displayed an increase of free CPT compared to the amount of free CPT present at time zero (+0.65% to +1.12%). Formulations containing buffer and salt displayed lower percent free CPT (avg. −0.30%) than their respective pH counterpart containing buffer only. Following 1 month at 20-25° C., all low pH formulations displayed a larger increase of free CPT compared to the amount of free CPT present at time zero (+2.14% to +4.18%). Formulations containing buffer and salt displayed lower percent free CPT (avg. −0.58%) than their respective pH counterpart containing buffer only. In addition, the lower the formulation pH, the lower the percent free CPT that was detected. Following 1 month at 20-25° C., all formulations showed no significant change in particle size and PDI compared to those values observed at time zero (−1.6 nm to 4.5 nm).

As shown in FIG. 30, following 7 days at 40° C., all formulations displayed a larger increase in release of free CPT compared to the amount of free CPT present at time zero (+3.06% to +5.00%). All formulations exhibited slight changes in particle size, compared to time zero, after 7 days at 40° C. (+15.9 nm).

As shown in FIG. 31, following 1 month at −20° C., all formulations displayed no significant change in release of free CPT (−0.02% to +0.11%) and no significant change in particle size (−2.1 nm to +9.6 nm) compared to those values at time zero. Following 3 months at −20° C., there was a slight increase in release of free CPT in formulations at pH 4.5 and 5.0 compared to those at pH 4.0. Compared to time zero, the percentage of free CPT increased from 0% to 0.27% in seven different formulations. There was no significant change in particle size (−5.6 nm to +7.4 nm) compared to time zero in the formulations.

As shown in FIG. 32, following 3 months at −80° C., all formulations displayed no significant change in release of free CPT (+0.05% to +0.15%) and no significant change in particle size (−6.0 nm to 5.8 nm) compared to those values at time zero.

As shown in FIG. 33, following 24 hours of agitation at 450 rpm, all formulations showed little significant change in release of free CPT compared to the amount of free CPT present at time zero (+0.07% to +0.39%), with higher pH formulations exhibiting a larger release in CPT compared to lower pH formulations. All formulations showed no significant change in particle size compared to size at time zero.

As shown in FIG. 34, following six consecutive rounds of uncontrolled freezing and thawing from −80° C. to room temperature, all formulations exhibited no significant change in release of free CPT or change in particle size compared to those values at time zero.

Conclusions from Steps 1-3

In the formulation buffer screen (Step 1), a clear benefit was seen using a low pH-based formulation buffer for chemical (CPT loss), physical (particle size and PDI), and solution properties. At 2-8° C., low pH formulations showed minimal CPT loss, at ˜0.03% per day, while the control (P7.4) displayed a 20-fold higher CPT loss, at ˜0.6% per day. All formulations, regardless of pH, showed no significant change in physical or solution stability. At 20-25° C., pH 4.0 formulations exhibited ˜0.03% CPT loss per day and pH 5 formulations displayed ˜0.2% CPT loss per day. The control (P7.4) exhibited a greater than 30-fold higher CPT loss at ˜3.6% per day. All formulations, regardless of pH, exhibited no significant change in physical or solution stability. At 40° C., low pH formulations displayed a higher percent CPT loss per day at ˜0.6% as well an increased particle size after 3 days. The control (P7.4) exhibited a significant percent CPT loss per day at ˜32.0% and rapid visible particle formation after 1 day. At −20° C. and −80° C., all formulations, with exception of the control (P7.4), exhibited minimal CPT loss per day, at ˜0.01%. The phosphate control exhibited ˜0.04% CPT loss per day. All formulations showed no significant change in physical or solution stability.

Following 24 hours of agitation at 450 rpm, all formulations displayed minimal effects on either chemical or physical stability. There was a slight increase in PDI following agitation. Freeze/thaw cycling displayed minimal effect on particle structure and chemical stability in all groups, except the control (P7.4) where gelation was observed upon thawing for a brief period. There was no significant difference seen for chemical or physical stability between 3 or 6 consecutive freeze/thaw cycles. At low pH, there was no significant change in physical stability and minimal change in chemical stability following oxidative stress. As pH and peroxide levels increased, significant CPT loss was observed. The control (P7.4) showed high physical structure change rapidly following oxidative stress, including visible particle formation.

Sodium acetate buffer systems showed some enhanced benefit, compared to other pH 4 systems, with reduction of chemical and physical degradation, so sodium acetate was chosen as the lead buffer system to be used in Step 2. Histidine was also assessed in Step 2 as a moderate pH option.

In the formulation excipient screen (Step 2), a wide variety of salts, sugars, amino acids, and surfactants were screened in both acetate buffer at pH 4.0 and histidine buffer at pH 5.0. At 28° C., formulations at pH 4.0 and 5.0 exhibited minimal CPT loss per day at ˜0.03% and showed no significant change in physical or solution stability. Excipient groups within the same buffer system showed no significant difference in chemical, physical, or solution stability. At 20-25° C., CPT loss per day was ˜0.1% to 0.2% at both pH 4.0 and pH 5.0, depending on excipient. Average particle size decreased as temperature exposure increased and plateaued around 7 days. There was no significant solution stability change at either pH. At 40° C., CPT loss per day increased to ˜0.6% and fluctuations in particle size were present after 3 days at both pH values. There were no significant changes in solution properties. At −20° C. and −80° C., formulations at pH 4.0 and 5.0 exhibited minimal CPT loss per day, at ˜0.01%, and showed no significant change in physical or solution stability.

Following 24 hours of agitation at 450 rpm, all formulations, except the control (P7.4), displayed minimal impact on physical stability, as evidenced by only small decreases in particle size. In addition, no significant effects on either chemical stability or solution properties were observed by visual inspection. Small increases in PDI were seen in some excipient groups but were less pronounced in pH 4.0 formulations. Freeze/thaw cycling showed minimal effect on particle structure and chemical stability in pH 4.0 and 5.0 formulations. Following addition of peroxide, all low pH formulations exhibited no significant change in physical stability, and a slight effect on chemical stability. Greater CPT loss was seen in pH 5.0 formulations compared to pH 4.0 formulations.

The use of salts as excipients showed added benefits of controlling product CQAs compared to other excipients. Specifically, salts were observed to help control polydispersity consistently among different stressed groups. Sodium chloride was selected to move into Step 3.

In the formulation confirmation screen (Step 3), acetate formulations with and without NaCl were tested at pH 4.0, 4.5, and 5.0. At 2-8° C., percent CPT loss per day was ˜0.03% to 0.05% at pH 4.0 to 5.0. There was no significant change in physical or solution stability at all pH values up to 1 month. W4N formulation exhibited a 0.54 pH unit decrease following 1 month at 2-8° C. compared to time zero, stressing the importance of using a buffered system. At 20-25° C., percent CPT loss per day was ˜0.1% to 0.2% depending on pH and presence of salt. The inclusion of salt showed significant protection against CPT loss compared with buffer alone in the same pH group for all three pH values. No significant change in physical or solution properties at all pH values with or without salt was observed for up to 1 month. At 40° C., percent CPT loss increased to ˜0.6% per day at all pH values. Fluctuations in particle size were observed after 3 days, where particle size often decreased in average size followed by increasing in size. The inclusion of salt at high temperatures may have minimally prevented increase in particle size. Following 1 day, formulations at pH 5.0 exhibited presence of visible particles, while formulations at pH 4.0 and 4.5 exhibited no visible particles up to 1 week. At −20° C. and −80° C., all formulations exhibited minimal CPT loss per day at ˜0.02% to 0.03% and showed no significant change in physical or solution stability.

Agitational stress at 450 rpm for 24 hours had minimal impact on chemical (0.1% CPT loss/day) and physical stability. Freeze/thaw cycling also had minimal impact on product quality in all formulations regardless of pH or salt presence. A minimal increase in PDI was observed in some groups and was more pronounced in those without salt.

A significant benefit can be seen with the inclusion of NaCl over buffer alone in most stress groups and data for pH 4.0 showed increased stability compared to pH 4.5 and 5.0.

The results of this initial formulation development study led to the selection of the target phase 1 clinical formulation and drug substance concentration of 20 mg/mL DAN-222 in 20 mM sodium acetate (equivalent to 6.8 mM sodium acetate anhydrous, 13.2 mM acetic acid), 200 mM sodium chloride, pH 4.3.

Follow-up formulation development stability studies have been performed using several development lots of DAN-222 material using the proposed clinical formulation and API concentration. The results of these studies indicate that the product is robust and maintains product quality when exposed to various conditions expected during manufacture and storage. The formulation of this GLP-toxicology study batch is identical to that of the proposed phase 1 clinical formulation for DAN-222 FBDS and drug product. To date, at the same proposed storage temperature of the phase 1 clinical drug product (−20±5° C.), the stability trends show that the product is very stable with respect to its critical quality attributes under this storage condition.

### Example 5-DAN-222 Formulated Bulk Drug Substance (FBDS) Properties

This example provides the methods and analysis used to develop and identify the properties of the DAN-222 FBDS which may be used to produce the DAN-222 sterile solution for infusion.

### Properties of DAN-222

The drug substance, DAN-222, consists of strands of 20 (S)-camptothecin (CPT) covalently bound to a mucic acid-based polymer carrier via a glycyl linkage (MAP-Gly-CPT). During manufacture, MAP-Gly-CPT is isolated as an amorphous solid. In aqueous solution, the MAP-Gly-CPT polymer-drug conjugate self-assembles into nanoparticles (NPs). Drug substance properties described here refer to either MAP-Gly-CPT amorphous or DAN-222 Formulated Bulk Drug Substance (FBDS), as specified. A listing of development batches from which these properties were derived is provided in Table 6.

TABLE 6
Development batches of DAN-222 FBDS
used to establish properties

| Lot No. | Material | Use |
|---|---|---|
| PS533 | Gly-CPT | Development batch |
| MAP.041B | MAP | Development batch |
| SKT-874-052-2^{ND}- | MAP-Gly-CPT | Development batch |
| 1.2CPT | amorphous | |
| SKT-874-067-1.2CPT- | MAP-Gly-CPT | Development batch |
| 25G-LYO | amorphous | |
| SKT-874-069 | DAN-222 FBDS | Development batch |
| SKT-874-073 | DAN-222 FBDS | Development batch |

When purified and isolated by ultrafiltration/diafiltration, concentrated MAP-Gly-CPT is lyophilized to provide a solid intermediate. The isolated conjugate material is a light yellow to yellow, amorphous low-density solid.

DAN-222 FBDS is obtained by dissolution of MAP-Gly-CPT amorphous in water for injection adjusted to pH 4.0, followed by addition of formulation buffer concentrate to meet the desired buffer concentration (pH 4.3). This solution is concentrated to the desired strength by ultrafiltration. As formulated, DAN-222 FBDS is a clear to slightly opalescent, colorless to light yellow liquid.

A series of tests were performed to determine the impact to aqueous pH resulting from the dissolution of MAP-Gly-CPT in purified water. Samples were tested using a Thermo Scientific Orion 2 Star benchtop PH meter with an Orion 8103BN probe at ambient temperature. Material from two different MAP-Gly-CPT amorphous development batches (SKT-874-052-2^{ND}-1.2CPT and SKT-874-067-1.2CPT-25GLYO) were assessed. MAP-Gly-CPT amorphous from these batches was dissolved at various concentrations in sterile filtered Milli-Q® water. Dissolution of MAP-Gly-CPT results in a small drop in aqueous pH. This may be due to the presence of some free carboxylic acid moieties that are not conjugated to CPT. As formulated, DAN-222 FBDS solution is buffered, with a target pH of 4.3±0.3.

The solubility of DAN-222 in water at ambient temperature was assessed. MAP-Gly-CPT amorphous from development batches SKT-874052-2^{ND}-1.2CPT and SKT-874-067-1.2CPT-25G-LYO was dissolved with sterile filtered Milli-Q® water. Solubility was determined by creating a saturated mixture above the solubility limit in a clear glass vial and visually assessing the impact of serial additions of water.

As DAN-222 incorporates a hydrophilic polymer backbone, its behavior at high concentration differs from that of soluble crystalline materials. No significant differences were observed in the solubility of the two batches studied. Starting at 400 mg/mL, the material is not fully soluble and the mixture is heterogeneous. The solid appears to absorb the initial aliquots of water, and exhibits a uniform, translucent gel-like state at around 265 mg/mL. Full solubility is attained at about 230 mg/mL, resulting in a clear, yellow highly viscous solution capable of little flow. Further addition of water results in decreased viscosity.

The viscosity of two different development batches of DAN-222 FBDS was measured at ambient temperature using a Brookfield DV-II+ Pro viscometer with a Brookfield CPA-40Z plate. The speed of the plate was set at 50 revolutions per minute (rpm) for both samples and the percent torque was measured. The two batches, SKT-874-069 and SKT-874-073 were formulated in 20 mM sodium acetate, 200 mM sodium chloride at pH 4.3 (formulation buffer). The results showed that viscosity is concentration-dependent. The formulated solution (target 20 mg/mL) is slightly more viscous than water (comparable to that of whole milk) and presents no impediment to removal from the vial by syringe.

Prior to formulation as the drug substance, MAP-Gly-CPT was isolated after concentration and lyophilization as an amorphous solid. This low-density, solid material is friable and somewhat sponge-like and does not visibly exhibit any signs of crystalline structure. The x-ray powder diffraction (XRPD)diffractograms of MAP-Gly-CPT as well as its precursors mucic acid polymer (MAP) and glycylcamptothecin (TFA salt; Gly-CPT) were obtained using a Rigaku SmartLab X-ray diffractometer. Data collection was performed with the instrument in a Grabb-Brentano reflection geometry using a line source X-ray beam.

The XRPD results demonstrate that Gly-CPT, as isolated for use in the synthesis of DAN-222, clearly exhibits a crystalline form. Conversely, neither MAP nor MAP-Gly-CPT exhibit characteristics of an orderly crystalline nature. The broad, diffuse nature of the diffractograms of both compounds show little crystalline organization. In the diffractograms for both MAP and MAP-Gly-CPT, the broad peaks at about Theta 19°, 24°, 27° and 36° suggest some potential “disordered” crystallinity, but the pattern confirms the gross visual assessment of both materials as amorphous. The XRPD pattern for MAP-Gly-CPT is very similar to that of MAP, strongly suggesting that the structure of the solid is dominated by the characteristics of MAP, and any tendency for the CPT moiety to crystallize is inhibited by its attachment to MAP.

### Development of Specifications for DAN-222 FBDS

The justifications for the specifications of the DAN-222 Formulated Bulk Drug Substance (FBDS) are provided in this section. Each method and initial specification is based on a combination of process capability as demonstrated through process development, and the capabilities of phase-appropriate, qualified and/or compendial analytical methods.

The specification for color of DAN-222 FBDS is colorless to yellow liquid≤Y3. This specification incorporates the range of colors observed in acceptable batches tested and used during development (including batches used for the dose range finding and IND-enabling GLP-toxicology studies); the color of the initial GMP drug substance batch (Lot 21-M007) conforms with this limit. The potential for a yellow solution is based on the presence of 20 (S)-camptothecin (CPT), which is known to be intensely yellow in color. In solid form, both MAP-Gly-CPT amorphous and free CPT are yellow and can impart a faint yellow tint to the solution. Color intensity greater than Y3 color standard (for example, Y2 or Y1) is indicative of potential degradation.

The specification for clarity of DAN-222 FBDS is clear to slightly opalescent liquid≤ Standard III. This specification incorporates the range of clarity observed in acceptable batches tested and used during development (including batches used for the dose range finding and GLP toxicology studies); the clarity of the initial GMP drug substance batch (Lot 21-M007) conforms with this limit. DAN-222 FBDS is generally clear, although visual assessment may be impacted by lighting, observation conditions, and reflections and refraction from containers.

Identifying DAN-222 FBDS was accomplished using a specific combination of four separate analytical results. This multi-analysis approach is necessary to ensure that one can distinguish between DAN-222 FBDS, MAP-Gly-CPT amorphous, and free 20 (S)-camptothecin (CPT). The four analytical methods for identity used to discriminate between DAN-222 FBDS, MAP-Gly-CPT amorphous, and free CPT in solution are: (i) identity by ultraviolet-visible spectroscopy (UV-Vis); (ii) appearance; (iii) percent free CPT (expressed as percentage of total CPT) as measured by reversed-phase high-performance liquid chromatography (RP-HPLC); and (iv) weight percent total CPT by RP-HPLC.

The identification of a DAN-222 FBDS sample by UV-Vis evaluates 2 different wavelength ratios to show identity (absorbance at 252 nm/356 nm ratio; and absorbance at 295 nm/356 nm ratio). These absorbance ratios were found to be unique to CPT-containing compounds. This specification is justified upon the use of development DAN-222 FBDS as a reference standard to establish these absorbance ratios and acceptable ranges.

As stated, the appearance analysis will differentiate a sample of MAP-Gly-CPT amorphous (a solid) from that of liquid MAP-Gly-CPT nanoparticles (DAN-222 FBDS). This specification is justified and sufficient to further prove identity of the FBDS, which is a liquid, compared to MAP-Gly-CPT amorphous solid.

Finally, the criteria for % free of total CPT and weight % total CPT values by RP-HPLC are used to differentiate between a liquid sample of CPT and DAN-222 FBDS. These tests are also used to demonstrate that the ratio of CPT present relative to the total drug substance quantity correlates with that expected for DAN-222 FBDS. These levels are justified based upon the DAN-222 FBDS specification limits provided herein.

Purity is assessed by the % free CPT (with respect to total CPT) in a DAN-222 FBDS sample. This value is determined from the calculated free and total CPT concentrations in the sample. The “% free of total CPT” value provides the clearest indicator of drug substance stability during storage and use. The DAN-222 construct is designed to limit the acute toxicity of CPT by attaching it to the nanoparticle (NP) polymer, thereby controlling the release of this active moiety. The release specification for % free of total CPT in the drug substance is ≤3%. The stability specification for % free of total CPT in the drug substance is ≤5%. These limits are well below prior studies using CPT administered directly as a free drug. In two separate 3-week mouse xenograft studies (ER2019-09-1B, ER2019-09-1C) CPT itself was dosed and well-tolerated at 3 mg/kg which has a human equivalent dose of 9 mg CPT/m2.

Additionally, these limits allow for a significantly reduced free CPT exposure for DAN-222 relative to the tolerated human equivalent dose of CPT at proposed clinical dosing levels. For example, a potential high-dose cohort for the phase 1 clinical study could be 16 mg CPT/m². At the proposed release specification, the total free CPT would not exceed 0.48 mg/m², well below the tolerated dose. The proposed stability specification of ≤5% free CPT results in a free CPT dose of 0.8 mg/m². Each of these free CPT exposures is more than one order of magnitude lower than the tolerated human equivalent dose of 9 mg/m² CPT described above.

The assessment of total CPT mass in proportion to the total drug substance mass (weight % total CPT) provides an indication of the performance and control of the MAP-Gly-CPT synthesis process. This criterion is assessed for manufacture of the drug substance but is not repeated for release of the drug product as the combined assessment of % free of total CPT, CPT concentration, and % CPT-related substances is sufficient to address drug product quality. The specification for weight % total CPT is 11-15%. This specification encompasses the middle-to-upper end of the range of CPT loading studied during process development. The upper end of this range is derived from the theoretical maximum CPT loading for MAP-Gly-CPT, assuming conjugation of Gly-CPT to each available site on a MAP polymer strand. The lower end of this specification is at a CPT loading level that has been studied in animal models and successfully demonstrated during process development.

Measuring the total CPT concentration in DAN-222 FBDS provides the basis for weight-based dosing of DAN-222 in the clinic. The total CPT concentration assay value represents the sum of all CPT species present in the vial, including CPT covalently bound to the nanoparticle polymer backbone; free CPT in solution; and possible trace contributions from unreacted Gly-CPT. The drug substance specification range for total CPT concentration is based on the allowable weight % total CPT in MAP-Gly-CPT. The specification limits for CPT concentration are derived from the lowest acceptable DAN-222 FBDS nanoparticle concentration multiplied by the lowest acceptable weight % total CPT value and the highest acceptable FBDS nanoparticle concentration multiplied by the highest acceptable weight % total CPT value. These calculations result in a specification range of 1.7 mg/mL-3.6 mg/mL. Additionally, this target range of CPT concentration is based on desired dose levels in the phase 1 clinical study and incorporates the range of CPT concentrations used in the GLP-toxicology studies.

Purity is also assessed by integration of as many as eight individual impurity peaks in the “total CPT” RP-HPLC profile that have been observed to date in development batches, and are believed to represent impurities related to CPT, but may also include process-related impurities.

As described above, eight small impurity peaks have been detected by UV absorbance, that elute on either side of the unconjugated CPT peak in the “total CPT” RP-HPLC chromatogram (between ca. 4-8.5 minutes retention time. For the initial phase 1 study material, these CPT-related substances have not yet been identified, nor have their response factors been determined. However, these impurities were present in batches used in both the dose range finding and IND-enabling GLP-toxicology studies. The specification calls for reporting any peak that has an area greater than 0.05% of the total CPT peak area. Any individual peak area must be≤2% of the total peak area, and the sum of these peak areas must be≤3% of the total peak area. Note that these limits are measured relative to CPT content, not the entire drug substance. When the weight % of total CPT is considered, the individual impurity limit represents ˜0.3 area % of DAN-222 drug substance, and the sum of impurities limit represents ˜0.45 area % of DAN-222 drug substance.

These limits are justified by the presence of these CPT-related substances in the dose range finding and GLP-toxicology DAN-222 FBDS batches. In the dose range finding study, the maximum tolerated dose (MTD) in dogs was determined to be 1.5 mg/kg (human equivalent dose of 30 mg CPT/m²). In the pivotal GLP-toxicology study, the highest non-severely toxic dose (HNSTD) in dogs was determined to be 0.64 mg/kg (human equivalent dose of 12.8 mg CPT/m²). A six-fold safety factor applied to the HNSTD was used to set the proposed initial clinical dose for DAN-222 at 2 mg CPT/m². The sum of CPT-related substances in the dose range finding and GLP-toxicology FBDS batches were 3% and 2%, resulting in human equivalent doses of 0.93 mg and 0.26 mg CPT-related substances per m2 respectively. These values exceed the CPT-related substance content for the proposed initial dose of DAN-222 drug product (2 mg CPT/m²).

The nanoparticle concentration was determined gravimetrically by drying a sample of the FBDS by lyophilization and correcting for the non-volatile components of the formulation. The specification value of 17-23 mg/mL was selected to provide a desired fill and extractable volume for the drug product given the planned phase 1 clinical protocol dosing and the targeted range for CPT loading (weight % total CPT). In-process and stability data indicate that the DAN-222 FBDS remains stable in solution as formulated at concentrations as low as 5 mg/mL and as high as 40 mg/mL. Concentrations outside this range may be feasible but have not yet been studied closely.

The specification for particle size of DAN-222 FBDS is 20-80 nm. This specification incorporates the range of particle sizes observed in batches tested and used during development. The lower end of the specification is set to ensure that a minimal amount of drug substance will be subject to removal by the kidneys and liver. The upper limit is intended to allow for efficient tumor penetration and tumor cell uptake of the nanoparticles. The nanoparticle sizes for the dose range finding and GLP-toxicology batches were 37 nm and 31 nm, respectively.

The specification for polydispersity index (PDI) of DAN-222 FBDS is: ≤0.9. This specification incorporates the range of PDIs observed in batches tested and used during development. The PDIs for the dose range finding and GLP-toxicology batches were 0.463 and 0.346, respectively.

Zeta potential is a measure of surface charge. The specification for zeta potential of DAN-222 FBDS is: −5 mV to +5 mV. This specification incorporates the range of zeta potentials observed in batches tested and used during development. All DAN-222 FBDS development batches exhibited zeta potentials close to neutral. The value is typically slightly negative, but can occasionally return a slightly positive charge measurement. The measured zeta potentials for the dose range finding and GLP-toxicology batches were-0.751 mV and −0.399 mV, respectively.

The specification for osmolality of DAN-222 FBDS is: 336-504 mOsm/kg. This specification for osmolality is based on results obtained from repeated measurement of various development batches of DAN-222 FBDS, including the GLP-toxicology batch. This range is representative of the osmolality of the formulation buffer, as the DAN-222 molecule has little effect on the osmolality result. The proposed specification range is typical of osmolality limits for drug substance and drug product with comparable buffer formulations.

The specification for pH of DAN-222 FBDS is: 4.3±0.3. This specification for pH was set based on a series of formulation development studies to determine the optimal pH for DAN-222 FBDS stability. By design, free CPT is slowly released from DAN-222 at elevated pH levels. Control of pH within the range of pH 4.3±0.3 provides consistent stability for handling and use of the dose form, as well as reasonable limits for drug substance and drug product storage. The proposed specification is within the usual range for parenteral drug substances and drug products.

The specification for endotoxin (≤6 endotoxin units (EU)/mg CPT) is based on the permitted one-hour endotoxin exposure per USP<85>. The recommended endotoxin limit for parenteral products that are dosed by patient surface area is 100 EU/m, where m is the maximum proposed dose per square meter over one hour. Asserting a higher-than-anticipated dose results in a value of 6.25 EU/mg CPT, which is rounded to 6 EU/mg CPT for the specification.

The specification for bioburden limits for aerobic and anaerobic bacteria and yeasts and molds are set per compendial recommendations as per USP<61>. The specified values for each (≤10 colony forming units (CFU)/10 mL) are set to demonstrate both microbial control of the process as well as to ensure that when the final drug product sterile filtration is performed immediately prior to vial filling, the potential microbial load is within the removal capabilities of the filtration apparatus.

Example 6-Method of Manufacturing DAN-222 Formulated Bulk Drug Substance (FBDS) This exemplary DAN-222 FBDS manufacturing process consists of four main steps: (i) derivatization of camptothecin (CPT) to yield Gly-CPT (as the trifluoracetic acid (TFA) salt); (ii) synthesis of the parent mucic acid polymer (MAP); (iii) covalent attachment of derivatized camptothecin to the parent polymer to yield solid, amorphous MAP-Gly-CPT polymer-drug conjugate; and (iv) aqueous formulation of MAP-Gly-CPT to form nanoparticles (NPs).

FIG. 35 provides a scheme for an exemplary method of DAN-222 FBDS manufacture. Starting materials are indicated in shaded boxes at the top of the drawing and manufacturing steps performed under GMP conditions for the Phase 1 clinical trial drug substance are indicated with text or are surrounded by box 3401.

### Step 1: Derivatization of Camptothecin to Yield Gly-CPT

FIG. 36 shows the derivatization of CPT to synthesize Gly-CPT.

Gly-CPT is isolated as a TFA salt. All reactions and chemical workups are performed in glass vessels, using equipment and techniques common to synthetic chemistry procedures. No specialized equipment or catalysts are required for synthesis of Gly-CPT. As both CPT and Gly-CPT are highly potent compounds, the management of this synthesis process is performed under safe handling procedures commensurate with good occupational safety practices.

FIGS. 37A-37B provide a summary flow chart of the Gly-CPT synthetic process, including control points. The flow chart begins on FIG. 37A and is continued on FIG. 37B. A glass reactor is charged with methylene chloride (DCM) and 3 eq of N-(tert-butoxycarbonyl)-Gly-OH (Boc-Gly) to achieve a Boc-Gly concentration of about 0.06 g Boc-Gly/mL. The solution is stirred for 5-10 minutes, 4-dimethylaminopyridine (DMAP; 2 eq) and 20 (S)-camptothecin (CPT; 1 eq) are added, and the reaction mixture is stirred at 25-30° C. for about 10 minutes. The reaction is chilled to 0-2° C., and 3 eq diisopropylcarbodiimide (DIC) is slowly added, maintaining the temperature at 0-2° C. The reaction is warmed to 25-30° C. and is stirred for about 6 hours.

The reaction mixture is filtered through a Buchner funnel fitted with a filter cloth to remove any suspended solids, and the filter is washed with DCM (about 0.5× of the initial DCM volume, represented here as V). The filtrate containing the desired product is transferred to a new glass vessel and extracted twice with water (each extraction is about 0.5× V). The organic layer is concentrated under vacuum on a rotary evaporator to remove 50-70% of the total starting volume. The distillate is transferred to a new vessel, and methanol (MeOH; about 0.6× V) is added to precipitate the intermediate product. The reaction is chilled to 5-10° C. and is stirred at 5-10° C. for approximately 1 hour. The reaction is filtered through a Buchner funnel fitted with a filter cloth and is washed with MeOH (about 0.2× V). The wet compound is dispensed from the filter to a new vessel, isopropyl alcohol (IPA; about 0.5× V) is added, and the reaction is stirred for approximately 30 minutes. The reaction mixture is filtered through a lined Buchner funnel. The collected solids are washed with IPA (about 0.2× V), dispensed from the filter, and dried under vacuum at ambient temperature for at least 4 hours to remove excess solvent. The purified Boc-Gly-CPT intermediate solid is stored at ambient temperature in low-density polyethylene (LDPE) bags contained in secondary high-density polyethylene (HDPE) containers.

The desired intermediate Gly-CPT is obtained via deprotection of Boc-Gly-CPT using trifluoroacetic acid (TFA) as the deprotecting agent. A reactor is charged with DCM and Boc-Gly-CPT to achieve a Boc-Gly concentration of about 0.5 g Boc-Gly-CPT/mL. The reaction is stirred for 5-10 minutes. An equal volume of TFA is added, and the reaction is stirred at 25-30° C. for about 2 hours.

The reaction is chilled to 0-5° C., and about 1.5 volumes of methyl tert-butyl ether (MTBE) are added, maintaining the temperature at 0-5° C. The reaction is stirred at 0-5° C. for about 30 minutes. The resulting suspension is filtered through a lined Buchner funnel under nitrogen atmosphere, and the filter cake washed with a 2:1 MTBE: DCM mixture (about 2× of volume used to dissolve Boc-Gly-CPT, represented here as V′), and washed with MTBE (about 2× V′). This step is repeated with a somewhat shorter stirring time, and the isolated solid is dried under nitrogen for about 1 hour and dried under vacuum at ambient temperature for at least 4 hours to remove excess solvent. The purified glycyl-camptothecin (TFA salt; Gly-CPT) solid is stored at ambient temperature in LDPE bags contained in secondary HDPE containers.

For initial phase I manufacture, three Gly-CPT-TFA batches were manufactured with a batch size of 145-150 g.

### Step 2: Synthesis of the Parent Polymer MAP

The preparation of MAP from copolymerization of MAM with diSPA-PEG3500 is shown in FIG. 38. All reactions are performed in glass vessels, using equipment and techniques common to synthetic chemistry procedures. No specialized equipment or catalysts are required for synthesis of MAP, but care is required to exclude water from the reaction mixtures to the extent practical.

FIG. 39 provides a summary flow chart of the MAP synthetic process, including control points. A reactor is charged with 1 eq (succinimidyl propionate) 2PEG3500 (diSPA-PEG3500) and 1.07 eq mucic acid monomer (MAM). The reactor is purged with argon, and dimethyl sulfoxide (DMSO) is added to achieve a diSPAPEG₃₅₀₀ concentration of about 0.17 g diSPA-PEG3500/mL. Trifluoroacetic acid (TFA) (1.8 eq) is added and the reaction is stirred for 10-15 minutes. N,N-diisopropylethylamine (DIPEA) (2.6 eq) is added and the reaction is stirred at 21-23° C. for about 3 hours. These parameters are designed to ensure that the resulting MAP molecular weight is within the target range (50,000-80,000 Da).

MAP is precipitated with isopropyl alcohol (IPA) (about 2× of the total DMSO volume). The reaction is chilled to 0±5° C. About 5 volumes of IPA are added, and the reaction is stirred at 0±5° C. for approximately 50 minutes. The polymer is filtered through a Buchner funnel under argon atmosphere, washed twice with IPA (each wash is about 3× of the total DMSO volume), washed twice with diethyl ether (each wash is about 3× of the total DMSO volume), dispensed from the filter into pyrex drying dishes, and dried under vacuum at ambient temperature for at least 12 hours to remove excess solvent. The solid is stored at ≤−20° C. in 1000 mL wide mouth glass bottles with HDPE-lined caps.

For phase I, MAP was manufactured under GMP conditions in three batches with an average isolated yield of about 185 g copolymer.

### Step 3: Synthesis of MAP-Gly-CPT Amorphous Polymer-Drug Conjugate

MAP-Gly-CPT amorphous drug substance showed no instability when exposed to typical laboratory lighting conditions. However, CPT is known to be photosensitive, so all manufacturing steps in the presence of CPT were performed with red-colored lighting filters in place.

The conjugation of MAP with Gly-CPT to synthesize MAP-Gly-CPT is shown in FIG. 40. MAP-Gly-CPT is isolated as an amorphous solid; nanoparticles (NPs) are formed when the amorphous MAP-Gly-CPT is reconstituted in aqueous media. All reactions and chemical workups are performed in glass vessels or polymer single-use bags, using equipment and techniques common to synthetic chemistry procedures. No unusually specialized equipment or catalysts are required for synthesis of MAP-Gly-CPT amorphous. As CPT is a highly potent compound, the management of this synthesis process is performed under safe handling procedures commensurate with good occupational safety practices.

FIGS. 41A-41B provide a summary flow chart of the MAP-Gly-CPT amorphous synthetic process, including control points. The flow chart begins on FIG. 41A and is continued on FIG. 41B. A reactor is charged with 1 eq MAP and purged with argon. Dimethyl sulfoxide (DMSO) is added to achieve a MAP concentration of about 0.01 g MAP/mL. Glycyl-camptothecin (TFA salt; Gly-CPT; 1.2 eq) and 2.2 eq (7-azabenzotriazol-1-yloxy)tripyrrolidinophosphonium hexafluorophosphate (PyAOP) are added, and the reaction is stirred for 5-10 minutes. N,N-diisopropylethylamine (DIPEA; 2.5 eq) is added and the reaction is stirred at 19-25° C. for 16-22 hours.

The reaction is quenched with slow addition of cold pH 3.0 water (formulated from water for injection (WFI), about 0.4× of the total DMSO volume), maintaining the reaction temperature below 30° C. The reactor temperature is set to 10° C. and additional cold pH 3.0 water (about 2.6× of the total DMSO volume) is charged to the reactor and stirred for about 30 minutes. The solution is transferred to a stirred polymer tank system with a polyethylene film tank liner and another 1× volume of cold pH 3.0 water is added. The total volume of water added is intended to decrease the concentration of DMSO to about 20%, as the TFF membranes used for further purification are stable at this DMSO concentration. At this point, the solution may be stored in a cold room, protected from light, if immediate purification is not desired.

The solution is purified by ultrafiltration/diafiltration using a tangential flow filtration (TFF) technique. The process is performed using a Sartorius FlexAct system equipped with a Sartocon self-contained filter loop assembly holding a 10,000 Da molecular weight cut-off TFF membrane. Alternatively, the FlexAct system can be run using individual Sartocon Hydrosart cassettes mounted in a conventional TFF membrane holder. The process begins with an ultrafiltration phase, during which the total volume of the product solution is reduced by about half. At this point, diafiltration is initiated against pH 3.0 water for at least eight diavolumes to significantly reduce the DMSO content as well as remove low molecular weight water-soluble impurities. When the diafiltration phase is complete, further ultrafiltration is performed to concentrate the solution to about 0.04 g/mL MAP-Gly-CPT. The concentrated solution is frozen and lyophilized for at least 48 hours to yield purified MAP-Gly-CPT drug substance intermediate as an amorphous solid. The solid is stored at ≤−20° C. in wide-mouth glass bottles with HDPE-lined caps.

In the event of a failure of the TFF membrane system which allows MAP-Gly-CPT drug substance to pass into the permeate collection vessels, the opportunity exists to perform reprocessing of the permeate. This is performed by installing a new TFF membrane unit and feeding the product-containing permeate back into the FlexAct system to concentrate the solution by ultrafiltration. When concentration is complete, additional diafiltration against pH 3 water is performed prior to concentration and lyophilization.

For phase I, MAP-Gly-CPT was made in two batches with a batch size of 250 g of MAP, with an average yield of about 250 g of conjugate.

### Step 4: Preparation of MAP-Gly-CPT NP Solution (DAN-222)

The aqueous formulation of MAP-Gly-CPT as an NP solution (DAN-222 Formulated Bulk Drug Substance (FBDS)) is shown in FIG. 42.

All steps are performed in glass vessels or polymer single-use bags, using equipment and techniques common to synthetic chemistry procedures. No unusually specialized equipment or catalysts are required for the formulation of MAP-Gly-CPT NP solution. As CPT is a highly potent compound, the management of this formulation process is performed under safe handling procedures commensurate with good occupational safety practices.

FIGS. 43A-43B provide a summary flow chart of the DAN-222 FBDS process, including control points. The flow chart begins on FIG. 43A and is continued on FIG. 43B. DAN-222 FBDS is formulated in a 20 mM acetate, 0.9% sodium chloride buffer, pH 4.3±0.3. To achieve this buffer concentration, a 50 L pallet tank with mixer is charged with WFI and sufficient sodium chloride, sodium acetate, and glacial acetic acid to obtain a 10× concentrate of the desired final buffer.

The 100 L reactor is washed with water and acetone, and subsequently heated at 120° C. under positive argon flow to sanitize the vessel. The reactor is charged with solid, amorphous MAP-Gly-CPT. Water for injection (WFI), adjusted to pH 4.0 with H₃PO₄, is added to achieve a MAP-Gly-CPT concentration of about 0.005 g/mL MAP-Gly-CPT, and the mixture is stirred for at least 12 hours. The MAP-Gly-CPT NP solution is transferred to a tank fitted with a polypropylene tank liner via tubing fitted with a 0.45 μm filter. The 100 L reactor is thoroughly rinsed, and the NP solution is returned to the reactor and stirred. The volume of the NP solution in the reactor is measured, and the appropriate volume of 10× formulation buffer is calculated and slowly added to the reactor to avoid splashing. The resulting solution is stirred for 2-3 hours and transferred to a HDPE tank fitted with a polypropylene tank liner.

The calculated concentration of MAP-Gly-CPT in the tank is determined by adjusting the total charge of amorphous MAP-Gly-CPT by the residual solvent value obtained from that material. This calculation provides the target weight of permeate to be removed during concentration by ultrafiltration. The Sartorius Flex-Act system is used, fitted with a Sartocube ECO tangential flow filtration cassette with a 10,000 Da MW cutoff. The solution is concentrated by ultrafiltration until the target weight of permeate is reached. The retentate is transferred to a 20 L Flexboy bag, sampled for in-process control (MAP-Gly-CPT concentration) and stored at 2-8° C. pending completion of analysis. The targeted concentration range for DAN-222 FBDS is 17-23 mg/mL MAP-Gly-CPT. If the range is achieved, the solution in the 20 L Flexboy bag is prepared for sterile filtration, sampling, and bottling in FBDS containers. If the concentration is too low, the solution is returned for further ultrafiltration, using the measured volume and concentration to calculate the additional amount of permeate to be removed. Should the concentration be found to be above the specified range, the appropriate amount of 1× formulation buffer to be added is calculated and added, and the container gently rocked to blend.

Transfer of DAN-222 FBDS into storage containers is performed in a biosafety cabinet (BSC) to improve microbial control. The solution is transferred to sterile, 4 L HDPE bottles using a MasterFlex pump and sterile tubing sets fitted with a 0.22 μm Sartopore 2 MidiCaps® filter. After sampling for release, stability, and retain purposes, the 4 L bottles are filled. Prior to filling each bottle, two or three 5 mL samples are dispensed into sterile Falcon tubes, two of which serve as satellite samples for their respective bottles. After this sampling, each tared bottle is filled with approximately 3 L of solution, capped, and tightly sealed and weighed to obtain the net weight of contents. All FBDS containers, including samples, are labeled.

DAN-222 FBDS containers are stored at a controlled temperature of ≤−70° C. except for samples receiving immediate analysis.

For phase I, DAN-222 FBDS was made as a single batch with a batch size of 400 g of amorphous MAP-Gly-CPT. The resulting formulation afforded 17.4 kg (17.4 L) of DAN-222 FBDS for further use, and 751 mL of DAN-222 FBDS taken as samples for release, stability, and as satellite samples for identification at the drug product filling site.

### Example 7-Improvements in Manufacturing DAN-222 Formulated Bulk Drug Substance (FBDS)

Process development for GMP manufacture of DAN-222 Formulated Bulk Drug Substance (FBDS) progressed and improved from its initial development to the exemplary method outlined in Example 6. The current process consists of four GMP steps. First, 20 (S)-camptothecin (CPT) is derivatized by linking glycine to the 20-hydroxyl group of CPT (Gly-CPT). Second, the parent mucic acid polymer (MAP) is synthesized via polymerization of the comonomers mucic acid monomer (MAM) and (succinimidyl propionate) 2-PEG (diSPA-PEG). In the third step, the derivatized CPT is covalently bonded to the parent polymer to yield the MAP-Gly-CPT amorphous polymer-drug conjugate. Last, the conjugate is formulated in aqueous media to form a nanoparticle (NP) solution.

Process development work continued following the confirmation of the initial processes to produce the DAN-222 (FBDS). This continued development provided process development, scale-up, and manufacture of DAN-222 FBDS for IND-enabling studies and phase 1 clinical studies.

### Step 1: Derivatization of Camptothecin to Yield Gly-CPT-Development

The production of the derivatized CPT is a two-step process whereby the hydroxyl group of CPT is esterified with a tert-butoxycarbonyl-protected glycine linker (Boc-Gly), followed by the removal of the Boc protection group and isolation to yield glycine-linked CPT (Gly-CPT) as a trifluoroacetic acid (TFA) salt.

The initial process at Step 1 involves dissolution of CPT, Boc-Gly, and 4-dimethylaminopyridine (DMAP) in dichloromethane (DCM). The reaction is initiated by the addition of diisopropylcarbodiimide (DIC), and the intermediate is isolated by precipitation with methanol (MeOH). To remove the Boc group, the intermediate is stirred with a mixture of trifluoroacetic acid (TFA) and DCM. The Gly-CPT product is isolated by precipitation as the TFA salt with methyl tert-butyl ether (MTBE). During early development, the Gly-CPT was synthesized at a research scale of about one gram per synthesis.

The development process led to changes during synthesis and isolation, primarily associated with optimizing solvent volumes and reaction temperatures at the initial GMP scale. While the quantity of this highly-potent intermediate needed for initial clinical studies is relatively low, after continued development, including a 10 gram synthesis, it was decided to implement a roughly 15-fold scaled-up synthesis (150× relative to research scale) for the initial GMP manufacture.

Over the course of the Step 1 development, several modifications were made to the process to make it more robust, scalable, safer, and/or to lower impurity levels. A summary of these changes is provided in Table 7. These changes primarily address reaction concentrations and solvent volumes, reaction temperatures, and some additional steps to improve intermediate purity.

TABLE 7
Summary of Process Development Changes for Gly-CPT

| | Development Status | |
|---|---|---|
| Step/Process | Development Scale | Initial GMP Process |
| Process Scale | ca. 1 g yield | 0.145-0.150 kg yield |
| Boc-Gly-CPT synthesis | 1 mol eq CPT | No changes |
| Reagent ratios | 3 mol eq Boc-Gly | |
| | 2 mol eq DMAP | |
| | 3 mol eq DIC | |
| Initial DCM charge | Target 0.3 g/mL Boc-Gly | Target 0.06 g/mL Boc-Gly |
| Reaction temperature | Reduced to 7-15° C. prior | 25-30° C., reduced to 0-2° |
| | to DIC addition, and | C. prior to DIC addition at 0- |
| | allowed to warm to 17-23 | 5° C., returned to 25-30° C. |
| | ° C. | after completion of addition |
| Stirring time | 2 hours | 6 hours |
| Filtration and aqueous wash of | NA | Post-reaction solution filtered |
| organic layer with product | | and subjected to 2x aqueous |
| | | extractions |
| Concentration of Boc-CPT | DCM removed under | 95% of reaction volume |
| | vacuum to give crude solid | removed by vacuum |
| | | distillation at <30° C. |
| Temperature for methanol | −9° C.−−5° C. | Add methanol at about 30° C. |
| addition | | and cool to 5° C.-10° C. |
| Methanol volumes used | 4x initial DCM charge for | 0.6x initial DCM charge for |
| | precipitation plus 2x | precipitation plus 2x volume |
| | volume wash | wash |
| Isopropanol washes | NA | 0.5x original DCM volume |
| Gly-CPT synthesis | 0.4x initial DCM charge | No change |
| DCM added | from | |
| TFA added | Boc-Gly-CPT synthesis | |
| | 0.4x initial DCM charge | No change |
| | from | |
| | Boc-Gly-CPT synthesis | |
| Reaction temperature and | ≤27° C. (25 ± 2° C.), | 25-30° C., 2 hours |
| stirring time | 2 hours | |
| Temperature for MTBE | Cool to 0° C. | Cool to 0-5° C. |
| addition | | |
| MTBE volume for | 6x initial DCM charge | 1.5x initial DCM charge from |
| precipitation | from Boc-Gly-CPT synthesis | Boc-Gly-CPT synthesis |
| Solvent volume for | 10 to 1 MTBE-DCM: | MTBE: |
| filter cake washes | 4x initial DCM charge | 2x initial DCM charge from |
| | from | Boc-Gly-CPT synthesis |
| | Boc-Gly-CPT synthesis | 2 to 1 MTBE-DCM: |
| | | 2x initial DCM charge from |
| | | Boc-Gly-CPT synthesis |
| Drying | 25° C. under vacuum, 6 | 25-30° C. under vacuum, 4 |
| | hours or to consistent | hours to meet residual water |
| | weight | in-process control |

For the Boc-Gly-CPT synthesis step, the relative ratios of reactants remained constant, but the reaction was found to proceed more smoothly when run under more dilute conditions at a higher reaction temperature. A new aqueous extraction step was added to improve removal of process impurities. While in earlier syntheses DCM was removed to afford a crude solid, vacuum distillation was performed to remove only 95% of the DCM volume and a significantly lower proportion of methanol was added for final precipitation at a higher temperature. An additional wash with isopropyl alcohol (IPA) was found to improve performance of the isolation.

For deprotection and isolation of Gly-CPT, the same reagent ratios were used as in previous syntheses. However, for the subsequent precipitation step, a significantly lower volume of methyl tert-butyl ether (MTBE) was used, and subsequent washes of the filter cake with an MTBE: DCM mixture were performed with a lower ratio of MTBE: DCM.

During the development of analytical methods for release of the Gly-CPT intermediate, it was discovered that levels of residual MTBE were not reduced by vacuum drying, suggesting that the intermediate crystallizes as a solvate. Analysis by 1H-NMR of a prior synthesis of Gly-CPT used in development determined that a comparable level of MTBE was present in that batch as well.

It was also determined that residual water could not be fully removed from the final product, even when subjected to drying agents (magnesium sulfate and sodium sulfate) and azeotropic distillation under mild conditions. In each case, conditions led to degradation of the glycyl ester. Given that the subsequent conjugation of Gly-CPT with MAP is sensitive to the presence of excess water, future process development for this step will include methods to reduce or eliminate the presence of water in the intermediate product.

### Step 2: Synthesis of the Parent Polymer MAP-Development

The production of the parent polymer, mucic acid polymer (MAP), is a step-growth polymerization of the two comonomers, MAM and diSPA-PEG, a linear PEG molecule with succinimide-activated propionyl esters at each end. The comonomers are dissolved in dimethyl sulfoxide (DMSO), and the reaction is initiated by the addition of N,N-diisopropylethylamine (DIPEA), triggering the amidation of free amines at each end of the MAM monomer. The reaction is quenched with water when the MAP molecular weight (MW) is expected to be at or near the target, and the MAP is isolated by dialysis against DMSO and water, followed by lyophilization.

The initial process aims to control the reaction kinetics and achieve a near plateau in polymer MW over time. While conditions for MAP production can be identified for approximately linear growth of the MW with time, this strategy requires precise in-process monitoring of MW and reaction quenching. Alternatively, given that the polymerization has been shown to follow known models for polymer step-growth, a strategy was developed based on polymerization theory (the Modified Carothers's Equation and Flory-Shulz Distribution) that stalls the reaction over time. This strategy achieves a plateau in growth by utilizing a stoichiometric imbalance of the comonomers and slows the reaction as the MAP approaches the targeted MW. Example polymerizations for which the MW of the MAP at late time points is controlled by the stoichiometric ratio of MAM to diSPA-PEG in the reaction mixture are shown in FIG. 44.

As shown in FIG. 44, using the plateaued growth strategy, an increase in the ratio of MAM: diSPA-PEG results in a lower MW polymer.

Based on the above observations, it was desired that monomer starting materials be prepared with a high degree of functionalization as specified herein, and that the reaction be performed under anhydrous conditions to retain stability of the activated SPA groups on diSPA-PEG to protect against early termination of the amidation reaction, allowing for a well-controlled stoichiometric ratio of the comonomers at or near the target.

Over the course of the Step 2 development, several modifications were made to the process to make it more robust, scalable, safer, and/or to lower impurity levels. These modifications included: (i) using MAM as its diTFA salt instead of its neutral species; (ii) MAM, diSPA-PEG, TFA, and DIPEA stoichiometry; and (iii) using dialysis instead of precipitation for purification.

Early development batches of MAP were made using MAM starting material that was intended to be isolated as the diTFA salt. Subsequently, it was found that isolation of MAM with a consistent amount of counterion TFA proved difficult. Analysis of the first few MAM development batches revealed that the equivalents of TFA: MAM varied from batch to batch by as much as ˜25% (e.g., 1.5:1 actual vs 2:1 target). This variability would likely cause significant inaccuracy in the amount of MAM added to the reaction, and thus significant variability in the resulting polymer MW given the sensitivity to the comonomer ratio described above. To reduce potential variability, it was decided to isolate MAM as the neutral species (lacking the TFA counterion) and allow for a more precise addition of TFA as a subsequent reaction step prior to polymerization. Chemical structures of the MAM starting materials are provided for comparison in FIG. 45.

Reactions were carried out using MAM isolated as the diTFA salt or as the neutral species (isolated with only a trace of TFA) to study the effect of the presence or absence of TFA in the monomer starting material on the reaction behavior. While the TFA group does not participate in the actual polymerization reaction, it was found to significantly increase the solubility of MAM in DMSO, and hence the reaction kinetics. Unlike the diTFA salt, which is generally soluble in DMSO, the neutral species was found to be minimally soluble in DMSO. Despite its poor solubility, investigation of the polymerization of the neutral species with diSPA-PEG showed the reaction to proceed without addition of TFA, though with generally linear kinetics regardless of the comonomer ratio. In contrast, polymerization of the neutral species with diSPA-PEG whereby approximately 1-2 eq. TFA was added to the reaction prior to initiation with DIPEA led to a recovery of the desired plateau behavior.

Based on the results obtained from the initial experiments, it was decided to pursue development of reaction conditions with MAM isolated as the neutral species as well as subsequent addition of TFA that would allow for plateau of the polymer MW at or near the target.

During early development work with the new MAM monomer and subsequent addition of TFA, several small-scale (˜250 mg diSPA-PEG) screening reactions were carried out to identify conditions that would allow for the desired plateau behavior for the MAP polymerization. The results from two sets (screen 1 and screen 2) of many experiments are provided as examples to demonstrate key learnings that informed the selection of the final reaction conditions.

An initial set of experiments (screen 1) was carried out to study the combined effects of different comonomer ratios as well as equivalents of TFA and DIPEA on the kinetics of the reaction. The reaction conditions investigated in screen 1 are provided in the lower portion of FIG. 46, where italicized entries highlight varied conditions. The MW values for MAP using the conditions specified in screen 1 are also shown in the upper portion of FIG. 46.

The results shown in FIG. 46 show that a reduced amount of DIPEA (˜2.5 equivalents) led to decreased reaction kinetics. As expected, a decrease in the MAM: diSPA-PEG ratio towards 1:1 led to increased MW. In addition, it was found that an increase in TFA led to an improved plateau in MW. The combined results of the initial screen suggested that the comonomer ratio, equivalents of TFA and DIPEA, and reaction time were sufficient handles to achieve the targeted MAP polymerization, with reaction 1-B demonstrating favorable behavior.

Based on the above observations, a second panel of conditions (screen 2) was evaluated to further probe the effects of TFA and DIPEA levels on the kinetics of the reaction at a slightly reduced comonomer ratio and optimize the conditions. The reaction conditions investigated in screen 2 are provided in the lower portion of FIG. 47, where italicized entries highlight varied conditions. The MW values for MAP using the conditions specified in screen 2 are shown in the upper portion of FIG. 47.

As shown in the results of FIG. 47, it was found that a small decrease in the comonomer ratio led to a modest increase in MW. In addition, increased TFA concentration again led to an improved plateau of the reaction, while decreased DIPEA concentration led to decreased reaction kinetics. As a result of screen 2, conditions were identified (reaction 2-C) for small-scale polymerization that allowed for plateau of the MW and provided MAP with MW in the targeted range of 50,000-80,000 Da over a broad window of time.

The MW dependence on comonomer ratio as well as equivalents of TFA and DIPEA was replicated on a 1 g scale, giving similar trends, though a subtle decrease in kinetics was observed for all reactions. This behavior was likely attributed to slightly less stringent anhydrous conditions under which the reactions were performed. As a result, a modest increase in DIPEA (2.6 vs 2.5 equivalents) was evaluated for scale-up reactions and provided kinetics similar to those observed in small-scale experiments.

The decision to change the purification process from dialysis and lyophilization to precipitation was made to allow for reduced processing time and to eliminate the need for large buffer volumes and long processing times during the isolation of MAP. During development MAP production, it was observed that large volumes of dialysis media and long drying periods on the lyophilizer were required to isolate the polymer as a solid. It was expected that increased volume requirements, longer processing times, and/or tangential flow filtration (TFF) method development would most likely be needed with increasing scale.

To mitigate these process risks, an assessment was made as to the feasibility of isolating MAP as a solid by precipitation to eliminate the need for dialysis and lyophilization during scale-up, and to allow for significant process time savings. Precipitations from DMSO, via addition to several anti-solvents, were carried out. Of the various solvents evaluated, isopropyl alcohol (IPA) was found promising. Precipitation of the polymer was also attempted with direct addition of IPA to the polymerization reaction mixture and was found to work equally well. MW analysis of precipitated and dialyzed MAP showed comparable chromatograms and resulting MW and PDI. In addition, 1H-NMR analysis of the precipitated MAP showed very low levels of impurities (<0.1% by mass). However, filtration of the precipitated MAP under vacuum in ambient atmosphere resulted in a sticky solid material, which was difficult to remove from the filter. Filtration under a nitrogen or argon head avoided this behavior and gave a reasonably dry solid that could be easily transferred from the filter for further drying.

Based on the above observations, it was decided to proceed with the IPA precipitation procedure including an argon head during filtration for the isolation of MAP in subsequent scale-up and GMP batches.

Subsequent to process development, scale-up reactions were carried out to confirm the optimized reaction conditions and the resulting reaction kinetics. As described previously, a slight increase in DIPEA was used for scale-up production, relative to the conditions developed during production of development batches. Additionally, a slight versus complete plateau towards the upper MAP molecular weight limit (80,000 Da) rather than at the midpoint (65,000 Da) was targeted to allow for a broad (several hour) window for quenching and eliminate the risk of undershooting the desired MW.

Scale-up reactions were carried out at 5 g and 50 g scale using the previously identified conditions (1.07:1 MAM: diSPA-PEG, 1.8 equivalents TFA, 2.6 equivalents DIPEA). Three development runs were executed on 5 g scale and allowed to proceed for 5 hours to monitor a long reaction time course. These triplicate runs showed good reproducibility and favorable reaction kinetics as well as informed a target quench of the reaction at ˜3.5 h to achieve a final MAP MW within the targeted MW range. A 50 g scale reaction was also executed to support GMP manufacture, with a quench at 3.5 h by addition of IPA. As expected, the large-scale reaction proceeded with similar kinetics and resulted in MAP MW within the target range. In addition, direct precipitation of the reaction mixture with slow addition of IPA gave the polymer as a white precipitate, which was successfully isolated by filtration under argon atmosphere and dried to afford the solid.

The MW data for the scale-up MAP reactions are provided in FIG. 48. GMP manufacture was conducted at 165 g, about 3-fold scale up of 50 g, as described in Example 6.

### Step 3: Synthesis of MAP-Gly-CPT Amorphous Polymer-Drug Conjugate-Development

The production of the polymer-drug conjugate, MAP-Gly-CPT, involves reaction of the carboxylic acids on the parent MAP polymer with the derivatized CPT amines to form amide bonds. The initial process utilizes EDC/NHS chemistry to perform this covalent coupling, according to the conditions described in Table 8, with the reaction dilution italicized.

TABLE 8
Initial development reaction conditions
for production of MAP-Gly-CPT

| Reagent | MAP | EDC | NHS | Gly-CPT | DIPEA | DMSO |
|---|---|---|---|---|---|---|
| Equivalents | 1 | 4 | 3 | 3 | 3 | 80 mL/g MAP |

MAP is dissolved in DMSO, and 1-ethyl-3-(3-dimethylaminopropyl) carbodiimide (EDC), N-hydroxysuccinimide (NHS), and Gly-CPT are added. The reaction is initiated by the addition of DIPEA. After reaction completion, the MAP-Gly-CPT is isolated by dialysis against DMSO and pH 3.0 water, followed by lyophilization.

Over the course of the Step 3 development, several modifications were made to the process to make it more robust, scalable, safer, and/or to lower impurity levels. The first was use of EDC/NHS-versus PyAOP-based coupling. The second was the use of dialysis versus TFF purification.

Early development batches of MAP-Gly-CPT were made using EDC/NHS coupling chemistry, which requires a moderate (2- to 3-fold) excess of the Gly-CPT starting material and reagents to achieve the desired drug loading level. As a result, it was found that large volumes of dialysis media and long processing times were required to sufficiently remove unreacted starting materials and other impurities from the product.

To reduce the excess in starting materials and reagents in the reaction, particularly that of the Gly-CPT, the use of the more active (7-azabenzotriazol-lyloxy)tripyrrolidinophosphonium hexafluorophosphate (PyAOP) coupling reagent to prepare MAP-Gly-CPT was evaluated. Given its increased reactivity, it was anticipated that PyAOP would allow for a higher coupling efficiency and hence reduced levels of Gly-CPT in the reaction relative to the previous EDC/NHS route.

An initial set of reactions was carried out to study the coupling efficiency with PyAOP across a range of Gly-CPT equivalents added to the reaction. As expected, nearly quantitative grafting efficiency (>80%) was achieved, defined as the efficiency of incorporation of Gly-CPT into the MAP-Gly-CPT conjugate material, across all conditions. In addition to the high coupling efficiency, the achieved drug loading levels showed a linear relationship with Gly-CPT equivalents added to the reaction, demonstrating significant control of drug loading in this process step. The equivalents (eq.) of Gly-CPT and resulting weight percent (wt. %) CPT and percent grafting efficiency for representative conjugation reactions are provided in Table 9.

TABLE 9
Drug loading and grafting efficiency for reactions with PyAOP

| Reaction | A | B | C | D | E | F |
|---|---|---|---|---|---|---|
| Eq. Gly-CPT | 0.57 | 0.69 | 0.77 | 0.88 | 0.95 | 1.20 |
| Wt. % CPT | 7.6 | 8.7 | 9.5 | 10.7 | 11.5 | 13.7 |
| Efficiency (%) | 94 | 89 | 87 | 85 | 85 | 81 |

The grafting efficiency and linear relationship is shown in FIG. 49. Data provided in the table for samples A-F match the results shown in FIG. 49.

However, later development batches revealed occasional unanticipated observations related to the reaction itself and/or NPs derived from PyAOP-coupled conjugate materials. In one experiment, PyAOP-coupled MAP-Gly-CPT formed NPs with larger-than-typical diameters (˜70 nm) that were also difficult to filter. In another attempt, gel formation was observed during the reaction. It was speculated that crosslinked conjugate materials may have been formed by possible reaction of MAP polymer amine end groups with activated carboxylic acid groups on other polymer strands, creating atypical high molecular weight species (HMWS). Preliminary assessments appeared to confirm this phenomenon though the observation has not yet been probed in detail. Additional modifications to the process were investigated to enable the use of PyAOP for Gly-CPT coupling.

To minimize the process risk of forming HMWS, it was decided to target high drug loading levels to minimize the number of unreacted carboxylic acid groups. The reaction was also modified so that it was performed at increased dilution, which is known to significantly reduce the likelihood of polymer chain end groups participating in the reaction. Additional development reactions were executed with increased dilution on both 2.5 and 4 g scale with 1.0, 1.1, and 1.2 equivalents of Gly-CPT. With the modified reaction conditions, all reactions proceeded well and gave no indication of formation of HMWS. It was concluded that these changes were sufficient to minimize the formation of HMWS.

A comparison of the initial and subsequently modified reaction conditions for PyAOPbased coupling is provided in Table 10, with the reaction dilutions italicized.

TABLE 10
Reaction conditions for initial and modified
PyAOP-based MAP-Gly-CPT synthesis

| | | | Gly- | | | |
|---|---|---|---|---|---|---|
| Reagent | MAP | PyAOP | CPT | DIPEA | DMSO | |
| Initial Equivalents | 1 | 2.5 | 2.5 | 2.5 | 20 | mL/g MAP |
| Modified | 1 | 2.2 | 1.2 | 2.5 | 100 | mL/g MAP |
| Equivalents | | | | | | |

(Initial GMP
Process)

Based on the above observations, it was decided to proceed with the modified PyAOPbased coupling procedure in subsequent scale-up and GMP batches to allow for reduced excess in starting materials and reagents as well as reduced buffer volumes and processing times required during the conjugate purification.

The decision to change the purification process from dialysis to ultrafiltration/diafiltration by TFF was based upon volume requirements and practicality. Scale-up versions of dialysis require much longer processing times as well as large solution volumes and are generally impractical at the scale required for GMP batches. Precipitation was also evaluated with several anti-solvents, though none were found initially promising. Instead, efforts were focused on development of a TFF process to purify the conjugate. It was determined that polyethersulfone (PESU) TFF membranes are compatible with aqueous solutions containing up to 10% DMSO, so it was decided to dilute the reaction mixtures 10-fold with pH 3.0 water prior to performing TFF. (Subsequently, it was determined that PESU membranes were resistant to up to 20% DMSO in water, and the dilution was commensurately reduced for GMP manufacturing.) TFF was performed on several of the 4 g MAP-Gly-CPT batches using scaled-down TFF membrane and cartridges comparable to those to be used in the scale-up GMP configuration. It was determined that 8 diafiltration volumes was sufficient to reduce DMSO and impurities to acceptable levels.

Based on the above observations, it was decided to proceed with TFF purification for the isolation of MAP-Gly-CPT in subsequent scale-up and GMP batches.

Subsequent to process development, scale-up reactions were carried out to confirm the modified reaction conditions and purification methods. Two scale-up reactions were executed on 25 g scale to support GMP manufacture using the previously identified conditions (1.2 equivalents Gly-CPT, 2.2 equivalents PyAOP, 2.5 equivalents DIPEA, 100 mL DMSO/g MAP). These duplicate runs showed good reproducibility and achieved drug loading within the targeted range for MAP-Gly-CPT. In addition, TFF purification showed comparable performance to the development runs, resulting in sufficiently low levels of DMSO and impurities after 8 diafiltration volumes of pH 3.0 water. The solid conjugate was successfully isolated by lyophilization. GMP manufacture was conducted at 250 g, a standard order-of-magnitude scale-up of 25 g, as described in Example 6.

### Step 4: Preparation of MAP-Gly-CPT NP Solution (DAN-222)

The preparation of MAP-Gly-CPT NPs requires dissolution of the amorphous polymer-drug conjugate in an aqueous medium, whereby the hydrophobic nature of the CPT drives self-assembly of the conjugate into NPs. The initially developed process involved dissolution of the MAP-Gly-CPT amorphous material in pH 4.0 water, addition of concentrated saline (9% NaCl, pH 4.0), concentration using a centrifugal ultrafiltration device, and 0.22-micron sterile filtration.

Over the course of the Step 4 development, several modifications were made to the process to make it more robust, scalable, safer, and/or to lower impurity levels, including: (i) a concentrated formulation buffer change; (ii) use of centrifugal ultrafiltration instead of TFF concentration; and (iii) a conditioning filtration step.

While the MAP-Gly-CPT NPs demonstrate good stability in unbuffered saline solution (0.9% NaCl, pH 4.0), the FBDS is pH-sensitive and a robust drug product formulation requires a buffer system to protect against pH drift and degradation during storage. To support the GLP-toxicology study and subsequent preparations, the decision to change from addition of concentrated saline to addition of concentrated formulation buffer (200 mM sodium acetate, 2 M NaCl, pH 4.16) was made based upon the previously described formulation development effort. The resulting target DP formulation for MAP-Gly-CPT NPs is 20 mM sodium acetate, 200 mM NaCl, pH 4.3±0.3. No impact to particle properties (e.g., size, zeta potential, filterability) was observed following the change in formulation buffer, and the FBDS demonstrates ample stability for in-process and in-use stability considerations.

The decision to change the concentration process from centrifugal ultrafiltration to TFF ultrafiltration was based upon practicality. Scale-up versions of centrifugal ultrafiltration require much longer processing times and generally cannot be performed at the scale sizes that are required for our GMP batches. TFF was performed on several of the 4 g MAP-Gly-CPT NP batches, again using the same membrane and cartridge types as to be used in the scale-up GMP configuration. Concentration by TFF yielded target MAP-Gly-CPT concentrations and had no impact on particle properties.

Based on the above observations, it was decided to proceed with TFF concentration for the preparation of MAP-Gly-CPT NPs in subsequent development, scale-up, and GMP batches.

The decision to change the filtration process from a single final sterile filtration at the end of FBDS production to the use of an intermediate conditioning filtration step was made to allow seamless and de-risked final sterile filtration. During development production of the FBDS, it was observed that the final sterile filtration process was often difficult, and multiple sterile filters would typically be needed to complete filtration.

To mitigate this process risk, after the MAP-Gly-CPT amorphous material is dissolved into the pH 4.0 water, a 0.45-micron filtration step was used to condition the material and remove some product-related particulates. Upon subsequent spiking in of the concentrated formulation buffer and TFF steps to formulate and concentrate the FBDS, the final 0.22-micron sterile filtration can proceed without risk of extended filtration times. Formulations involving this conditioning filtration step gave a comparatively easy and fast final sterile filtration.

Based on the above observations, it was decided to proceed with the conditioning filtration step for the preparation of MAP-Gly-CPT NPs in subsequent scale-up and GMP batches.

Subsequent to process development, scale-up formulations were carried out to confirm the optimized methods. Two scale-up formulations of MAP-Gly-CPT NPs were executed on 25 g scale to support GMP manufacture using the scale-up conjugate materials described above. Dissolution, formulation, and subsequent concentration by TFF showed comparable performance to the development runs, achieving targeted levels for formulation components and MAP-Gly-CPT concentration. In addition, final sterile filtration performed well.

GMP manufacture was conducted at 400 g, a 16-fold scale-up of 25 g, as described in above. This somewhat higher-than-typical process scaleup was assessed as a low risk, as the formation of MAP-Gly-CPT NPs had been shown to be consistent and repeatable at a wide variety of scales.

Lot 21-M007 was the first batch of DAN-222 FBDS manufactured under GMP conditions as phase I clinical trial material. This lot was manufactured according to the initial manufacturing process described in Example 6. Manufacturing differences between this lot and the lots used in the dose range finding (lot SKT-757-077-NPS) and toxicology (lot SKT-874-031-NPS) studies are summarized in FIG. 50.

The primary differences relate to the monomer type and isolation method used for MAP, the coupling agent and purification methods used for synthesis of MAP-Gly-CPT, and lastly the formulation buffer as well as concentration and filtration methods used for MAP-Gly-CPT NPs (DAN-222 FBDS). Analytical data for these batches demonstrate that the quality of GMP DAN-222 FBDS and DAN-222 drug product is comparable to that used in IND-enabling toxicology studies.

### Example 8-DAN-222 Sterile Solution for Infusion and Pharmaceutical Development

DAN-222 sterile solution for infusion is supplied as a clear to slightly opalescent, colorless to light yellow solution. The drug product is stored frozen at −20±5° C. and is thawed prior to administration.

DAN-222 sterile solution for infusion is delivered as a nanoparticle construct, consisting of a hydrophilic polymer backbone conjugated with multiple units of the active moiety 20 (S)-camptothecin via a glycyl linker. When dissolved in aqueous media, this polymer conjugate spontaneously forms nanoparticles consisting of the hydrophobic active moiety surrounded by the hydrophilic polymer. When administered, 20 (S)-camptothecin is slowly released from the nanoparticle construct.

Following the formulation screening process, described above, a DAN-222 sterile solution for infusion was chosen. The DAN-222 sterile solution for infusion is available as a single strength, with a target concentration of 20±3 mg/mL of DAN-222. At this concentration, DAN-222 sterile solution for infusion delivers 2.6±0.3 mg/mL of 20 (S)-camptothecin active ingredient upon which dosing is based.

The composition of DAN-222 sterile solution for infusion is provided in Table 11.

TABLE 11
Composition of DAN-222 sterile solution for infusion

| | | | Quantity | |
|---|---|---|---|---|
| | | | per dose | |
| Component | Grade | Function | unit (vial) | |
| DAN-222 | In-house | Active | 62 | mg¹ |
| | | ingredient | | |
| Ortho-Phosphoric acid, 85% | Ph Eur, BP, JPE, NF, E 338 | Excipient | 0.033 | mg |
| Sodium acetate, anhydrous | USP | Excipient | 1.73 | mg |
| Sodium chloride | USP Multi-compendial | Excipient | 36.2 | mg |
| Acetic acid, glacial | Ph Eur, JPE, USP | Excipient | 2.46 | mg |
| Water for injection | USP-NF | Solvent | 3.1 | mL qs |

BP = British Pharmacopoeia;
JPE = Japanese Pharmaceutical Excipients;
JP = Japanese Pharmacopeia;
mg = milligram;
NF = National Formulary;
Ph Eur = European Pharmacopoeia;
qs = sufficient quantity;
USP = United States Pharmacopeia
¹Equivalent to 8.1 mg 20(S)-camptothecin.

No reconstitution is required for administration of DAN-222 sterile solution for infusion, and no reconstitution diluent is provided.

The physicochemical characteristics of DAN-222 drug substance necessitate its delivery as a parenteral solution. Consequently, pharmaceutical development focused on the creation of a drug product formulation for intravenous infusion. The formulation had to meet several criteria: sterility; acceptable stability during bulk drug substance and drug product manufacture; acceptable stability over long-term storage; and acceptable stability during handling and administration. As an aqueous solution is required for formation of DAN-222 nanoparticles, formulation design focused on identification of excipients that provide both isotonicity as well as stability within a desired pH range (e.g., buffered pH).

The use of sodium acetate anhydrous and acetic acid at their respective concentrations (6.8 mM and 13.2 mM) is critical in controlling the pH of the drug product. These excipients were chosen due to their ability to buffer the formulation in a pH range that is conducive to product stability, their ability to maintain consistent pH over a wide range of temperatures, and their common use in pharmaceuticals. These excipients were selected for use based upon data obtained in the formulation development screening studies on multiple buffer systems, which were described in the previous examples.

The combined 20 mM concentration of this acetate buffer system is a common composition used in the pharmaceutical industry. The respective concentrations of each excipient are such to target a pH of 4.3, which provides acceptable stability properties to the drug as demonstrated in formulation development studies.

The use of sodium chloride at 200 mM in the formulation serves as a tonicity agent to prevent the solution from being hypotonic. Infusion of hypotonic solutions can cause pain and safety issues when delivered. Sodium chloride was also chosen due to it being a common excipient in the pharmaceutical industry. It was also observed in formulation screening studies that the presence of sodium chloride appears to provide additional stability to the formulated product.

Phosphoric acid is used in trace amounts for pH regulation of the nanoparticle solution during DAN-222 drug substance manufacture to provide a low-pH environment that is conducive to the stability of DAN-222 in solution. The concentration of the acetate buffer is designed to target a final pH of 4.3 when added to the somewhat more acidic aqueous solution during final formulation.

DAN-222 drug substance solution is fully formulated at the time of its manufacture as DAN-222 Formulated Bulk Drug Substance (FBDS). Consequently, the composition of the DAN-222 drug product solution in vials is identical to that of the DAN-222 FBDS. This approach to drug product manufacture was made to minimize the amount of handling, and associated product risks, of performing additional formulation or compounding activities at the drug product vial filling site. This approach also provides a stable formulation for storage and handling of the DAN-222 FBDS prior to filling.

The physiochemical properties for the DAN-222 drug product are the same as those for the drug substance and have been discussed in detail the previous examples. Additional properties are detailed below.

### Compatibility

Formulation stability studies were performed in a vial/stopper configuration comparable to that selected for the GMP drug product. At no point during the development of DAN-222 has there been any evidence of incompatibility or loss of material to these surfaces. Furthermore, these studies provide evidence of the suitability of these vials during long-term storage frozen at −20±5° C. From a practical perspective, use of aseptically filled glass vials with rubber stoppers and crimp cap seals is a standard packaging configuration in pharmaceutical manufacturing.

Packaging and shipping considerations for liquid-filled vials are well-established and easy to configure.

All polymer components used in the manufacture of DAN-222 sterile solution for infusion are of pharmaceutical grades and have been qualified for use in parenteral filling operations.

The compatibility of development batches representative of DAN-222 Drug Product was also assessed, via challenges with a variety of clinical administration components. The DAN-222 drug product formulation was found to be compatible with a wide range of clinical administration components and dose preparation procedures. DAN-222 FBDS Lot SKT-757-077-NPS (the development lot used for dose range finding animal studies) was reformulated by dialysis to match the phase 1 clinical formulation for the drug product. This reformulated drug substance was filled into Type 1 borosilicate glass vials with rubber stoppers to replicate the clinical drug product container closure system. Syringes with needles were used to withdraw drug from vials for injection into IV bags.

Compatibility of the Drug Product was assessed using commercially available 100 mL IV bags containing either 0.9% (w/v) saline or 5% (w/v) dextrose. IV bag materials that were evaluated were Polyvinyl chloride (PVC), polyolefin (PO), and dual polyethylene-polypropylene (PE-PP). An IV administration set with a 0.22-μm PES (polyethersulfone) in-line filter was used for each diluent and bag material combination. Drug Product was added to the IV bag to target CPT concentrations in the IV bag of 0.022 mg/mL (low dose) and 0.400 mg/mL (high dose) to bracket the planned range of IV clinical dose concentrations that could be observed in the IV bag (0.025-0.36 mg/mL) per the clinical protocol. The high dose configuration requires use of more than one vial of drug product per IV bag.

To perform the clinical compatibility tests, Drug Product was injected into in each bag tested using a 25-gauge, ½ inch needle and silicone-free syringe. The bags were placed on a laboratory cart and pushed around the laboratory for 0.5 hour to represent the transport of IV bags from a clinical pharmacy preparation site to the patient for administration. The Drug Product was sequentially held in the IV bag under stagnant conditions at ambient room temperature (approximately 22-26° C.) for 12 hours, which represents a worst-case hold period. The total amount of time that the product was held in the IV bag before mock infusion was 12.5 hours (0.5 hour transport simulation, plus 12-hours stagnant hold). The IV bags were protected from light during the entire process.

Samples were collected after passage through the IV administration set with a 25G needle. In-process sample collection was performed through the IV bag port site with a syringe and expelled through a 25-gauge needle. IV bag port samples were taken and analyzed for information purposes only immediately following: DP injection into the bag; after the 0.5-hour transportation event; and after the subsequent 12-hour hold in the IV bag before mock infusion. The samples were analyzed for visual appearance (to look for visible particulates), CPT concentration (to determine possible adsorption to IV bags and administration set by comparing the actual CPT concentration to the calculated theoretical CPT concentration), % of total CPT as free CPT (purity), sub-visible particles (HIAC), particle size, polydispersity index, pH, and osmolality.

The results are provided in FIG. 51 for IV bag administration in the low dose (0.022 mg/mL) and in FIG. 52 for high dose (0.400 mg/mL) formats. These results demonstrate that the drug product is stable under the conditions tested and is recovered after mock infusion through the administration set following the 0.5-hour transportation simulation and 12-hour hold period. All samples exhibited an acceptable level of product adsorption, with 90-110% of theoretical target CPT concentration remaining in the solution over the course of the study. Based on these results, the compatibility study supports the use of the drug product as specified in the clinical protocol.

The drug product is to be injected into the IV bag using a silicone-free syringe, and an IV administration set with a 0.22 μm filter will be used during clinical studies to match the compatibility study conditions. Acceptable IV bag diluents, as determined in the study, are 0.9% (w/v) saline and 5% (w/v) dextrose. Use of 100 mL IV bags is specified; IV bag materials of construction that are acceptable are PVC, PO, and PE-PP. The maximum allowable drug product hold in the IV bag for product compatibility is 12.5 hours at room temperature.

### Microbiological Attributes

DAN-222 sterile solution for infusion is provided in type 1 borosilicate glass vials with chlorobutyl rubber stoppers and aluminum crimp caps. Each vial is intended for single use, and no preservative is added to the formulation.

DAN-222 is not stable to terminal sterilization by heat. It is likely that the polyethylene glycol components of MAP would be subject to free-radical degradation if subjected to sterilizing levels of gamma radiation. Consequently, microbial safety of both the drug substance and drug product is provided by sterile filtration. DAN-222 FBDS is passed through a 0.22 μm filter as it is transferred into sterile HDPE bottles for storage. Sterility of the drug product is achieved by passage of the FBDS through two 0.22 μm sterilizing filters in series immediately prior to vial filling under aseptic conditions.

To ensure continued protection from microbial contamination, container closure integrity qualification testing (CCIT) of the selected container closure system has been performed. CCIT by deterministic vacuum decay analysis is specified for the end-of-study stability time point.

### Example 9-DAN-222 Dose-Escalation Study of the Safety and Pharmacology of DAN-222 in Subjects with Metastatic Breast Cancer

This example provides the protocol for a Dose-escalation Study of the Safety and Pharmacology of DAN-222 in Subjects with Metastatic Breast Cancer using DAN-222. The study investigates the use of DAN-222 in subjects presenting Human epidermal growth factor receptor 2 (HER2)-negative metastatic breast cancer (mBC).

This is an open-label, multicenter, dose-escalation study designed to assess the safety, tolerability, and pharmacokinetics (PK) of intravenous (IV) administered DAN-222 followed by a dose escalation of DAN-222 in combination with niraparib. This clinical program is designed to evaluate DAN-222 as monotherapy and for combination use with a PARP inhibitor, which is expected to increase efficacy without significant increase in toxicity.

There are 2 stages within this study. Stage 1 has two parts. Part A analyzes dose escalation of single agent DAN-222. Part B analyzes dose escalation of DAN-222 in combination with niraparib 100 mg. Stage 2 involves expansion of three separate HER2-negative mBC cohorts: one group for single-agent DAN-222 in subjects with either homologous recombination repair deficient (HRD)-positive or HRD-negative tumors, and one cohort each for DAN-222 combined with niraparib in subjects with either HRD—positive tumors or HRD-negative tumors. DAN-222 is administered IV on Days 1, 8, 15, and 22 of 28-day cycles. Niraparib dose is taken orally once daily on Days 1 to 28. Subjects are treated for cycles defined as 4 weeks. Subjects exhibiting acceptable safety and evidence of clinical benefit (SD, PR, or CR) continue to receive DAN-222 until confirmed objective disease progression or unacceptable toxicity. Initial assessments include MRI/CT at 6-week intervals through the first 6 cycles then at 8-week intervals after week 24.

This study scheme is depicted in FIG. 53. Subjects exhibiting acceptable safety and evidence of clinical benefit (stable disease [SD], partial response [PR], or complete response [CR]) continue to receive DAN-222 until confirmed objective disease progression or unacceptable toxicity.

### Dose Escalation Stage 1

Stage 1, Part A—The dose-escalation phase of the study assesses the safety, tolerability, and PK of DAN-222 as a single agent administered by IV infusion. A classical 3±3 design is employed and subjects are evaluated for DLTs. If no dose limiting safety signals are detected during the 28-day DLT window, the study serially progresses to higher dose levels.

Stage 1, Part B—The starting dose of DAN-222 administered IV QW in combination with niraparib 100 mg will begin after the monotherapy has cleared the same dose level DLT safety window or higher (e.g. the 2 mg/m² DAN-222±100 mg niraparib begins only after 2 mg/m² single agent clears the DLT safety window). The combination dose escalation follows the same dose escalation intervals as the single agent escalation, employing a classical 3±3 design, and subjects are evaluated for DLTs. The escalation proceeds until a maximal dose level seen in Part A or until a safety threshold is met. If no dose limiting safety signals are detected during the 28-day DLT window, the study serially progresses to higher dose levels.

Enrollment into dose-escalation cohorts based on the 3+3 dose-escalation design is conducted in accordance with the following rules. First, a minimum of 3 subjects are initially enrolled in each cohort unless the first 2 enrolled subjects experience a protocol-defined DLT, in which case enrollment into the cohort is terminated. If none of the first 3 DLT-evaluable subjects experiences a DLT, enrollment of the next cohort at the next higher dose level proceeds with the dose interval increase being≤100% of the preceding dose. If 1 of the first 3 DLT-evaluable subjects experiences a DLT, the cohort expands to 6 subjects. All subjects are evaluated for DLTs before any dose-escalation decision. If DLTs are observed in <17% of subjects in a given cohort (e.g., DLTs observed in 1 of 6 DLT-evaluable subjects), enrollment of the next dose-escalation cohort proceeds. The interval increase between successive cohorts is ≤50% of the preceding dose level. If DLTs are observed in ≥17% of subjects in a given cohort, further enrollment at that dose level and dose escalation is halted, and that dose is declared as exceeding the MTD. Additional subjects are also enrolled at the same dose-escalation cohort to further describe safety, PK, pharmacodynamics (PD), or efficacy at that dose level.

The MTD is defined as the highest dose level resulting in DLTs in <17% of a minimum of 6 subjects.

Additional intermediate dose cohorts between two dose levels that have been demonstrated to not exceed the MTD are evaluated to further characterize dose-dependent toxicities or pharmacodynamic changes.

If the dose level at which the MTD is exceeded is >25% higher than the preceding tested dose level, additional dose cohorts of at least 6 subjects are evaluated at intermediate dose levels to determine the MTD.

Safety information from intermediate dose level cohorts is used to guide dose-escalation decisions by the SRT, e.g., if the frequency of DLT-defined events observed in the intermediate dose level cohorts is greater than expected based on escalation cohort data, the SRT may recommend interrupting or stopping further dose escalation

Enrollment of cohorts to evaluate intermediate dose levels in either Part A or Part B may occur concurrently with enrollment of dose-escalation cohorts in the other Part to identify the MTD. If the MTD is not exceeded at any dose level, the highest dose administered in this study is declared the MAD.

Dose-escalation decisions are made based on the recommendations of the SRT and in consultation with the study Investigators. Relevant demographic, AE, laboratory, dose administration, and available PK data will be reviewed prior to each dose-escalation decision.

On the basis of a review of real-time safety data and available preliminary PK data, dose escalation may be halted or modified by the SRT as deemed appropriate. Dose escalation continues until a dose level is reached at which DLTs are observed in ≥17% of >6 subjects is reached.

Dose interruption and/or reduction is implemented per the Investigator's judgment after Cycle 1 in subjects enrolled in Stage 1 (Dose Escalation) and at any time for subjects enrolled in Stage 2 (Dose Expansion) due to any grade toxicity considered intolerable by the subject.

The toxicity of each dose is recorded prior to the administration of a subsequent dose and graded according to the NCI CTCAE v5.0. All dose reductions are based on the worst preceding toxicity and follow the reduction schedule listed in Table 12 below.

TABLE 12
DAN-222 Dose Reduction Schedule

| Event | Dose |
|---|---|
| Initial dose | RP2D or current cohort dose |
| First reduction | Go down one dose level |
| Second dose reduction | Go down second dose level |
| Third dose reduction | Discontinue dose |

In the case of dose interruptions, the next dose follows the subject's original calendar schedule. Cycle timing is not delayed for treatment interruptions, and tumor assessment occurs according to this schedule regardless of whether study drug is interrupted. Once the dose of DAN-222 is reduced, any re-escalation is discussed with the Medical Monitor.

For subjects in Stage 1, the dose of DAN-222 may be increased to a higher dose level that has been found to be safe during the dose-escalation phase after the subject has completed the 12-week tumor assessment and following discussion with the Medical Monitor.

Subjects who do not experience a DLT during the DLT observation period are eligible to receive additional cycles of study treatment with DAN-222. Subjects exhibiting acceptable safety and evidence of clinical benefit (SD, PR, or CR) may continue to receive DAN-222 until objective disease progression or unacceptable toxicity.

Any treatment delay not attributed to study treatment may not require study treatment discontinuation but must be approved by the Medical Monitor. Dose reductions of DAN-222 may be allowed if it is determined that clinical benefit may be maintained.

Subjects who complete study treatment without disease progression continue to be monitored, including regularly scheduled tumor assessments until discontinuation from the post-treatment follow-up or until initiation of a different therapy or study.

All adverse events, including DLTs, are reported and graded according to NCI CTCAE v5.0 unless otherwise indicated. Dose limiting toxicities are treated according to clinical practice and are monitored through resolution. Adverse events meeting the DLT criteria are reported to the Sponsor as soon as possible.

For dose-escalation purposes, the DLT assessment period for Stage I-Parts A and B is defined to be during the first 28 days of treatment (i.e., Cycle 1, Day 1 through Cycle 1, Day 28).

Dose-limiting toxicity criteria are as follows: (i) any treatment-related Grade 3 or Grade 4 non-hematologic clinical (i.e., non-laboratory) AE: except for fatigue, nausea, vomiting, diarrhea, and electrolyte imbalances which can be controlled by supportive care; (ii) any treatment-related Grade 3 or Grade 4 non-hematologic laboratory abnormality if: medical intervention is required to treat the subject, or the abnormality leads to hospitalization, or the abnormality persists for ≥7 days; (iii) any treatment-related hematologic toxicity specifically defined as: thrombocytopenia Grade 4 for ≥7 days, or Grade 3 or Grade 4 associated with bleeding or requiring platelet transfusion, neutropenia Grade 4 for >7 days, or Grade 3 or Grade 4 associated with infection or febrile neutropenia, or anemia Grade 4 for ≥7 days, or Grade 3 or Grade 4 requiring blood transfusion.

Exceptions to DLTs include Grade 3 abnormalities in laboratory values that are asymptomatic and deemed not clinically significant.

Determination of whether a subject is evaluable for DLT assessment will be made in accordance with the following rules. First, subjects who receive study treatment and remain on study through the DLT assessment window are considered DLT-evaluable. Subjects who receive at least 1 dose and have a DLT are evaluable; subjects without a DLT must receive at least 4 doses to be evaluable. Subjects who discontinue from study treatment prior to completing the DLT assessment window for reasons other than a DLT are considered non-evaluable for dose-escalation decisions and MTD determination and will be replaced by an additional subject at the same dose level. Subjects who have pre-existing conditions during the DLT assessment window that confounds the evaluation of DLTs may be replaced at the discretion of the Medical Monitor.

### Stage 2

The RP2D of DAN-222 monotherapy may be selected based on one or more of the following: (i) the MAD or the MTD (the highest dose level in which the rate of DLTs is <17% in a minimum of 6 subjects); (ii) PK and/or PD results; (iii) the occurrence, nature, and severity of toxicities occurring after the DLT evaluation period (i.e., first 28 days of treatment); and (iv) anti-tumor activity.

The dose-expansion stage of this study is designed to obtain additional safety, tolerability, PK, and preliminary clinical activity data with study treatment at the RP2D dose. The initiation of the expansion cohorts is at the Sponsor's discretion. The Sponsor, in consultation with the Investigators and the SRT, evaluates all available safety data from the expansion cohorts on an ongoing basis to assess the tolerability of the dose levels studied. At no time does a DAN-222 dose level studied in the expansion stage exceed the highest dose level that qualifies as an MTD for a given schedule in the dose-escalation stage. Additionally, for each expansion cohort, the data is reviewed continuously in order to guide potential early stopping of enrollment in the event of excess toxicity or fatality.

The expansion cohorts further evaluate anti-tumor activity, safety, and pharmacodynamics. Enrollment in one or more of the expansion cohorts is initiated following review of the above and recommendation by the SRT following consultation with study Investigators. After the single agent DAN-222 dose escalation defines the RP2D, an expansion of 20 subjects is opened for subjects with HER2-negative mBC and with either HRD-positive or HRD-negative status. After the RP2D dose of DAN-222 in combination with niraparib is selected, the 2 expansion cohorts of HRD-positive (n=20) and HRD-negative (n=20) subjects is opened.

The single agent expansion cohort may open concurrently with ongoing dose escalation in the combination arm. In that event, the dose for the expansion cohort(s) is adjusted based on SRT review of cumulative safety, laboratory, and efficacy data. Additional subjects per cohort may be enrolled based upon cumulative data and additional cohorts may be added with a protocol amendment. Data from expansion cohorts is further support for RP2D selection per review by SRT.

### Study Subjects

Approximately 96 subjects will be enrolled into this study. The Stage 1 dose escalation study, Part A, single agent DAN-222 includes approximately 18 subjects. The Stage 1 dose escalation study, Part B, combination DAN-222 and niraparib includes approximately 18 subjects.

During the expansion phase, the study enrollment is as follows: Group A: single agent DAN-222 in subjects with HER2-negative mBC with HRD-positive or HRD-negative tumors, n=20; Group B: combination DAN-222 with niraparib in subjects with HER2-negative mBC with HRD-positive tumors, n=20; and Group C: combination DAN-222 with niraparib in subjects with HER2-negative mBC with HRD-negative tumors, n=20.

In the Dose Escalation Stage (Parts A and B), subjects who do not complete the DLT evaluation period for reasons other than a DLT are considered not DLT-evaluable and are replaced. In the Dose Expansion phase, subjects who do not have at least one post-baseline radiographic tumor evaluation for reasons other than treatment discontinuation due to toxicity, or death due to disease progression are considered not evaluable for efficacy and may be replaced.

### Subject Eligibility

Subjects must have histologically documented, metastatic, HER2-negative breast cancer that has progressed after two prior lines of therapy (including adjuvant therapy if progressed within the last 12 months, and aromatase inhibitors will be considered a line of therapy) or for which standard therapy has proven to be ineffective or intolerable, or is considered inappropriate. HER2 positivity is defined by standard of care fluorescence in situ hybridization (FISH) and/or 3+ staining by immunohistochemistry (IHC) according to the American Society of Clinical Oncology/College of American Pathologists (ASCO/CAP) Clinical Practice Guideline Focused

### Update.

A minimum of 2 weeks or 5 half-lives from any prior therapy (whichever is longer) will be required for mBC. Therapies include chemotherapy, immunotherapy and/or radiation therapy. In addition, recovery to Grade≤1 from all reversible toxicities, except alopecia, is required at study entry.

Subjects must have measurable disease as per RECIST v1.1. Subject must be females, age 18 years or older. Subjects must possess an ECOG performance status≤2.

Subjects with previously treated brain metastases (surgery, whole or stereotactic brain radiation) are allowed provided the lesions have been stable for at least 4 weeks and the subject is off steroids for at least 7 days prior to first dose of study treatment, and any neurologic symptoms have returned to baseline (without evidence of progression by imaging using the identical imaging modality for each assessment, either MRI or CT scan).

Subjects with brain metastases should not require use of enzyme-inducing antiepileptic drugs (e.g., carbamazepine, phenytoin, or phenobarbital) within 14 days before first dose of study treatment and during study. Use of newer antiepileptics that do not produce enzyme induction drug-drug interactions is allowed.

Subjects must have normal organ and marrow function, defined as: absolute neutrophil count >1.5×10⁹/L without growth factor support in the last 7 days; platelets ≥100×10⁹/L without growth factor support in the last 7 days; hemoglobin ≥9 g/dL and no blood transfusion within the preceding 4 weeks; total bilirubin≤1.5× the upper limit of normal (ULN) (unless Gilbert's Disease); AST (SGOT)/ALT (SGPT)≤2.5×ULN (≤5×ULN if liver metastases); and creatinine <1.5× ULN OR creatinine clearance ≥50 mL/min (calculated using the Cockroft-Gault formula) for subjects with creatinine levels above institutional normal

Subjects of childbearing potential must have had a negative serum pregnancy test within 72 hours prior to the first dose of study medication or agree to abstain from activities that could result in pregnancy from the screening visit through 120 days after the last dose of study treatment, or be of non-childbearing potential. Non-childbearing potential is defined as (by other than medical reasons): a subject 45 years of age or older that has not had menses for >1 year; amenorrheic at least 2 years without a hysterectomy and oophorectomy and a follicle stimulating hormone (FSH) value in the postmenopausal range upon pre-study (screening) evaluation; or a subject who has had a post hysterectomy, bilateral oophorectomy, or tubal ligation. Documented hysterectomy or oophorectomy must be confirmed with medical records of the actual procedure or confirmed by an ultrasound. Tubal ligation must be confirmed with medical records of the actual procedure, otherwise the subject must be willing to use 2 adequate barrier methods throughout the study, starting with the screening visit through 120 days after the last dose of study therapy.

Stage 2 inclusion additionally requires documentation of DNA repair defects status (e.g., BRCA1, BRCA2, ATM, BARD1, BRIP1, CDK12, CHEK2, FANCA, NBN, PALB2, RAD51, RAD51B, RAD51C, RAD51D, RAD54L) validated by plasma testing through the central laboratory or from archival tumor tissue or germ line testing from any Clinical Laboratory Improvement Amendments (CLIA) approved lab.

The subject exclusion criteria include: (i) any significant medical condition or laboratory abnormalities which place the subject at unacceptable risk if he/she were to participate in the study at clinician's discretion and not otherwise stated below; (ii) for the DAN-222 and niraparib combination cohorts, subjects cannot have known sensitivity to FD&C Yellow No. 5 (tartrazine). Subjects are also excluded if they have had an allergic reaction to irinotecan, topotecan, or govitecan. Excluded subject also include those who have had concurrent administration or received cytochrome P450 3A4 (CYP3A4) enzyme inducers or inhibitors within 2 weeks prior to the first day of study treatment. Subjects taking medications know to prolong the QT interval or associated with torsades de pointes, are also excluded unless the subject can safely discontinue these medications or change to comparable medications that do not significantly prolong the QT interval, at least 5 half-lives or 7 days (whichever is longer) prior to the first dose of DAN-222. Subjects with a history of myelodysplasia, or having a known additional malignancy that progressed or required active treatment within the last 3 years are excluded. Exceptions include non-melanoma skin cancer and carcinoma in situ. Subjects who have been diagnosed with carcinomatous meningitis are also excluded. Excluded subjects also include subjects that are pregnant, breastfeeding, or expecting to conceive children within the projected duration of the study, starting with the screening visit through 120 days after the last dose of study treatment. Thus, excluded subjects also include those with an inability to comply with study procedures or unwilling to use adequate birth control. Excluded subjects further include those with uncontrolled intercurrent illness including, but not limited to, ongoing or active infection, symptomatic congestive heart failure, unstable angina pectoris, cardiac arrhythmia, or other conditions that would limit compliance with study requirements.

Also excluded are subjects having a heart-rate corrected QT interval (QTc) prolongation of >470 msec at screening. If a subject has a prolonged QT interval and the prolongation is deemed to be due to a pacemaker upon Investigator evaluation (i.e., the subject otherwise has no cardiac abnormalities), the subject may be eligible to participate in the study following discussion with the Medical Monitor.

Finally, excluded subjects also include those having any serious social, psychosocial, or medical condition or abnormality in clinical laboratory tests that, in the Investigator's judgment, precludes the subject's safe participation in and through a minimum of 4 cycles of treatment, or which could affect compliance with the protocol or interpretation of results.

### Estimated Study Duration and Study Completion

The planned study duration is three years. Subject participation will include screening, treatment, and follow-up. Screening can be conducted up to 30 days before first dose of study drug, during which the subject's eligibility and baseline characteristics are determined. Subjects with confirmed progressive disease terminate the study at earlier time points. In subjects with SD, PR, or CR, treatment with DAN-222 is continued until an unacceptable drug-related toxicity occurs or until disease progression. Post treatment, subjects are observed for disease progression and new anticancer therapies, or until withdrawal of consent or the end of the study, whichever occurs first. The planned duration of the study treatment for individual subjects is approximately 12 months. The end of this study is defined as the date when the last subject visit occurs, or the date at which the last data point required for statistical analysis or protocol defined safety monitoring is received from the last subject, whichever occurs later.

DAN-222 & Niraparib administration

DAN-222 is supplied by the Sponsor. DAN-222 is a polymer-small molecule conjugate NP formulated as a sterile aqueous solution.

Both DAN-222 drug substance and drug product are manufactured under current Good Manufacturing Practices. DAN-222 drug product is a sterile, clear to slightly opalescent, colorless to light yellow preservative-free liquid intended for IV infusion. DAN-222 is supplied in single-use vials containing 3.1 mL of a solution of DAN-222 containing 2.6 mg/mL of CPT. The formulation consists of an aqueous solution of 20 mM sodium acetate and 200 mM sodium chloride, with the pH adjusted to 4.3. Vials are to be stored frozen at −20° C.±5° C. and thawed at either 2 to 8° C. or ambient temperature immediately prior to use. The labeling complies with the requirements of the applicable regulatory agencies.

DAN-222 is administered IV once weekly. The dose of DAN-222 for each subject depends on their dose level assignment. The dose is based on the subject's actual weight at baseline (Cycle 1, Day 1 or during screening). If the subject's weight changes by >10% during the course of the study, the body surface area and drug doses are recalculated. Recalculation of drug dose on each treatment day regardless of percentage of body weight change is also allowed, per institutional dosing policy.

DAN-222 is administered to subjects by IV infusion using IV bags. Testing has shown that DAN-222 is compatible with PVC extension sets and non-siliconized polypropylene syringes. DAN-222 is administered in a setting with immediate access to trained critical care personnel and facilities equipped to respond to and manage medical emergencies.

Subjects must be well-hydrated throughout DAN-222 treatment and are encouraged to drink at least 2 liters of water per day. DAN-222 is infused over 1 hour±15 minutes. The infusion may be slowed or interrupted for subjects experiencing infusion-associated symptoms. For the first infusion, the subjects are observed for 90 minutes post-infusion for any infusion-related reactions. Following each subsequent DAN-222 dose, the duration of dosing may be successively reduced by up to 30 minutes to a minimum infusion time of 30 minutes. In the absence of infusion-related adverse events the post-administration observation time may also be successively reduced by up to 30 minutes, to a minimum observation period of 30 minutes.

Subjects who undergo intra-subject dose escalation or re-treatment receive the first higher infusion of DAN-222 over a minimum of 1 hour±15 minutes.

Any overdose or incorrect administration of DAN-222 are noted on the Study Drug Administration eCRF. Adverse events associated with an overdose or incorrect administration of study drug are recorded on the Adverse Event eCRF.

Niraparib (2-{4-[(3S)-piperidin-3-yl]phenyl}-2H indazole 7-carboxamide 4-methylbenzenesulfonate hydrate) (1:1:1) is an orally available, potent, highly selective PARP1 and PARP2 inhibitor. The excipients for niraparib are lactose monohydrate and magnesium stearate. Niraparib is supplied as 100-mg capsules and is administered orally once daily (QD) continuously starting on Cycle 1, Day 1. The daily dose administered is a single 100 mg capsule. Subjects are instructed to take their dose at the same time each day, preferably in the morning. Subjects must swallow and not chew the capsule. Niraparib capsules are dispensed to subjects on Cycle 1, Day 1 and on Day 1 of every 28-day cycle thereafter until the subject discontinues study treatment.

### Study Drug Administration-Day 1

Prior to treatment subjects are required to complete all medical history, physical exam, and laboratory assessments. Evaluations will be performed as indicated in the Schedule of Assessments (Table 13).

Additionally, vital signs will be measured within approximately 5 minutes (+5 min) before and 15 minutes (+5 min) after the start of infusion, then every 30 minutes (+10 min) for the next 2 hours. Vital signs may continue to be monitored after this point, until subject is stable and as clinically indicated.

Subjects enrolled in dose escalation cohorts will require clinical monitoring post-infusion for 8 hours during the PK assessments, following the Cycle 1, Day 1 DAN-222 administration.

Ongoing Study Treatment DAN-222 is administered once weekly. Subjects will return to the clinic for safety evaluations and to receive the DAN-222 infusion. Evaluations are performed as indicated in the Schedule of Assessments (Table 13).

### Maintenance Treatment

Subjects who continue to tolerate DAN-222 and who have not had progression of their malignancy may continue on study treatment. Subjects will return to the clinic once a week for safety evaluations and to receive the DAN-222 infusion.

### End of Treatment

The End-of-Treatment (EOT) visit must occur within 28 days of treatment discontinuation and prior to initiation of any new anticancer therapy/regimen. All subjects discontinuing study treatment for any reason should undergo the evaluations as indicated in the Schedule of Assessments (Table 13).

### Early Withdrawal

All subjects should return to the clinic to have the EOT visit assessments performed, irrespective of when treatment is completed. Adverse events are assessed until resolution, return to baseline, or are stabilized, per the Investigator's assessment.

TABLE 13
Schedule of Assessments and Procedures

| | | Treatment Period | | | | | | | | | | | | |
|---|---|---|---|---|---|---|---|---|---|---|---|---|---|---|
| | | Cycle | | | | | | | | | | | | Study |
| | Screening^{a} | 1 | | | | 2 | | | | 3+ | | | | Completion/ |
| | Weeks | | | | | | | | | | | | | Early |
| | −4 to 0 | 1 | 2 | 3 | 4 | 1 | 2 | 3 | 4 | 1 | 2 | 3 | 4 | Termination^{b} |
| Informed consent | x | | | | | | | | | | | | | |
| Review of | x | | | | | | | | | | | | | |
| eligibility | | | | | | | | | | | | | | |
| criteria | | | | | | | | | | | | | | |
| Medical history | x | | | | | | | | | | | | | |
| and demographic | | | | | | | | | | | | | | |
| characteristics | | | | | | | | | | | | | | |
| Complete | x | | | | | | | | | | | | | x |
| physical | | | | | | | | | | | | | | |
| examination | | | | | | | | | | | | | | |
| Limited physical | | x^{d} | x | x | x | x | x | x | x | x | | | | |
| examination | | | | | | | | | | | | | | |
| Height | x | | | | | | | | | | | | | |
| Body weight | x | x^{d} | | | | x | | | | x | | | | x |
| Vital signs^{e} | x | x | x | x | x | x | x | x | x | x | | | | x |
| ECOG | x | x^{d} | | | | x | | | | x | | | | x |
| Performance | | | | | | | | | | | | | | |
| Status | | | | | | | | | | | | | | |
| 12-lead ECG^{f} | x^{g} | | x | | | | | | | | (x^{f}) | | | x |
| Tumor | xⁱ | | | | | | x^{j} | | | | (x^{j}) | | (x^{j}) | x^{k} |
| assessment^{h} | | | | | | | | | | | | | | |
| Hematology^{l} | x^{g} | x^{d} | x | x | x | x | x | x | x | x | | | | x |
| Chemistry^{m} | x^{g} | x^{d} | x | | | x | x | | | x | | | | x |
| Coagulation | x^{g} | | | | | | | | | xⁿ | | | | x |
| (aPTT and PT) | | | | | | | | | | | | | | |
| | Plasma PK Samples (Stage 1) ^{v}Refer to Table | | | | | | | | | | | | | |
| | Plasma PK Samples (Stage 2) ^{v}Refer to Table | | | | | | | | | | | | | |
| Serum | x^{g} | | | | | | | | | | | | | |
| pregnancy test^{o} | | | | | | | | | | | | | | |
| Urinalysis^{p} | x^{g} | | | | | x | | | | x | | | | x |
| DAN-222 | | x | x | x | x | x | x | x | x | x | x | x | x | |
| administration | | | | | | | | | | | | | | |
| Niraparib | | x | x | x | x | x | x | x | x | x | x | x | x | |
| administration^{q} | | | | | | | | | | | | | | |
| Adverse event | | x | x | x | x | x | x | x | x | x | x | x | x | x^{s} |
| monitoring^{r} | | | | | | | | | | | | | | |
| Concomitant | x^{t} | x | x | x | x | x | x | x | x | x | x | x | x | x |
| medications | | | | | | | | | | | | | | |
| Circulating | x | | | | | x | | | | | | | | |
| tumor cell | | | | | | | | | | | | | | |
| (CTC) sample^{u} | | | | | | | | | | | | | | |
| HRD blood | x | | | | | | | | | | | | | |
| sample^{u, v} | | | | | | | | | | | | | | |
| Immunogenicity | x | x | | | | | | | | (x) | | | | x |
| sample^{u, w} | | | | | | | | | | | | | | |

aPTT = activated partial thromboplastin time; ALT = alanine aminotransferase; AST = aspartate aminotransferase; BUN = blood urea nitrogen; CBC = complete blood count; CT = computed tomography; ECG = electrocardiogram; ECOG = Eastern Cooperative Oncology Group; INR = international normalized ratio; MRI = magnetic resonance imaging; PK = pharmacokinetic; RBC = red blood cell; RECIST = Response Evaluation Criteria in Solid Tumors; WBC = white blood cell.
Notes:
All visits must occur within ±3 day from the scheduled date unless otherwise noted. All assessments will be performed on the day of the specified visit unless a time window is specified. Assessments scheduled on a day of study drug administration should be performed prior to study drug dosing unless otherwise specified.
^{a}Informed consent must be obtained before any study specific screening assessments are performed. Screening assessments are to be performed within 28 days preceding Cycle 1 Day 1. Assessments performed as standard of care within the screening window may be used for screening.
^{b}Subjects will return to the clinic within 28 days after their last dose of study treatment for a study completion/early termination visit.
^{c}Limited and complete physical examinations are described infra.
^{d}Screening assessments performed ≤96 hours prior to Cycle 1 Day 1 do not have to be repeated for Cycle 1 Day 1.
^{e}Includes heart rate, systolic and diastolic blood pressures while the subject is in a seated position, and oral or tympanic temperature. Oxygen saturation will be assessed by pulse oximetry after the subject has been in a seated position for ≥5 minutes.
^{f}12-lead ECG should be performed at screening, on Cycle 1 week 2 prior to dosing, Cycle 3 week 2 prior to dosing, Cycle 6 week 2 prior to dosing at end of study, and as clinically indicated during study. Subjects should be resting in a supine position for ≥5 minutes prior to ECG recording.
^{g}Perform ≤14 days prior to Cycle 1 Day 1.
^{h}Any evaluable and measurable disease must be documented at screening and re-assessed at each subsequent tumor evaluation. Systemic response will be assessed by the Investigator per RECIST v1.1. Screening systemic assessments must include CT or MRI scans of the chest, abdomen, pelvis and a baseline bone scan to assess bone metastases. If necessary, screening assessments of brain must be assessed by CT or magnetic resonance imaging. At the Investigator's discretion, other methods of assessment of measurable disease per RECIST v1.1 may be used in addition to those listed above. Additional procedures for tumor assessment should be performed as clinically indicated. The same imaging methods used at screening must be used throughout the study for each subject.
ⁱA documented standard-of-care tumor assessment performed within 28 days prior to Cycle 1 Day 1 may be used for the screening assessment if it includes chest, abdomen, and pelvis.
^{j}Perform during Cycle 2 Weeks 2 to 3 and every 6 weeks thereafter, after six months perform every 8 weeks. Results must be reviewed prior to dosing at next cycle.
^{k}Perform only if tumor assessments were not performed within the previous 28 days.
^{l}Includes CBC, including RBC count, hemoglobin, hematocrit, WBC count with differential (neutrophils, eosinophils, basophils, lymphocytes, monocytes, and other cells), and platelet count.
^{m}Includes glucose, urea/BUN, creatinine, sodium, potassium, magnesium, chloride, bicarbonate, calcium, phosphorus, total protein, albumin, direct and total bilirubin, alkaline phosphatase, AST, and ALT.
ⁿObtain every 3 cycles thereafter (e.g., Cycle 3 Day 1, Cycle 6 Day 1, Cycle 9 Day 1, etc.).
^{o}For women of childbearing potential, a serum pregnancy test is required.
^{p}Tests to be performed are pH, specific gravity, glucose, ketones, blood, and protein.
^{q}Niraparib is administered only in the combination Stage 1, Part B and in the combination expansion cohorts (Stage 2, Groups B and C). Subject diary to be collected on Day 1 of each cycle.
^{r}After initiation of study treatment, all adverse events and serious adverse events, regardless of attribution, will be recorded until 30 days after the last dose of study treatment or until initiation of another anticancer therapy, whichever occurs first. After this period, only treatment-related serious adverse events will be recorded.
^{s}Any ongoing adverse event thought to be related to study treatment will be followed until the event has resolved to baseline grade, the event is assessed by the Investigator as stable, new anti-tumor treatment is initiated, the subject is lost to follow-up, the subject withdraws consent, or it has been determined that study treatment or participation is not the cause of the adverse event. Subjects will be contacted 30 days after the last dose of study treatment to determine whether any treatment-related serious adverse events have occurred.
^{t}Including prescription medications and over-the-counter preparations used by the subject between 7 days preceding the screening evaluation and the end of the study.
^{u}Detailed information regarding the collection, handling, and shipment to the central lab is provided elsewhere herein.
^{v}Needed for subjects in Stage 2 who do not have prior results of HRD testing available in the medical record.
^{w}Serum samples to be collected and stored for future testing (if clinically indicated) of anti-DAN-222 antibodies will be drawn at baseline (pre-infusion) on Day 1 of Cycle 1 pre-infusion of Cycles 3 and every subsequent third cycle and at the treatment completion/early termination visit.

TABLE 14
DAN-222 Pharmacokinetic Assessments During Stage 1

| | | | | | | | | | | | | Study |
|---|---|---|---|---|---|---|---|---|---|---|---|---|
| | | | Post-end of infusion | | | | | | | | | Completion/ |
| | | | | | | | | Day | Day | Day | Day | Early |
| | | 0-4 hr | Day 1 | | | | | 2 | 3 | 5 | 6 | Termination |
| Time | | Pre- | 30 | 2 | 4 | 6 | 8 | 24 | 48 | 96 | 120 | During |
| Point | Sample | dose | min | hr | hr | hr | hr | hr | hr | hr | hr | visit |
| Cycle 1 | PK | x | x | x | x | x | x | x | x | x | x | |
| | plasma | | | | | | | | | | | |
| | sample | | | | | | | | | | | |
| Cycle 2 | PK | x | x | | x | | | | | | | x |
| | plasma | | | | | | | | | | | |
| | sample | | | | | | | | | | | |
| Cycles 3+ | PK | x | x | | (x)^{a} | | | | | | | x |
| | plasma | | | | | | | | | | | |
| | sample | | | | | | | | | | | |

PK = pharmacokinetic, (PK samples taken at pre-dose, 30 min, 2 hr should be taken ±5 min form scheduled EOI. PK Samples taken 4+ hr should be taken ±15 min)
^{a}In cycles 3+, the 4 hour PK sample will be taken every 3rd cycle

TABLE 15
DAN-222 Pharmacokinetic Assessments during Stage 2

| | | DAN-222 |
|---|---|---|
| | | PK |
| Time Point | | Sampling |
| Cycle 1, Day 1 | Pre-dose (0 to 4 hours prior to infusion, | x |
| | 30 minutes (±5 min) and 4 hours (±15 | |
| | min) postEOI | |
| Cycle 2, Day 1 | Pre-dose (0 to 4 hours prior to infusion) | x |
| | and 30 minutes (±5 min) post-EOI | |
| Cycle 3+ and | Pre-dose (0 to 4 hours prior to infusion, | x |
| every subsequent | 30 minutes (±5 min) and 4 hours (±15 | |
| 3 cycles, Day 1 | min) postEOI | |

### Physical Exam, Vital Signs, and Performance Status

A complete physical exam is performed at the Screening visit. A complete physical examination includes an evaluation of head, eye, ear, nose, and throat; and cardiovascular, dermatological, musculoskeletal, respiratory, gastrointestinal, and neurological systems. Changes from baseline are recorded at each subsequent physical examination. New or worsened abnormalities are recorded as adverse events if appropriate. A limited physical examination is performed at other visits to assess changes from baseline abnormalities, any new abnormalities, and to evaluate patient-reported symptoms. New or worsened abnormalities are recorded as adverse events if appropriate.

Vital signs, including blood pressure, heart rate, respiratory rate, and temperature, are monitored and recorded at screening and at times outlined in the Schedule of Assessments (Table 13). In addition to the time points outlined in the Schedule of Assessments, vital signs are monitored as clinically indicated, and if there are any new or worsening clinically significant changes since the last exam, changes are reported on the appropriate eCRF page. Performance status as measured by the ECOG scale is performed to quantify the subject's general well-being and ability to perform activities of daily life.

### Cardiac Function

Baseline 12-lead ECG is performed prior to the first infusion (i.e., Day 1) of every third 4-week cycle. The ECG should be performed prior to blood draws for PK. Subjects are to be supine and rested for approximately 5 minutes before ECGs are recorded.

### Neurological Examinations

A neurological exam is performed at the Screening visit, and any abnormalities of the following are recorded: cranial nerves, motor system, reflexes, coordination, sensory system, and neuropsychological findings (e.g., speech, cognition, and emotion). Thereafter, symptom-directed neurological assessment is performed before infusion. Subjects are specifically asked about changes in neurological status since the previous examination, as noted in the Schedule of Assessments (Table 13).

### Neurological Imaging

Subjects with neurological signs or symptoms undergo a screening brain MRI to rule out central nervous system metastasis during the screening period of the study. If an MRI is contraindicated, then a brain CT scan may be performed instead. Subsequent brain scans are performed at the planned imaging intervals if detected during screening.

### Disease Assessment

Subjects are evaluated for disease response by the Investigator at times indicated in the Schedule of Assessments. Disease assessments are evaluated per the RECIST v1.1. As part of the disease assessment, physical examinations include an evaluation of the presence and degree of enlarged lymph nodes, hepatomegaly, and splenomegaly.

### Imaging at Baseline

To confirm eligibility of having a measurable lesion as per RECIST v1. 1 and/or to establish a baseline, MRI or CT scans of chest, abdomen and pelvis, along with the appropriate imaging of all other sites of disease (including brain and bone) are required at screening. An MRI or CT performed following the subject's last line of therapy and prior to signing the ICF may be used for confirmation of eligibility if performed within 28 days prior to the screening visit and no other anticancer treatment has been administered. If MRI or CT is performed >28 days prior to the screening visit, the MRI or CT scan must be repeated to establish a new baseline. An MRI or CT is performed as close to the screening visit as possible.

Subjects who cannot have contrast MRI or CT scans due to contrast allergy or impaired renal function may be evaluated with non-contrast MRI or CT scans.

The same radiographic assessment modality as was used at baseline is preferably used for all response evaluations. Imaging should use the same protocol for consistency across different time points (e.g., MRI or CT with the same contrast protocol).

### Following Initiation of Study Drug Treatment

The first on-study imaging assessment is performed at 6 weeks (42±7 days) from the date of first dose of study treatment; in the case of equivocal progression of disease a confirmatory image is required <4 weeks later. If SD or better response, subsequent tumor imaging is performed every 6 weeks (42±7 days) or more frequently if clinically indicated and at the time of suspected disease progression. After 6 months of radiographic assessments, subjects have imaging performed every 8 weeks (56±7 days). Imaging should not be delayed for delays in cycle starts or extension of combination treatment cycle intervals. The same modality (i.e., CT or MRI) should be used for a given subject throughout the study.

Subjects with CR or PR will have the response confirmed by a repeat tumor imaging assessment performed at the next scheduled scan (i.e., 6 weeks later).

### Clinical Laboratory Analyses

Screening and other laboratory evaluations are performed according to the Schedule of Assessments. The Investigator may choose to repeat any abnormal test once in order to rule out laboratory or sample collection error. Assessments listed in Table 16 are performed at the local laboratory at the time points indicated in the Schedule of Assessments (Table 13).

TABLE 16
Clinical Laboratory Parameters

| Serum | | | |
|---|---|---|---|
| Chemistries | Hematology | Urinalysis | Other |
| Sodium | CBC with differential^{b} | Specific | Pregnancy test |
| Potassium | Mean corpuscular | Gravity | |
| Chloride | volume WBC count: | Leukocyte | |
| Bicarbonate, total | Neutrophils | esterase | |
| BUN or urea^{a} | Lymphocytes | Nitrite | |
| Creatinine | Monocytes | Blood | |
| Glucose | Basophils | Bilirubin | |
| Calcium, total | Eosinophils | Protein | |
| Phosphorus | RBC count | Glucose | |
| Magnesium, total | Hemoglobin | Ketones | |
| Albumin | Hematocrit | Urobilinogen | |
| Bilirubin direct | Platelets | | |
| Bilirubin total | PT | | |
| ALP | aPTT | | |
| ALT/SGPT | | | |
| AST/SGOT | | | |
| Total Protein | | | |

ALP = alkaline phosphatase; ALT = alanine aminotransferase; aPTT = activated partial thromboplastin time; AST = aspartate aminotransferase; BUN = blood urea nitrogen; CBC = complete blood count; PT = prothrombin time; SGOT = serum glutamic-oxaloacetic transaminase; SGPT = serum glutamic-pyruvic transaminase; WBC = white blood cell.
^{a}If BUN test cannot be analyzed by the local lab, urea should be analyzed.
^{b}Per institutional guidelines

### Research Laboratory Analyses

Analyses are performed on blood to evaluate PK and PD markers for DAN-222. In general, testing and analysis of the samples follows the Schedule of Assessments. However, allocation of samples to specific testing may be modified where sample material is limited.

Clinical biospecimens (e.g., serum) are sent from the clinical site to the central laboratory for sample processing, accessioning, and distribution to specialty laboratories or Dantari, Inc. All samples, as well as any derivatives from these samples, may be stored up to 5 years to address exploratory scientific questions related to the treatment or disease under study.

Multiple specialty laboratories may be employed for specific analyses.

### Pharmacokinetic Assessments (Plasma)

Assessment of DAN-222 in plasma is analyzed using liquid chromatography with mass spectroscopic detection (LC-MS-MS) specific for DAN-222.

### Stage 1 (Dose Escalation): Pharmacokinetic Assessments (Plasma)

Plasma samples are obtained for PK analysis at the following time points: On Cycle 1, Day 1 samples will be drawn pre-dose and at 30 minutes, 2, 4, 6, 8, 24, 48, 96, and 120 hours post-end of infusion. PK samples taken at pre-dose, 30 min, 2 hr are taken±5 min from scheduled post end of infusion. PK Samples taken 4+hr are taken±15 min post-end of infusion. On Cycle 2, Day 1 samples are drawn pre-dose and 30 minutes (±5 min) and 4 hours (±15 min) post-end of infusion. On Cycles 3+, Day 1 samples are drawn pre-dose and 30 minutes (±5 min) post-end of infusion. In addition, in cycle 3 and every subsequent 3 cycles, a 4 hours (±15 min) PK sample post-end of infusion is taken.

PK sample after treatment discontinuation or at early termination is collected at any time during the visit. Additional PK samples may be collected in the event of significant toxicities, or inconsistent drug exposure.

The post-dose PK samples are drawn from a location that is different from the site of study drug administration. For example, if the study drug is administered via a vein in the arm, the post-dose PK samples are drawn from a vein in the contralateral arm.

### Stage 2 (Dose Expansion): Pharmacokinetic Assessments (Plasma)

Plasma samples are obtained for PK analysis at the following time points: Cycle 1, Day 1 samples are drawn pre-dose, 0.5 hour (±5 min) and 4 hours (±15 min) post-end of infusion. Cycle 2 Day 1 samples are drawn pre-dose and 0.5 hour (±5 min) post-end of infusion. In Cycle 3 and every third cycle following, Day 1 samples are drawn pre-dose, 0.5 hour (±5 min) and 4 hours (±15 min) post-end of infusion.

PK sample after treatment discontinuation or early termination is collected during visit. Additional PK samples may be collected in the event of significant toxicities, or inconsistent drug exposure.

The post-dose PK samples are drawn from a location that is different from the site of study drug administration. For example, if the study drug is administered via a vein in the arm, the post-dose PK samples are drawn from a vein in the contralateral arm.

### Pharmacodynamic Assessments (Plasma)

Key assessments by central laboratory include circulating tumor count (CTC) measurements.

### Statistical Considerations

This is an open-label clinical study and, in general, descriptive statistics are employed to analyze the data. The data is tabulated and analyzed with respect to subject enrollment and disposition, demographic and baseline characteristics, prior and concomitant medications, efficacy, and safety measures.

### Sample Size Consideration

Approximately 18 to 36 subjects are enrolled in the study during the dose escalation stage (Stage 1) and approximately 60 subjects during the dose expansion stage (Stage 2). Overall sample size for this study is estimated to be between 18 and 96 subjects.

In the dose escalation part of the study, a standard 3+3 dose escalation design is separated into two parts. Part A is employed to explore the MTD of DAN-222 as a single agent and Part B is employed to explore the MTD of DNA-222 in combination with niraparib. There are up to 5 pre-planned dose cohort levels per part, with an estimated total of 18 DLT-evaluable subjects per part. Subjects who are not evaluable for DLT are replaced.

The sample size of 20 evaluable subjects in each Stage 2 group (Group A, Group B, and Group C) is based on practical considerations and clinical judgment to obtain sufficient information on the totality of study objectives including the characterization of safety, initial clinical effectiveness, and PK data for DAN-222 administered as monotherapy or in combination with niraparib in selected populations. For example, if there are 4 responders in a Group consisting of 20 evaluable subjects (i.e., an ORR of 20%), the 95% exact CI for the ORR would be (5.7%, 43.7%); similarly, if the ORR is 45% (9 responders out of 20 evaluable subjects) the 95% exact CI would be (23.1%, 68.5%).

### Statistical Methods

Unless stated otherwise, data is summarized by stage and dose level (Stage 1) or cohort (Stage 2), as appropriate, and all data is listed for all subjects. Data is also be presented overall within each Stage. Data may also be pooled in support of additional, aggregated analyses as described elsewhere herein.

Descriptive summaries for categorical variables include counts and percentages. Continuous variables are summarized using standard summary statistics (N, mean, standard deviation, median, minimum, and maximum). Medians, as well as 25th and 75th percentiles (where evaluable), are presented for survival data. Where appropriate, the 95% CI around a point is presented.

### Pharmacodynamic Analysis

Circulating tumor cell reduction is assessed in all subjects in the FAS at 4 weeks compared to baseline visit.

### Pharmacokinetic Analysis

The PK Analysis Set (PKAS) includes all subjects in the FAS from whom PK blood samples are collected during the study and who have measurable concentrations of DAN-222. For subjects with sufficient data, the following PK parameters are estimated from the individual concentration-time profiles using a non-compartmental analysis approach: total exposure (AUC₀₋ₗₐₛₜ); Cₘₐₓ; Cₘᵢₙ (trough concentration) and time to minimum observed plasma concentration; CL, V, and t1/2, if data allow; and PK parameters of unbound CPT.

Parameters are calculated using data following the first and fifth doses, if sufficient data are available. Additional analysis may be conducted if deemed useful and appropriate.

Pharmacokinetic parameter summary statistics are presented by dose group and may include mean, standard deviation, geometric mean and standard deviation, median, 25th to 75th percentile, minimum and maximum. All concentrations below the limit of detection or quantitation, or missing data, are labeled as such in the concentration data listings. Concentrations below the limit of detection are treated as zero in summary statistics.

### Efficacy Analyses

Response assessments are conducted by the Investigator based on RECIST v1.1. Unless otherwise specified, efficacy summaries are based on the EAS. Selected summaries may also be presented for the FAS and PPAS.

### Objective Response Rate

The ORR is defined as the proportion of subjects identified to have a best overall response (BOR) of complete response (CR) or partial response (PR) according to RECIST v1.1. the proportion of subjects with ORR in the EAS, along with a 95% CI, based on the Clopper-Pearson exact method, are reported descriptively. Overall response is summarized at each visit; BOR is also summarized. Secondary analyses of ORR and BOR may be repeated in the FAS and PPAS.

### Additional Secondary Efficacy Endpoints

PFS: Progression-free survival is defined as time from first dose of study drug to first documentation of progressive disease (per RECIST v1.1) or death due to any cause. Subjects who do not experience progressive disease and are alive will be censored at the time of last evaluable tumor assessment. Subjects who do not experience progressive disease and start new anticancer therapy are censored at the last evaluable tumor assessment on or prior to the time the new anticancer therapy begins. For any additional analyses conducted in the FAS, subjects with no evaluable post-baseline tumor assessments are censored at the time of receipt of first study drug. Subjects who are lost to follow-up for assessment of progressive disease are censored at their last evaluable tumor assessment. The analysis of PFS will be based on KaplanMeier methods.

DCR: Disease control rate is defined as the percentage of subjects with BOR of CR, PR, or SD. The analysis of DCR is performed in the same manner as ORR.

CBR: Clinical benefit rate is defined as the percentage of subjects with BOR of CR, PR, or documented SD over a continuous period of at least 6 months. The analysis of CBR is performed in the same manner as ORR.

DOR: Duration of response includes subjects with an objective disease response (PR or CR) and is defined as the time from the first tumor assessment that supports the subject's objective disease response to the time of progressive disease or death due to any cause. Subjects who do not experience progressive disease or death at the time of analysis are censored using the same rules as described for PFS. The analysis of DOR is performed using Kaplan-Meier methods.

### Homologous Recombination Repair Deficiency Analyses

Special attention is devoted to subsets of subjects who may have distinct underlying DNA repair pathway deficiency (HR, nucleotide excision repair, or mismatch repair deficiency) and distinct drug responses. There may be circumstances in which a decision is made to stop biomarker sample collection, or not perform or discontinue the analysis of blood or tumor due to either practical or sample quality reasons. In such circumstances, the number of samples may be insufficient to perform a complete data analysis, and as such, the available data is listed and summarized for the FAS. Additionally, exploratory efficacy analyses may also be conducted based on subgroups identified using HRD and/or other biomarkers.

### Safety Analysis

All safety analyses are conducted in the FAS. Adverse events are coded according to the current version of MedDRA. The severity of adverse events is graded according to the United States NCI CTCAE, v5.0.

TEAEs: Treatment-emergent adverse events are defined as any adverse event with onset (or worsening of a pre-existing condition) after the first dose of study drug through 30 days following the last dose of study drug. Events including TEAEs, adverse events leading to dose reduction/interruption, adverse events related to study drug, serious adverse events, adverse events leading to study drug discontinuation, and fatal adverse events are summarized by system organ class and preferred term for each treatment group. A summary of adverse events of NCI CTCAE Grade 3 or higher, as well as the most frequent adverse events (by preferred term), and adverse events by relationship to study treatment, is provided.

For Stage 1 the incidence of DLTs is summarized. For Stage 1 and Stage 2 the incidence of infusion-related reactions is also summarized. Values and changes from baseline in clinical laboratory results are summarized by visit. Clinical laboratory values are graded according to the NCI CTCAE, for applicable tests. Shifts in toxicity grades from baseline grade are summarized. Shifts from baseline in ECOG performance status are also summarized. Vital sign, ECG, and concomitant medication data is summarized.

### Managing Adverse Events

Dose interruptions and modifications may be implemented per the Investigators judgment after Cycle 1 in subjects enrolled in Stage I (Dose Escalation) and at any time in Stage 2 (Dose Expansion). The dose interruptions and reduction instructions provided in this section are intended to serve as recommended guidelines to allow ongoing treatment for subjects without signs or symptoms of progression while ensuring subject safety. In addition to these guidelines, more conservative drug interruptions or dose reductions for the management of adverse events are permitted at the discretion of the treating Investigator when deemed to be in the best interest of the subject and to ensure subject safety.

Subjects who discontinue niraparib treatment because of intolerability may continue in the study and receive continued treatment with DAN-222 at the discretion of the Investigator.

After Cycle 1 subjects may temporarily suspend all study drugs for up to 28 consecutive days if they experience toxicity that is considered related to study treatment and that requires a study treatment hold. Subjects who miss >28 consecutive days of scheduled study treatment will be discontinued from the study.

5 Treatment with DAN-222 must be interrupted for any treatment-related non-hematologic CTCAE Grade 3 or Grade 4 event. Once resolved to Grade≤1, the subject may restart treatment with DAN-222 with a dose level reduction (Table 17) unless prophylaxis is considered feasible. The dose interruption/modification criteria for DAN-222 for hematologic toxicities will be based on blood counts and the guidance below in Table 17. If clinically indicated, use of granulocyte colony-stimulating factor (G-CSF) is allowed according to current ASCO guidelines. The reason 10 for interruption, reduction, or discontinuation of study drug should be recorded in the eCRF.

TABLE 17
DAN-222 Dose Modifications/Interruptions for Adverse Events

| | | |
|---|---|---|
| | During a Cycle: | Next Dose Modification |
| NCI CTCAE | Recommended Guidelines for Management | Based on Worst Toxicity |
| Grade | and Supportive Care | in Prior Dose |

Thrombocytopenia/Neutropenia

| | | |
|---|---|---|
| Grade 1 | | Maintain dose level |
| Grade 2 or 3 | Platelets: consider platelet transfusion if | ↓ 1 dose level after 1^{st} |
| | active bleeding, or platelet stimulator. Hold | occurrence. |
| | treatment until toxicity resolves to: platelet | ↓ 1 dose level after 2^{nd} |
| | counts ≥100,000/μL and monitor blood | occurrence. |
| | counts weekly until platelet counts return | Discontinue subject after |
| | to ≥100,000/μL; for a maximum of 28 days. | 3^{rd} occurrence. |
| | ANC: consider growth factor support (e.g., | |
| | G-CSF) in accordance with local guidelines. | |
| | Hold treatment until toxicity resolves to: | |
| | ANC ≥1.5 × 10⁹/L; platelets ≥100,000/μL, | |
| | for a maximum of 28 days | |
| Grade 4 | Platelets: consider platelet transfusion if | ↓ 1 dose level after 1^{st} |
| | platelets <20,000/μL or with active | occurrence. |
| | bleeding. | ↓ 1 dose level after 2^{nd} |
| | ANC: consider growth factor support in | occurrence. |
| | accordance with local guidelines. | Discontinue subject after |
| | Antibiotic (fluoroquinolones) should be | 3^{rd} occurrence. |
| | initiated even in the absence of fever or | |
| | diarrhea. | |
| | Hold treatment for a week or until toxicity | |
| | resolves to: ANC >1.5 × 10⁹/L; | |
| | platelets ≥100,000/μL, for a maximum of | |
| | 28 days. | |

Febrile Neutropenia

| | | |
|---|---|---|
| Any Grade | Subject should be hospitalized immediately | ↓ 1 dose level after 1^{st} |
| | for IV antibiotic therapy if neutropenic fever | occurrence. |
| | or sepsis develops (e.g., growth factor support; | ↓ 1 dose level after 2^{nd} |
| | antibiotics). | occurrence. |
| | | Discontinue subject after |
| | | 3^{rd} occurrence. |

Diarrhea

| | | |
|---|---|---|
| Any Grade | Institute supportive care upon first loose stool | Confirm with the subject |
| | (Unless contraindicated, use loperamide). | that diarrhea is no longer |
| | Monitor bowel function; if diarrhea continues | present for ≥7 days |
| | with first supportive care agent, consider | without having received |
| | switching to a second agent or add a second | supportive care prior to |
| | agent (unless contraindicated, use | retreatment. |
| | diphenoxylate/atropine). | Treatment may be delayed |
| | Monitor for dehydration, electrolyte | up to 28 days; after this, |
| | abnormalities; correct if present. Administer | contact Medical Monitor. |
| | antibiotic therapy (oral fluoroquinolones) if | |
| | the subject develops ileus, fevers or Grade 3 | |
| | or Grade 4 neutropenia. | |
| | Subject should be immediately hospitalized | |
| | for IV antibiotics therapy if evidence of colitis | |
| | or ileus, even in the absence of neutropenia or | |
| | fever. | |
| | If diarrhea worsens, octreotide may be | |
| | attempted. | |
| | Monitor bowel function for continued need of | |
| | supportive care. | |
| | Stop supportive care after the subject is without | |
| | diarrhea for 48 hours. | |
| Grade 1 | If still present within 7 days prior to treatment | Maintain dose level; |
| | hold treatment until diarrhea is no longer | consider prophylactic anti- |
| | present for ≥7 days without having received | diarrheal supportive care |
| | supportive care. | |
| Grade 2 | If still present within 7 days prior to treatment | ↓ 1 dose level after 1^{st} |
| | hold treatment until diarrhea is no longer | occurrence. |
| | present for ≥7 days without having received | ↓ 1 dose level after 2^{nd} |
| | supportive care. | occurrence. |
| | | Discontinue subject after |
| | | 3^{rd} occurrence. |
| Grade 3 or | If still present within 7 days prior to treatment | ↓ 2 dose levels after 1^{st} |
| Grade 4 | hold treatment until diarrhea is no longer | occurrence. |
| | present for ≥7 days without having received | Provided that adequate |
| | supportive care. | supportive care has been |
| | | given, discontinue subject |
| | | after 2^{nd} occurrence. |
| | | If adequate supportive care |
| | | has not been given, |
| | | retreatment may be |
| | | attempted for a 2^{nd} episode |
| | | of Grade 3 diarrhea (re- |
| | | instruct subject on |
| | | supportive care). |

Nausea/Vomiting

| | | |
|---|---|---|
| Grade 1 or | Consider anti-emetic therapy. | Consider prophylactic anti- |
| Grade 2 | If Grade 2 present on a treatment day, hold | emetic therapy. |
| | treatment for a week or until toxicity resolves | Maintain dose level. |
| | to baseline or Grade ≤1, for a maximum of 28 | |
| | days. | |
| Grade 3 or | Use anti-emetic therapy. Consider | After 1^{st} occurrence use |
| Grade 4 | administration of IV fluids. | prophylactic anti-emetic |
| | If present on a treatment day, hold treatment | therapy and maintain dose |
| | for a week or until toxicity resolves to | level. |
| | baseline or Grade ≤1, for a maximum of 28 | ↓ 1 dose level after 2^{nd} |
| | days. | occurrence. |
| | | Discontinue subject after |
| | | 3^{rd} occurrence |

Pulmonary Toxicity

| Suspected | If subject develops radiographic changes potentially consistent with | |
|---|---|---|
| ILD/Pneumonitis | ILD/pneumonitis or develops an acute onset of new or worsening | |
| | pulmonary or other related signs/symptoms such as dyspnea, cough, or | |
| | fever, rule out ILD/pneumonitis. | |
| | If the AE is confirmed to have an etiology other than ILD/pneumonitis, | |
| | follow the management guidance dose modification section of the protocol. | |
| | If the AE is suspected to be ILD/pneumonitis, treatment with study drug | |
| | should be interrupted pending further evaluations. Obtain a pulmonary | |
| | consult. | |
| | If the AE is confirmed to be ILD/pneumonitis, follow the management | |
| | guidance as outlined below. The administration of DAN-222 must be | |
| | interrupted for any ILD/pneumonitis events regardless of grade. | |
| | All events of ILD/pneumonitis regardless of severity or seriousness will | |
| | be followed until resolution including after drug discontinuation. | |
| Grade 1 | Monitor and closely follow-up in 2 to 7 days | DAN-222 can be |
| | for onset of clinical symptoms and pulse | restarted only if the event |
| | oximetry. | is fully resolved to Grade |
| | Consider follow-up imaging in 1 to 2 weeks | 0. |
| | (or as clinically indicated). | If resolved <28 days from |
| | Consider starting systemic steroids (e.g., at | day of onset reduce dose 1 |
| | least 0.5 mg/kg/day prednisone, or | level. |
| | equivalent) until improvement, followed | However, if Grade 1 |
| | by gradual taper over at least 4 weeks. | ILD/pneumonitis occurs |
| | If diagnostic observations worsen despite | beyond Cycle 1 Day 28 |
| | initiation of corticosteroids, follow Grade 2 | and has not resolved |
| | guidelines, unless asymptomatic. | within 28 days from the |
| | | last infusion, discontinue |
| | | subject. |
| Grade 2 | Promptly start systemic steroids (e.g., at least | Permanently discontinue |
| | 1 mg/kg/day prednisone or equivalent) for at | subject from study |
| | least 14 days or until complete resolution of | treatment. |
| | clinical and chest CT findings, followed by a | |
| | gradual taper over at least 4 weeks. | |
| | Monitor symptoms closely. | |
| | Re-image as clinically indicated. | |
| | If worsening or no improvement in clinical or | |
| | diagnostic observations in 5 days: | |
| | Consider increasing dose of steroids (e.g., 2 | |
| | mg/kg/day prednisone or equivalent) and | |
| | administration may be switched to IV (e.g., | |
| | methylprednisolone). | |
| | Re-consider additional work-up for | |
| | alternative etiologies as described above. | |
| | Escalate care as clinically indicated. | |
| Grade 3 and 4 | Hospitalization required. | Permanently discontinue |
| | Promptly initiate empiric high-dose | subject from study |
| | methylprednisolone IV treatment (e.g., 500 to | treatment. |
| | 1000 mg/day for 3 days), followed by at least | |
| | 1.0 mg/kg/day of prednisone (or equivalent) | |
| | until clinical improvement, or at least | |
| | 14 days or until complete resolution of clinical | |
| | and chest CT findings, then followed by a | |
| | gradual taper over at least 4 weeks. | |
| | Re-image as clinically indicated. | |
| | If still no improvement within 3 to 5 days, re- | |
| | consider additional work-up for alternative | |
| | etiologies as described above. | |
| | Consider other immune-suppressants and/or | |
| | treat per local practice. | |

Cystitis/Bladder Toxicity

| | | |
|---|---|---|
| Grade 1 | Subject to continuously hydrate orally with ≥3 | Maintain dose |
| | L/Day and void at least every 2 hours. | |
| Grade 2 to | Subject to continuously hydrate orally with ≥3 | Withhold dose until |
| Grade 4 | L/Day and void at least every 2 hours. | toxicity resolves to |
| | | baseline or Grade ≤1, for |
| | | a maximum of 28 days. |

Other Drug-Related Toxicities

| | | |
|---|---|---|
| Grade 1 or | Consider supportive care as appropriate. | Maintain dose level (for |
| Grade 2 | If Grade 2 present on a treatment day, hold | Grade 2 toxicity, the |
| | treatment for a week or until toxicity resolves | Investigator may |
| | to baseline or Grade ≤1, for a maximum of 28 | use discretion to ↓ 1 dose |
| | days. | level after 1^{st} occurrence |
| | | depending on the nature of |
| | | the toxicity). |
| Grade 3 or | Use supportive care as appropriate. | ↓ 1 dose level after 1^{st} |
| Grade 4 | If present on a treatment day, hold treatment | occurrence; supportive care |
| | for a week or until toxicity resolves to baseline | as appropriate. |
| | or Grade ≤1, for a maximum of 28 days. | ↓ 1 dose level after 2^{nd} |
| | | occurrence. |
| | | Discontinue subject after |
| | | 3^{rd} occurrence. |

AE = adverse event; ANC = absolute neutrophil count; CT = computed tomography; D5W = 5% dextrose in water; G-CSF = granulocyte colony stimulating factor; ILD = interstitial lung disease; IV = intravenous; NCI CTCAE = National Cancer Institute Common Terminology Criteria for Adverse Events; IV = intravenous.

### Study Screening

Subjects who have fully consented to participation in the study undergo screening assessments within 28 days (4 weeks) prior to administration of the first infusion of DAN-222 (unless otherwise stated). The screening period begins on the date the subject signs the IRB/IEC-approved informed consent form (ICF). Informed consent must be obtained before completion of any non-standard of care study-specific procedure.

All subjects who meet eligibility criteria and are enrolled into the study are scheduled for Day 1 of study treatment. If at any time before enrollment the subject fails to meet the eligibility criteria, the subject should be designated as a screen failure, and the reason(s) for failing screening should also be recorded.

The initial screening assessment includes: obtaining informed consent; assessing eligibility per inclusion/exclusion criteria; obtaining disease history, including disease diagnosis and prior treatments for the malignancy (chemotherapy, radiation, and surgeries) and any history of toxicities related to prior treatments, and allergies; obtaining an ECOG performance status assessment; a complete physical examination; and recording all adverse events and serious adverse events related to protocol mandated procedures and concomitant medications taken at that time.

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### EQUIVALENTS

Various modifications of the invention and many further embodiments thereof, in addition to those shown and described herein, will become apparent to those skilled in the art from the full contents of this document, including references to the scientific and patent literature cited herein. The subject matter herein contains important information, exemplification, and guidance that can be adapted to the practice of this invention in its various embodiments and equivalents thereof.

What is claimed is:

1. A process for producing a formulation of a compound of Formula (I): 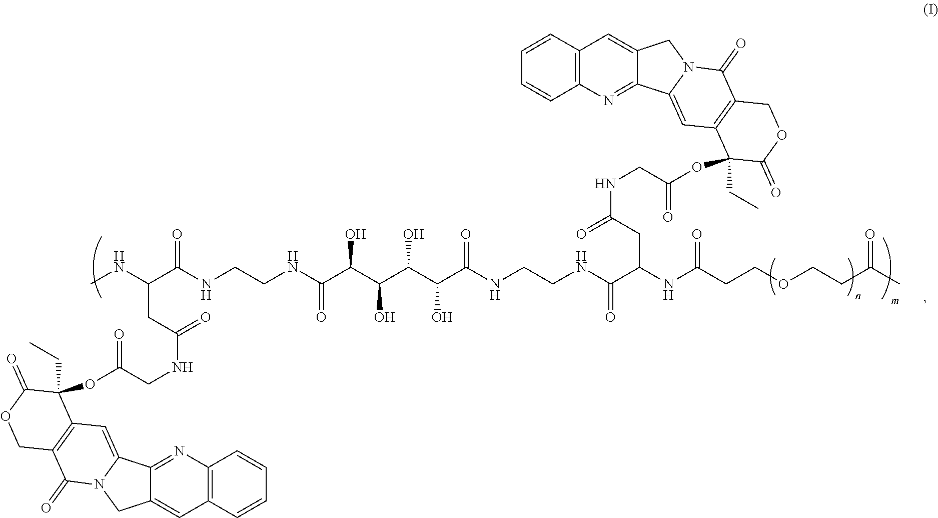
wherein n is a number in a range from 20 to 200; and
wherein m is a number in a range from 5 to 200,
the process comprising:
(i) performing a derivatization of camptothecin (CPT) to yield Gly-CPT as the trifluoracetic acid (TFA) salt;
(ii) synthesizing a parent mucic acid polymer (MAP);
(iii) covalently attaching the Gly-CPT to the parent MAP to yield solid, amorphous MAP-Gly-CPT; and
(iv) preparing an aqueous formulation of the amorphous MAP-Gly-CPT, thereby producing an aqueous formulation of nanoparticles comprising the compound of Formula (I).

2. The process of claim 1, wherein step (i) comprises:
dissolving CPT, N-(tert-butoxycarbonyl)Gly-OH (Boc-Gly), and 4-dimethylaminopyridine (DMAP) in methylene chloride (DCM) to form a reaction mixture; and
adding diisopropylcarbodiimide (DIC) to the reaction mixture thereby producing a solution comprising a Boc-Gly-CPT intermediate.

3. The process of claim 2, wherein the step (i) further comprises removing between 90% and 99% of the DCM from the solution comprising the Boc-Gly-CPT intermediate.

4. The process of claim 3, wherein the DCM is removed via vacuum distillation.

5. The process of claim 3, wherein the step (i) further comprises precipitating the Boc-Gly-CPT intermediate using methanol.

6. The process of claim 5, wherein the step (i) further comprises washing the precipitated Boc-Gly-CPT intermediate using isopropyl alcohol (IPA).

7. The process of claim 6, wherein the step (i) further comprises treating the Boc-Gly-CPT intermediate with TFA in DCM to yield Gly-CPT.

8. The process of claim 1, wherein step (ii) comprises:
charging a reactor with (succinimidyl propionate) 2PEG3500 (diSPA-PEG3500) and a mucic acid monomer (MAM) neutral species of Formula (II): 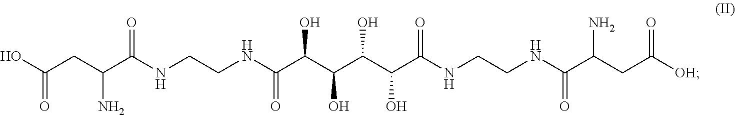 [SMILES: NC(CC(=O)O)C(=O)NCCNC(=O)[C@@H](O)[C@H](O)[C@H](O)[C@@H](O)C(=O)NCCNC(=O)C(N)CC(=O)O]
adding dimethyl sulfoxide (DMSO) to the reactor;
initiating the reaction by adding TFA to the reactor; and
adding DIPEA to the reaction,
thereby yielding the parent MAP.

9. The process of claim 8, wherein the comonomer ratio of DIPEA: MAM used in step (ii) is at least about 2.5.

10. The process of claim 9, wherein the comonomer ratio is at least 2.6.

11. The process of claim 8, wherein step (ii) further comprises precipitating the parent MAP from the DMSO via addition of at least one anti-solvent.

12. The process of claim 11, wherein the at least one anti-solvent is IPA.

13. The process of claim 11, wherein the precipitating step is followed by a filtration step under an inert atmosphere.

14. The process of claim 1, wherein step (iii) comprises:
adding the parent MAP and DMSO to a reaction vessel;
adding the Gly-CPT (TFA salt) and 7-azabenzotriazol-1-yloxy)tripyrrolidinophosphonium hexafluorophosphate (PyAOP) to the reaction vessel; and
adding DIPEA to the reaction vessel, thereby producing a solution that comprises MAP-Gly-CPT.

15. The process of claim 14, wherein step (iii) further comprises purifying amorphous MAP-Gly-CPT from the solution using tangential flow filtration (TFF).

16. The process of claim 15, wherein prior to the tangential flow filtration, the solution is diluted with pH 3 water and wherein the tangential flow filtration uses polyethersulfone (PESU) TFF filters.

17. The process of claim 1, wherein step (iv) comprises:
adding water with a pH of around pH 4 to the amorphous MAP-Gly-CPT to produce a nanoparticle (NP) solution; and
adding a formulation buffer to the NP solution.

18. The process of claim 17, wherein the formulation buffer comprises sodium acetate.

19. The process of claim 18, wherein the formulation buffer comprises NaCl and has a pH of around pH 4.16.

20. The process of claim 18, wherein after the step of adding the water with a pH of around pH 4, the NP solution is filtered.

21. The process of claim 20, wherein after the step of adding the formulation buffer to the NP solution, the NP solution is concentrated by ultrafiltration.

22. A formulation comprising nanoparticles, said nanoparticles comprising a mucic acid polymer (MAP) camptothecin (CPT) conjugate compound of Formula (I): 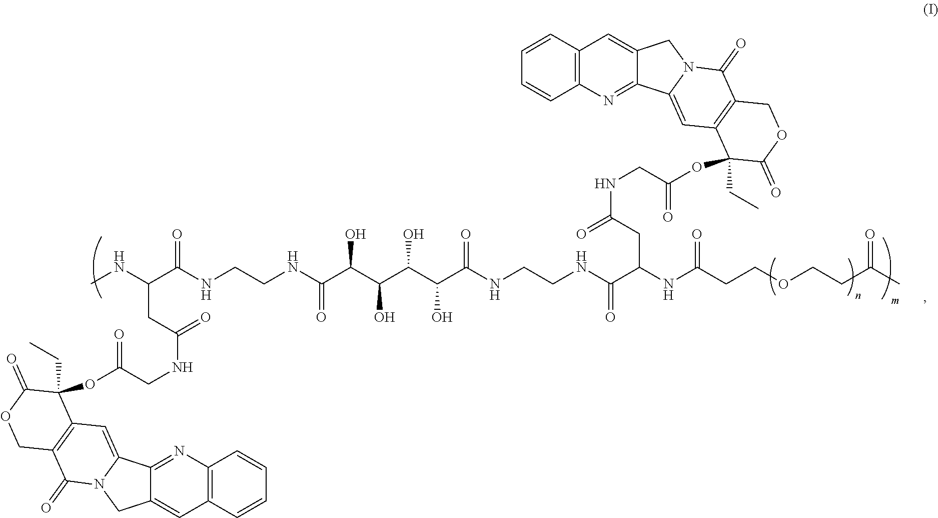
wherein n is a number in a range from 20 to 200; and
wherein m is a number in a range from 5 to 200, formulated as an aqueous formulation.

23. The formulation of claim 22, wherein n is a number in a range from about 75 to 85.

24. The formulation of claim 23, wherein m is a number in a range from about 12 to 20.

25. The formulation of claim 24, wherein the compound of Formula (I) has a molecular weight of about 75,000 Da.

26. The formulation of claim 25, wherein the nanoparticles of the formulation comprise on average two strands comprising the compound of Formula (I) and wherein the nanoparticles have an average molecular weight of about 150,000 Da.

27. The formulation of claim 26, wherein the nanoparticles of the formulation have an average particle size of about 20 nm to 80 nm.

28. The formulation of claim 27, wherein the nanoparticles have an average particle size of about 30 to 40 nm.

29. The formulation of claim 28, wherein the formulation has a nanoparticle concentration of about 17-23 mg/mL.

30. The formulation of claim 24, wherein the formulation has a total concentration of CPT of between 1.7 and 3.6 mg/mL.

31. The formulation of claim 30, wherein the formulation has a total concentration of CPT of about 2.6 mg/mL.

32. The formulation of claim 31, wherein the formulation comprises a concentration of about 20 mg/mL of the compound of Formula (I).

33. The formulation of claim 24, wherein the formulation has a pH between pH 4 and pH 5.

34. The formulation of claim 33, wherein the formulation has a pH between pH 4 and pH 4.6.

35. The formulation of claim 34, wherein the formulation has a pH of about 4.3.

36. The formulation of claim 35, wherein the formulation further comprises at least one buffer selected from sodium succinate, sodium citrate, sodium acetate, phosphoric acid, histidine-HCl, and sodium phosphate.

37. The formulation of claim 36, wherein the at least one buffer is a sodium acetate buffer.

38. The formulation of claim 37, wherein the formulation further comprises at least one tonicity modifier selected from KCl, NaCl, Proline, Arginine-HCl, sucrose, and glycine.

39. The formulation of claim 38, wherein the at least one tonicity modifier is NaCl.

40. The formulation of claim 39, wherein the formulation comprises about 20 mg/mL of the compound of Formula (I).

41. The formulation of claim 40, wherein the formulation comprises about 20 mM sodium acetate, about 200 mM NaCl, and has a pH of 4.3±0.3.

42. A method for treating cancer in a subject, the method comprising providing to a subject having cancer at least one dose of a composition comprising a compound of Formula (I): 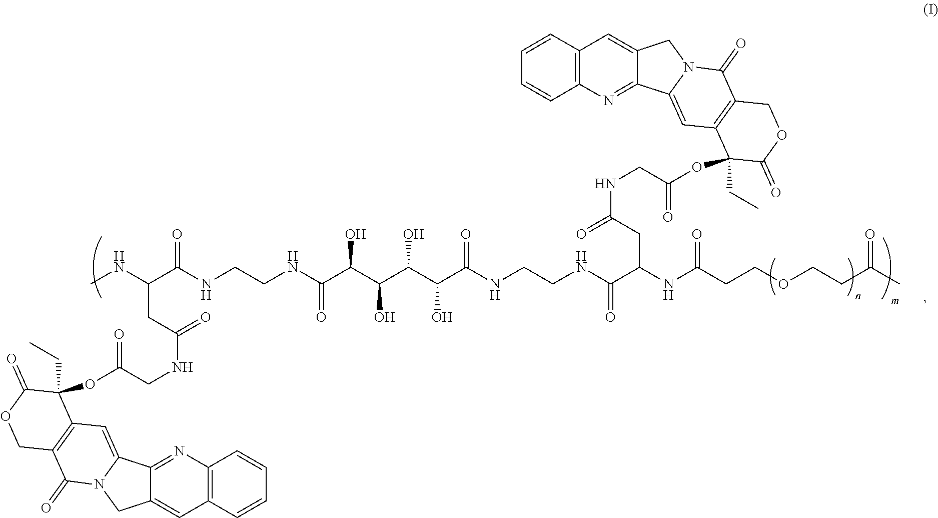
wherein n is a number in a range from 20 to 200; and
wherein m is a number in a range from 5 to 200.

43. The method of claim 42, wherein:
n is a number in a range from about 75 to 85;
m is a number in a range from about 12 to 20; and
the composition has a pH between pH 4 and pH 4.6.

44. The method of claim 43, wherein the composition is provided in solution for infusion.

45. The method of claim 44, wherein the composition comprises between 2 mg/ml and 3 mg/mL of camptothecin (CPT).

46. The method of claim 42, wherein the dose of the composition comprises between 2 mg/m² and 16 mg/m² of the compound of Formula (I).

47. The method of claim 42, wherein the dose of the composition comprises about 2 mg/m², about 4 mg/m², about 6 mg/m², about 8 mg/m², about 10 mg/m², about 12 mg/m², about 14 mg/m², or about 20 mg/m² of the compound of Formula (I).

48. The method of claim 42, wherein a plurality of doses are provided to a subject during a treatment cycle.

49. The method of claim 48, wherein the plurality of doses are provided as weekly doses.

50. The method of claim 49, wherein each dose comprises between 2 mg/m² and 16 mg/m² of the compound of Formula (I).

51. The method of claim 42, wherein the method further comprises providing at least one dose of a poly (ADP-ribose) polymerase (PARP) inhibitor.

52. The method of claim 51, wherein the PARP inhibitor is selected from olaparib, rucaparib, niraparib, and talazoparib.

53. The method of claim 52, wherein the PARP inhibitor is niraparib.

54. The method of claim 53, wherein the dose of the composition comprising the compound of Formula (I) comprises between 2 mg/m² and 16 mg/m² of the compound of Formula (I).

55. The method of claim 54, wherein a plurality of doses of the composition comprising the compound of Formula (I) are provided to a subject during a treatment cycle.

56. The method of claim 55, wherein the plurality of doses of the composition are provided as weekly doses.

57. The method of claim 56, wherein a dose of niraparib is provided to the subject daily during the treatment cycle.

58. The method of claim 57, wherein each dose of niraparib is a 100 mg dose.

59. The method of claim 42, wherein the cancer is one or more cancers of the breast, ovary brain, lung, testicle, head, neck, esophagus, central nervous system, peripheral nervous system, bladder, stomach, pancreas, liver, oral mucosa, anus, kidney, bladder, uroepithelium, prostate, endometrium, uterus, or fallopian tube; or is a colorectal cancer, a mesothelioma, a melanoma, a myeloma, a leukemia, a lymphoma or a Kaposi's sarcoma.

60. The method of claim 59, wherein the cancer is a breast cancer.

61. The method of claim 60, wherein the breast cancer is a homologous recombination repair deficiency (HRD) positive or HRD-negative breast cancer.

62. The method of claim 42, wherein the cancer is a brain metastasis of a breast cancer.
